SEQUENCE LISTING (192 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 302 aa, Severe acute respiratory syndrome coronavirus 2
QNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPW

SEQ ID NO: 3 - protein, 397 aa, Chlamydia trachomatis
MKKLLKSVLVFAALSSASSLQALPVGNPAEPSLMIDGILWEGFGGDPCDPCTTWCDAISMRVGYYGDFVFDRVLKTDVNKEFQMGAAPTTSDVAGLQNDPTINVARPNPAYGKHMQDAEMFTNAAYMALNIWDRFDVFCTLGATTGYLKGNSASFNLVGLFGTKTQSSSFNTAKLIPNTALNEAVVELYINTTFAWSVGARAALWECGCATLGASFQYAQSKPKVEELNVLCNASEFTINKPKGYVGAEFPLNITAGTEAATGTKDASIDYHEWQASLALSYRLNMFTPYIGVKWSRVSFDADTIRIAQPKLAEAILDVTTLNRTTAGKGSVVSAGTDNELADTMQIVSLQLNKMKSRKSCGIAVGTTIVDADKYAVTVEARLIDERAAHVNAQFRF

SEQ ID NO: 4 - protein, 857 aa, Lymphocryptovirus humangamma4
MTRRRVLSVVVLLAALACRLGAQTPEQPAPPATTVQPTATRQQTSFPFRVCELSSHGDLFRFSSDIQCPSFGTRENHTEGLLMVFKDNIIPYSFKVRSYTKIVTNILIYNGWYADSVTNRHEEKFSVDSYETDQMDTIYQCYNAVKMTKDGLTRVYVDRDGVNITVNLKPTGGLANGVRRYASQTELYDAPGWLIWTYRTRTTVNCLITDMMAKSNSPFDFFVTTTGQTVEMSPFYDGKNKETFHERADSFHVRTNYKIVDYDNRGTNPQGERRAFLDKGTYTLSWKLENRTAYCPLQHWQTFDSTIATETGKSIHFVTDEGTSSFVTNTTVGIELPDAFKCIEEQVNKTMHEKYEAVQDRYTKGQEAITYFITSGGLLLAWLPLTPRSLATVKNLTELTTPTSSPPSSPSPPAPSAARGSTPAAVLRRRRRDAGNATTPVPPTAPGKSLGTLNNPATVQIQFAYDSLRRQINRMLGDLARAWCLEQKRQNMVLRELTKINPTTVMSSIYGKAVAAKRLGDVISVSQCVPVNQATVTLRKSMRVPGSETMCYSRPLVSFSFINDTKTYEGQLGTDNEIFLTKKMTEVCQATSQYYFQSGNEIHVYNDYHHFKTIELDGIATLQTFISLNTSLIENIDFASLELYSRDEQRASNVFDLEGIFREYNFQAQNIAGLRKDLDNAVSNGRNQFVDGLGELMDSLGSVGQSITNLVSTVGGLFSSLVSGFISFFKNPFGGMLILVLVAGVVILVISLTRRTRQMSQQPVQMLYPGIDELAQQHASGEGPGINPISKTELQAIMLALHEQNQEQKRAAQRAAGPSVASRALQAARDRFPGLRRRRYHDPETAAALLGEAETEF

SEQ ID NO: 5 - protein, 904 aa, Simplexvirus humanalpha1
MRQGAPARGRRWFVVWALLGLTLGVLVASAAPSSPGTPGVAAATQAANGGPATPAPPAPGAPPTGDPKPKKNKKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 6 - protein, 698 aa, Simplexvirus humanalpha2
MHAIAPRLLLLFVLSGLPGTRGGSGVPGPINPPNNDVVFPGGSPVAQYCYAYPRLDDPGPLGSADAGRQDLPRRVVRHEPLGRSFLTGGLVLLAPPVRGFGAPNATYAARVTYYRLTRACRQPILLRQYGGCRGGEPPSPKTCGSYTYTYQGGGPPTRYALVNASLLVPIWDRAAETFEYQIELGGELHVGLLWVEVGGEGPGPTAPPQAARAEGGPCVPPVPAGRPWRSVPPVWYSAPNPGFRGLRFRERCLPPQTPAAPSDLPRVAFAPQSLLVGITGRTFIRMARPTEDVGVLPPHWAPGALDDGPYAPFPPRPRFRRALRTDPEGVDPDVRAPLTGRRLMALTEDASSDSPTSAPEKTPLPVSATAMAPSVDPSAEPTAPATTTPPDEMATQAATVAVTPEETAVASPPATASVESSPLPAAAATPGAGHTNTSSAPAAKTPPTTPAPTTPPPTSTHATPRPTTPGPQTTPPGPATPGPVGASAAPTADSPLTASPPATAPGPSAANVSVAATTATPGTRGTARTPPTDPKTHPHGPADAPPGSPAPPPPEHRGGPEEFEGAGDGEPPDDDDSATGLAFRTPNPNKPPPARPGPIRPTLPPGILGPLAPNTPRPPAQAPAKDMPSGPTPQHIPLFWFLTASPALDILFIISTTIHTAAFVCLVALAAQLWRGRAGRRRYAHPSVRYVCLPPERD

SEQ ID NO: 7 - protein, 51 aa, Human papillomavirus
SQPLKQHYQIVTCCCGCDSNVRLVVQCTETDIREVQQLLLGTLNIVCPICA

SEQ ID NO: 8 - protein, 158 aa, Human papillomavirus
MHQKRTAMFQDPQERPRKLPQLCTELQTTIHDIILECVYCKQQLLRREVYDFAFRDLCIVYRDGNPYAVCDKCLKFYSKISEYRHYCYSVYGTTLEQQYNKPLCDLLIRCINCQKPLCPEEKQRHLDKKQRFHNIRGRWTGRCMSCCRSSRTRRETQL

SEQ ID NO: 9 - protein, 566 aa, Influenza A virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNNSFFSRLNWLTHLKFKYPALNVTMPNNEKFDKLYIWGVHHPGTDNDQISLYAQASGRITVSTKRSQQTVIPSIGSRPRIRDVPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 10 - protein, 584 aa, Influenza B virus
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAASSLAVTLMIAIFIVYMVSRDNVSCSICL

SEQ ID NO: 11 - RNA, 1704 nt, synthetic construct
  source 1..1704: mol_type = other RNA
atgaaaacaattatcgctctcagttatattttctgccttcctttggggcaagacttgccgggcaatgataatagtacagcgactctctgtctcggacaccacgcagtaccgaacggcacacttgtgaagacaatcacagacgatcaaatcgaagtgacgaatgcaacagagcttgttcaatcatcatctaccggcaaaatctgcaataatccgcatagaatccttgatggaatcgactgcaccctgattgacgcactgttgggagatcctcattgcgatgtgtttcagaatgagacatgggacctttttgtcgagaggagcaaagcgttttccaactgctacccgtatgacgtgcctgattacgcgtcactccgctcacttgtagcatcaagcggtactctggagttcatcaccgaaggattcacctggacgggcgtaactcagaacggcggttctaacgcatgtaagagggggccagggtccggcttcttctcacggctcaactggttgactaagtctggttcaacatacccggtccttaacgttacaatgccgaataatgacaactttgataaactctacatctggggcattcaccatccatccacaaatcaagaacagacaagtttgtacgttcaggcgtcagggcgcgttaccgtgagtacaagaagatcacagcaaacaattatacccaatattgggtcccgaccctgggtaagaggactgtcctctcgcatctccatatactggaccattgtcaaaccgggcgacgtcctggttatcaatagtaatggaaaccttattgctccgcgcggctatttcaaaatgcgaactggaaagtcaagtataatgcgctcagacgcaccgatcgatacttgtatcagtgaatgcatcacccctaacgggtccataccgaacgataagcccttccagaatgtgaataaaatcacgtatggagcatgccccaaatacgtgaagcaaaacaccctcaagttggctacgggtatgcgcaacgtcccagaaaaacaaacgcgaggcttgtttggggcgatagcaggttttatcgagaacggctgggaaggaatgatcgatgggtggtacggctttcgccatcaaaactcagaaggaactgggcaggccgcagatcttaagtctacgcaagcggcgatagatcaaattaatggcaagttgaatagggtgatagagaagacgaacgagaagttccatcaaatagagaaagaattcagtgaagtagaggggcgaattcaggatttggaaaaatatgtcgaggatactaagatcgacctgtggagctataacgcagagcttctggtagcacttgagaaccagcatactattgatctcaccgattccgagatgaacaagctttttgagaagaccaggagacagttgcgcgagaatgccgaagaaatgggtaacggttgttttaagatttatcacaaatgtgacaacgcgtgtattgagtccattaggaatggtacatatgatcacgatgtgtatcgcgatgaagcacttaacaatcggttccaaataaagggcgtggagctcaagagtgggtataaagattggatcctctggatctcattcgcgatttcctgcttcctcctgtgcgttgtcttgctgggctttattatgtgggcatgtcagagaggtaacatccggtgcaacatatgtatctgataa

SEQ ID NO: 12 - protein, 566 aa, Influenza A virus
MKTIIALSYIFCLPLGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEEMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 13 - protein, 741 aa, Mycobacterium tuberculosis
MTDRVSVGNLRIARVLYDFVNNEALPGTDIDPDSFWAGVDKVVADLTPQNQALLNARDELQAQIDKWHRRRVIEPIDMDAYRQFLTEIGYLLPEPDDFTITTSGVDAEITTTAGPQLVVPVLNARFALNAANARWGSLYDALYGTDVIPETDGAEKGPTYNKVRGDKVIAYARKFLDDSVPLSSGSFGDATGFTVQDGQLVVALPDKSTGLANPGQFAGYTGAAESPTSVLLINHGLHIEILIDPESQVGTTDRAGVKDVILESAITTIMDFEDSVAAVDAADKVLGYRNWLGLNKGDLAAAVDKDGTAFLRVLNRDRNYTAPGGGQFTLPGRSLMFVRNVGHLMTNDAIVDTDGSEVFEGIMDALFTGLIAIHGLKASDVNGPLINSRTGSIYIVKPKMHGPAEVAFTCELFSRVEDVLGLPQNTMKIGIMDEERRTTVNLKACIKAAADRVVFINTGFLDRTGDEIHTSMEAGPMVRKGTMKSQPWILAYEDHNVDAGLAAGFSGRAQVGKGMWTMTELMADMVETKIAQPRAGASTAWVPSPTAATLHALHYHQVDVAAVQQGLAGKRRATIEQLLTIPLAKELAWAPDEIREEVDNNCQSILGYVVRWVDQGVGCSKVPDIHDVALMEDRATLRISSQLLANWLRHGVITSADVRASLERMAPLVDRQNAGDVAYRPMAPNFDDSIAFLAAQELILSGAQQPNGYTEPILHRRRREFKARAAEKPAPSDRAGDDAAR

SEQ ID NO: 14 - protein, 299 aa, Mycobacterium tuberculosis
MKGRSALLRALWIAALSFGLGGVAVAAEPTAKAAPYENLMVPSPSMGRDIPVAFLAGGPHAVYLLDAFNAGPDVSNWVTAGNAMNTLAGKGISVVAPAGGAYSMYTNWEQDGSKQWDTFLSAELPDWLAANRGLAPGGHAAVGAAQGGYGAMALAAFHPDRFGFAGSMSGFLYPSNTTTNGAIAAGMQQFGGVDTNGMWGAPQLGRWKWHDPWVHASLLAQNNTRVWVWSPTNPGASDPAAMIGQTAEAMGNSRMFYNQYRSVGGHNGHFDFPASGDNGWGSWAPQLGAMSGDIVGAIR

SEQ ID NO: 15 - protein, 450 aa, Lyssavirus rabies
MDADKIVFKVNNQVVSLKPEIIVDQYEYKYPAIKDLKKPCITLGKAPDLNKAYKSVLSCMSAAKLDPDDVCSYLAAAMQFFEGTCPEDWTSYGILIARKGDKITPGSLVEIKRTDVEGNWALTGGMELTRDPTVPEHASLVGLLLSLYRLSKISGQNTGNYKTNIADRIEQIFETAPFVKIVEHHTLMTTHKMCANWSTIPNFRFLAGTYDMFFSRIEHLYSAIRVGTVVTAYEDCSGLVSFTGFIKQINLTAREAILYFFHKNFEEEIRRMFEPGQETAVPHSYFIHFRSLGLSGKSPYSSNAVGHVFNLIHFVGCYMGQVRSLNATVIAACAPHEMSVLGGYLGEEFFGKGTFERRFFRDEKELQEYEAAELTKTDVALADDGTVNSDDEDYFSGETRSPEAVYTRIIMNGGRLKRSHIRRYVSVSSNHQARPNSFAEFLNKTYSSDS

SEQ ID NO: 16 - protein, 11 aa, Lyssavirus rabies
MIDPGEVYDDP

SEQ ID NO: 17 - protein, 524 aa, Lyssavirus rabies
MVPQALLFVPLLVFPLCFGKFPIYTIPDKLGPWSPIDIHHLSCPNNLVVEDEGCTNLSGFSYMELKVGYISAIKTNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPMADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETGL

SEQ ID NO: 18 - protein, 297 aa, Lyssavirus rabies
MSKIFVNPSAIRAGLADLEMAEETVDLINRNIEDNQAHLQGEPIEVDNLPEDMGRLHLDDGKSPNPGEMAKVGEGKYREDFQMDEGEDPSLLFQSYLDNVGVQIVRQIRSGERFFKIWSQTVEEIISYVAVNFPNPPGKSSEDKSTQTTGRELKKETTPTPSQRESQSSKARMAAQTASGPPALEWSATNEEDDLSVEAEIAHQIAESFSKKYKFPSRSSGILLYNFEQLKMNLDDIVKEAKNVPGVTRLARDGSKLPLRCVLGWVALANSKKFQLLVESNKLSKIMQDDLNRYTSC

SEQ ID NO: 19 - protein, 202 aa, Lyssavirus rabies
MNFLRKIVKNCRDEDTQKPSPVSAPLDDDDLWLPPPEYVPLKELTSKKNRRNFCINGGVKVCSPNGYSFGILRHILRSFDEIYSGNHRMIGLVKVVIGLALSGAPVPEGMNWVYKLRRTLIFQWADSRGPLEGEELEYSQEITWDDDTEFVGLQIRVSAKQCHIRGRIWCINMNSRAGQLWSDMSLQTQRSEEDKDSSLLLE

SEQ ID NO: 20 - RNA, 900 nt, synthetic construct
  source 1..900: mol_type = other RNA
atgtccaaaaataaagaccagcgaacggccaagactctggagaggacttgggataccctgaatcatctgttgtttatttcaagttgcctttataaattgaatctcaaatccgtcgcgcaaatcacgctgagcatactcgcaatgattatctcaacttccttgataattgccgccataatcttcattgcaagtgcaaatcataaagtgactcctacaaccgcaataatacaggacgcgaccagccaaattaagaacacgactcccacgtatctcacccaaaatccccaactcggaattagcccaagtaatccgtcagagattacttcacaaatcaccaccatactggcgagcacaactccaggcgtaaaatccaccctccaatctactaccgtcaagactaaaaataccactactactcagacacaacctagtaaacctacgactaagcagcgccagaataaacccccaagcaaaccaaacaacgacttccattttgaagttttcaacttcgtgccttgttctatctgctctaataatccaacatgctgggccatctgcaagcgcattccgaataagaaacccggtaagaaaactacaacgaaacctaccaagaagcctaccctgaaaacgacaaagaaagacccgaaaccgcaaacaaccaaaagtaaagaagtgcccacaactaaacccacagaagaaccaacgataaacacgaccaaaaccaacataataactacgctgcttaccagcaacacgacaggcaacccggaattgaccagtcagatggaaacttttcattcaaccagcagcgaaggtaatccaagtcctagtcaggtgtctacgacatctgaatatccatctcagcctagttcccctccgaacacgccgcgccagtgataa

SEQ ID NO: 21 - protein, 298 aa, Orthopneumovirus hominis
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 22 - RNA, 1725 nt, synthetic construct
  source 1..1725: mol_type = other RNA
atggagctgctcatccttaaagcgaatgctataactactatacttacagctgtaaccttttgcttcgcctctggccaaaacattactgaggagttttatcaatccacttgcagcgcagtctcaaaaggatatctgtcagcattgcgcaccgggtggtacaccagcgttatcactatcgagctttccaacattaaagagaataagtgtaacggcacagacgcgaaagtcaaactcattaagcaagaacttgataaatacaaaaatgcagttactgaacttcagttgctcatgcaatcaaccccagcaacgaacaatagggccaggagagaattgccgaggtttatgaactacacacttaataacgctaaaaagactaacgttacgctctctaagaagcggaagcgcaggtttctcgggttccttctgggggttggcagtgcaatagcatccggcgtcgcggtatcaaaggtgcttcatcttgaaggagaagttaacaaaatcaaaagcgccttgctttcaactaataaggcagtagtatcattgtctaacggtgtcagcgtcttgacttccaaggttttggatttgaaaaactatatcgacaaacaacttctcccgatcgttaacaagcagtcatgcagtatctccaacatcgaaaccgtcatcgaattccaacagaaaaacaatagactgcttgaaattactagagagttttcagtgaacgctggcgttaccactccggtatccacttatatgctcactaatagcgaactgctgtctctgataaatgacatgccaataactaacgaccagaagaaacttatgagtaacaacgtccagatcgtgagacagcaatcatatagcattatgagcataattaaggaggaggtccttgcatacgtagtccagctcccactgtacggggttatcgacacgccatgttggaagcttcatacttcccccttgtgcaccacgaacacgaaggaagggtctaacatttgtctcacgcgcactgatcgggggtggtactgtgacaacgccgggtcagtgtcatttttccctcaggccgagacctgcaaggtccaatcaaaccgggtattctgtgatactatgaactccctgactctgccttctgaagttaacctgtgtaatgtagatatattcaatcctaaatacgactgcaagataatgaccagcaaaaccgacgtgtcctcatctgtcatcacttcccttggtgctatagtaagctgctatggcaaaacgaaatgcaccgcgagtaataaaaatcgcggtatcatcaagacatttagtaacggctgcgattatgtttccaacaaaggtgttgacaccgtatctgtggggaataccctgtattacgtaaataagcaggaagggaaatctctctacgtgaagggggaaccgataatcaacttttatgacccgctggtttttccgtccgacgaatttgacgcgagtatctcccaagtcaacgagaaaattaaccaaagcctcgcgttcataagaaaatccgatgagctgctgcataatgtaaacgcgggcaaatcaactaccaacatcatgattacaacaataatcatcgtgataatcgtgatcctgctttcacttatcgctgtcgggcttcttctctattgtaaggcccggagtactcccgtgaccttgtctaaggatcagctctcaggtatcaacaatattgcattcagcaattga

SEQ ID NO: 23 - protein, 166 aa, Orthopneumovirus hominis
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNK

SEQ ID NO: 24 - protein, 574 aa, Orthopneumovirus hominis
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVSKVLHLEGEVNKIKNALQSTNKAVVSLSNGVSVLTSKVLDLKNYINNQLLPIVNKQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 25 - RNA, 3843 nt, synthetic construct
  source 1..3843: mol_type = other RNA
atgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatga

SEQ ID NO: 26 - protein, 1260 aa, synthetic construct
MFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCG

SEQ ID NO: 27 - RNA, 3846 nt, synthetic construct
  source 1..3846: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 28 - protein, 1280 aa, synthetic construct
MFLLTTKRTMFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGLTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKNFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 29 - RNA, 3846 nt, synthetic construct
  source 1..3846: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 30 - protein, 1280 aa, synthetic construct; identical to SEQ ID NO: 28

SEQ ID NO: 31 - RNA, 3837 nt, synthetic construct
  source 1..3837: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaataagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagacgacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagatcagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattcccatcggcgagcacgatctgtagcatcccagtctattattgcctacactatgtcattgggcgccgagaatagcgtcgcatattcaaataattctattgcaatacccatcaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttggcccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactcacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 32 - protein, 1282 aa, synthetic construct
MFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHAGQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 33 - RNA, 3846 nt, synthetic construct
  source 1..3846: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcaggccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 34 - protein, 1280 aa, synthetic construct; identical to SEQ ID NO: 28

SEQ ID NO: 35 - RNA, 3837 nt, synthetic construct
  source 1..3837: mol_type = other RNA
atgtttctgctcacaaccaaacgcactatgtttgttttcctcgtgctgctccctttggtaagttctcagtgtgtaaacttcacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgccaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcggggccttccccaaggcttttctgctctcgaacccctcgtagacttgccaattgggataaatatcactcgctttcaaactttgcacatcagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaacattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttaagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgtggagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 36 - protein, 1279 aa, synthetic construct
MFLLTTKRTMFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 37 - RNA, 3843 nt, synthetic construct; identical to SEQ ID NO: 25
  source 1..3843: mol_type = other RNA

SEQ ID NO: 38 - protein, 1282 aa, synthetic construct
MFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 39 - RNA, 3847 nt, synthetic construct
  source 1..3847: mol_type = other RNA
cgccaccatgtttctgttgacgaccaagcgaacgatgttcgttttcttggtgcttttgccacttgtcagttcccagtgcgtcaatctgacgacacgaacacagctgcctcctgcgtacactaacagttttacgcgaggagtgtattaccctgacaaagttttccgctctagtgtcctccatagcacacaggacttgtttctccccttcttttccaacgttacgtggttccatgtgattagtggaactaacggtactaaaagattcgacaatccagtattgcctttcaacgatggggtctatttcgcgtccatcgagaaatcaaatatcattcgcggttggatttttggaacgacactcgattcaaagacgcaatccctccttattgtcaacaacgccactaacgtagtcattaaggtttgtgagttccagttttgtaatgatcccttttttgaccacaagaataacaagagctggatggaaagcgagttcagagtgtatagctctgcaaacaactgtacttttgaatacgtgagtcaacctttccttatggaccttgaaggtaaacagggtaactttaagaatttgcgcgaatttgttttcaaaaacattgatggttactttaaaatctatagtaagcacactcctatcattgtaagagagccggaggaccttccacagggttttagtgcgctcgagcccctcgttgacctgcccattgggatcaacataactcgattccaaacattgctcgcccttcatcggtcctatctgactcccggtgactcctctagcggatggacggcaggtgccgccgcatactacgtggggtaccttcaacctcggacatttttgttgaaatacaatgagaatggcactataactgacgcggttgattgcgcgctcgacccattgtccgaaactaagtgtactttgaagtcatttacagtggagaaaggaatatatcagactagcaattttcgggtacagcccacggagtctatcgtacggtttcctaacatcacgaatctgtgcccttttgatgaggtctttaatgcaacacggttcgcctccgtctatgcgtggaatagaaagcgcatctcaaattgtgtagctgattattccgtactttacaacttggccccgttcttcacatttaagtgctacggtgtaagccctactaaactgaacgatttgtgtttcaccaacgtctatgcagatagctttgttattcgaggcgatgaggtacgccagattgcgcctggtcaaacgggtaatatcgccgactacaattataaattgccagacgattttactggttgtgtcatcgcttggaatagtaataagttggacagtaaggtatccggcaattacaactatctctaccgattgttccggaagtctaacctcaagccgtttgaaagagacatatccactgagatataccaagcaggcaataaaccatgcaacggagttgctggtttcaactgctatttcccgttgcggtcttattccttcagacctacttacggagtcggacaccaaccctacagggtcgtcgttttgagttttgaattgttgcatgctccagcaaccgtgtgtggacctaaaaagtccacgaatctcgtgaagaataagtgcgtaaacttcaatttcaacggtctgaaagggactggtgtattgacagaaagcaacaagaagtttctgccattccagcaatttggtagggatatagcggatacaactgatgccgttcgggatcctcaaacattggagatcttggacatcacaccgtgttcttttgggggtgtctccgttatcacaccgggtacaaatacgagcaatcaggttgcggtcctttaccaaggcgttaattgtaccgaggttccagtagcaatacacgcggatcaactcacgcccacatggagggtttacagtacaggcagtaatgttttccaaacgagagcgggatgcctcatcggggcagaatacgtaaataattcttacgaatgcgacatccctattggcgcaggaatttgcgcaagttaccaaacccagaccaagtctcataggcgggcgcggtctgttgcaagccaatctataatagcgtacactatgtccctcggcgcggagaacagtgtcgcatattccaacaactctattgcgatacctactaatttcactattagcgtcacaactgagatccttcccgtcagtatgaccaaaacgtctgtcgactgtactatgtatatttgcggcgacagtaccgaatgctctaatcttttgttgcagtatggttctttttgcacgcaacttaagagagctttgacggggatagctgtggaacaagataaaaacacacaggaggtatttgcacaagtgaaacagatctataaaactccaccgatcaagtactttggcggctttaacttctcccagatcttgcccgacccgtctaaaccaagtaaacggagttttatagaggaccttctcttcaataaggtaacattggcagacgccggcttcattaaacaatacggagattgccttggagacatcgctgcgcgcgacttgatctgcgcacaaaaatttaaaggcttgacggtcctccctcctttgctcacagacgagatgatagcacaatacacttccgcactgcttgctggaaccatcacctctggttggacattcggtgcgggagcggctttgcagattccgtttgcgatgcaaatggcttatcggtttaacggcattggagtaacacagaatgtgctctacgagaatcaaaagcttattgcgaatcaattcaactctgcgattggcaaaattcaagattcattgagtagcaccgccagtgctcttggcaagcttcaggatgtcgtaaaccacaatgcacaagctctgaatacactggttaaacaattgtccagtaaatttggggcaatctcttcagtgctgaacgacattttctcaagattggatccacccgaagcggaggtacagattgaccgcctgataaccgggaggttgcaaagccttcagacttatgttacacaacagttgattcgggcagcagagataagagcctcagcaaacctcgcagctacgaagatgtcagagtgtgtccttgggcaatctaagcgggtagatttctgcggcaaaggatatcatttgatgagctttccccaatcagccccacatggagtagtttttcttcatgtcacttacgttccggcgcaggaaaagaacttcaccacagcgccagccatttgtcatgatgggaaggcgcatttcccaagagaaggtgttttcgtgtctaacggtacccactggttcgttacgcagcggaatttctacgaaccacagatcatcactaccgacaacacgtttgtctctggaaattgtgacgttgtcatagggatagtgaacaatacagtatatgatccacttcagcctgaacttgactcttttaaggaggagctggacaaatatttcaaaaatcatacaagcccggacgtcgatcttggagatatttcaggtatcaacgcaagtgtcgtaaatattcagaaggagatcgatcgattgaacgaggttgcaaaaaaccttaatgagagccttatagatcttcaagagctggggaagtatgaacaatatatcaagtggccttggtacatttggctcgggttcattgccggacttatcgcgatcgtaatggtaacaatcatgctctgctgtatgacttcttgctgttcatgcctcaaaggttgttgctcctgtgggtcttgctgtaaatttgacgaggatgattctgaaccagtgcttaaaggcgtgaagctccactatacctga

SEQ ID NO: 40 - protein, 1279 aa, synthetic construct
MFLLTTKRTMFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFFDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 41 - protein, 302 aa, synthetic construct
QNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPW

SEQ ID NO: 42 - protein, 1255 aa, Severe acute respiratory syndrome-related coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIVIVMVTILLCCMTSCCSCLKGACSCGSCCKFDEDDSEPVLKGVKLHYT

SEQ ID NO: 43 - RNA, 1993 nt, synthetic construct
  source 1..1993: mol_type = other RNA
cgccaccatgttctatgaagctttgaaggccgaacttgtatacacccgagccgtgcatggtttccgcccgagagcgaactgcgtcgtactgtcagattacatccccagagtcgcgtgcaacatgggtacggttaataagcccgtggtgggggtgctgatgggttttgggataatcaccggaactctgcgcatcacaaatcctgtaagagcgtcagtgctgaggtatgacgatttccacatagacgaggacaagctggataccaattccgtttacgaaccatattatcactccgatcacgccgaatccagctgggtaaatcgaggagagtcctcacggaaggcatacgaccataactccccatatatatggcctcgcaatgattatgacggattcctggaaaacgcccatgagcaccacggcgtttataaccagggtcgcggtatcgattctggggaaaggttgatgcaaccaactcagatgtccgctcaggaagatttgggggatgacaccggaatccatgtgatacctacactcaatggtgatgatcgacacaaaattgtaaacgtggaccagaggcaatacggggacgtttttaagggagacctgaatccaaaaccacaaggacaacgactcatcgaggtttctgtagaagaaaaccatccgtttacgctccgcgcccccatacagcgcatatatggcgtgcgctatacggagacctggtcttttctccccagtctgacatgcactggtgatgcagctccagcaattcagcacatatgtctcaagcataccacctgtttccaagatgtagtagtggatgtggattgcgccgaaaacacgaaagaagaccagcttgccgagatttcataccggtttcaaggaaaaaaagaagccgaccagccgtggatagtagtgaatacgtccacgctcttcgatgaactggaattggaccctcctgaaattgaacccggagtcctgaaagttttgcggactgagaaacagtatctcggtgtgtacatctggaatatgaggggcagtgatggaacaagcacatacgcgacctttctggtgacatggaaaggtgacgaaaagactagaaacccaaccccagcagtgaccccccaacctagaggtgctgagttccacatgtggaattaccactcccatgtattctctgtcggagacaccttcagcttggcgatgcacctccaatacaagatccatgaagccccgttcgacctcttgttggagtggctctacgtaccaatagaccctacctgtcaaccaatgaggctttactctacatgcctttaccacccaaacgcaccacaatgtctttctcatatgaatagcggttgtacatttacatctccccatctcgcgcaaagagtcgcgagtacagtttaccaaaattgtgagcatgcggacaattacacagcatattgtcttgggatctcccacatggagccctcttttgggcttatacttcacgacgggggtacaacgcttaagttcgtcgatacgccagaatccctctctggcttgtatgtattcgtggtttattttaacggtcacgttgaggctgtggcctatactgttgttagcactgtagaccactttgttaacgccatagaagagagggggttcccgcccaccgctggccagccaccggccacaacgaagcctaaagagattacgccggtgaatcccggcacaagtccccttatccggtacgccgcatggacaggaggactggcggccgtcgtcctcctttgtctggttatattccttatatgcacggcaaagcgaatgcgggttaaggcttaccgagtggataaatctccatataatcagagcatgtattacgcagggctccctgtggacgactttgaggatagcgagtccacggatacagaagaggaatttggaaacgccatcggtgggagccatggagggtcatcttacaccgtttacattgacaaaactagatga

SEQ ID NO: 44 - protein, 661 aa, Varicellovirus humanalpha3
MFYEALKAELVYTRAVHGFRPRANCVVLSDYIPRVACNMGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 45 - protein, 931 aa, Varicellovirus humanalpha3
MSPCGYYSKWRNRDRPEYRRNLRFRRFFSSIHPNAAAGSGFNGPGVFITSVTGVWLCFLCIFSMFVTAVVSVSPSSFYESLQVEPTQSEDITRSAHLGDGDEIREAIHKSQDAETKPTFYVCPPPTGSTIVRLEPTRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 46 - protein, 841 aa, Varicellovirus humanalpha3
MFALVLAVVILPLWTTANKSYVTPTPATRSIGHMSALLREYSDRNMSLKLEAFYPTGFDEELIKSLHWGNDRKHVFLVIVKVNPTTHEGDVGLVIFPTYLLSPYHFKAEHRAPFPAGRFGFLSHPVTPDVSFFDSSFAPYLTTQHLVAFTTFPPNPLVWHLERAETAATAERPFGVSLLPARPTVPKNTILEHKAHFATWDALARHTFFSAEAIITNSTLRIHVPLFGSVWPIRYWATGSVLLTSDSGRVEVNIGVGFMSSLISLSSGLPIELIVVPHTVKLNAVTSDTTWFQLNPPGPDPGPSYRVYLLGRGLDMNFSKHATVDICAYPEESLDYRYHLSMAHTEALRMTTKADQHDINEESYYHIAARIATSIFALSEMGRTTEYFLLDEIVDVQYQLKFLNYILMRIGAGAHPNTISGTSDLIFADPSQLHDELSLLFGQVKPANVDYFISYDEARDQLKTAYALSRGQDHVNALSLARRVIMSIYKGLLVKQNLNATERQALFFASMILLNFREGLENSSRVLDGRTTLLLMTSMCTAAHATQAALNIQEGLAYLNPSKHMFTIPNVYSPCMGSLRTDLTEEIHVMNLLSAIPTRPGLNEVLHTQLDESEIFDAAFKTMMIFTTWTAKDLHILHTHVPEVFTCQDAAARNGEYVLILPAVQGHSYVITRNKPQRGLVYSLADVDVYNPISVVYLSKDTCVSEHGVIETVALPHPDNLKECLYCGSVFLRYLTTGAIMDIIIIDSKDTERQLAAMGNSTIPPFNPDMHGDDSKAVLLFPNGTVVTLLGFERRQAIRMSGQYLGASLGGAFLAVVGFGIIGWMLCGNSRLREYNKIPLT

SEQ ID NO: 47 - protein, 160 aa, Varicellovirus humanalpha3
MASHKWLLQMIVFLKTITIAYCLHLQDDTPLFFGAKPLSDVSLIITEPCVSSVYEAWDYAAPPVSNLSEALSGIVVKTKCPVPEVILWFKDKQMAYWTNPYVTLKGLAQSVGEEHKSGDIRDALLDALSGVWVDSTPSSTNIPENGCVWGADRLFQRVCQ

SEQ ID NO: 48 - protein, 87 aa, Varicellovirus humanalpha3
MGSITASFILITMQILFFCEDSSGEPNFAERNFWHASCSARGVYIDGSMITTLFFYASLLGVCVALISLAYHACFRLFTRSVLRSTW

SEQ ID NO: 49 - RNA, 1072 nt, synthetic construct
  source 1..1072: mol_type = other RNA
cgccaccatgttccttattcaatgcctcatatccgcagtcatcttctatattcaagttactaatgcacttatcttcaagggcgatcatgtcagcctgcaagtgaattcaagtcttacgtccattttgattccaatgcagaatgataactataccgaaattaagggacagctggtttttattggagaacaactgccgaccggtacaaactacagcgggaccctcgagctgctttacgcagacactgtggcattctgctttcggtcagtacaagttatcagatacgacggatgcccacgcattaggacatctgccttcatttcttgccgatacaagcatagttggcattacggaaactctaccgatagaatttcaactgaaccagatgccggtgtgatgctcaagataaccaaacctgggatcaacgacgcaggggtgtatgtcctcttggtgagattggaccattcaaggagtacggacgggtttatactgggcgtgaacgtctataccgcaggaagtcatcataacattcacggtgtcatttataccagccccagtctccagaatgggtacagcactcgagccctgttccagcaagcaagattgtgtgaccttccagccactcctaagggatcaggcacaagtctttttcaacatatgttggatctcagagcagggaaaagtcttgaggacaacccgtggctccatgaagacgtggttactactgaaacaaagtcagtggtcaaggagggaatcgagaaccatgtgtacccaactgacatgagcacgctgcctgaaaaatcactgaacgacccaccagagaatctgctgataataatacctattgtagcgagtgttatgattttgaccgcaatggtcatagttattgtaataagcgtgaaaaggagacgaatcaaaaaacatccgatatacaggccgaatacgaagacaagaagagggattcagaacgcgactccggagagtgatgtaatgctcgaagcagccatcgctcaacttgccaccattcgcgaagaaagccctccgcattccgtcgtaaatccttttgtcaagtga

SEQ ID NO: 50 - protein, 354 aa, Varicellovirus humanalpha3
MFLIQCLISAVIFYIQVTNALIFKGDHVSLQVNSSLTSILIPMQNDNYTEIKGQLVFIGEQLPTGTNYSGTLELLYADTVAFCFRSVQVIRYDGCPRIRTSAFISCRYKHSWHYGNSTDRISTEPDAGVMLKITKPGINDAGVYVLLVRLDHSRSTDGFILGVNVYTAGSHHNIHGVIYTSPSLQNGYSTRALFQQARLCDLPATPKGSGTSLFQHMLDLRAGKSLEDNPWLHEDVVTTETKSVVKEGIENHVYPTDMSTLPEKSLNDPPENLLIIIPIVASVMILTAMVIVIVISVKRRRIKKHPIYRPNTKTRRGIQNATPESDVMLEAAIAQLATIREESPPHSVVNPFVK

SEQ ID NO: 51 - RNA, 2079 nt, synthetic construct
  source 1..2079: mol_type = other RNA
atgcggagaggagcagacacatccgtgggaatcgtgggcctgctgctgaccacagcaatggcagccgaggtgaccaggagaggcagcgcctactatatgtacctggacagaaatgatgccggcgaggccatctcctttcccaccacactgggcatgaacaagtgctacatccagatcatggacctgggccacatgtgcgatgccaccatgagctatgagtgtccaatgctggacgagggcgtggagcccgacgatgtggattgctggtgtaataccacatctacatgggtggtgtacggcacctgtcaccacaagaagggagaggcacggcgcagcaggagagcagtgacactgccttcccactctaccaggaagctgcagacaagaagccagacctggctggagtccagggagtatacaaagcacctgatcagggtggagaactggatctttagaaatccaggattcgcactggctgccgccgccatcgcatggctgctgggcagctccaccagccagaaagtgatctacctggtcatgatcctgctgatcgcccctgcctattctatccggtgcatcggcgtgagcaatagggacttcgtggagggaatgtccggaggcacctgggtggatgtggtgctggagcacggcggctgcgtgacagtgatggcccaggacaagccaaccgtggatatcgagctggtgaccacaaccgtgtccaacatggccgaggtgaggtcttactgctatgaggccagcatctccgacatggcctctgatagcagatgtcccacccagggcgaggcctacctggacaagcagtccgatacacagtacgtgtgcaagcggaccctggtggacaggggatggggaaatggatgtggcctgtttggcaagggctctctggtgacatgcgccaagttcgcctgtagcaagaagatgaccggcaagtccatccagccagagaacctggagtaccggatcatgctgtctgtgcacggctcccagcactctggcatgatcgtgaacgacacaggccacgagacagatgagaatcgggccaaggtggagatcacacctaactccccacgcgccgaggccaccctgggaggatttggctctctgggcctggactgcgagcctaggacaggcctggacttctccgatctgtactatctgaccatgaacaataagcactggctggtgcacaaggagtggtttcacgacatcccactgccatggcacgcaggagcagatacaggcaccccacactggaacaataaggaggccctggtggagttcaaggacgcccacgccaagcggcagacagtggtggtgctgggcagccaggagggagcagtgcacaccgccctggcaggcgccctggaggccgagatggacggagcaaagggccgcctgtctagcggccacctgaagtgcaggctgaagatggataagctgagactgaagggcgtgtcctactctctgtgcacagccgccttcaccttcaccaagatccctgccgagacactgcacggcacagtgaccgtggaggtgcagtatgccggcacagacggcccctgtaaggtgcctgcccagatggccgtggatatgcagacactgacccctgtgggcaggctgatcaccgccaatccagtgatcacagagtctaccgagaacagcaagatgatgctggagctggacccccctttcggcgatagctatatcgtgatcggcgtgggcgagaagaagatcacacaccactggcacagaagcggctccacaatcggcaaggcctttgaggcaaccgtgcggggagcaaagaggatggccgtgctgggcgacaccgcatgggatttcggatctgtgggaggcgccctgaacagcctgggcaagggcatccaccagatcttcggcgccgcctttaagtccctgttcggcggcatgagctggttttcccagatcctgatcggcacactgctgatgtggctgggcctgaacaccaagaatggctctatcagcctgatgtgcctggccctgggaggcgtgctgatcttcctgtccaccgccgtgtctgcc

SEQ ID NO: 52 - protein, 3181 aa, Orthoflavivirus zikaense
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRAPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPTSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTWKRSGALWDVPASKEVKKGETTDGVYRVMTRRLLGSTQVGVGIMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTILEDSVPAEVWTRHGEKRVLKPRWMDARVCSDHAALKSFKEFAAGKRGAAFGVMEALGTLPGHMTERFQEAIDNLAVLMRAEIGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSCIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMA

SEQ ID NO: 53 - protein, 455 aa, synthetic construct
QVQLVQSGAEVKKPGSSVKVSCKASGGTFSNYAISWVRQAPGQGLEWMGRIIPILGIANYAQKFQGRVTITADKSTSTAYMELSSLRSEDTAVYYCARGYYEARHYYYYYAMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 214 aa, synthetic construct
DIQMTQSPSSLSASVGDRVTITCRASQSISSYLSWYQQKPGKAPKLLIYAASSLQSGVPSRFSGSGSGTDFTLTITSLQPEDFATYYCQQSYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 449 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 214 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 457 aa, synthetic construct
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 58 - protein, 216 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 59 - protein, 457 aa, synthetic construct; identical to SEQ ID NO: 57

SEQ ID NO: 60 - protein, 214 aa, synthetic construct
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 8 aa, synthetic construct
GFTFKNYG

SEQ ID NO: 62 - protein, 8 aa, synthetic construct
VRYDGNNK

SEQ ID NO: 63 - protein, 12 aa, synthetic construct
ARDPETFGGFDY

SEQ ID NO: 64 - protein, 6 aa, synthetic construct
ESVSSN

SEQ ID NO: 65 - skipped

SEQ ID NO: 66 - protein, 9 aa, synthetic construct
QQYYYSPRT

SEQ ID NO: 67 - RNA, 10571 nt, synthetic construct
  source 1..10571: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagttcggtcttagctgggtgtttcttgtcgccctgttcagaggggtacaatgccaagtgcaacttgtagagagtggcggcggcgtagttcgaccaggtgggagtctgaggctgtcatgtgcagcatccgggttcacctttaaaaactacgggattcactgggtgaggcaggctcctggtaagggaccagagtgggtcgccttcgtgcgctatgatgggaataacaaatattacgctgactccgtcaaaggtcgcttcacaatatccagggacaatgcaaaaaatacactgagcttgcaaatgaattctttgcgggtggaagatactgctgtatatttctgtgctcgggatccagaaacttttggagggtttgattattgggggcaagggacactcgttactgtcagtagcgcctccacaaagggtccaagtgtcttcccactggctcccagcagcaagagtacttcaggtgggactgcagctctcgggtgcctggtcaaggactactttcccgagcccgtaacagtatcttggaactccggtgctctgacaagtggagtgcatactttcccagctgtgttgcagtcaagcgggttgtactccctcagtagtgtagttactgtcccttcatcttcactggggactcaaacctacatttgtaacgtgaatcacaaaccaagcaatactaaagtagataagaaggtggagccaaaaagttgtgataaaactcatacttgtcccccctgtcctgcaccagagctgttgggcggtcccagtgtatttttgtttccccctaaacccaaagacacactgatgatttctcgaactcccgaagtgacctgcgtcgtcgttgatgtaagtcacgaagaccccgaggtaaaattcaattggtacgtagacggcgtagaggtgcataacgctaaaaccaaaccaagggaggaacaatacaacagcacttacagagtcgtatctgtactgactgttctccaccaagactggcttaatgggaaggagtataaatgcaaggtgtcaaacaaggctctgcctgcccccatagagaaaaccataagtaaagctaaaggacaacctagagagccccaggtttatactcttcccccctcccgagaagagatgaccaagaaccaagtttccttgacctgtctggttaagggtttttatccaagcgatatagccgtagaatgggagagcaacggacaacctgagaataactacaaaacaactcccccagtgctggactcagatggctcatttttcctgtattcaaagctcaccgtggacaaatctcggtggcagcaagggaatgtattctcctgttccgtcatgcacgaggcgctgcataatcactatacccagaaatctctgtccctttctcctggaaagtgataatctagacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggacatgcgagtccctgcccaactgctgggtcttctcctcctttggctgtcaggagcccgctgcgaaattgtaatgacacaatctccagcaacactttccgtcagtccaggcgagagagggaccttgtcatgcagggcttccgaaagcgtttccagcaaccttgcttggtatcaacaaaaacctggaaaggcccccagactgctgatctatggcgcttccacacgcgcaactggtattcctgacagattctccgggtctgggtctggcactgagttcacactcacaatttccagtctgcagtccgaggattttgctgtatattattgccaacagtattactatagtcctaggacctttggtcaagggactaaggtcgaggtaaagcggaccgtggctgcaccatccgtttttatttttccaccaagtgatgagcagcttaaaagtggtactgcctccgtggtgtgcttgttgaacaacttctacccacgcgaggccaaggtgcaatggaaggtagataatgccttgcagagtggaaattctcaagagtcagtcaccgaacaggatagtaaagactctacatattctcttagctctaccctcactttgtctaaagcagattatgaaaagcataaagtgtatgcatgcgaagtgacccaccaggggctgaggtctcctgtcaccaaaagttttaacaggggagagtgttgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 68 - RNA, 1407 nt, synthetic construct
  source 1..1407: mol_type = other RNA
atggagttcggtcttagctgggtgtttcttgtcgccctgttcagaggggtacaatgccaagtgcaacttgtagagagtggcggcggcgtagttcgaccaggtgggagtctgaggctgtcatgtgcagcatccgggttcacctttaaaaactacgggattcactgggtgaggcaggctcctggtaagggaccagagtgggtcgccttcgtgcgctatgatgggaataacaaatattacgctgactccgtcaaaggtcgcttcacaatatccagggacaatgcaaaaaatacactgagcttgcaaatgaattctttgcgggtggaagatactgctgtatatttctgtgctcgggatccagaaacttttggagggtttgattattgggggcaagggacactcgttactgtcagtagcgcctccacaaagggtccaagtgtcttcccactggctcccagcagcaagagtacttcaggtgggactgcagctctcgggtgcctggtcaaggactactttcccgagcccgtaacagtatcttggaactccggtgctctgacaagtggagtgcatactttcccagctgtgttgcagtcaagcgggttgtactccctcagtagtgtagttactgtcccttcatcttcactggggactcaaacctacatttgtaacgtgaatcacaaaccaagcaatactaaagtagataagaaggtggagccaaaaagttgtgataaaactcatacttgtcccccctgtcctgcaccagagctgttgggcggtcccagtgtatttttgtttccccctaaacccaaagacacactgatgatttctcgaactcccgaagtgacctgcgtcgtcgttgatgtaagtcacgaagaccccgaggtaaaattcaattggtacgtagacggcgtagaggtgcataacgctaaaaccaaaccaagggaggaacaatacaacagcacttacagagtcgtatctgtactgactgttctccaccaagactggcttaatgggaaggagtataaatgcaaggtgtcaaacaaggctctgcctgcccccatagagaaaaccataagtaaagctaaaggacaacctagagagccccaggtttatactcttcccccctcccgagaagagatgaccaagaaccaagtttccttgacctgtctggttaagggtttttatccaagcgatatagccgtagaatgggagagcaacggacaacctgagaataactacaaaacaactcccccagtgctggactcagatggctcatttttcctgtattcaaagctcaccgtggacaaatctcggtggcagcaagggaatgtattctcctgttccgtcatgcacgaggcgctgcataatcactatacccagaaatctctgtccctttctcctggaaagtga

SEQ ID NO: 69 - RNA, 714 nt, synthetic construct
  source 1..714: mol_type = other RNA
atggacatgcgagtccctgcccaactgctgggtcttctcctcctttggctgtcaggagcccgctgcgaaattgtaatgacacaatctccagcaacactttccgtcagtccaggcgagagagggaccttgtcatgcagggcttccgaaagcgtttccagcaaccttgcttggtatcaacaaaaacctggaaaggcccccagactgctgatctatggcgcttccacacgcgcaactggtattcctgacagattctccgggtctgggtctggcactgagttcacactcacaatttccagtctgcagtccgaggattttgctgtatattattgccaacagtattactatagtcctaggacctttggtcaagggactaaggtcgaggtaaagcggaccgtggctgcaccatccgtttttatttttccaccaagtgatgagcagcttaaaagtggtactgcctccgtggtgtgcttgttgaacaacttctacccacgcgaggccaaggtgcaatggaaggtagataatgccttgcagagtggaaattctcaagagtcagtcaccgaacaggatagtaaagactctacatattctcttagctctaccctcactttgtctaaagcagattatgaaaagcataaagtgtatgcatgcgaagtgacccaccaggggctgaggtctcctgtcaccaaaagttttaacaggggagagtgttgataa

SEQ ID NO: 70 - RNA, 942 nt, synthetic construct
  source 1..942: mol_type = other RNA
atgccgctggagcagcgatcccaacattgcaaaccagaggaaggcctcgaggcaagaggtgaggccctggggctcgtcggagctcaggcaccggcaacggaggagcaagaagcggcctctagcagctccacactcgtcgaagtgacactcggcgaggtccccgctgcggaatctcccgatcctccgcaaagccctcaaggggcttcctccctccccactactatgaactatccgttgtggagccaaagctatgaggattcctcaaaccaagaggaggaaggcccctccacattcccggacctcgaatctgagttccaggcagcgctcagcaggaaagtggcggaacttgtacagtttttgttgcttaaataccgggctcgggagccagtcacgaaagccgagatgttaggcagcgtggtgggtaattggcagtacttcttccccgtcatcttttccaaggcttcctcttctttgcaactcgtttttggcatagagcttatggaagtagacccgacaggtcatctttacatttttgcaacgtgtctcggcctcagttatgacgggctgctcggtgacaatcaaattatgcctaaggccggactgcttatcatcgtcttggctataattgcccgagagggcgactgtgctcccgaagagaaaatatgggaggagctctccgtgctggaggtatttgagggaagagaagatagcatactgggagaccctaagaaattgcttacacaacactttgtacaagagaactatttggagtatcgacaggtccccggatctgacccagcatgttatgagttcctgtgggggccaagggcactcgtagagacgtcatatgtcaaggtgctgcaccacatggtcaaaataagcggcggcccccacataagctacccacccctccatgaatgggttttgagggagggtgaagaa

SEQ ID NO: 71 - RNA, 942 nt, synthetic construct; identical to SEQ ID NO: 70
  source 1..942: mol_type = other RNA

SEQ ID NO: 72 - RNA, 1611 nt, synthetic construct
  source 1..1611: mol_type = other RNA
atgaaatcttacaacgtcctccccctagcctatatctcccttttcctgatgctgttttatcaggtttgggctcagtttccacgagagtgtgccaatattgaggctctgagacgtggggtgtgttgcccagacctgctcccttcctctggaccggggactgacccttgtggctcatcatcaggaagaggcaggtgtgtggctgtgattgcagactcccgaccccacagccgccattatccccacgatggtaaagatgaccgagaagcctggcctctgaggttctttaatagaacatgtcagtgcaatgataatttctcaggacacaactgtgggacttgccgtcctgggtggagaggagctgcatgcaaccagaaaattctcacagtcaggagaaatcttctagacttaagtccagaagaaaagagccactttgtcagggccttggatatggcgaagcgcacaactcaccctcaatttgtcattgccacaaggaggttagaagacatactgggaccagatggcaacacaccacaatttgagaacatttccgtttataactactttgtttggacacactattattcagtcaaaaaaaccttcctcgggacaggacaggaaagctttggggatgtggatttctctcacgaaggacccgcttttctcacatggcacaggtaccatctgctgcagctggagagagacatgcaggagatgctgcaggagccttctttctcccttccttactggaattttgcaactgggaaaaacgtctgcgatgtctgcactgatgacttgatgggatccagaagcaacttcgattctactcttataagccccaactctgtcttttctcaatggagagtggtctgtgaatccttggaagagtacgataccctgggaacactttgtaacagcactgagggtggaccaatcaggagaaacccagctggaaatgtagggagaccagcagtgcagcgtcttcctgagccacaggatgtcactcagtgcttggaggtccgtgtatttgacacacctcctttttattccaattctacagacagttttcgaaatacagtggaaggttacagtgctcccacgggaaaatatgaccctgctgttcgaagccttcacaacctggcccacctcttcctgaatggaacgggaggacaaacccatttgtctcccaatgatcctatttttgtcctcctgcacactttcactgatgcggtctttgacgaatggctaaggaggtataacgccgatatttctaccttcccgttggaaaacgcacctattggacataacaggcaatacaacatggtgccattctggcctccagttaccaacacagaaatgtttgttactgctccagacaatctgggatatatgtatgaagttcaatggccaggtcaggagtttactgtatctgaaatcattaccattgctgtagtggctgcgttgttacttgtagctgccattttcggggttgcttcttgtctgatccgttctagaagcaccaagaatgaagccaaccagcctctcctcactgatcactatcaacgctatgctgaggactatgaggagctcccgaatcctaaccactccatggtc

SEQ ID NO: 73 - protein, 314 aa, synthetic construct
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVQFLLLKYRAREPVTKAEMLGSVVGNWQYFFPVIFSKASSSLQLVFGIELMEVDPTGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 74 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 75 - protein, 537 aa, synthetic construct
MKSYNVLPLAYISLFLMLFYQVWAQFPRECANIEALRRGVCCPDLLPSSGPGTDPCGSSSGRGRCVAVIADSRPHSRHYPHDGKDDREAWPLRFFNRTCQCNDNFSGHNCGTCRPGWRGAACNQKILTVRRNLLDLSPEEKSHFVRALDMAKRTTHPQFVIATRRLEDILGPDGNTPQFENISVYNYFVWTHYYSVKKTFLGTGQESFGDVDFSHEGPAFLTWHRYHLLQLERDMQEMLQEPSFSLPYWNFATGKNVCDVCTDDLMGSRSNFDSTLISPNSVFSQWRVVCESLEEYDTLGTLCNSTEGGPIRRNPAGNVGRPAVQRLPEPQDVTQCLEVRVFDTPPFYSNSTDSFRNTVEGYSAPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYMYEVQWPGQEFTVSEIITIAVVAALLLVAAIFGVASCLIRSRSTKNEANQPLLTDHYQRYAEDYEELPNPNHSMV

SEQ ID NO: 76 - protein, 553 aa, Homo sapiens
MVQRLWVSRLLRHRKAQLLLVNLLTFGLEVCLAAGITYVPPLLLEVGVEEKFMTMVLGIGPVLGLVCVPLLGSASDHWRGRYGRRRPFIWALSLGILLSLFLIPRAGWLAGLLCPDPRPLELALLILGVGLLDFCGQVCFTPLEALLSDLFRDPDHCRQAYSVYAFMISLGGCLGYLLPAIDWDTSALAPYLGTQEECLFGLLTLIFLTCVAATLLVAEEAALGPTEPAEGLSAPSLSPHCCPCRARLAFRNLGALLPRLHQLCCRMPRTLRRLFVAELCSWMALMTFTLFYTDFVGEGLYQGVPRAEPGTEARRHYDEGVRMGSLGLFLQCAISLVFSLVMDRLVQRFGTRAVYLASVAAFPVAAGATCLSHSVAVVTASAALTGFTFSALQILPYTLASLYHREKQVFLPKYRGDTGGASSEDSLMTSFLPGPKPGAPFPNGHVGAGGSGLLPPPPALCGASACDVSVRVVVGEPTEARVVPGRGICLDLAILDSAFLLSQVAPSLFMGSIVQLSQSVTAYMVSAAGLGLVAIYFATQVVFDKSDLAKYSA

SEQ ID NO: 77 - protein, 9 aa, synthetic construct
KCDICTDEY

SEQ ID NO: 78 - protein, 9 aa, synthetic construct
YMDGTMSQV

SEQ ID NO: 79 - protein, 9 aa, synthetic construct
MLLAYLYQL

SEQ ID NO: 80 - protein, 9 aa, synthetic construct
AFLPWHRLF

SEQ ID NO: 81 - protein, 9 aa, synthetic construct
SEIWRDIDF

SEQ ID NO: 82 - protein, 9 aa, synthetic construct
YLEPGPVTA

SEQ ID NO: 83 - protein, 9 aa, synthetic construct
KTWGQUWQV

SEQ ID NO: 84 - protein, 9 aa, synthetic construct
ITDQVPFSV

SEQ ID NO: 85 - protein, 10 aa, synthetic construct
VLYRYGSFSV

SEQ ID NO: 86 - protein, 10 aa, synthetic construct
LLDGTATLRL

SEQ ID NO: 87 - protein, 9 aa, synthetic construct
ALLAVGATK

SEQ ID NO: 88 - protein, 9 aa, synthetic construct
MLGTHTMEV

SEQ ID NO: 89 - protein, 9 aa, synthetic construct
LIYRRRLMK

SEQ ID NO: 90 - protein, 9 aa, synthetic construct
ALNFPGSQK

SEQ ID NO: 91 - protein, 9 aa, synthetic construct
AAGIGILTV

SEQ ID NO: 92 - protein, 9 aa, synthetic construct
ILTVILGVL

SEQ ID NO: 93 - protein, 9 aa, synthetic construct
MSLQRQFLR

SEQ ID NO: 94 - protein, 9 aa, synthetic construct
SVYDFFVWL

SEQ ID NO: 95 - protein, 9 aa, synthetic construct
LLGPGRPYR

SEQ ID NO: 96 - protein, 9 aa, synthetic construct
YLSGANLNL

SEQ ID NO: 97 - protein, 9 aa, synthetic construct
KIFGSLAFL

SEQ ID NO: 98 - protein, 10 aa, synthetic construct
VMAGVGSPYV

SEQ ID NO: 99 - protein, 9 aa, synthetic construct
IISAVVGIL

SEQ ID NO: 100 - protein, 9 aa, synthetic construct
LLHETDSAV

SEQ ID NO: 101 - protein, 9 aa, synthetic construct
ALFDIESKV

SEQ ID NO: 102 - protein, 9 aa, synthetic construct
EADPTGHSY

SEQ ID NO: 103 - protein, 9 aa, synthetic construct
SLFRAVITK

SEQ ID NO: 104 - protein, 9 aa, synthetic construct
SAYGEPRKL

SEQ ID NO: 105 - protein, 9 aa, synthetic construct
KMVELVHFL

SEQ ID NO: 106 - protein, 10 aa, synthetic construct
YLOLVFGIEV

SEQ ID NO: 107 - protein, 9 aa, synthetic construct
EVDPIGHLY

SEQ ID NO: 108 - protein, 9 aa, synthetic construct
FLWGPRALV

SEQ ID NO: 109 - protein, 10 aa, synthetic construct
MEVDPIGHLY

SEQ ID NO: 110 - protein, 9 aa, synthetic construct
AARAVFLAL

SEQ ID NO: 111 - protein, 8 aa, synthetic construct
YRPRPRRY

SEQ ID NO: 112 - protein, 9 aa, synthetic construct
VLPDVFIRC

SEQ ID NO: 113 - protein, 9 aa, synthetic construct
QLSLLMWIT

SEQ ID NO: 114 - protein, 9 aa, synthetic construct
SLLMWITQC

SEQ ID NO: 115 - protein, 10 aa, synthetic construct
ASGPGGGAPR

SEQ ID NO: 116 - protein, 10 aa, synthetic construct
QDLTMKYQIF

SEQ ID NO: 117 - protein, 10 aa, synthetic construct
  VARIANT 1: note = May be absent
EAYGLDFYIL

SEQ ID NO: 118 - protein, 9 aa, synthetic construct
SYLDSGIHF

SEQ ID NO: 119 - protein, 9 aa, synthetic construct
ETVSEQSNV

SEQ ID NO: 120 - protein, 9 aa, synthetic construct
FPSDSWCYF

SEQ ID NO: 121 - protein, 9 aa, synthetic construct
EEKLIVVLF

SEQ ID NO: 122 - protein, 9 aa, unidentified
SYLDSGIHS

SEQ ID NO: 123 - protein, 9 aa, unidentified
ETVSEESNV

SEQ ID NO: 124 - protein, 9 aa, unidentified
EEKLSVVLF

SEQ ID NO: 125 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 129 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 443 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 449 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 448 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 449 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 451 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 139 - protein, 449 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 222 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 141 - protein, 456 aa, synthetic construct
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 220 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERK

SEQ ID NO: 143 - protein, 446 aa, synthetic construct
VQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - protein, 226 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 145 - protein, 446 aa, synthetic construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - RNA, 103 nt, synthetic construct
  source 1..103: mol_type = other RNA
ccatcctgtttttttccctttttttttttcttttttttttttttttttttttttttttttttttctcctttttttttcctctttttttccttttctttccttt

SEQ ID NO: 149 - RNA, 60 nt, synthetic construct
  source 1..60: mol_type = other RNA
cccccccccccccccccccccccccccccccccccccccccccccccccccccccccccc

SEQ ID NO: 150 - RNA, 100 nt, synthetic construct
  source 1..100: mol_type = other RNA
gggggggggggggggggggggggggggggggggggggggggggggggggggggggggggggggggggggggggggggggggggggggggggggggggggg

SEQ ID NO: 151 - DNA, 165 bp, synthetic construct
  source 1..165: mol_type = other DNA
  misc_feature 1..30: note = DNA
  misc_feature 31..165: note = RNA
tgctgctgcttgcaagcagcttgataccagacaaagctgggaatagaaacttcgtattttcaaagttttctttaatatattgcaaataatgcctaaccacctagggcaggattagggttccggagttcaaccaattagtccttaatcagggcactgtatccgact

SEQ ID NO: 152 - RNA, 140 nt, synthetic construct
  source 1..140: mol_type = other RNA
agtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 153 - RNA, 32 nt, synthetic construct
  source 1..32: mol_type = other RNA
  modified_base 1: mod_base = OTHER; note = Triphosphate modified nucleotide
ggatcgatcgatcgttcgcgatcgatcgatcc

SEQ ID NO: 154 - RNA, 99 nt, synthetic construct
  source 1..99: mol_type = other RNA
gacgaagaccacaaaaccagataaaaaaaaaaaaaaaaaaaaaaaaaataattttttttttttttttttttttttttatctggttttgtggtcttcgtc

SEQ ID NO: 155 - RNA, 24 nt, synthetic construct
  source 1..24: mol_type = other RNA
  modified_base 1: mod_base = OTHER; note = Triphosphate modified nucleotide
ggacgtacgtttcgacgtacgtcc

SEQ ID NO: 156 - RNA, 87 nt, synthetic construct
  source 1..87: mol_type = other RNA
  modified_base 1: mod_base = OTHER; note = Triphosphate modified nucleotide
agcaaaagcagggtgacaaagacataatggatccaaacactgtgtcaagctttcaggtagattgctttctttggcatgtccgcaaac

SEQ ID NO: 157 - DNA, 122 bp, synthetic construct
  source 1..122: mol_type = other DNA
  modified_base 1: mod_base = OTHER; note = Triphosphate modified nucleotide
taatacgactcactataggccatcctgtttttttcccttttttttttctttttttttttttttttttttttttttttttttttctcctttttttttcctctttttttccttttctttccttt

SEQ ID NO: 158 - protein, 10 aa, synthetic construct
VPGVGVPGVG

SEQ ID NO: 159 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 160 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 161 - protein, 236 aa, Mus musculus
MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 162 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 163 - protein, 534 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLILLHAFRIRAVTIDRVMSYLNAS

SEQ ID NO: 164 - protein, 538 aa, Mus sp.
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSVPGVGVPGVGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 165 - RNA, 1643 nt, Mus sp.
  source 1..1643: mol_type = genomic RNA
ctgagatcaccgggacgcgtcaacatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagagtccgatcggttcctggagtaggggtacctggagtgggcagggtcataccggtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaaag

SEQ ID NO: 166 - RNA, 1646 nt, Homo sapiens
  source 1..1646: mol_type = genomic RNA
ctgagatcaccggtcaacatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccg

SEQ ID NO: 167 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 168 - protein, 155 aa, Mus musculus
MNATHCILALQLFLMAVSGCYCHGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 169 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 170 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 171 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 172 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 173 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 174 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 175 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 176 - RNA, 7561 nt, Venezuelan equine encephalitis virus
  source 1..7561: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 177 - protein, 51 aa, Venezuelan equine encephalitis virus
RELPVLDSAAFNVECFKKYACNNEYWETFKENPIRLTEENVVNYITKLKGP

SEQ ID NO: 178 - protein, 41 aa, Venezuelan equine encephalitis virus
TQMRELPVLDSAAFNVECFKKYACNNEYWETFKENPIRLTE

SEQ ID NO: 179 - protein, 536 aa, Venezuelan equine encephalitis virus
MKAITARRILQGLGHYLKAEGKVECYRTLHPVPLYSSSVNRAFSSPKVAVEACNAMLKENFPTVASYCIIPEYDAYLDMIDGASCCLDTASFCPAKLRSFPKKHSYLEPTIRSAVPSAIQNTLQNVLAAATKRNCNVTQMRELPVLDSAAFNVECFKKYACNNEYWKTFKENPIRLTEENVINYITKLKGPKAAALYAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTAMMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILPELCKAVESRYETVGTSVIVMAMATLASSVKSFSYLRGAPITLYG

SEQ ID NO: 180 - RNA, 2585 nt, Human immunodeficiency virus 1
  source 1..2585: mol_type = genomic RNA
tgcgcgttatgggtatccaaagaaattgtcagcacctgttccggtgggggacaatgattctcggcatgattatcatatgctccgctgcggagaatttgtgggtgacagtttactacggcgttcctgtgtggaaagacgccgaaacgacattgttttgcgcgagtgatgcaaaggcatacgaaactgaaaaacacaacgtctgggctacgcacgcctgcgtgccgaccgatccgaatccgcaagagatccatctggagaacgttaccgaagaatttaatatgtggaagaacaatatggtggaacaaatgcacaccgatataatttccttgtgggatcaaagtttgaaaccttgcgtcaaactcactcctttgtgtgtcactctgcaatgtactaacgtcacaaacaatatcaccgacgatatgaggggagagttgaaaaattgtagctttaatatgacgacagaactgcgggataagaaacagaaagtctacagcctcttctacaggctggatgtcgtgcagatcaatgagaaccagggcaataggtcaaacaactctaacaaagaataccggctcataaattgtaacaccagcgccatcactcaagcttgccccaaggtgtcattcgagccgatcccaatccattactgtgctcccgcaggcttcgcaattttgaagtgtaaggataaaaaatttaacggtactggtccttgtccctccgtctctactgtgcagtgtactcatggtatcaagccggtggtgtcaacccagttgctcttgaatggaagcctcgcagaggaggaagtcatgatccgcagcgagaacataacgaacaatgcgaagaatattctcgtccaatttaatacgcctgttcagatcaactgcactcgccccaataataatacgcgcaagtcaattaggatcggccctggccaagctttttatgcaacaggtgatatcattggggatatccgccaagcgcactgtactgtcagtaaagcaacctggaatgagactttgggcaaggttgtcaagcaactgcggaaacatttcgggaacaatacaatcataagatttgccaatagctctggaggtgatctggaagtcacgacgcacagctttaattgcggaggggaattcttttactgtaatacctccgggctgttcaatagtacgtggatcagcaatacgtccgttcaaggatctaatagtacggggtccaacgatagcattactttgccatgtcgcattaaacagattatcaatatgtggcagagaattggtcaagccatgtatgcacctccaatccaaggcgttatacgctgcgtctcaaacatcaccggtttgatcctgaccagagacggcggatccacgaactccactaccgaaacattccgccccggaggcggagacatgagagacaattggcggtctgaactgtataaatacaaggtggttaaaatcgaaccgctgggtgtcgcacccacgagggctaaacgccgcgtcgttggcagagaaaagcgggcagttgggattggcgctgtgttcttggggttcctgggggcagcggggtctactatgggcgctgcaagcatgactttgacggtccaagccaggaacttgctcagtggtatcgtccaacagcagtccaacctgctgagggcgattgaggcacaacagcatctgttgaagctcactgtttggggaataaagcaattgcaagcaagagtgctggcggttgaaaggtatctgcgggatcaacagttgctcggtatctgggggtgctctgggaaactcatatgtacaactaatgttccatggaactcctcatggtctaataggaacctgagcgaaatatgggataatatgacctggctgcagtgggacaaagaaattagtaactacacacaaatcatttatgggctcctggaggagtctcagaatcagcaggagaaaaatgaacaagacttgttggcactcgataagtgggccagcttgtggaactggtttgacatctccaactggctgtggtacataaaaatattcatcatgatagttggaggtctgatcggactgaggatcgtgtttgctgtgttgtccgtcatccaccgggttcgccaaggttacagtccactcagttttcaaacacacacgcctaatcctagaggcctcgaccgccctgaaagaattgaagaggaggatggtgaacaagatcggggtcgcagtactaggctcgtttctggtttcttggcactcgcgtgggatgatttgagatcactctgtttgttttgttatcaccgcttgcgcgactttattctcattgctgcacgcattgtggagttgctcggacactccagcttgaagggcctgaggttgggttgggagggcttgaagtatctgtggaatctcctggcctactgggggagagagttgaaaatatctgcgataaacttgtttgatactatagcgatcgctgttgcagaatggacagatagagtcatagagatcggtcagcgcttgtgccgggcttttctgcacatccctagacgcatacgccaggggctcgaaagagcattgctgtgataa

SEQ ID NO: 181 - RNA, 12285 nt, synthetic construct
  source 1..12285: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgcgcgttatgggtatccaaagaaattgtcagcacctgttccggtgggggacaatgattctcggcatgattatcatatgctccgctgcggagaatttgtgggtgacagtttactacggcgttcctgtgtggaaagacgccgaaacgacattgttttgcgcgagtgatgcaaaggcatacgaaactgaaaaacacaacgtctgggctacgcacgcctgcgtgccgaccgatccgaatccgcaagagatccatctggagaacgttaccgaagaatttaatatgtggaagaacaatatggtggaacaaatgcacaccgatataatttccttgtgggatcaaagtttgaaaccttgcgtcaaactcactcctttgtgtgtcactctgcaatgtactaacgtcacaaacaatatcaccgacgatatgaggggagagttgaaaaattgtagctttaatatgacgacagaactgcgggataagaaacagaaagtctacagcctcttctacaggctggatgtcgtgcagatcaatgagaaccagggcaataggtcaaacaactctaacaaagaataccggctcataaattgtaacaccagcgccatcactcaagcttgccccaaggtgtcattcgagccgatcccaatccattactgtgctcccgcaggcttcgcaattttgaagtgtaaggataaaaaatttaacggtactggtccttgtccctccgtctctactgtgcagtgtactcatggtatcaagccggtggtgtcaacccagttgctcttgaatggaagcctcgcagaggaggaagtcatgatccgcagcgagaacataacgaacaatgcgaagaatattctcgtccaatttaatacgcctgttcagatcaactgcactcgccccaataataatacgcgcaagtcaattaggatcggccctggccaagctttttatgcaacaggtgatatcattggggatatccgccaagcgcactgtactgtcagtaaagcaacctggaatgagactttgggcaaggttgtcaagcaactgcggaaacatttcgggaacaatacaatcataagatttgccaatagctctggaggtgatctggaagtcacgacgcacagctttaattgcggaggggaattcttttactgtaatacctccgggctgttcaatagtacgtggatcagcaatacgtccgttcaaggatctaatagtacggggtccaacgatagcattactttgccatgtcgcattaaacagattatcaatatgtggcagagaattggtcaagccatgtatgcacctccaatccaaggcgttatacgctgcgtctcaaacatcaccggtttgatcctgaccagagacggcggatccacgaactccactaccgaaacattccgccccggaggcggagacatgagagacaattggcggtctgaactgtataaatacaaggtggttaaaatcgaaccgctgggtgtcgcacccacgagggctaaacgccgcgtcgttggcagagaaaagcgggcagttgggattggcgctgtgttcttggggttcctgggggcagcggggtctactatgggcgctgcaagcatgactttgacggtccaagccaggaacttgctcagtggtatcgtccaacagcagtccaacctgctgagggcgattgaggcacaacagcatctgttgaagctcactgtttggggaataaagcaattgcaagcaagagtgctggcggttgaaaggtatctgcgggatcaacagttgctcggtatctgggggtgctctgggaaactcatatgtacaactaatgttccatggaactcctcatggtctaataggaacctgagcgaaatatgggataatatgacctggctgcagtgggacaaagaaattagtaactacacacaaatcatttatgggctcctggaggagtctcagaatcagcaggagaaaaatgaacaagacttgttggcactcgataagtgggccagcttgtggaactggtttgacatctccaactggctgtggtacataaaaatattcatcatgatagttggaggtctgatcggactgaggatcgtgtttgctgtgttgtccgtcatccaccgggttcgccaaggttacagtccactcagttttcaaacacacacgcctaatcctagaggcctcgaccgccctgaaagaattgaagaggaggatggtgaacaagatcggggtcgcagtactaggctcgtttctggtttcttggcactcgcgtgggatgatttgagatcactctgtttgttttgttatcaccgcttgcgcgactttattctcattgctgcacgcattgtggagttgctcggacactccagcttgaagggcctgaggttgggttgggagggcttgaagtatctgtggaatctcctggcctactgggggagagagttgaaaatatctgcgataaacttgtttgatactatagcgatcgctgttgcagaatggacagatagagtcatagagatcggtcagcgcttgtgccgggcttttctgcacatccctagacgcatacgccaggggctcgaaagagcattgctgtgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaagcggccgctcgagcgtcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgacgcgttaatacgactcactatag

SEQ ID NO: 182 - protein, 860 aa, synthetic construct
MRVMGIQRNCQHLFRWGTMILGMIIICSAAENLWVTVYYGVPVWKDAETTLFCASDAKAYETEKHNVWATHACVPTDPNPQEIHLENVTEEFNMWKNNMVEQMHTDIISLWDQSLKPCVKLTPLCVTLQCTNVTNNITDDMRGELKNCSFNMTTELRDKKQKVYSLFYRLDVVQINENQGNRSNNSNKEYRLINCNTSAITQACPKVSFEPIPIHYCAPAGFAILKCKDKKFNGTGPCPSVSTVQCTHGIKPVVSTQLLLNGSLAEEEVMIRSENITNNAKNILVQFNTPVQINCTRPNNNTRKSIRIGPGQAFYATGDIIGDIRQAHCTVSKATWNETLGKVVKQLRKHFGNNTIIRFANSSGGDLEVTTHSFNCGGEFFYCNTSGLFNSTWISNTSVQGSNSTGSNDSITLPCRIKQIINMWQRIGQAMYAPPIQGVIRCVSNITGLILTRDGGSTNSTTETFRPGGGDMRDNWRSELYKYKVVKIEPLGVAPTRAKRRVVGREKRAVGIGAVFLGFLGAAGSTMGAASMTLTVQARNLLSGIVQQQSNLLRAIEAQQHLLKLTVWGIKQLQARVLAVERYLRDQQLLGIWGCSGKLICTTNVPWNSSWSNRNLSEIWDNMTWLQWDKEISNYTQIIYGLLEESQNQQEKNEQDLLALDKWASLWNWFDISNWLWYIKIFIMIVGGLIGLRIVFAVLSVIHRVRQGYSPLSFQTHTPNPRGLDRPERIEEEDGEQDRGRSTRLVSGFLALAWDDLRSLCLFCYHRLRDFILIAARIVELLGHSSLKGLRLGWEGLKYLWNLLAYWGRELKISAINLFDTIAIAVAEWTDRVIEIGQRLCRAFLHIPRRIRQGLERALL

SEQ ID NO: 183 - RNA, 12956 nt, synthetic construct
  source 1..12956: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggtgctcaggtaaccaggcagcaaaccggtactcatgaaaatgccaacatagctactaatggctcccatattacgtacaatcaaatcaatttctacaaggatagttacgctgcgtccgcttctaagcaggacttcagtcaggatcctagcaagtttacggaacccgtagttgaaggccttaaggcaggggcacctgtccttaagtcaccgagtgcggaggcttgcggttactctgaccgagtactgcagcttaagctcgggaactctgccatagttacgcaggaagcggcaaactattgctgcgcgtacggggagtggccgaactacctgccagaccatgaggcggtcgctatagacaagccaacacaacctgagacagccacggatcggttctatactcttaaaagcgtaaaatgggagactggctccacaggatggtggtggaagctcccagatgcccttaacaatatcgggatgtttggccagaatgttcaacaccattacctgtatcgcagtggcttcctcattcacgtccagtgtaatgccacaaagtttcatcagggggctctccttgtggtggcgatcccagagcatcagaggggtgcacataatactaatactagtcctggtttcgatgatataatgaaaggggaggaaggagggacgtttaatcatccttatgtcctggatgacgggacctcattggcgtgtgcgacgatcttccctcaccagtggattaatctccggaccaataacagtgcgactatcgtacttccatggatgaacgcggctccgatggattttcccctgaggcataatcagtggacattggctattattccggtcgtacccctgggtactagaaccactagctcaatggttcccataactgtatctattgcgccaatgtgctgtgaatttaatgggctccggcacgctatcacacaaggcgttcctacgtatctcttgccaggctcaggtcagttcctcactactgatgaccatagctccgcacctgccctcccctgttttaacccaacacccgagatgcatatcccagggcaagtccgaaacatgcttgaggttgttcaggtagaatctatgatggagatcaataacacagagagtgcggtagggatggagcgccttaaggttgacatctccgcattgaccgatgttgaccaacttttgtttaacattcccctggatatacagctcgatggccccttgcggaacacgttggtcggaaatatctccaggtactacactcattggtccggcagtctcgaaatgacatttatgttttgcggcagtttcatggcagcgggcaaactgatcctgtgttatacacccccaggcggtagttgtccaacgacgcgagagacggcgatgctcggcacacatatagtgtgggattttggcttgcaatcctcagttaccctcatcataccgtggataagcggcagccattatagaatgttcaacaatgacgctaaaagtacgaacgccaatgtgggatatgtgacctgctttatgcagacgaatctcatcgtaccttctgagtcctcagacacatgcagtttgataggtttcatagccgcaaaggacgatttcagtcttagacttatgcgggacagtccggacattggtcaactggatcaccttcatgctgcggaggcagcatatcagatcgaatcaataattaaaactgctaccgacacagtcaagtccgagataaacgctgaactgggcgtcgtcccgagtcttaatgcagtggaaaccggagccacttctaatactgagccagaagaagcaattcaaactcgaactgtgatcaaccaacacggtgtaagcgagactttggtagaaaatttcctctccagagccgccttggtatcaaaaagaagttttgagtataaagaccacacgagctctacagcacgcgcagacaagaacttctttaaatggacgataaataccagaagttttgtacagctccgcaggaaattggagctcttcacatacctccgatttgacgcggaaataacaattttgaccacagttgcggttaatggtagtggaaataacacgtacgtaggcttgcctgatctgacactgcaggccatgtttgtccctactggtgcactcactccggagaaacaggactccttccattggcagagcgggtcaaatgcgtcagtgttcttcaaaatctccgatccccccgcgaggatcactattccctttatgtgtataaatagcgcctatagcgttttttacgatggctttgccggctttgaaaagaatgggttgtacgggattaatccggccgatacgataggtaacctgtgtgtacgcatagttaacgaacaccagccagtgggtttcactgtaaccgttcgagtgtacatgaaacctaagcacatcaaggcttgggcaccaaggccaccgagaaccctcccatacatgagcattgctaatgcaaattataagggaaaagagagagcaccgaacgcgttgtctgcaattatcggcaatcgggactcagtcaagactatgccacataatatagtcaatacctgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggtccaggctttgactttgctcaggcgataatgaagaaaaacacggttatcgcacgaactgaaaagggcgaatttaccatgctcggcgtgtacgatcgagtcgcggttatcccgacacacgcttccgtgggggaaaccatatatatcaacgatgtagaaaccaaagtcctcgacgcgtgtgcactgagggatcttacagacactaacctggagatcacaatagtgaagctggatcgaaatcagaagttccgagacatccgccattttttgcctagatatgaagacgactacaatgatgctgtactgtccgtgcatacttcaaagtttcctaacatgtacatccctgttggccaggtcactaattacggttttcttaacctgggtgggacgcctactcataggatactgatgtacaattttcctactagagctggacaatgtggtggcgtagtgactaccaccgggaaagtcattggcatacacgtaggaggtaacggggcgcagggattcgcggccatgcttctgcacagctatttctccgacacacaaggtgaaatagtttcatcagagaaatccggtgtgtgcattaacgctcccgcgaaaactaaacttcagcccagcgtgtttcatcaagtattcgaaggaagcaaggaaccggctgtactgaaccccaaggacccccggcttaaaacggatttcgaagaagcgatattttcaaaatatactggtaacaaaatcatgctgatggatgagtatatggaagaagctgtggaccactatgtagggtgcctggaaccgctcgacatctctgtggacccgatcccactcgagtccgctatgtacggcatggacggcctcgaggctttggaccttacaactagcgcgggctttccgtatcttttgcaaggtaagaaaaagcgcgacatctttaaccgccacaccagggatacgagcgagatgacaaaaatgcttgaaaaatatggggtcgatcttccttttgtcactttcgtgaaggacgaattgagatcccgagaaaaggtcgagaaaggtaagtctcgcctcattgaagccagttcacttaatgatagtgttgcgatgcgagttgcttttggtaacctttacgcaacatttcataacaatccaggcacggctacaggatcagcagtaggttgcgacccagacatcttttggtcaaaaatccccattctgctggacggtgaaatttttgcctttgactataccggatacgacgcatccttgtcccctgtatggttcgcatgtctcaaaaaagtcctgataaaactcggttacactcaccagactagttttatagactatctgtgtcatagtgttcacctctacaaagataaaaaatatattgtgaacggtggtatgccgtctggtagttccggaacttccatatttaacacaatgattaataatatcattataaggacgcttctcatcagggtctacaagggtatcgatctggatcaattcaagatgatagcatacggcgacgacgtcattgcttcttacccccataagattgatccaggtctgctggcggaagccggcaagcaatatggactggttatgacacccgctgacaaaggaaccagtttcatcgacacgaattgggaaaacgtgacgttcctgaagcgatacttcagagcagacgatcaatatccctttcttatccatcccgttatgccaatgaaggagatacacgagtcaatccgatggacaaaagacccacggaacacacaagatcacgtccgatcactctgttatcttgcctggcacaatggggaggaggcgtataatgagttctgccggaagattcgaagcgtaccagtaggccgagcactgactctccctgcttattcaagtctgcggcggaagtggttggattccttctagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 184 - RNA, 109 nt, synthetic construct
  source 1..109: mol_type = other RNA
ggccatcctgtttttttccctttttttttttcttttttttttttttttttttttttttttttttttctcctttttttttcctctttttttccttttctttccttttcta

SEQ ID NO: 185 - DNA, 900 bp, synthetic construct
  source 1..900: mol_type = other DNA
atgtccaaaaataaagaccagcgaacggccaagactctggagaggacttgggataccctgaatcatctgttgtttatttcaagttgcctttataaattgaatctcaaatccgtcgcgcaaatcacgctgagcatactcgcaatgattatctcaacttccttgataattgccgccataatcttcattgcaagtgcaaatcataaagtgactcctacaaccgcaataatacaggacgcgaccagccaaattaagaacacgactcccacgtatctcacccaaaatccccaactcggaattagcccaagtaatccgtcagagattacttcacaaatcaccaccatactggcgagcacaactccaggcgtaaaatccaccctccaatctactaccgtcaagactaaaaataccactactactcagacacaacctagtaaacctacgactaagcagcgccagaataaacccccaagcaaaccaaacaacgacttccattttgaagttttcaacttcgtgccttgttctatctgctctaataatccaacatgctgggccatctgcaagcgcattccgaataagaaacccggtaagaaaactacaacgaaacctaccaagaagcctaccctgaaaacgacaaagaaagacccgaaaccgcaaacaaccaaaagtaaagaagtgcccacaactaaacccacagaagaaccaacgataaacacgaccaaaaccaacataataactacgctgcttaccagcaacacgacaggcaacccggaattgaccagtcagatggaaacttttcattcaaccagcagcgaaggtaatccaagtcctagtcaggtgtctacgacatctgaatatccatctcagcctagttcccctccgaacacgccgcgccagtgataa

SEQ ID NO: 186 - DNA, 1731 bp, synthetic construct
  source 1..1731: mol_type = other DNA
atggagctgcttatcctgaaagcgaacgctattactactattcttacggcggtaacattctgtttcgcctccgggcagaacataaccgaggagttttatcagagcacatgctctgctgtgtcaaaggggtacctctcagcgttgcgcacggggtggtacactagtgtcatcactatcgagcttagcaatataaagaaaaacaagtgcaacggaacagacgccaaggtaaaattgataaaacaggaactggacaaatataaaaacgctgtaaccgaactccagctcttgatgcaaagcacgcaagcaaccaacaacagggccagaagagagctccctcgcttcatgaactacacactgaataatgccaaaaaaacaaatgtgacgctttcaaagaagcgcaaaagacgctttcttggtttcttgctcggtgtgggatcagccatagcatccggggtcgctgttagtaaagttctccatcttgagggagaggtcaacaagataaaatccgccctcctgtccactaataaagccgtagtctcattgagcaacggagtgagtgttctgacctctaaggtccttgacctcaagaactatatcgacaaacagttgttgcccatcgtaaataaacagtcttgtagcatctccaatatcgaaaccgtgattgaatttcaacaaaagaacaaccggcttctcgaaattacccgcgagttttctgtcaatgcgggtgttactacgccagtgtcaacttacatgctgacaaactcagagttgttgagtctgatcaatgacatgccgataaccaacgaccagaagaaattgatgtccaataacgtgcaaatcgtacggcaacagtcatactccattatgtcaataataaaagaggaagtgttggcatatgtagtccagctgccactttacggagtgatagacacgccatgttggaagttgcacacaagtccattgtgcactacgaacacaaaagagggaagcaacatttgccttacgcgaacagatagaggatggtactgtgacaatgccggcagcgtgtccttcttcccgcaggctgaaacatgcaaggtccagagcaatcgggtgttctgcgatactatgaactccctgactttgccttctgaggtaaacctctgtaacgtagacatatttaacccaaagtacgattgcaaaataatgacatccaagactgacgtgtctagtagcgtaatcacctctcttggagccattgtcagctgttatggtaaaacaaaatgcacagcaagtaacaaaaaccgcggtatcatcaagaccttcagcaacggatgtgactatgtttctaataaaggcgtggatactgtgagtgttggtaataccctttattatgtaaataaacaagaggggaaaagcctgtacgtcaaaggagaaccgatcataaatttctatgatccgctcgtatttccaagcgatgagttcgacgccagcatctctcaggtcaatgagaaaatcaatcagtcattggcatttatccggaaaagcgacgagctcctgcacaacgtaaatgcggggaagtctacgacaaacataatgatcacgacgataataattgttatcattgttattctcctttcacttatagcggtagggttgctgctctattgcaaggctaggagtacccccgtcacattgtctaaggatcaactctctggtataaataacatagcattcagcaattgatgataa

SEQ ID NO: 187 - RNA, 5101 nt, synthetic construct
  source 1..5101: mol_type = other RNA
atgggtgctcaggtaaccaggcagcaaaccggtactcatgaaaatgccaacatagctactaatggctcccatattacgtacaatcaaatcaatttctacaaggatagttacgctgcgtccgcttctaagcaggacttcagtcaggatcctagcaagtttacggaacccgtagttgaaggccttaaggcaggggcacctgtccttaagtcaccgagtgcggaggcttgcggttactctgaccgagtactgcagcttaagctcgggaactctgccatagttacgcaggaagcggcaaactattgctgcgcgtacggggagtggccgaactacctgccagaccatgaggcggtcgctatagacaagccaacacaacctgagacagccacggatcggttctatactcttaaaagcgtaaaatgggagactggctccacaggatggtggtggaagctcccagatgcccttaacaatatcgggatgtttggccagaatgttcaacaccattacctgtatcgcagtggcttcctcattcacgtccagtgtaatgccacaaagtttcatcagggggctctccttgtggtggcgatcccagagcatcagaggggtgcacataatactaatactagtcctggtttcgatgatataatgaaaggggaggaaggagggacgtttaatcatccttatgtcctggatgacgggacctcattggcgtgtgcgacgatcttccctcaccagtggattaatctccggaccaataacagtgcgactatcgtacttccatggatgaacgcggctccgatggattttcccctgaggcataatcagtggacattggctattattccggtcgtacccctgggtactagaaccactagctcaatggttcccataactgtatctattgcgccaatgtgctgtgaatttaatgggctccggcacgctatcacacaaggcgttcctacgtatctcttgccaggctcaggtcagttcctcactactgatgaccatagctccgcacctgccctcccctgttttaacccaacacccgagatgcatatcccagggcaagtccgaaacatgcttgaggttgttcaggtagaatctatgatggagatcaataacacagagagtgcggtagggatggagcgccttaaggttgacatctccgcattgaccgatgttgaccaacttttgtttaacattcccctggatatacagctcgatggccccttgcggaacacgttggtcggaaatatctccaggtactacactcattggtccggcagtctcgaaatgacatttatgttttgcggcagtttcatggcagcgggcaaactgatcctgtgttatacacccccaggcggtagttgtccaacgacgcgagagacggcgatgctcggcacacatatagtgtgggattttggcttgcaatcctcagttaccctcatcataccgtggataagcggcagccattatagaatgttcaacaatgacgctaaaagtacgaacgccaatgtgggatatgtgacctgctttatgcagacgaatctcatcgtaccttctgagtcctcagacacatgcagtttgataggtttcatagccgcaaaggacgatttcagtcttagacttatgcgggacagtccggacattggtcaactggatcaccttcatgctgcggaggcagcatatcagatcgaatcaataattaaaactgctaccgacacagtcaagtccgagataaacgctgaactgggcgtcgtcccgagtcttaatgcagtggaaaccggagccacttctaatactgagccagaagaagcaattcaaactcgaactgtgatcaaccaacacggtgtaagcgagactttggtagaaaatttcctctccagagccgccttggtatcaaaaagaagttttgagtataaagaccacacgagctctacagcacgcgcagacaagaacttctttaaatggacgataaataccagaagttttgtacagctccgcaggaaattggagctcttcacatacctccgatttgacgcggaaataacaattttgaccacagttgcggttaatggtagtggaaataacacgtacgtaggcttgcctgatctgacactgcaggccatgtttgtccctactggtgcactcactccggagaaacaggactccttccattggcagagcgggtcaaatgcgtcagtgttcttcaaaatctccgatccccccgcgaggatcactattccctttatgtgtataaatagcgcctatagcgttttttacgatggctttgccggctttgaaaagaatgggttgtacgggattaatccggccgatacgataggtaacctgtgtgtacgcatagttaacgaacaccagccagtgggtttcactgtaaccgttcgagtgtacatgaaacctaagcacatcaaggcttgggcaccaaggccaccgagaaccctcccatacatgagcattgctaatgcaaattataagggaaaagagagagcaccgaacgcgttgtctgcaattatcggcaatcgggactcagtcaagactatgccacataatatagtcaatacctgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggtccaggctttgactttgctcaggcgataatgaagaaaaacacggttatcgcacgaactgaaaagggcgaatttaccatgctcggcgtgtacgatcgagtcgcggttatcccgacacacgcttccgtgggggaaaccatatatatcaacgatgtagaaaccaaagtcctcgacgcgtgtgcactgagggatcttacagacactaacctggagatcacaatagtgaagctggatcgaaatcagaagttccgagacatccgccattttttgcctagatatgaagacgactacaatgatgctgtactgtccgtgcatacttcaaagtttcctaacatgtacatccctgttggccaggtcactaattacggttttcttaacctgggtgggacgcctactcataggatactgatgtacaattttcctactagagctggacaatgtggtggcgtagtgactaccaccgggaaagtcattggcatacacgtaggaggtaacggggcgcagggattcgcggccatgcttctgcacagctatttctccgacacacaaggtgaaatagtttcatcagagaaatccggtgtgtgcattaacgctcccgcgaaaactaaacttcagcccagcgtgtttcatcaagtattcgaaggaagcaaggaaccggctgtactgaaccccaaggacccccggcttaaaacggatttcgaagaagcgatattttcaaaatatactggtaacaaaatcatgctgatggatgagtatatggaagaagctgtggaccactatgtagggtgcctggaaccgctcgacatctctgtggacccgatcccactcgagtccgctatgtacggcatggacggcctcgaggctttggaccttacaactagcgcgggctttccgtatcttttgcaaggtaagaaaaagcgcgacatctttaaccgccacaccagggatacgagcgagatgacaaaaatgcttgaaaaatatggggtcgatcttccttttgtcactttcgtgaaggacgaattgagatcccgagaaaaggtcgagaaaggtaagtctcgcctcattgaagccagttcacttaatgatagtgttgcgatgcgagttgcttttggtaacctttacgcaacatttcataacaatccaggcacggctacaggatcagcagtaggttgcgacccagacatcttttggtcaaaaatccccattctgctggacggtgaaatttttgcctttgactataccggatacgacgcatccttgtcccctgtatggttcgcatgtctcaaaaaagtcctgataaaactcggttacactcaccagactagttttatagactatctgtgtcatagtgttcacctctacaaagataaaaaatatattgtgaacggtggtatgccgtctggtagttccggaacttccatatttaacacaatgattaataatatcattataaggacgcttctcatcagggtctacaagggtatcgatctggatcaattcaagatgatagcatacggcgacgacgtcattgcttcttacccccataagattgatccaggtctgctggcggaagccggcaagcaatatggactggttatgacacccgctgacaaaggaaccagtttcatcgacacgaattgggaaaacgtgacgttcctgaagcgatacttcagagcagacgatcaatatccctttcttatccatcccgttatgccaatgaaggagatacacgagtcaatccgatggacaaaagacccacggaacacacaagatcacgtccgatcactctgttatcttgcctggcacaatggggaggaggcgtataatgagttctgccggaagattcgaagcgtaccagtaggccgagcactgactctccctgcttattcaagtctgcggcggaagtggttggattccttctagtaa

SEQ ID NO: 188 - DNA, 5101 bp, synthetic construct
  source 1..5101: mol_type = other DNA
atgggtgctcaggtaaccaggcagcaaaccggtactcatgaaaatgccaacatagctactaatggctcccatattacgtacaatcaaatcaatttctacaaggatagttacgctgcgtccgcttctaagcaggacttcagtcaggatcctagcaagtttacggaacccgtagttgaaggccttaaggcaggggcacctgtccttaagtcaccgagtgcggaggcttgcggttactctgaccgagtactgcagcttaagctcgggaactctgccatagttacgcaggaagcggcaaactattgctgcgcgtacggggagtggccgaactacctgccagaccatgaggcggtcgctatagacaagccaacacaacctgagacagccacggatcggttctatactcttaaaagcgtaaaatgggagactggctccacaggatggtggtggaagctcccagatgcccttaacaatatcgggatgtttggccagaatgttcaacaccattacctgtatcgcagtggcttcctcattcacgtccagtgtaatgccacaaagtttcatcagggggctctccttgtggtggcgatcccagagcatcagaggggtgcacataatactaatactagtcctggtttcgatgatataatgaaaggggaggaaggagggacgtttaatcatccttatgtcctggatgacgggacctcattggcgtgtgcgacgatcttccctcaccagtggattaatctccggaccaataacagtgcgactatcgtacttccatggatgaacgcggctccgatggattttcccctgaggcataatcagtggacattggctattattccggtcgtacccctgggtactagaaccactagctcaatggttcccataactgtatctattgcgccaatgtgctgtgaatttaatgggctccggcacgctatcacacaaggcgttcctacgtatctcttgccaggctcaggtcagttcctcactactgatgaccatagctccgcacctgccctcccctgttttaacccaacacccgagatgcatatcccagggcaagtccgaaacatgcttgaggttgttcaggtagaatctatgatggagatcaataacacagagagtgcggtagggatggagcgccttaaggttgacatctccgcattgaccgatgttgaccaacttttgtttaacattcccctggatatacagctcgatggccccttgcggaacacgttggtcggaaatatctccaggtactacactcattggtccggcagtctcgaaatgacatttatgttttgcggcagtttcatggcagcgggcaaactgatcctgtgttatacacccccaggcggtagttgtccaacgacgcgagagacggcgatgctcggcacacatatagtgtgggattttggcttgcaatcctcagttaccctcatcataccgtggataagcggcagccattatagaatgttcaacaatgacgctaaaagtacgaacgccaatgtgggatatgtgacctgctttatgcagacgaatctcatcgtaccttctgagtcctcagacacatgcagtttgataggtttcatagccgcaaaggacgatttcagtcttagacttatgcgggacagtccggacattggtcaactggatcaccttcatgctgcggaggcagcatatcagatcgaatcaataattaaaactgctaccgacacagtcaagtccgagataaacgctgaactgggcgtcgtcccgagtcttaatgcagtggaaaccggagccacttctaatactgagccagaagaagcaattcaaactcgaactgtgatcaaccaacacggtgtaagcgagactttggtagaaaatttcctctccagagccgccttggtatcaaaaagaagttttgagtataaagaccacacgagctctacagcacgcgcagacaagaacttctttaaatggacgataaataccagaagttttgtacagctccgcaggaaattggagctcttcacatacctccgatttgacgcggaaataacaattttgaccacagttgcggttaatggtagtggaaataacacgtacgtaggcttgcctgatctgacactgcaggccatgtttgtccctactggtgcactcactccggagaaacaggactccttccattggcagagcgggtcaaatgcgtcagtgttcttcaaaatctccgatccccccgcgaggatcactattccctttatgtgtataaatagcgcctatagcgttttttacgatggctttgccggctttgaaaagaatgggttgtacgggattaatccggccgatacgataggtaacctgtgtgtacgcatagttaacgaacaccagccagtgggtttcactgtaaccgttcgagtgtacatgaaacctaagcacatcaaggcttgggcaccaaggccaccgagaaccctcccatacatgagcattgctaatgcaaattataagggaaaagagagagcaccgaacgcgttgtctgcaattatcggcaatcgggactcagtcaagactatgccacataatatagtcaatacctgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggtccaggctttgactttgctcaggcgataatgaagaaaaacacggttatcgcacgaactgaaaagggcgaatttaccatgctcggcgtgtacgatcgagtcgcggttatcccgacacacgcttccgtgggggaaaccatatatatcaacgatgtagaaaccaaagtcctcgacgcgtgtgcactgagggatcttacagacactaacctggagatcacaatagtgaagctggatcgaaatcagaagttccgagacatccgccattttttgcctagatatgaagacgactacaatgatgctgtactgtccgtgcatacttcaaagtttcctaacatgtacatccctgttggccaggtcactaattacggttttcttaacctgggtgggacgcctactcataggatactgatgtacaattttcctactagagctggacaatgtggtggcgtagtgactaccaccgggaaagtcattggcatacacgtaggaggtaacggggcgcagggattcgcggccatgcttctgcacagctatttctccgacacacaaggtgaaatagtttcatcagagaaatccggtgtgtgcattaacgctcccgcgaaaactaaacttcagcccagcgtgtttcatcaagtattcgaaggaagcaaggaaccggctgtactgaaccccaaggacccccggcttaaaacggatttcgaagaagcgatattttcaaaatatactggtaacaaaatcatgctgatggatgagtatatggaagaagctgtggaccactatgtagggtgcctggaaccgctcgacatctctgtggacccgatcccactcgagtccgctatgtacggcatggacggcctcgaggctttggaccttacaactagcgcgggctttccgtatcttttgcaaggtaagaaaaagcgcgacatctttaaccgccacaccagggatacgagcgagatgacaaaaatgcttgaaaaatatggggtcgatcttccttttgtcactttcgtgaaggacgaattgagatcccgagaaaaggtcgagaaaggtaagtctcgcctcattgaagccagttcacttaatgatagtgttgcgatgcgagttgcttttggtaacctttacgcaacatttcataacaatccaggcacggctacaggatcagcagtaggttgcgacccagacatcttttggtcaaaaatccccattctgctggacggtgaaatttttgcctttgactataccggatacgacgcatccttgtcccctgtatggttcgcatgtctcaaaaaagtcctgataaaactcggttacactcaccagactagttttatagactatctgtgtcatagtgttcacctctacaaagataaaaaatatattgtgaacggtggtatgccgtctggtagttccggaacttccatatttaacacaatgattaataatatcattataaggacgcttctcatcagggtctacaagggtatcgatctggatcaattcaagatgatagcatacggcgacgacgtcattgcttcttacccccataagattgatccaggtctgctggcggaagccggcaagcaatatggactggttatgacacccgctgacaaaggaaccagtttcatcgacacgaattgggaaaacgtgacgttcctgaagcgatacttcagagcagacgatcaatatccctttcttatccatcccgttatgccaatgaaggagatacacgagtcaatccgatggacaaaagacccacggaacacacaagatcacgtccgatcactctgttatcttgcctggcacaatggggaggaggcgtataatgagttctgccggaagattcgaagcgtaccagtaggccgagcactgactctccctgcttattcaagtctgcggcggaagtggttggattccttctagtaa

SEQ ID NO: 189 - RNA, 5098 nt, synthetic construct
  source 1..5098: mol_type = other RNA
atgggtgctcaagtcactcgccagcaaaccgggacacacgaaaatgctaacatcgcaactaatggcagccacataacgtacaaccagatcaatttttataaggattcatacgcagcatcagccagtaagcaagacttctcacaagacccatcaaagttcaccgaaccagtagtagagggactgaaagcgggtgctccggtactgaagtccccctcagctgaagcttgtggctacagcgatagggttcttcaactcaaacttgggaatagcgcgattgtcacacaggaagcggccaactactgctgcgcatatggggaatggcccaattatttgcctgatcatgaggcagtagctattgacaaacctactcagccagaaaccgccactgatcgattttatactctgaaaagcgtaaagtgggagactgaaagcacaggatggtggtggaaattgcctgatgcgctcaataatattggaatgtttgggcaaaatgttcaataccactacctctatagatccggcttcctcattcacgttcagtgtaacgcgactaagttccaccaaggcgcccttttggtcgtggcaattcccgagcaccaacgaggggcttataacactacaacttcacccgggttcacggacataatgaaaggtgaggaggggggaacgttctcccatccgtatgtcttggacgacggcacgtccctcgcctgcgctacgatctttcctcaccaatggataaatctccggacgaacaatagcgccacaatagtgttgccttggatgaacgcggcccctatggatttccctctgcgccacaatcagtggacccttgcaataatcccggttgtaccacttggaacccgcacggttagctctatggtgccgattacagtatccattgcgccgatgtgctgcgaattcaatggactgcgacacgcgatcactcaaggagtccctacctatctgctgccgggctccggacagttcctcacgacggacgaccatagctcagcacctgtactgccttgctttaatccaactcccgagatgcatatcccaggccaggttcgaaacatgctggaagttgtacaagtcgagagtatgatggaaataaataatactgagaatgcggtcggcatgcaaagactgaaagtcgatatctccgtacttaccgatgtcgaccagctcctctttaacattccacttgatatacagctggacggcccgcttcgaaatacccttgtcgggaatatatcccggtattatacgcattggtctgggtctctcgaaatgacctttatgttttgtgggagctttatggcaaccggaaagctgattttgtgctatactccaccaggcggtagttgccccacgacacgcgaaactgcaatgttggggacgcatatcgtttgggatttcggccttcaatcctcagtcacccttgtgattccgtggattagtggttctcattatcgcatgtttaataatgatgccaagtctactaatgctaacgttggctatgtgacgtgctttatgcagaccaatctcatcgtacccagtgaaagtagcaacacgtgctctctgatcggtttcgtggctgcgaaggacgatttcagccttcgacttatgagggattcccccgatatccggcaattggaccatcttgatgcagccgaagctgcataccagatagagtcaatcattaaaacggcaaccgataccgtgaaatcagaaattaatgccgaactgggggtggtcccctcactgaatgctgttgaaacgggagcatcaagcaacaccgagccagaggaggcgatacagacgcggaccgtgataaatcagcacggggtctccgaaactttggtcgagaacttcctttctcgagcagctctggtttcaaagaggtcctttgaatataagaaccacgccagttcagaagcgcagacagataagaacttcttcaaatggacaataaatacgaagagcttcgtgcagctcagacggaaactggaactctttacttacttgagattcgatgcggaggtgactatactgactacagtggcagtcagcagtagcaatagtacctataccggtctccctgacttgacccttcaagcgatgttcgtaccgacgggagcgcttacaccagagaaacaggacagtttccactggcaatctgcatcaaacgcgagtgttttctttaaaatttccgatcctccggcaagaatgactatcccattcatgtgtattaactccgcatactctgtcttctacgatggattcgccggtttcgaaaagagcggactctatggaattaacccagctgatacaataggtaacctctgtgtgcgcattgttaacgaacaccagcccatcggtttcactgtcacagtccgggtatacatgaaaccaaagcatataaaagcgtgggcgcccagaccaccccggaccctgccgtacatgagcatcgccaatgccaattaccgaggcaaagagagggcccccaacgcgctcaacgcaataatcggcaacagggacagcgtcaaaacaatgccccataacattgtgactacgtgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggggcctggctttgatttcgcacaggccataatgaagaaaaacaccgttattgcacgaaccgaaaaaggtgagttcacaatgctcggtatttacgacagagttgccgtcatacctacccatgcatccgtaggggaaacaatttatataaatgacgtggagactaaagttctcgatgcctgcgccctccgcgatttgactgacaccaacttggaaataacaattgttaagatggaccgcaatcaaaaattccgagacataaggcactttctcccccgatacgaggacgactataacgatgcggtgctcagtgtccacacctcaaaattccctaatatgtacatccccgtcggtcaagtgactaactatggatttttgaatttgggaggcacaccgactcaccgcatactcatgtacaatttccccacacgggcagggcagtgtggtggtgtagtcacgacaacaggaaaggtgatcgggatccatgtcggtggtaacggcgcacaagggtttgctgcaatgcttctccactcttattttactgacactcagggcgaaatcgtcagctccgaaaagagtggagtttgtataaatgcacccgctaaaacgaagctgcaaccgagtgtctttcaccaagtgttcgagggttcaaaagaacccgctgtactcaacagtaaggactccaggctcaaaacagattttgaggaagctatcttctctaaatacacgggaaataaaatcatgctgatggatgaatatatggaagaagccgttgaccattacgttgggtgtctggagccgttggatatctcaattgatcccatcccccttgaatctgctatgtatggcatggacggtctcgaggcgcttgatttgacgacatccgctggattcccttatttgttgcaaggtaaaaagaagcgcgacatatttaacagacagactagggataccactgagatgactcgcatgttggagaaatacggtgttgatcttccatttgttacttttgtgaaagacgaattgcgaagcagggaaaaggttgaaaagggtaagtctagacttatcgaagcctcttcattgaatgattcagtggcaatgagggttgcctttggaaacttgtacgccacgtttcataataatcccggaacggcgacagggtctgcggttgggtgtgatcccgacgtattttggagcaaaatacccatcttgctcaacggtgaaatatttgccttcgattacaccgggtatgacgcgtctctgagcccagtgtggtttgcgtgcctgaagaaggttttgataaaattgggctatacacaccagacttcttttattgactacctgtgccattctgttcacctctataaagaccgaaagtatattgttaatggaggaatgccgtcaggatcctctggtacttccatcttcaatactatgatcaataatatcattatacgcacactgctcatcagagtgtacaaaggtatagacctggatcaatttaagatgattgcctatggtgacgacgtgattgcgagctacccgcataagatcgacccaggcttgttggctgaggctggaaagcattacggactcatcatgactccagcggataagggaacgtcctttgtggataccaattgggaaaatgttacgtttcttaaacgatatttcagggcagacgatcaatacccgttccttatccaccccgtgatgccgatgaaagaaatacacgaaagcattagatggactaaagacccaagaaacacgcaagatcacgttaggagtctttgttacttggcttggcataatggtgaagaggcatacaatgaattctgtaggaagatacgaagtgtcccagtcggtcgagcactcacactccctgcatacagctctctgagacgcaaatggctcgatagtttctgataa

SEQ ID NO: 190 - DNA, 5098 bp, synthetic construct
  source 1..5098: mol_type = other DNA
atgggtgctcaagtcactcgccagcaaaccgggacacacgaaaatgctaacatcgcaactaatggcagccacataacgtacaaccagatcaatttttataaggattcatacgcagcatcagccagtaagcaagacttctcacaagacccatcaaagttcaccgaaccagtagtagagggactgaaagcgggtgctccggtactgaagtccccctcagctgaagcttgtggctacagcgatagggttcttcaactcaaacttgggaatagcgcgattgtcacacaggaagcggccaactactgctgcgcatatggggaatggcccaattatttgcctgatcatgaggcagtagctattgacaaacctactcagccagaaaccgccactgatcgattttatactctgaaaagcgtaaagtgggagactgaaagcacaggatggtggtggaaattgcctgatgcgctcaataatattggaatgtttgggcaaaatgttcaataccactacctctatagatccggcttcctcattcacgttcagtgtaacgcgactaagttccaccaaggcgcccttttggtcgtggcaattcccgagcaccaacgaggggcttataacactacaacttcacccgggttcacggacataatgaaaggtgaggaggggggaacgttctcccatccgtatgtcttggacgacggcacgtccctcgcctgcgctacgatctttcctcaccaatggataaatctccggacgaacaatagcgccacaatagtgttgccttggatgaacgcggcccctatggatttccctctgcgccacaatcagtggacccttgcaataatcccggttgtaccacttggaacccgcacggttagctctatggtgccgattacagtatccattgcgccgatgtgctgcgaattcaatggactgcgacacgcgatcactcaaggagtccctacctatctgctgccgggctccggacagttcctcacgacggacgaccatagctcagcacctgtactgccttgctttaatccaactcccgagatgcatatcccaggccaggttcgaaacatgctggaagttgtacaagtcgagagtatgatggaaataaataatactgagaatgcggtcggcatgcaaagactgaaagtcgatatctccgtacttaccgatgtcgaccagctcctctttaacattccacttgatatacagctggacggcccgcttcgaaatacccttgtcgggaatatatcccggtattatacgcattggtctgggtctctcgaaatgacctttatgttttgtgggagctttatggcaaccggaaagctgattttgtgctatactccaccaggcggtagttgccccacgacacgcgaaactgcaatgttggggacgcatatcgtttgggatttcggccttcaatcctcagtcacccttgtgattccgtggattagtggttctcattatcgcatgtttaataatgatgccaagtctactaatgctaacgttggctatgtgacgtgctttatgcagaccaatctcatcgtacccagtgaaagtagcaacacgtgctctctgatcggtttcgtggctgcgaaggacgatttcagccttcgacttatgagggattcccccgatatccggcaattggaccatcttgatgcagccgaagctgcataccagatagagtcaatcattaaaacggcaaccgataccgtgaaatcagaaattaatgccgaactgggggtggtcccctcactgaatgctgttgaaacgggagcatcaagcaacaccgagccagaggaggcgatacagacgcggaccgtgataaatcagcacggggtctccgaaactttggtcgagaacttcctttctcgagcagctctggtttcaaagaggtcctttgaatataagaaccacgccagttcagaagcgcagacagataagaacttcttcaaatggacaataaatacgaagagcttcgtgcagctcagacggaaactggaactctttacttacttgagattcgatgcggaggtgactatactgactacagtggcagtcagcagtagcaatagtacctataccggtctccctgacttgacccttcaagcgatgttcgtaccgacgggagcgcttacaccagagaaacaggacagtttccactggcaatctgcatcaaacgcgagtgttttctttaaaatttccgatcctccggcaagaatgactatcccattcatgtgtattaactccgcatactctgtcttctacgatggattcgccggtttcgaaaagagcggactctatggaattaacccagctgatacaataggtaacctctgtgtgcgcattgttaacgaacaccagcccatcggtttcactgtcacagtccgggtatacatgaaaccaaagcatataaaagcgtgggcgcccagaccaccccggaccctgccgtacatgagcatcgccaatgccaattaccgaggcaaagagagggcccccaacgcgctcaacgcaataatcggcaacagggacagcgtcaaaacaatgccccataacattgtgactacgtgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggggcctggctttgatttcgcacaggccataatgaagaaaaacaccgttattgcacgaaccgaaaaaggtgagttcacaatgctcggtatttacgacagagttgccgtcatacctacccatgcatccgtaggggaaacaatttatataaatgacgtggagactaaagttctcgatgcctgcgccctccgcgatttgactgacaccaacttggaaataacaattgttaagatggaccgcaatcaaaaattccgagacataaggcactttctcccccgatacgaggacgactataacgatgcggtgctcagtgtccacacctcaaaattccctaatatgtacatccccgtcggtcaagtgactaactatggatttttgaatttgggaggcacaccgactcaccgcatactcatgtacaatttccccacacgggcagggcagtgtggtggtgtagtcacgacaacaggaaaggtgatcgggatccatgtcggtggtaacggcgcacaagggtttgctgcaatgcttctccactcttattttactgacactcagggcgaaatcgtcagctccgaaaagagtggagtttgtataaatgcacccgctaaaacgaagctgcaaccgagtgtctttcaccaagtgttcgagggttcaaaagaacccgctgtactcaacagtaaggactccaggctcaaaacagattttgaggaagctatcttctctaaatacacgggaaataaaatcatgctgatggatgaatatatggaagaagccgttgaccattacgttgggtgtctggagccgttggatatctcaattgatcccatcccccttgaatctgctatgtatggcatggacggtctcgaggcgcttgatttgacgacatccgctggattcccttatttgttgcaaggtaaaaagaagcgcgacatatttaacagacagactagggataccactgagatgactcgcatgttggagaaatacggtgttgatcttccatttgttacttttgtgaaagacgaattgcgaagcagggaaaaggttgaaaagggtaagtctagacttatcgaagcctcttcattgaatgattcagtggcaatgagggttgcctttggaaacttgtacgccacgtttcataataatcccggaacggcgacagggtctgcggttgggtgtgatcccgacgtattttggagcaaaatacccatcttgctcaacggtgaaatatttgccttcgattacaccgggtatgacgcgtctctgagcccagtgtggtttgcgtgcctgaagaaggttttgataaaattgggctatacacaccagacttcttttattgactacctgtgccattctgttcacctctataaagaccgaaagtatattgttaatggaggaatgccgtcaggatcctctggtacttccatcttcaatactatgatcaataatatcattatacgcacactgctcatcagagtgtacaaaggtatagacctggatcaatttaagatgattgcctatggtgacgacgtgattgcgagctacccgcataagatcgacccaggcttgttggctgaggctggaaagcattacggactcatcatgactccagcggataagggaacgtcctttgtggataccaattgggaaaatgttacgtttcttaaacgatatttcagggcagacgatcaatacccgttccttatccaccccgtgatgccgatgaaagaaatacacgaaagcattagatggactaaagacccaagaaacacgcaagatcacgttaggagtctttgttacttggcttggcataatggtgaagaggcatacaatgaattctgtaggaagatacgaagtgtcccagtcggtcgagcactcacactccctgcatacagctctctgagacgcaaatggctcgatagtttctgataa

SEQ ID NO: 191 - RNA, 5101 nt, synthetic construct
  source 1..5101: mol_type = other RNA
atgggtgcacaagtaacccggcagcagactggaacgcacgagaatgccaacattgcgacgaacggttcccatattacctataaccaaataaatttctataaagactcttacgcggctagcgcaagtaaacaggacttttcccaggatccatcaaaatttacagaaccagttgtggaggggttgaaggcaggagcccccgtgctcaagtccccatccgccgaagcttgtggctattctgatcgcgtcttgcaactgaaactcggaaactcagcgatcgttactcaagaggctgctaactattgttgtgcctacggtgagtggcccaactacttgccagatcacgaagccgtagcaatcgacaaaccaacacagcctgaaactgctaccgacaggttttatactctcaaatccgtcaagtgggaagccggatcaaccggatggtggtggaaactcccggacgcgctgaataatataggtatgtttggccagaatgtgcaacaccactatctgtatcggtctggattcttgatccatgtgcagtgcaacgcgactaaattccatcaaggagctctcttggtcgttgccatacccgagcatcagaggggcgcatataatacgaacacttctcctgggtttgatgatatcatgaagggagaggaaggcggaacgttcaatcacccctatgtgttggatgatggcacttcacttgcatgcgcgaccatctttccccatcaatggataaacctgcgcacaaataacagtgctacgattgtgcttccatggatgaacgcggcgccgatggatttccctcttcggcataaccagtggacacttgcgattatcccggtagttcctttgggaacccggacgatgtctagcatggtccccattaccgtgtcaattgcccccatgtgttgtgagttcaatggcctccgccacgcaattacgcagggtgttcccacttatctcctcccgggatccggacaattcttgaccacggacgatcactcaagtgcacctgtcctgccttgcttcagtcccactcctgaaatgcatataccgggccaagttcgaaatatgttggaagttgtccaagtcgaaagtatgatggagattaacaacacggaatccgctgtaggcatggaacgactgaaagtggacatatccgccctcacggacgtcgatcaactcctgttcaacatcccccttgatattcagctcgatggccctctcaggaatacgctggtaggtaatatatctcgatattatacacactggtctggtagcctggagatgacgtttatgttttgtggctcattcatggcgacgggtaaactgatactgtgctacacgccgcctggtggcagctgcccaactacacgcgagacagcgatgctggggacccatatcgtgtgggatttcggtctgcaaagttctgtgacactcataatcccttggataagtgggagtcactacaggatgtttaacaacgatgcaaaatctactaacgccaatgtgggttacgtgacctgctttatgcaaacgaacttgattgttccgagcgaatcaagtgatacgtgttctctcattgggttcattgcggctaaagacgatttctccctccgacttatgcgcgattccccagacattggtcagctggatcatcttgatgctgctgaggcagcttatcaaattgagagcatcatcaagaccgcgaccgacactgtgaaatccgagataaatgcggagcttggagttgtaccgtccttgaatgcggtcgagactggtgctaccagtaatacggagcctgaagaggccatacaaacgaggaccgttatcaatcaacatggcgtttccgaaaccctcgttgaaaatttcctcagccgcgcggcacttgtctctaagaggtctttcgaatacaaaaaccacacgtccagtgaagcgcaaaccgataaaaatttcttcaagtggactatcaatactcgaagcttcgttcagctccggcggaagctggaactgtttacctacctccgcttcgatgcagaaattactatactgaccacagtggcagtaaatggctcaagtagtaacgcgtacatggggctgcctgaccttacgttgcaagcgatgttcgttcctacaggcgcgctcacacccaaacagcaggatagcttccactggcagagtggcagcaacgcatctgtgtttttcaaaataagcgacccccctgcaaggatgacaattccattcatgtgtataaattctgcttattccatcttttacgacggttttgccggtttcgaaaagtctgggctctatggcatcaacccagcggatacgatcggcaatctttgtgtccggatagtaaatgagcaccaacccataggcttcactgtcacagtgcgggtctacatgaaaccgaaacacatcaaagcatgggctccacgaccaccccgaacgctcccttatatgtccattgcgaatgccaactacagaggaaaggaacgggcgcctaacgcgctcaatgcaattataggaaacagggatagtgtcaagactatgccgcataatatagtaacgacctgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggaccgggattcgactttgcgcaagcgattatgaagaagaacacagtagtcgcacgaaccgagaaaggcgagtttactatgttgggtgtgtatgatagggttgccgtaataccaacacacgcaagcgtcggtgagacagtctatatcaacgacgtcgagaccaaagttttggatgcatgcgcactccgcgatttgacagacacaaacttggagattacaatagttaagctcgacagaaaccagaaattccgggatattcggcactttttgcccaaatatgaagatgactacaatgacgcggtcctttcagttcacactagcaagttcccaaacatgtatattcccgtaggtcaggtaaccaattatggatttctcaacttgggaggcactccaacgcacaggatactcatgtataacttcccaacccgcgctggacagtgcggaggggttgtcacaacgaccggtaaggtcatcggcatccacgttggaggtaatggagcgcagggttttgccgccatgttgctccattcctacttcacagatacccaaggagagattgtaagcaacgagaaaagtggggtatgcataaatgcgcctgtgaagacgaaacttcaaccctccgtctttcaccaggtctttgaaggaagtaaagagccagctgtccttaattctaaagaccctagactcaaaacggactttgaggaggcgattttttcaaagtacactggaaacaagattatgcttatggacgaatatatggaggaagccgtagaccattacgttggatgtctggaaccgttggatatttctgttgatcctattcctttggaatcagctatgtacgggatggacgggcttgaggcactcgacctgaccacctccgctgggtttccctatctgctgcaagggaaaaaaaagcgggatatttttaatagacatactagagacacgacggaaatgactaaaatgctggagaagtatggcgtggatctgcctttcgtcaccttcgtaaaagatgaactccggagcaaggaaaaagtcgaaaagggaaaaagtcggcttattgaggcttcttctctcaacgactcagtggcgatgcgagttgcttttggcaacctctacgccacatttcacaataatccgggaacggcaactggcagtgccgtaggctgcgaccccgacattttctggagtaaaattccgatactcttggacggtgaaatttttgccttcgactatacgggatatgacgcgtccttgtccccggtttggtttgcatgtctgaaaaaggtccttgttaagttgggttacacccatcaaacttctttcatagattatttgtgccattctgttcatttgtataaggacaggaagtacattgtaaacggggggatgccttctggttcctctggtacctccattttcaatacaatgattaataacataattatccggactctccttataaaagtttacaagggaatcgacttggatcagtttaagatgatagcctatggagatgacgtaattgcgtcttatcctcacaagatcgaccccggcctgttggcggaagcgggtaagcattatggtctggttatgacgcctgctgataaggggaccagtttcgtggatacgaactgggaaaacgtaaccttcttgaaacggtattttagggctgatgaccaatatccgttcctcattcatcccgtaatgccgatgaaggagatacatgagagtattcggtggacgaaagatccgcgaaatacacaagaccacgtacggtcactgtgttacctggcatggcataacggcgaagaggcctacaatgaattctgtcggaaaattcgaagtgtgcccgtcggacgagctttgactcttccggcttattcctctctcagaaggaaatggcttgatagtttctgataa

SEQ ID NO: 192 - DNA, 5101 bp, synthetic construct
  source 1..5101: mol_type = other DNA
atgggtgcacaagtaacccggcagcagactggaacgcacgagaatgccaacattgcgacgaacggttcccatattacctataaccaaataaatttctataaagactcttacgcggctagcgcaagtaaacaggacttttcccaggatccatcaaaatttacagaaccagttgtggaggggttgaaggcaggagcccccgtgctcaagtccccatccgccgaagcttgtggctattctgatcgcgtcttgcaactgaaactcggaaactcagcgatcgttactcaagaggctgctaactattgttgtgcctacggtgagtggcccaactacttgccagatcacgaagccgtagcaatcgacaaaccaacacagcctgaaactgctaccgacaggttttatactctcaaatccgtcaagtgggaagccggatcaaccggatggtggtggaaactcccggacgcgctgaataatataggtatgtttggccagaatgtgcaacaccactatctgtatcggtctggattcttgatccatgtgcagtgcaacgcgactaaattccatcaaggagctctcttggtcgttgccatacccgagcatcagaggggcgcatataatacgaacacttctcctgggtttgatgatatcatgaagggagaggaaggcggaacgttcaatcacccctatgtgttggatgatggcacttcacttgcatgcgcgaccatctttccccatcaatggataaacctgcgcacaaataacagtgctacgattgtgcttccatggatgaacgcggcgccgatggatttccctcttcggcataaccagtggacacttgcgattatcccggtagttcctttgggaacccggacgatgtctagcatggtccccattaccgtgtcaattgcccccatgtgttgtgagttcaatggcctccgccacgcaattacgcagggtgttcccacttatctcctcccgggatccggacaattcttgaccacggacgatcactcaagtgcacctgtcctgccttgcttcagtcccactcctgaaatgcatataccgggccaagttcgaaatatgttggaagttgtccaagtcgaaagtatgatggagattaacaacacggaatccgctgtaggcatggaacgactgaaagtggacatatccgccctcacggacgtcgatcaactcctgttcaacatcccccttgatattcagctcgatggccctctcaggaatacgctggtaggtaatatatctcgatattatacacactggtctggtagcctggagatgacgtttatgttttgtggctcattcatggcgacgggtaaactgatactgtgctacacgccgcctggtggcagctgcccaactacacgcgagacagcgatgctggggacccatatcgtgtgggatttcggtctgcaaagttctgtgacactcataatcccttggataagtgggagtcactacaggatgtttaacaacgatgcaaaatctactaacgccaatgtgggttacgtgacctgctttatgcaaacgaacttgattgttccgagcgaatcaagtgatacgtgttctctcattgggttcattgcggctaaagacgatttctccctccgacttatgcgcgattccccagacattggtcagctggatcatcttgatgctgctgaggcagcttatcaaattgagagcatcatcaagaccgcgaccgacactgtgaaatccgagataaatgcggagcttggagttgtaccgtccttgaatgcggtcgagactggtgctaccagtaatacggagcctgaagaggccatacaaacgaggaccgttatcaatcaacatggcgtttccgaaaccctcgttgaaaatttcctcagccgcgcggcacttgtctctaagaggtctttcgaatacaaaaaccacacgtccagtgaagcgcaaaccgataaaaatttcttcaagtggactatcaatactcgaagcttcgttcagctccggcggaagctggaactgtttacctacctccgcttcgatgcagaaattactatactgaccacagtggcagtaaatggctcaagtagtaacgcgtacatggggctgcctgaccttacgttgcaagcgatgttcgttcctacaggcgcgctcacacccaaacagcaggatagcttccactggcagagtggcagcaacgcatctgtgtttttcaaaataagcgacccccctgcaaggatgacaattccattcatgtgtataaattctgcttattccatcttttacgacggttttgccggtttcgaaaagtctgggctctatggcatcaacccagcggatacgatcggcaatctttgtgtccggatagtaaatgagcaccaacccataggcttcactgtcacagtgcgggtctacatgaaaccgaaacacatcaaagcatgggctccacgaccaccccgaacgctcccttatatgtccattgcgaatgccaactacagaggaaaggaacgggcgcctaacgcgctcaatgcaattataggaaacagggatagtgtcaagactatgccgcataatatagtaacgacctgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggaccgggattcgactttgcgcaagcgattatgaagaagaacacagtagtcgcacgaaccgagaaaggcgagtttactatgttgggtgtgtatgatagggttgccgtaataccaacacacgcaagcgtcggtgagacagtctatatcaacgacgtcgagaccaaagttttggatgcatgcgcactccgcgatttgacagacacaaacttggagattacaatagttaagctcgacagaaaccagaaattccgggatattcggcactttttgcccaaatatgaagatgactacaatgacgcggtcctttcagttcacactagcaagttcccaaacatgtatattcccgtaggtcaggtaaccaattatggatttctcaacttgggaggcactccaacgcacaggatactcatgtataacttcccaacccgcgctggacagtgcggaggggttgtcacaacgaccggtaaggtcatcggcatccacgttggaggtaatggagcgcagggttttgccgccatgttgctccattcctacttcacagatacccaaggagagattgtaagcaacgagaaaagtggggtatgcataaatgcgcctgtgaagacgaaacttcaaccctccgtctttcaccaggtctttgaaggaagtaaagagccagctgtccttaattctaaagaccctagactcaaaacggactttgaggaggcgattttttcaaagtacactggaaacaagattatgcttatggacgaatatatggaggaagccgtagaccattacgttggatgtctggaaccgttggatatttctgttgatcctattcctttggaatcagctatgtacgggatggacgggcttgaggcactcgacctgaccacctccgctgggtttccctatctgctgcaagggaaaaaaaagcgggatatttttaatagacatactagagacacgacggaaatgactaaaatgctggagaagtatggcgtggatctgcctttcgtcaccttcgtaaaagatgaactccggagcaaggaaaaagtcgaaaagggaaaaagtcggcttattgaggcttcttctctcaacgactcagtggcgatgcgagttgcttttggcaacctctacgccacatttcacaataatccgggaacggcaactggcagtgccgtaggctgcgaccccgacattttctggagtaaaattccgatactcttggacggtgaaatttttgccttcgactatacgggatatgacgcgtccttgtccccggtttggtttgcatgtctgaaaaaggtccttgttaagttgggttacacccatcaaacttctttcatagattatttgtgccattctgttcatttgtataaggacaggaagtacattgtaaacggggggatgccttctggttcctctggtacctccattttcaatacaatgattaataacataattatccggactctccttataaaagtttacaagggaatcgacttggatcagtttaagatgatagcctatggagatgacgtaattgcgtcttatcctcacaagatcgaccccggcctgttggcggaagcgggtaagcattatggtctggttatgacgcctgctgataaggggaccagtttcgtggatacgaactgggaaaacgtaaccttcttgaaacggtattttagggctgatgaccaatatccgttcctcattcatcccgtaatgccgatgaaggagatacatgagagtattcggtggacgaaagatccgcgaaatacacaagaccacgtacggtcactgtgttacctggcatggcataacggcgaagaggcctacaatgaattctgtcggaaaattcgaagtgtgcccgtcggacgagctttgactcttccggcttattcctctctcagaaggaaatggcttgatagtttctgataa

(19) United States
(12) Patent Application Publication
(10) Pub. No.: US 2026/0041754 A1
(43) Pub. Date: Feb. 12, 2026

(54) NANOPARTICLE COMPLEXES FOR ENHANCED SAFETY

(71) Applicant: HDT Bio Corp., Seattle, WA (US)

(72) Inventors: Taishi Kimura, Seattle, WA (US); Bryan J. Berube, Issaquah, WA (US); Jesse H. Erasmus, Port Orchard, WA (US); Adrian C. Simpson, Shoreline, WA (US); Joseph Leal, Seattle, WA (US); Amit P. Khandhar, Issaquah, WA (US); Malcolm S. Duthie, Sammamish, WA (US)

(21) Appl. No.: 19/259,545

(22) Filed: Jul. 3, 2025

Related U.S. Application Data
(63) Continuation of application No. PCT/US24/10326, filed on Jan. 4, 2024.
(60) Provisional application No. 63/478,819, filed on Jan. 6, 2023.

(51) Int. Cl. A61K 39/12; A61K 9/51; A61K 31/7105; A61K 38/45; A61K 39/00

(52) U.S. Cl. CPC A61K 39/12; A61K 9/5192; A61K 31/7105; A61K 38/45; A61K 2039/53; A61K 2039/54; A61K 2039/55555

(57) ABSTRACT

The disclosure provides compositions, methods of treatment, and methods of making and using compositions to deliver a nucleic acid to a subject that, optionally, have reduced reactogenicity and promotes a local innate immune response in the subject while promoting an adaptive immune response. Compositions described herein include nanoparticles, optionally including an inorganic particle, capable of admixing with nucleic acids encoding proteins, antibodies, or immunomodulators. Methods of using the compositions as a therapeutic vaccine for the treatment of an infection or cancer are also provided.

### CROSS-REFERENCE TO RELATED APPLICATION

This application is a continuation of International Application No. PCT/US2024/10326, filed Jan. 4, 2024, which claims the benefit of priority to U.S. Provisional Patent Application No. 63/478,819, filed Jan. 6, 2023, the contents of each are incorporated herein by reference in their entirety.

### STATEMENT AS TO FEDERALLY SPONSORED RESEARCH

This invention was made with government support under Contract number R61AI161811 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jan. 4, 2024, is named 201953-729601_SL.xml and is 345,677 bytes in size.

### BACKGROUND

A challenge with nanoparticle carriers used in conventional RNA-based vaccine compositions is that certain types of nanoparticles, while useful for RNA delivery, can cause unwanted and sometimes serious side effects such as fever, headache, muscle pain, myocarditis and pericarditis due to the over-stimulation of the host immune system systemically. Therefore, there is a great unmet need for safer nanoparticle compositions that yield therapeutically effective amounts of RNA without unwanted reactions to vaccination.

### BRIEF SUMMARY

Provided herein are methods for generating a local immune response, wherein the methods comprise: administering to a tissue in a subject a composition, wherein the composition comprises: nanoparticles, wherein the nanoparticles comprise: a hydrophobic core comprising lipids in liquid phase at 25 degrees Celsius; and nucleic acids, wherein the nucleic acids are complexed to the nanoparticles to form nucleic acid-nanoparticle complexes, and wherein the administering of the composition provides for a local immune response within a tissue.

Provided herein are methods wherein the methods comprise: administering to a tissue in a subject a composition, wherein the composition comprises: administering to a tissue in a subject a composition, wherein the composition comprises: nanoparticles, wherein the nanoparticles comprise: a hydrophobic core comprising lipids in liquid phase at 25 degrees Celsius; and nucleic acids encoding for a protein, wherein the nucleic acids encoding for a protein are complexed to the nanoparticles to form nucleic acid-nanoparticle complexes, wherein the composition comprises a nucleic acid biodistribution ratio between 0 and 0.1 (0<nucleic acid biodistribution ratio ≤0.1) when administered to a subject, and wherein the nucleic acid biodistribution ratio is determined by the formula:

$$\frac{x 1}{x 2} = nucleic acid biodistribution ratio$$

wherein x1 is the level of the nucleic acids in a tissue that is not within or not adjacent to the tissue that was administered the composition,
wherein x2 is the level of the nucleic acids in the tissue administered the composition.

Provided herein are methods wherein the methods comprise: administering to a tissue in a subject a composition, wherein the composition comprises: administering to a tissue in a subject a composition, wherein the composition comprises: nanoparticles, wherein the nanoparticles comprise: a hydrophobic core comprising lipids in liquid phase at 25 degrees Celsius; and nucleic acids encoding for a protein, wherein the nucleic acids encoding for a protein are complexed to the nanoparticles to form nucleic acid-nanoparticle complexes, wherein the composition comprises a nucleic acid biodistribution ratio between 0 and 1 (0<nucleic acid biodistribution ratio ≤1) when administered to a subject, and wherein the nucleic acid biodistribution ratio is determined by the formula:

$$\frac{x 1}{x 2} = nucleic acid biodistribution ratio$$

wherein x1 is the level of the nucleic acids in a tissue that is not within or not adjacent to the tissue that was administered the composition,
wherein x2 is the level of the nucleic acids in the tissue administered the composition.

Provided herein are methods wherein the methods comprise: administering to a tissue in a subject a composition, wherein the composition comprises: administering to a tissue in a subject a composition, wherein the composition comprises: nanoparticles, wherein the nanoparticles comprise: a hydrophobic core comprising lipids in liquid phase at 25 degrees Celsius; and nucleic acids encoding for a protein, wherein the nucleic acids encoding for a protein are complexed to the nanoparticles to form nucleic acid-nanoparticle complexes, wherein the composition comprises a nucleic acid biodistribution ratio that is less than the biodistribution ratio of a reactogenic nanoparticle composition (nucleic acid biodistribution ratio ≤nucleic acid biodistribution ratio of the reactogenic nanoparticle composition) when administered to a subject, and wherein the nucleic acid biodistribution ratio is determined by the formula:

$$\frac{x 1}{x 2} = nucleic acid biodistribution ratio$$

wherein x1 is the level of the nucleic acids in a tissue that is not within or not adjacent to the tissue that was administered the composition,
wherein x2 is the level of the nucleic acids in the tissue administered the composition.

Provided herein are methods for reducing the reactogenicity of a vaccine composition in a subject, the method comprising: administering to a subject a vaccine composition, wherein the vaccine composition comprises: nanoparticles, wherein the nanoparticles comprise: a hydrophobic core comprising lipids in liquid phase at 25 degrees Celsius; and nucleic acids encoding for a microbial protein antigen or nucleic acids encoding for a cancer-associated protein, wherein the nucleic acids encoding for the microbial protein antigen are complexed to the nanoparticles to form nucleic acid-nanoparticle complexes, and wherein the administering to the subject reduces systemic inflammation compared to administration of a reactogenic nanoparticle composition, thereby reducing the reactogenicity of the vaccine composition.

Provided herein are methods for treating a disease or a condition in a subject, the method comprising: administering to a tissue in a subject a composition, wherein the composition comprises: nanoparticles, wherein the nanoparticles comprise: a hydrophobic core comprising lipids in liquid phase at 25 degrees Celsius; and nucleic acids, wherein the nucleic acids are complexed to the nanoparticles to form nucleic acid-lipid nanoparticle complexes, and wherein the administering of the composition provides for a local immune response within a tissue, thereby treating the disease of the condition in the subject.

Provided herein are methods for generating an immune response in a subject to a protein antigen, the method comprising: administering to a tissue in a subject a composition, wherein the composition comprises: nanoparticles, wherein the nanoparticles comprise: a hydrophobic core comprising lipids in liquid phase at 25 degrees Celsius; and nucleic acids, wherein the nucleic acids encode for at least one protein antigen, and wherein the nucleic acids are complexed to the nanoparticles to form nucleic acid-lipid nanoparticle complexes, and wherein the administering of the composition provides for a local immune response within a tissue, thereby generating an immune response to the protein antigen in the subject.

Provided herein are compositions, wherein the compositions comprise: nanoparticles, wherein the nanoparticles comprise: a hydrophobic core comprising lipids in liquid phase at 25 degrees Celsius; and nucleic acids encoding for a human immunodeficiency virus-1 (HIV-1) envelope (env) protein or a functional variant thereof, wherein the nucleic acids encoding for an HIV-1 env protein or a functional variant thereof are complexed to the nanoparticles to form nucleic acid-nanoparticle complexes.

Additional features of the present invention will be apparent to persons of ordinary skill in the art in view of the following disclosure, as well as the accompanying drawings and claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1I show schematic representations of nanoparticle (NP) carriers. FIG. 1A shows an oil-in-water emulsion and nucleic acids. FIG. 1B shows a nanostructured lipid carrier and nucleic acids. FIG. 1C shows a lipid inorganic nanoparticle and nucleic acids. FIG. 1D shows a nanoparticle comprising a cationic lipid membrane, a hydrophobic core, and nucleic acids. FIG. 1E shows a nanoparticle comprising a cationic lipid membrane, a hydrophobic core, inorganic nanoparticles within the membrane of the nanoparticle, and a plurality of nucleic acids. FIG. 1F shows a nanoparticle having a cationic lipid membrane, a liquid oil core (e.g., squalene), and two or more RNA or DNA molecules. FIG. 1G shows a nanoparticle having a cationic lipid membrane, inorganic particles, a liquid oil core, and two or more RNA or DNA molecules. FIG. 1H shows a nanoparticle having a cationic lipid membrane, a solid core (e.g., glyceryl trimyristate-dynasan), and two or more RNA or DNA molecules. FIG. 1I shows a nanoparticle having a cationic lipid membrane (e.g., phospholipids, PEG-lipid), a solid core (e.g., cholesterol, ionizable cationic lipid), and two or more RNA or DNA molecules. Schematics are not to scale.

FIGS. 2A-2D show that IM injection of repRNA/NP-30 induces only minor systemic reactogenicity in mice. FIG. 2A shows a graph of serum type I IFNs in mice received 10 μg of repRNA/NP-30 and repRNA/LNP at indicated time points determined by ELISA. X-axis: hours after intramuscular (IM) injection; Y-axis: serum interferon alpha 2 levels (pg/ml). Conditions: Naïve mice (black), repRNA/NP-30 treated mice (white), and repRNA/LNP treated mice (gray). FIG. 2B shows a graph of the cytokine profiling of sera isolated from 10 μg of repRNA/NP-30 and repRNA/LNP-injected mice at 14 hours were determined by cytokine array. The results from calculation of the values of repRNA/NP-30 divided by the values of repRNA/LNP are shown. X-axis: Log₂ (fold change). Y-axis: −log 10(p value). Cytokines identified in the graph: Intercellular Adhesion Molecule 1 (ICAM-1), interferon-lambda (IFN-k), interleukin 27 p28 (IL-27p28, IL30), pentraxin 3 (PTX3), macrophage colony-stimulating factor (M-CSF), c-reactive peptide (CRP), tumor necrosis factor (TNF), chemokine ligand 12 (CCL12), chemokine ligand 2 (CCL2), chemokine ligand (CCL6), chemokine ligand (CCL9), chemokine (C-X-C motif) ligand 10 (CXCL10), cluster of differentiation 93 (CD93), P-selectin and E-selectin. FIG. 2C shows a graph of cardiac troponin-I (cTNI) levels in the sera of mice that received 10 μg of repRNA/NP-30 and repRNA/LNP determined by ELISA. X-axis: Hours after IM injection; Y-axis: Cardiac Troponin Levels (cTNI) in picograms/milliliter. FIG. 2D shows the epifluorescence of various tissues from MX1-eGFP transgenic mice that received 10 μg of repRNA/NP-30 or repRNA/LNP. Tissues measured: muscle, Delphian lymph nodes (dLN), liver, spleen, and heart. All FIG. 2A-2D graphs: P<0.05*, <0.01**, <0.001***, <0.0001****, ns=not significant.

FIGS. 3A-3G show IM injection of repRNA/NP-30 activates the local muscle innate immune response in mice. FIG. 3A shows a heatmap of NanoString analysis and expression of muscle genes included in the Host Response Panel 14 hours post-IM injection. FIG. 3B shows a volcano plot of genes of special interest from FIG. 3A in repRNA/NP-30-treated naïve mice (left) and in repRNA/LNP treated naïve mice (right) are indicated in dark grey. X-axis: Log₂ fold change; Y-axis: −log₁₀padj. Cytokines identified in the graphs: interleukin 6 (IL6), interleukin-1 receptor antagonist (IL1RN), interleukin 1 alpha (IL1A), interferon beta 1 (IFNB1), interleukin 1 beta (IL1B) CCL3, CCL4, CCL5, CCL77, CCL22, CXCL2, CXCL3, CXCL5, CXCL9, CXCL10, CXCL11, and CXCL13. FIG. 3C shows a volcano plot of the differential expression of genes in muscle genes between repRNA/NP-30 and repRNA/LNP from FIG. 3A. X-axis: Log₂ fold change; Y-axis: −log 10(padj). Genes indicated in the graph: lysozyme 2 (LYZ2), CCR2, Integrin Subunit Beta 2 (ITGB2), Integrin alpha M (ITGAM), CCR5, histocompatibility 2, class II antigen A, beta 1 (H2-AB1), histocompatibility 2, class II, locus Mb1 (H2-DMB1), histocompatibility 2, class II, locus DMa (H2-DMA), CD69, CD86, integrin subunit alpha L (ITGAL), Fos proto-oncogene, AP-1 transcription factor subunit (FOS), IL6, and colony stimulating factor 2 (CSF2). FIG. 3D shows a table showing PanglaoDB cell type analysis for differentially-expressed genes in the muscles of repRNA/NP-30 vs repRNA/LNP. FIG. 3E shows a table from NanoString annotation analysis for the top ranked differentially-expressed genes in the muscles of repRNA/NP-30 vs repRNA/LNP.

FIG. 3F shows graphs of the normalized expression of the co-stimulatory molecule genes for CD40, CD80, and CD86 in the muscles of indicated mice. FIG. 3G shows a graph of normalized expression of type I interferon mRNA in the muscles of indicated mice. X-axis: Conditions; Y-Axis: log 2 normalized IFN mRNA expression. FIG. 3H shows a graph of normalized expression of interferon alpha 2 (Ifna2) mRNA in the muscles of indicated mice. X-axis: Conditions; Y-Axis: log 2 normalized Ifna2 mRNA expression. FIG. 3I shows a graph of normalized expression of interferon alpha 4 (Ifna4) mRNA in the muscles of indicated mice. X-axis: Conditions; Y-Axis: log 2 normalized Ifna4 mRNA expression.

FIGS. 4A-4E show IM injection of repRNA/NP-30 restricts transgene expression to the muscle. FIG. 4A shows images from C57BL/6 mice that received IM injection of 10 μg of repRNA formulated with NP-30-DiR or LNP-DiR. Lipid biodistribution was analyzed by IVIS using XenoLight-DiR-conjugated formulations. FIG. 4B shows a schematic representation of reporter mouse models used in FIG. 4C, FIGS. 4D and 4E. FIG. 4C shows images from LSL-Luc Tg reporter mice that received IM injection of 10 μg of repRNA expressing Cre recombinase. At day 7, luciferase expression was analyzed in vivo by IVIS. Luciferase expression was quantified in the right panel. X-axis: Conditions; Y-axis: Total flux (p/s). FIG. 4D shows images from Ai9 Tg mice that received IM injection of 10 μg of repRNA expressing Cre recombinase. At the indicated time points, individual organs were isolated and tdTomato expression was analyzed by IVIS. FIG. 4E shows images from Ai9 mice that received IM injection of 10 μg of NP-30/repRNA and LNP/repRNA expressing Cre recombinase. 7 days later, the muscle was isolated and the nuclei and tdTomato were stained with Hoechst, and anti-RFP antibody, respectively. P<0.05*, <0.01**, <0.001***, <0.0001****, ns=not significant.

FIGS. 5A-5I shows IM injection of repRNA/NP-30 recruits immune cells to the vicinity of transfected myocytes. C57BL/6 mice received IM injection of 10 μg of repRNA/NP-30. At the indicated time point, mice were sacrificed, and their muscles were processed by skeletal Muscle dissociation kit. FIG. 5A shows a graph of the frequencies of CD11b+ cells in the dLN and muscle for each condition. X-axis: Conditions; Y-axis: % of non-myocytes. Left graph: CD11b+ cells in the lymph nodes. Right graph: CD11b+ cells in the muscle. FIG. 5B shows graphs of the frequencies of the indicated cell types in the isolated non-myocytes as determined by flow cytometry and shown as Means±SD. X-axis: Conditions; Y-axis: % of non-myocytes. Left graph: MHC-IIIo macrophages in the muscle; Center graph: plasmacytoid dendritic cells in the muscle; Right graph: monocyte dendritic cells in the muscle. FIG. 5C shows graphs of the frequencies of the indicated cell types in isolated muscle. X-axis: Conditions; Y-axis: % of non-myocytes. Left graph: conventional dendritic cells type I (DC1) in the muscle; right graph: conventional dendritic cells type 2 (DC2) in the muscle. FIG. 5D shows graphs of the frequencies of the indicated cell types in the muscle tissue. X-axis: Conditions; Y-axis: % of non-myocytes. Left graph: neutrophils; center graph: Natural Killer (NK) cells; right graph: T-cells. FIG. 5E shows a heatmap of the composition of dendritic cells (DC) in the muscles and dLNs of indicated mice. FIG. 5F and FIG. 5G show graphs of the surface CD86 expression levels of the single cells in the muscles (FIG. 5F) and dLNs (FIG. 5G) were determined by flow cytometry, and shown as Means±SD. X-axis: Conditions; Y-axis CD89MFI. FIG. 5H shows graphs of the surface CD86 expression levels of the single cells in the lymph nodes (dLNs). X-axis: Conditions; Y-axis: CD86 MFI. Left graph: MHC-II^{lo} macrophages in the lymph nodes; center graph: plasmacytoid dendritic cells in the muscle; Right graph: monocyte dendritic cells in the lymph nodes (dLNs). FIG. 5I shows graphs of the surface CD86 expression levels of the single cells in the lymph nodes (dLNs). X-axis: Conditions; Y-axis: CD86 MFI. Left graph: conventional dendritic cells type 1 (DC1); right graph: conventional dendritic cells type 2 (DC2). All graphs in FIGS. 5A-5I: P<0.05*, <0.01**, <0.001***, <0.0001****, ns=not significant.

FIGS. 6A-6D show antibody responses to repRNA-encoded antigen delivered to mice with NP-30 and LNP in the presence and absence of interferon receptor 1 antibodies (α-IFNR1). FIG. 6A shows images of reporter gene expression in the muscle-injected site at day 1 in response to blockade of type I IFN signaling in repRNA/NP-30 and repRNA/LNP-injected mice. A graph is shown on the right of quantified firefly luciferase flux after repRNA injection. X-axis: days after injection; Y-axis: total flux (p/s). Conditions: no antibody+repRNA/NP-30; repRNA/NP-30 +IFNAR1 antibody blockade; no antibody+repRNA/LNP; repRNA/LNP+IFNAR1 antibody blockade, naïve untreated mice. FIG. 6B shows the dosing schedule for assaying antibody responses four weeks after the prime and two weeks after the second dose of repRNA encoding SARS-CoV2 Spike protein delivered via NP-30 or LNP. FIG. 6C shows a graph of the total IgG titer at day 28 post-prime for mice treated with repRNA/NP-30 and repRNA/LNP. X-axis: conditions; Y-axis: Total IgG titer (micrograms/milliliter, pg/ml). FIG. 6D shows a graph of the total IgG titer at day 49 (two weeks after the boost). X-axis: Conditions; Y-axis: Total IgG titer (micrograms/milliliter, pg/ml). All graphs in FIGS. 6A-6D: P<0.05*, <0.01**, <0.001***, <0.0001****, ns=not significant.

FIGS. 7A-7G show that intramuscular (IM) injection of repRNA encoding the SARS-CoV-2 Spike induces only minor systemic responses compared to IM injection of repRNA/LNP. FIG. 7A shows a graph of serum IFN-alpha levels 14 hours following treatment as indicated. X-Axis: dose; Y-axis: serum interferon-alpha 2 levels (picograms/milliliter, pg/ml). FIG. 7B shows a graph of serum IFN-alpha 2 levels in Ai9 Tg mice measured at the indicated time points after receiving IM injection of 10 μg of repRNA expressing Cre formulated with NP-30 or LNP. X-Axis: hours following IM injection; Y-axis: serum interferon-alpha 2 levels (picograms/milliliter, pg/ml). Conditions: Naïve mice; repRNA/NP-30 treated mice; and repRNA/LNP treated mice. FIG. 7C shows a graph of serum IFN-alpha levels at 14 hours following IM injection of 10 μg of 5′-triphosphate RNA (5′-pppRNA) formulated with NP-30 or LNP in C57BL/6 mice. X-Axis: conditions; Y-axis: serum interferon-alpha 2 levels (picograms/milliliter, pg/ml). FIG. 7D shows a graph of serum IFN-alpha levels at indicated time points in C57BL/6 mice that received IM injection of 10 μg of mRNA expressing secreted enzyme alkaline phosphatase (SEAP) formulated with NP-30 or LNP. X-Axis: hours following IM injection; Y-axis: serum interferon-alpha 2 levels (picograms/milliliter, pg/ml). FIG. 7E shows a heatmap of cytokine profiling in sera 14 hours after IM injection in C57BL/6 mice that received IM injection of 10 μg of repRNA expressing SEAP formulated with NP-30 or LNP. FIG. 7F shows a graph of serum cardiac troponin I (cTNI) levels at various time points from C57BL/6 mice that received IM injection of 10 μg of repRNA expressing Cre recombinase formulated with NP-30 or LNP. X-Axis: hours following IM injection; Y-axis: serum cardiac troponin levels (picograms/milliliter, pg/ml). FIG. 7G shows images from hearts that were isolated, sectioned, and stained with hematoxylin and Eosin from Ai9 Tg mice that received IM injection of 10 μg of repRNA formulated with NP-30 or LNP. All graphs in FIGS. 7A-7G: P<0.05*, <0.01**, <0.001***, <0.0001****, ns=not significant.

FIG. 8 shows graphs of the change in expression of the genes for co-stimulatory molecules in after IM injection in C57BL/6 mice as analyzed by qRT-PCR. Left graph: CD40, Center graph: CD80, Right graph: CD86. Conditions: Naïve mice, isotype, and mice treated with IFNAR1 antibody. X-axis: Conditions; Y-axis: fold change relative to naive control mice. P<0.05*, <0.01**, <0.001***, <0.0001****, ns=not significant.

FIGS. 9A-9C show that LNP transfects repRNA to multiple cells in vitro and transfects mRNA to mice in vivo, respectively. FIG. 9A shows images of tdTomato signals in the extracted extramuscular tissues of Ai9 mice 21 days after the delivery of repRNA expressing Cre recombinase by NP-30 or LNP. FIG. 9B shows a graph of secreted enzyme alkaline phosphatase (SEAP) levels in the supernatants at 24 hours after the transfection in RD (muscle), Huh7.5 (liver), and THP-1 (monocyte) cells were transfected with repRNA/NP-30 at indicated N:P ratio, or repRNA/LNP. X-axis: Cell types and conditions; Y-axis: SEAP activity (RLU). FIG. 9C shows a graph of serum SEAP levels in C57BL/6 mice that received IM injection of 10 μg of mRNA or pseudouridine modified mRNA (modRNA) delivered by NP-30 or LNP. X-Axis: hours following IM injection; Y-axis: SEAP activity (RLU).

FIGS. 10A-10F show muscle and dLNs show different innate immune cells composition. C57BL/6 mice received an IM injection of repRNA/NP-30 or repRNA/LNP (10 pg). At the indicated time point, mice were sacrificed, and their muscles and dLNs were processed by Skeletal Muscle dissociation kit and Collagenase-based cell isolation, respectively. FIG. 10A shows the frequencies of the indicated innate immune cells in dLNs after the repRNA delivery analyzed by flow cytometry. X-axis: Conditions; Y-axis: % of non-myocytes. Left graph: MHC-II^{lo} macrophages in the dLN; Center graph: plasmacytoid dendritic cells in the dLN; Right graph: monocyte dendritic cells in the dLN. FIG. 10B shows the frequencies of the indicated innate immune cells in dLNs after the repRNA delivery analyzed by flow cytometry. X-axis: Conditions; Y-axis: % of non-myocytes. Left graph: neutrophils; Center graph: NK cells; right graph: T-cells.

FIG. 10B shows the frequencies of the indicated innate immune cells in dLNs after the repRNA delivery analyzed by flow cytometry. X-axis: Conditions; Y-axis: % of non-myocytes. Left graph: monocyte DCs in the dLN; Center graph: conventional dendritic cells type I (DC1) in the dLN; right graph: conventional dendritic cells type 2 (DC2) in the dLN. FIG. 10D shows the frequencies of MHC-II^{hi} population in CD11b+ CD64− cells in the muscle and dLNs of mice that received repRNA determined by flow cytometry, and are shown as means±SD. X-axis: Conditions; Y-axis: % of MHC-II (hi) macrophages in CD11b+ CD64− cells. FIG. 10E shows the fold change in CD86 expression levels in non-myocytes isolated from the muscles of mice administered repRNAs complexed with either LNP or NP-30 were determined by qRT-PCR and shown as a box plot. X-axis: Conditions; Y-axis: fold change (relative to control animals). FIG. 10F shows IL-1 family gene expression in non-myocytes of repRNA formulated with NP-30 or LNP. X-axis: Conditions; Y-axis: fold change (relative to control animals). Left graph: IL-1 alpha (IL-1α) mRNA; Center graph: IL-1 beta (IL-1β) mRNA; Right graph: IL-RN mRNA. For all graphs in FIGS. 10A-10F: All bar graphs show means±SD, and each plot indicates individual values. Box plots show min to max values with all data points. P<0.05*, <0.01**, <0.001***, <0.0001****, ns=not significant.

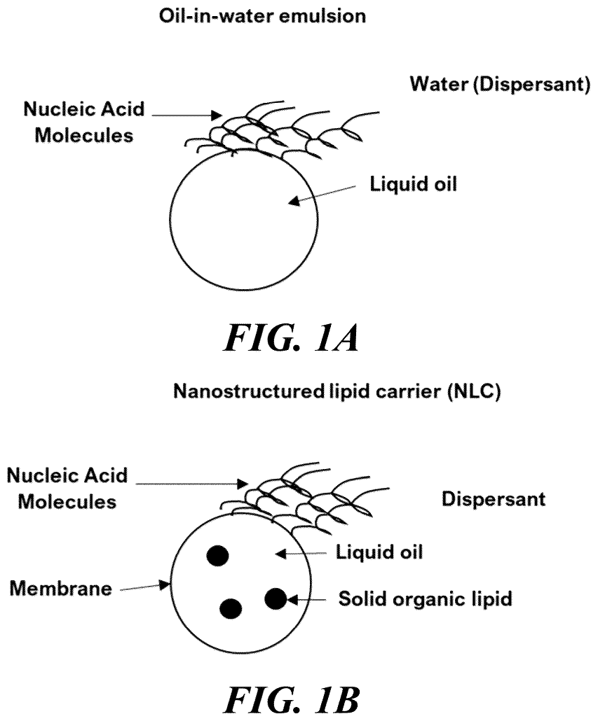

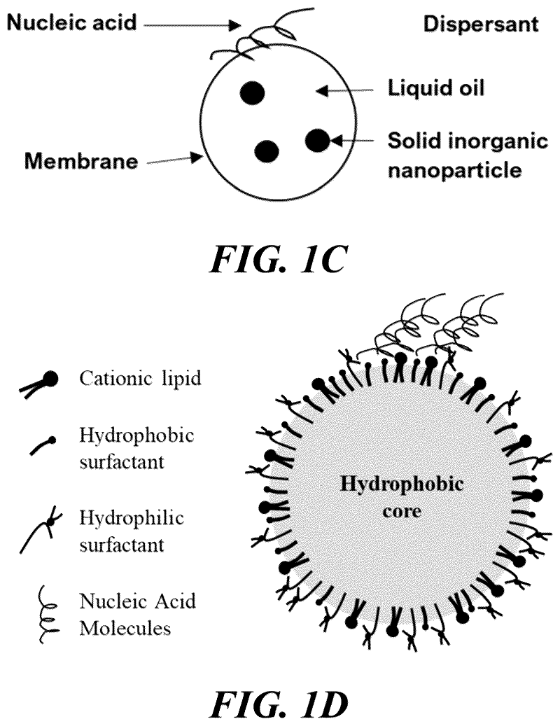

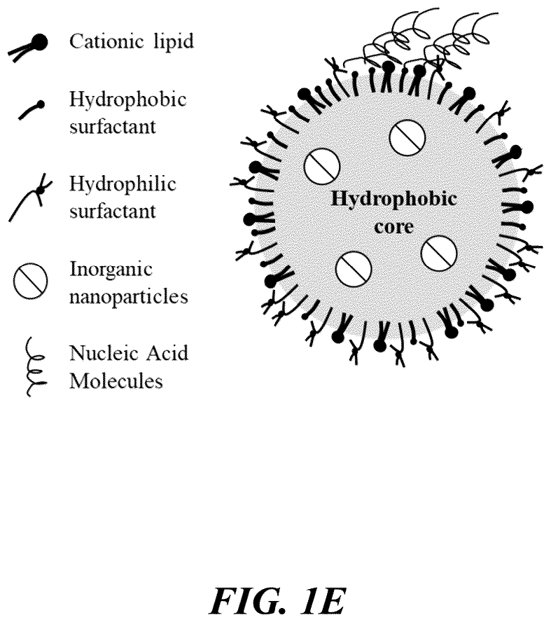

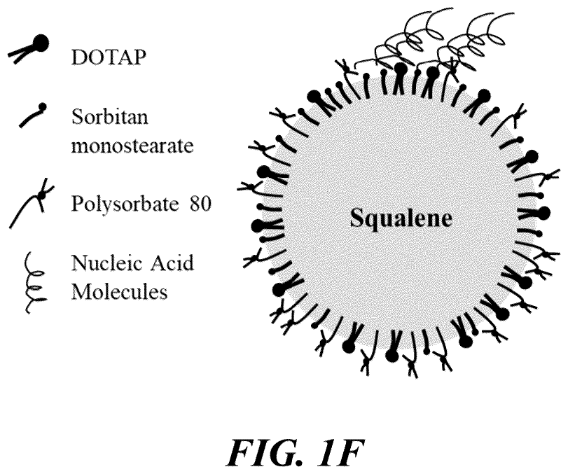

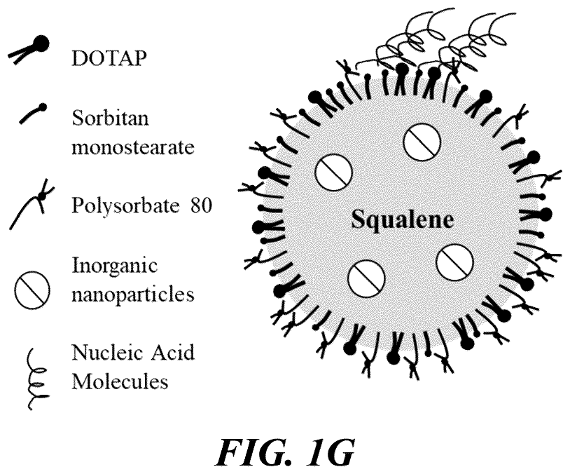

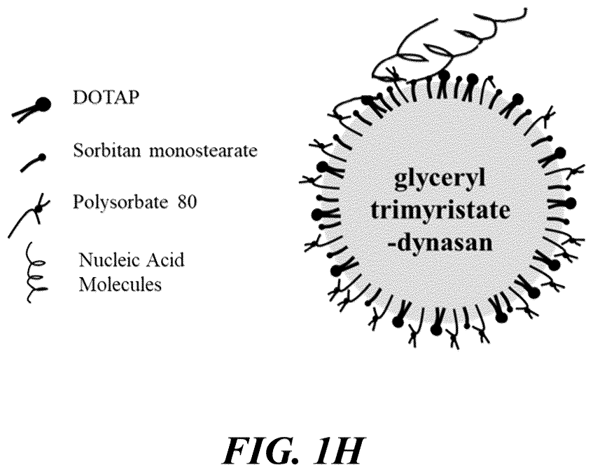

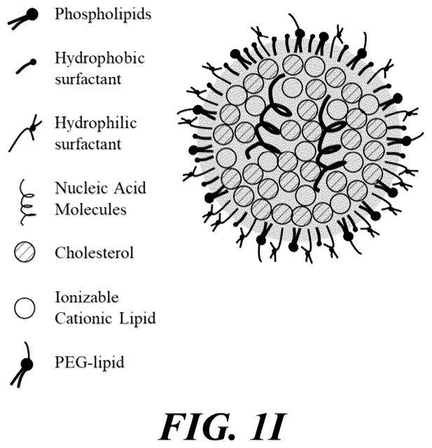

FIG. 1I shows recombinant Interferon-α (IFN-α) inhibits transgene expression from repRNA/NP-30 in vitro. A graph of SEAP activity after preconditioning of multiple cell types. RD (muscle), Huh7.5 (liver), and THP-1 (monocyte) cells were pre-treated or left untreated with IFN-α alone or IFN-α, and IFNAR neutralizing antibody (nAb), then transfected with repRNA/NP-30. 24 hours later, SEAP activity in the supernatant was measured by SEAP assay, and shown as a graph. X-axis: Cell types and conditions; Y-axis: SEAP activity (RLU).

FIG. 12 shows a schematic of the mechanism of innate immune responses in animals treated with NP-30-repRNA (left) as compared with animals treated with LNP-repRNA (right).

FIG. 13 shows a graph comparing serum interferon-α2 levels in mice treated with naïve mRNA, mRNA encapsulated in an LNP, mRNA complexed with NP-30, 5mouRNA encapsulated in LNP, and 5mouRNA complexed with NP-30 post prime (left) and post boost (right). X-axis: Conditions; Y-axis: serum mIFN-α2 (pg/ml).

FIG. 14 shows a graph comparing serum cardiac troponin (cTNI) levels in mice treated with naïve mRNA, mRNA encapsulated in an LNP, mRNA complexed with NP-30, 5mouRNA encapsulated in LNP, and 5mouRNA complexed with NP-30 post prime (left) and post boost (right). X-axis: Conditions; Y-axis: serum cTNI (pg/ml).

FIG. 15 shows a graph comparing post-prime and post-boost serum cTNI (4 hours after IM injection) and IFN-alpha2 (14 hours after IM injection) levels in mice immunized with LNP/unmod-mRNA. X-axis: Conditions; Y-axis: cTNI (pg/ml).

FIGS. 16A-16B show a comparison of interferon and antibody production in animals treated with either LNP/repRNA or NP-30/repRNA. FIG. 16A shows a graph of interferon levels 14 hours post-immunization for NP-30 and LNP-treated animals. X-axis: Conditions; Y-axis: serum IFN-α2 (pg/ml). FIG. 16B shows a graph of anti-gp140 IgG log₁₀ AUC 11 weeks post-immunization for NP-30 and LNP-treated animals. X-axis: Conditions; Y-axis: anti-gp140 IgG, log₁₀ area under the curve (AUC).

FIG. 17 shows graphs showing the mean particle size and distribution (PDI) of the NP-30 formulation before and after complexing with repRNA. Left graph: Z average diameter for groups 1, 3, and 4. X-axis: Conditions; Y-axis: Z-average (nanometers, nm). Right graph: polydispersity index (PDI) for groups 1, 3, and 4. X-axis: Conditions; Y-axis: PDI.

FIG. 18 shows a graph of serum IFN-α2 levels in non-human primates (pigtail macaques) 16 hours after IM injection. Statistical comparison of mean values between groups and across time points was performed by 2-way ANOVA and Tukey's multiple comparisons test. Both individual values and geometric mean with geometric SD shown. X-axis: time after intramuscular injection; Y-axis: serum IFN-α2 (pg/ml).

FIGS. 19A-19B show graphs of formulation-dependent T cell responses measured in tissue collected from biopsies of the lower gastrointestinal tract of pigtail macaques treated with NP-30 or LNP formulations of the nanoparticles (standard and large complex). FIG. 19A shows formulation-dependent T cell responses on week 0. Top graph: Percentage of antigen-specific CD4+ T cells. X-axis: Conditions; Y-axis: % of CD4+ T cells. Bottom graph: Percentage of antigen-specific CD8+ T cells. X-axis: Conditions; Y-axis: % of CD8+ T cells. FIG. 19B shows formulation-dependent T cell responses on week 14 following treatment with NP-30 or LNP as indicated. Top graph: Percentage of antigen-specific CD4+ T cells. X-axis: Conditions; Y-axis: % of CD4+ T cells. Bottom graph: Percentage of antigen-specific CD8+ T cells. X-axis: Conditions; Y-axis: % of CD8+ T cells. T cell markers are shown right and include: cluster of differentiation 107a granzyme B (CD107a GzB), interferon gamma (IFNg), interleukin 2 (IL-2), and tumor necrosis factor alpha (TNFa).

FIG. 20 shows a graph of Spearman's rank correlations between serum IFN-α2 levels 16 hour post immunization and adaptive immune responses. *Indicates correlations that were considered significant (p<0.05). X-axis: Cell markers; Y-axis: Spearman coefficient.

FIG. 21 shows a graph showing replicon copy numbers in different organ tissues per pg RNA 7 days after intramuscular injection for repRNA/NP-1. X-axis: organ tissue; Y-axis: replicon copies/microgram of total RNA. Samples with no detectable RNA were plotted as ½ the lower limit of quantification (LLOQ). X-axis: tissue type; Y-axis: Replicon copies per microgram (pg) of total RNA.

FIG. 22 shows graphs of serum IFN-alpha2 concentration [pg/mL] approximately 14 hours after mice received prime and boost immunization for NP-30 and LNP formulations. Left graph: 14 hours post-prime, Right graph: 14 hours post-boost. X-axis: conditions (RNA concentration and N:P ratios); Y-axis: IFNα2 [pg/ml].

FIG. 23 shows a correlation plot between serum mIFNα2 and size of NP-30/repRNA-VZV/gE complexes. The Pearson correlation coefficient (r) was −0.65 with a significance probability (p) of 0.02. X-axis: complex size; Y-axis: IFNα2 [pg/ml].

FIG. 24 shows a graph of post-boost serum anti-VZV gE/gI IgG titers in mice immunized with repRNA-VZV/gE formulated with NP-30 or LNP. X-axis: conditions (RNA concentration and N:P ratios); Y-axis: anti-VZV gE/g IgG, log₁₀ endpoint titer.

FIG. 25 shows a graph of post-boost serum anti-VZV gE/gI IgG titers in mice immunized with NP-30/repRNA-VZV/gE complexed at the indicated N:P ratios and RNA concentration. Statistical comparisons of log 10-transformed data between groups were done by two-way ANOVA and Sidak's multiple comparisons test. X-axis: N:P ratio; Y-axis: anti-VZV gE/g IgG, log 10 endpoint titer. RNA concentrations are labeled as indicated in the graph.

FIG. 26 shows a schematic and graph of binding IgG responses against BG505 SOSIP.664 trimer in non-human primates (NHPs) measured by ELISA. Top: A schematic of the vaccine schedule and sampling time points. Bottom graph: IgG responses over time post-injection. X-axis: weeks after intramuscular injection; Y-axis: anti-gp140 IgG log₁₀ AUC.

FIG. 27 show a graph demonstrating EV-D68 neutralizing antibody response in mice on experiment day 42 in NP-30 and LNP-formulated multivalent vaccine compositions. Data are presented as mean and SEM. X-axis: conditions; Y-axis: 50% reciprocal neutralization titer.

FIG. 28A-28C shows graphs of the effect of vaccination on systemic gene expression profiles in non-human primates (NHPs) treated with repRNA/NP-30 or repRNA/LNP. FIG. 28A shows the vaccination schedule and antigens encoded by repRNA, which include EV-D68/A1 (SEQ ID NO: 191); EV-D68/B1 (SEQ ID NO: 187), EV-D68 C (SEQ ID NO: 189); RSV-F (SEQ ID NO: 22) and RSV-G (SEQ ID NO: 20). FIG. 28B shows a graph of the systemic innate response in non-human primates. X-axis: days after IM injection; Y-axis: Serum interferon alpha 2 levels (picograms per milliliter, pg/ml). FIG. 28C shows a volcano plot of the upregulated an downregulated genes in non-human primate PBMCs from animals treated with repRNA/NP-30. Downregulated genes include: PLCB1, FOXJ1, FCER1A, CD1B, MUC1, CD7, and JUN. Upregulated genes: CXCL11, MMP3, HAMP, CCL3, IFN1, OASL, SIGLEC1, IL1RN, IFIT1, C2. X-axis: Log₂ (fold change). Y-axis: −log 10(p value). FIG. 28D shows a volcano plot of the upregulated and downregulated genes in non-human primate PBMCs in animals treated with repRNA/LNP. CLEC4E was down regulated in LNP-treated NHPs. Upregulated genes: CXCL12, CCL3, MR1, RNASEL, FCGR2A, CSF1, CD80, CXCL11, IL6, IFIT1, IFIH1, CXCL10, CCL8, IRF7, OASL, IL1RN, CD163. X-axis: Log₂ (fold change). Y-axis: −log 10(p value).

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION OF THE INVENTION

Non-viral in vivo RNA delivery has become an appealing modality for therapeutic and prophylactic medicine. The most broadly used formulation are lipid nanoparticles or LNP, that is used to deliver up to 100 μg doses of nucleoside-modified and column-purified conventional mRNA in humans. Nevertheless, when alphavirus-derived replicon RNA is delivered by LNP, the formulation can result in cases of severe systemic adverse events, including an objective parameter fever. Although repRNA demonstrates dose-sparing potency, compared to conventional mRNA platforms, the apparent dose ceiling of LNP-delivered repRNA limits the utility of RNA medicine.

Provided herein are compositions, kits, methods, and uses thereof for modulating an immune response in a subject that reduces reactogenicity associated with nucleic acid compositions. The compositions provided herein can elicit protective immunity, including high neutralizing antibody titers and T cell responses to antigens and also remain tolerable at higher doses of repRNA (e.g., at or above 10 micrograms (pg) in humans).

Briefly, further described herein are (1) nanoparticles, complexes, and aggregates; (2) nucleic acids; (3) adjuvants; (4) combination compositions; (5) lyophilized compositions; (6) pharmaceutical compositions; (7) dosing; (8) administration; (9) safety and efficacy; (10) therapeutic applications; and (11) kits.

### Definitions

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

As used herein, the term “about” or “approximately” means a range of up to ±20% of a given value. Where particular values are described in the application and claims, unless otherwise stated, the term “about” is implicit and in this context means within an acceptable error range for the particular value.

The term “effective amount” or “therapeutically effective amount” refers to an amount that is sufficient to achieve the desired effect.

### (1) Nanoparticles, Complexes, and Aggregates

Provided herein are various compositions comprising a nanoparticle. Nanoparticles are abbreviated as NPs herein. Further provided herein are compositions comprising nanoparticles and carriers that when administered to a subject: (1) induce local immune responses to a nucleic acid encoding a protein in a tissue that is on or adjacent to the site of administration; (2) reduce reactogenic effects of the nucleic acid encoding a protein systemically; and (3) are tolerable to a subject at nucleic acid doses of about 5 μg or higher. In some embodiments, the nanoparticle comprises a lipid carrier. Nanoparticles provided herein may be an organic, inorganic, or a combination of inorganic and organic materials that are less than about 1 micrometer (μm) in diameter. In some embodiments, nanoparticles provided herein are used as a delivery system for a bioactive agent provided herein, for example, a nucleic acid comprising or encoding for an antigen, a pattern recognition receptor (PRR) agonist, or an antibody. In some embodiments, nanoparticles provided herein are used as a delivery system for a plurality of bioactive agents. In some embodiments, compositions provided herein are multivalent vaccine compositions.

Further provided herein are various compositions comprising lipid carrier complexes or nanoparticle-complexes, wherein a plurality of lipid carriers or a plurality of nanoparticles interact physically, chemically, and/or covalently with a nucleic acid provided herein and/or other nanoparticles. The specific type of interaction between lipid carriers or between nanoparticles will depend upon the characteristic shapes, sizes, chemical compositions, physical properties, and physiologic properties. Nanoparticles provided herein can include but are not limited to: oil in water emulsions, nanostructured lipid carriers (NLCs), cationic nanoemulsions (CNEs), vesicular phospholipid gels (VPG), polymeric nanoparticles, cationic lipid nanoparticles, liposomes, gold nanoparticles, solid lipid nanoparticles (LNPs or SLNs), mixed phase core NLCs, ionizable lipid carriers, magnetic carriers, polyethylene glycol (PEG)-functionalized carriers, cholesterol-functionalized carriers, polylactic acid (PLA)-functionalized carriers, and polylactic-co-glycolic acid (PLGA)-functionalized lipid carriers.

Various nanoparticles and formulations of nanoparticles (i.e., nanoemulsions) are employed. Exemplary nanoparticles are illustrated in FIGS. 1A-1H. Oil in water emulsions, as illustrated in FIG. 1A (not to scale), are stable, immiscible fluids containing an oil droplet dispersed in water or aqueous phase. FIG. 1B (not to scale) illustrates a nanostructured lipid carrier (NLCs) which can comprise a blend of solid organic lipids (e.g., trimyristin) and liquid oil (e.g., squalene). In NLCs, the solid lipid is dispersed in the liquid oil. The entire nanodroplet is dispersed in the aqueous (water) phase. In some embodiments, the nanoparticle comprises inorganic nanoparticles, as illustrated in FIG. 1C (not to scale), as solid inorganic nanoparticles (e.g., iron oxide nanoparticles) dispersed in liquid oil. The entire nanodroplet is then dispersed as a colloid in the aqueous (water) phase. FIG. 1D (not to scale), illustrates a nanostructured lipid carrier (NLCs) comprising cationic lipids, hydrophobic surfactants, hydrophilic surfactants forming a hydrophobic core. A surface of the NLC forms a complex with a plurality of nucleic acids (nucleic acid-nanoparticle complexes and). The entire nanodroplet is dispersed in the aqueous (water) phase. FIG. 1E (not to scale), illustrates NLCs of FIG. 1D comprising solid inorganic nanoparticles within the hydrophobic core. FIG. 1F (not to scale), illustrates a nanoparticle comprising a cationic lipid membrane (e.g., DOTAP), a liquid oil core (e.g., squalene) without an inorganic particle, and one or more nucleic acids, wherein the one or more nucleic acids are in complex with the membrane. In some embodiments, nanoparticle of FIG. 1F further comprises iron oxide nanoparticles within the core as shown in FIG. 1G (not to scale). In some embodiments, a nanoparticle provided herein comprises a solid core comprising glyceryl trimyristate-dynasan (FIG. 1H). In some embodiments, a nanoparticle provided herein comprises a solid core comprising an ionizable cationic lipid and cholesterol (FIG. 1I).In some embodiments, the nanoparticles provided herein are dispersed in an aqueous solution. Non-limiting examples of aqueous solutions include water (e.g., sterilized, distilled, deionized, ultra-pure, RNAse-free, etc.), saline solutions (e.g., Kreb's, Ascaris, Dent's, Tet's saline), or 1% (w/v) dimethyl sulfoxide (DMSO) in water.

In some embodiments, nanoparticles provided herein comprise a hydrophilic surface. In some embodiments, the hydrophilic surface comprises a cationic lipid. In some embodiments, the hydrophilic surface comprises an ionizable lipid. In some embodiments, the nanoparticle comprises a membrane. In some embodiments, the membrane comprises a cationic lipid. In some embodiments, the nanoparticles provided herein comprise a cationic lipid. Exemplary cationic lipids for inclusion in the hydrophilic surface include, without limitation: 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA), 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxy-dodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis(azanetriyl))tetrapropionate, 9A1P9, decyl (2-(dioctylammonio)ethyl) phosphate, A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate, ALC-0315, ((4-hydroxybutyl)azanediyl)bis(hexane-6,1-diyl)bis(2-hexyldecanoate), ALC-0159, 2-[(polyethylene glycol)-2000]-N,N-ditetradecylacetamide, 0-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol, BAME-016B, bis(2-(dodecyldisulfanyl)ethyl) 3,3′-((3-methyl-9-oxo-10-oxa-13,14-dithia-3,6-diazahexacosyl)azanediyl)dipropionate, BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide, cKK-E12, 3,6-bis(4-(bis(2-hydroxydodecyl)amino)butyl)piperazine-2,5-dione, DC-Cholesterol, 30-[N-(N′,N′-dimethylaminoethane)-carbamoyl]cholesterol, DLin-MC3-DMA, (6Z,9Z,28Z,31Z)-heptatriaconta-6,9,28,31-tetraen-19-yl 4-(dimethylamino) butanoate, DOPE, 1,2-dioleoyl-sn-glycero-3-phosphoethanolamine, DOSPA, 2,3-dioleyloxy-N-[2-(sperminecarboxamido)ethyl]-N,N-dimethyl-1-propanaminium trifluoroacetate, DSPC, 1,2-distearoyl-sn-glycero-3-phosphocholine, ePC, ethylphosphatidylcholine, FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate, Lipid H (SM-102), heptadecan-9-yl 8-((2-hydroxyethyl)(6-oxo-6-(undecyloxy)hexyl)amino) octanoate, OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate), PEG2000-DMG, (R)-2,3-bis(myristoyloxy)propyl-1-(methoxy poly(ethylene glycol)2000) carbamate, or N1,N3,N5-tris(3-(didodecylamino)propyl)benzene-1,3,5-tricarboxamide (TT3). Other examples for suitable classes of lipids include, but are not limited to, the phosphatidylcholines (PCs), phosphatidylethanolamines (PEs), phosphatidylglycerol (PGs), and PEGylated lipids including PEGylated version of any of the above lipids (e.g., DSPE-PEGs)), and a combination thereof. In some embodiments, the nanoparticle provided herein comprises DOTAP.

In some embodiments, the nanoparticle provided herein comprise a hydrophobic core. In some embodiments, the hydrophobic core comprises an oil. In some embodiments, the hydrophobic core comprises a lipid in liquid phase at 20° C., 25° C., 30° C., 35° C., 40° C., 45° C., or 50° C. In some embodiments, the nanoparticle provided herein comprises an oil. In some embodiments, the oil is in liquid phase. Non-limiting examples of oils that can be used include α-tocopherol, coconut oil, dihydroisosqualene (DHIS), farnesene, grapeseed oil, lauroyl polyoxylglyceride, mineral oil, monoacylglycerol, palmkernal oil, olive oil, paraffin oil, peanut oil, propolis, squalene, squalane, solanesol, soy lecithin, soybean oil, sunflower oil, a triglyceride, vitamin E, or combinations thereof. In some embodiments, the nanoparticle provided herein comprises a triglyceride. Exemplary triglycerides include but are not limited to: capric triglycerides, caprylic triglycerides, a caprylic and capric triglycerides, triglyceride esters, and myristic acid triglycerins. In some embodiments, the nanoparticle comprises a triglyceride ester of saturated coconut or palmkernel oil derived caprylic and capric fatty acids and plant derived glycerol, for example, Miglyol 812 N.

In some embodiments, the hydrophobic core comprises a lipid in solid phase at 20° C., 25° C., 30° C., 35° C., 40° C., 45° C., or 50° C. In some embodiments, the hydrophobic core comprises glyceryl trimyristate-dynasan or a derivative thereof.

In some embodiments, the nanoparticles provided herein comprise a liquid organic material and a solid inorganic material. In some embodiments, the nanoparticle provided herein comprises an inorganic particle. In some embodiments, the inorganic particle is a solid inorganic particle. FIG. 1E illustrates an embodiment where the solid inorganic particle is within the hydrophobic core. In some embodiments, the nanoparticle provided herein comprises the inorganic particle within the hydrophobic core. In some embodiments, the nanoparticle provided herein comprises a metal. In some embodiments, the nanoparticle provided herein comprises a metal within the hydrophobic core. The metal can be without limitation, a metal salt, a metal oxide, a metal hydroxide, a metal phosphate, or a combination thereof. In some embodiments, the nanoparticle provided herein comprises aluminum oxide (Al₂O₃), aluminum oxyhydroxide, iron oxide (Fe₃O₄, Fe₂O₃, FeO, or combinations thereof), titanium dioxide, silicon dioxide (SiO₂), aluminum hydroxyphosphate (Al(OH)ₓ(PO₄)_{y}), calcium phosphate (Ca₃(PO₄)₂), calcium hydroxyapatite (Ca₁₀(PO₄)₆(OH)₂), iron gluconate, iron sulfate, or a combination thereof. The inorganic particles may be formed from one or more same or different metals (any metals including transition metal).

In some embodiments, the inorganic particle is a transition metal oxide. In some embodiments, the transition metal is magnetite (Fe₃O₄), maghemite (y-Fe₂O₃), wüstite (FeO), hematite (alpha (α)-Fe₂O₃), or a combination thereof. In some embodiments, the metal is aluminum hydroxide or aluminum oxyhydroxide, and a phosphate-terminated lipid or a surfactant, such as oleic acid, oleylamine, SDS, TOPO or DSPA is used to coat the inorganic solid nanoparticle, before it is mixed with the liquid oil to form the hydrophobic core. In some embodiments, the metal can comprise a paramagnetic, a superparamagnetic, a ferrimagnetic or a ferromagnetic compound. In some embodiments, the metal is a superparamagnetic iron oxide (Fe₃O₄).

In some embodiments, nanoparticles provided herein comprise a cationic lipid, an oil, and an inorganic particle. In some embodiments, the nanoparticle provided herein comprises DOTAP; squalene and/or glyceryl trimyristate-dynasan; and iron oxide. In some embodiments, the nanoparticle provided herein further comprises a surfactant.

In some embodiments, nanoparticles provided herein comprise a cationic lipid, an oil, an inorganic particle, and a surfactant.

Surfactants are compounds that lower the surface tension between two liquids or between a liquid and a solid component of the nanoparticles provided herein. Surfactants can be hydrophobic, hydrophilic, or amphiphilic. In some embodiments, the nanoparticle provided herein comprises a hydrophobic surfactant. Exemplary hydrophobic surfactants that can be employed include but are not limited to: sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85).

Suitable hydrophobic surfactants include those having a hydrophilic-lipophilic balance (HLB) value of 10 or less, for instance, 5 or less, from 1 to 5, or from 4 to 5. For instance, the hydrophobic surfactant can be a sorbitan ester having an HLB value from 1 to 5, or from 4 to 5. In some embodiments, nanoparticles provided herein comprise a ratio of the esters that yields a hydrophilic-lipophilic balance between 8 and 11. HLB is used to categorize surfactants as hydrophilic or lipophilic. The HLB scale provides for the classification of surfactant function calculated, for example, by Griffin's method:

$$H L B = \frac{20 {M}_{h}}{M} ,$$

where Mₕ is the molecular mass of the hydrophilic portion of the lipid carrier and M is the molecular mass of the lipid carrier. The HLB scale is provided below:
HLB=0: fully lipophilic/hydrophobic carrier;
HLB between 0 and 6 is an oil soluble carrier;
HLB between 6 and 9 is a water dispersible carrier;
HLB between 9 and 20 is a hydrophilic, water soluble carrier;
HLB=20: fully hydrophilic/lipophobic carrier.

In some embodiments, a nanoparticle or a lipid carrier provided herein comprises a hydrophilic surfactant, also called an emulsifier. In some embodiments, a nanoparticle or a lipid carrier provided herein comprises polysorbate. Polysorbates are oily liquids derived from ethoxylated sorbitan (a derivative of sorbitol) esterified with fatty acids. Exemplary hydrophilic surfactants that can be employed include but are not limited to: polysorbates such as TWEEN®, Kolliphor, Scattics, Alkest, or Canarcel; polyoxyethylene sorbitan ester (polysorbate); polysorbate 80 (polyoxyethylene sorbitan monooleate, or TWEEN® 80); polysorbate 60 (polyoxyethylene sorbitan monostearate, or TWEEN® 60); polysorbate 40 (polyoxyethylene sorbitan monopalmitate, or TWEEN® 40); and polysorbate 20 (polyoxyethylene sorbitan monolaurate, or TWEEN® 20). In some embodiments, the hydrophilic surfactant is polysorbate 80.

In some embodiments, nanoparticles and lipid carriers provided herein comprise a hydrophobic core surrounded by a lipid membrane, for example, a cationic lipid such as DOTAP. In some embodiments, the hydrophobic core comprises: one or more inorganic particles; a phosphate-terminated lipid; and a surfactant.

Inorganic solid nanoparticles provided herein can be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle. In some embodiments, the inorganic particle is coated with a capping ligand, the phosphate-terminated lipid, and/or the surfactant. In some embodiments the hydrophobic core comprises a phosphate-terminated lipid. Exemplary phosphate-terminated lipids that can be employed include but are not limited to: trioctylphosphine oxide (TOPO) or distearyl phosphatidic acid (DSPA). In some embodiments, the hydrophobic core comprises a surfactant such as a phosphorous-terminated surfactant, a carboxylate-terminated surfactant, a sulfate-terminated surfactant, or an amine-terminated surfactant. Exemplary carboxylate-terminated surfactants include oleic acid. Typical amine terminated surfactants include oleylamine. In some embodiments, the surfactant is distearyl phosphatidic acid (DSPA), oleic acid, oleylamine or sodium dodecyl sulfate (SDS). In some embodiments, the inorganic solid nanoparticle is a metal oxide such as an iron oxide, and a surfactant, such as oleic acid, oleylamine, SDS, DSPA, or TOPO, is used to coat the inorganic solid nanoparticle, before it is mixed with the liquid oil to form the hydrophobic core.

In some embodiments, the hydrophobic core comprises: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core comprises: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the hydrophobic core consists of: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core consists of: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80. In some embodiments, the nanoparticle provided herein can comprise from about 0.2% to about 40% w/v squalene, from about 0.001% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80. In some embodiments the nanoparticle provided herein from about 2% to about 6% w/v squalene, from about 0.010% to about 1% w/v iron oxide nanoparticles, from about 0.2% to about 1% w/v DOTAP, from about 0.25% to about 1% w/v sorbitan monostearate, and from about 0.5%) to about 5% w/v polysorbate 80. In some embodiments, the nanoparticle provided herein can comprise from about 0.2% to about 40% w/v squalene, from about 0.001% to about 10% w/v aluminum hydroxide or aluminum oxyhydroxide nanoparticles, from about 0.2% to about 10% w/v DOTAP, from about 0.25% to about 5% w/v sorbitan monostearate, and from about 0.5% to about 10% w/v polysorbate 80. In some embodiments, the nanoparticle provided herein can comprise from about 2% to about 6% w/v squalene, from about 0.01% to about 1% w/v aluminum hydroxide or aluminum oxyhydroxide nanoparticles, from about 0.2% to about 1% w/v DOTAP, from about 0.25% to about 1% w/v sorbitan monostearate, and from about 0.5%) to about 5% w/v polysorbate 80.

In some embodiments, a composition provided herein comprises at least one nanoparticle formulation as described in Table 1. In some embodiments, a composition provided herein comprises any one of NP-1 to NP-31. In some embodiments, a composition provided herein comprises any one of NP-1 to NP-37.

TABLE 1
Nanoparticle Formulations.

| | Cationic Lipid(s) | Oil(s) %(w/v) | Surfactant(s) | Additional Ingredients |
|---|---|---|---|---|
| Name | %(w/v) or mg/ml | or mg/ml | %(w/v) or mg/ml | %(w/v), mg/ml, or mM |
| NP-1 | 30 mg/ml 1,2-dioleoyl- | 37.5 mg/ml | 37 mg/ml sorbitan | 0.2 mg Fe/ml 12 nm |
| | 3-trimethylammonium- | squalene | monostearate, (2R)- | oleic acid-coated |
| | propane (DOTAP) | | 2-[(2R,3R,4S)-3,4- | iron oxide nanopar- |
| | chloride | | Dihydroxyoxolan-2- | ticles |
| | | | yl]-2-hydroxyethyl | 10 mM sodium |
| | | | octadecenoate, | citrate dihydrate. |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-2 | 30 mg/ml 1,2-dioleoyl- | 37.5 mg/ml | 37 mg/ml sorbitan | 1 mg Fe/ml 15 nm |
| | 3-trimethylammonium- | squalene | monostearate (2R)- | oleic acid-coated |
| | propane (DOTAP) | | 2-[(2R,3R,4S)-3,4- | iron oxide nanopar- |
| | chloride | | Dihydroxyoxolan-2- | ticles |
| | | | yl]-2-hydroxyethyl | 10 mM sodium |
| | | | octadecenoate | citrate dihydrate |
| | | | C₂₄H₄₆O₆ | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-3 | 30 mg/ml 1,2-dioleoyl- | 37.5 mg/ml Miglyol | 37 mg/ml sorbitan | 0.2 mg Fe/ml 15 nm |
| | 3-trimethylammonium- | 812N | monostearate, (2R)- | oleic acid-coated |
| | propane (DOTAP) | (triglyceride ester | 2-[(2R,3R,4S)-3,4- | iron oxide nanopar- |
| | chloride | of saturated | Dihydroxyoxolan-2- | ticles |
| | | coconut/palmkernel | yl]-2-hydroxyethyl | 10 mM sodium |
| | | oil derived caprylic | octadecenoate | citrate dihydrate |
| | | and capric fatty | C₂₄H₄₆O₆ | |
| | | acids and plant | (SPAN ® 60) | |
| | | derived glycerol) | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-4 | 30 mg/ml 1,2-dioleoyl- | 37.5 mg/ml Miglyol | 37 mg/ml sorbitan | 1 mg Fe/ml 15 nm |
| | 3-trimethylammonium- | 812N | monostearate, (2R)- | oleic acid-coated |
| | propane (DOTAP) | (triglyceride ester | 2-[(2R,3R,4S)-3,4- | iron oxide nanopar- |
| | chloride | of saturated | Dihydroxyoxolan-2- | ticles |
| | | coconut/palmkernel | yl]-2-hydroxyethyl | 10 mM sodium |
| | | oil derived caprylic | octadecenoate, | citrate dihydrate. |
| | | and capric fatty | C₂₄H₄₆O₆) | |
| | | acids and plant | (SPAN ® 60) | |
| | | derived glycerol) | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-5 | 30 mg/ml DOTAP | 37.5 mg/ml | 37 mg/ml sorbitan | 1 mg/ml |
| | chloride | squalene | monostearate | trioctylphosphine |
| | | | (SPAN ® 60) | oxide (TOPO)-coated |
| | | | 37 mg/ml | aluminum hydroxide |
| | | | polysorbate 80 | (Alhydrogel ® 2%) |
| | | | (TWEEN ® 80) | particles |
| | | | | 10 mM sodium citrate |
| | | | | dihydrate |
| NP-6 | 30 mg/ml DOTAP | 37.5 mg/ml Solaneso | 37 mg/ml sorbitan | 0.2 mg Fe/ml oleic |
| | chloride | (Cayman chemicals) | monostearate | acid-coated iron |
| | | | (SPAN ® 60) | oxide nanoparticles |
| | | | 37 mg/ml | 10 mM sodium citrate |
| | | | polysorbate 80 | |
| | | | (TWEEN ® 80) | |
| NP-7 | 30 mg/ml DOTAP | 37.5 mg/ml squalene | 37 mg/ml sorbitan | 10 mM sodium citrate |
| | chloride | 2.4 mg/ml glyceryl | monostearate | |
| | | trimyristate-dynasan | (SPAN ® 60) | |
| | | (DYNASAN 114 ®) | 37 mg/ml | |
| | | | polysorbate 80 | |
| | | | (TWEEN ® 80) | |
| NP-8 | 4 mg/ml DOTAP | 43 mg/ml squalene | 5 mg/ml sorbitan | 10 mM sodium citrate |
| | chloride | | trioleate | |
| | | | (SPAN ® 85) | |
| | | | 5 mg/ml polysorbate | |
| | | | 80 (TWEEN ® 80) | |
| NP-9 | 7.5 mg/ml 1,2-dioleoyl- | 9.4 mg/ml squalene | 9.3 mg/ml sorbitan | 0.05 mg/ml 15 |
| | 3-trimethylammonium- | ((6E,10E,14E,18E)- | monostearate (2R)- | nanometer |
| | propane (DOTAP) | 2,6,10,15,19,23- | 2-[(2R,3R,4S)-3,4- | superparamagnetic |
| | chloride | Hexamethyltetracosa | Dihydroxyoxolan-2- | iron oxide |
| | | 2,6,10,14,18,22- | yl]-2-hydroxyethyl | (Fe₃O₄) |
| | | hexaene, C₃₀H₅₀) | octadecenoate, | 10 mM sodium |
| | | 0.63 mg/ml glyceryl | C₂₄H₄₆O₆) | citrate dihydrate |
| | | trimyristate-dynasan | (SPAN ® 60) | |
| | | (DYNASAN 114 ®) | 9.3 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-10 | 0.4% DOTAP | 0.25% glyceryl | 0.5% sorbitan | |
| | | trimyristate-dynasan | monostearate | |
| | | (DYNASAN 114 ®) | (SPAN ® 60) | |
| | | 4.75% Squalene | 0.5% polysorbate 80 | |
| | | | (TWEEN ® 80) | |
| NP-11 | 3.0% DOTAP | 0.25% glyceryl | 3.7% sorbitan | |
| | | trimyristate-dynasan | monostearate | |
| | | (DYNASAN 114 ®) | (SPAN ® 60) | |
| | | 3.75% Squalene | 3.7% polysorbate 80 | |
| | | | (TWEEN ® 80) | |
| NP-12 | 0.4% DOTAP | 4.3% Squalene | 0.5% sorbitan | |
| | | | trioleate | |
| | | | (SPAN ® 85) | |
| | | | 0.5% polysorbate 80 | |
| | | | (TWEEN ® 80) | |
| NP-13 | 0.4% DOTAP | 0.25% glyceryl | 2.0% polysorbate 80 | |
| | | trimyristate-dynasan | (TWEEN ® 80) | |
| | | (DYNASAN 114 ®) | | |
| | | 4.08% squalene | | |
| NP-14 | 0.4% DOTAP | 0.25% glyceryl | 0.5% sorbitan | |
| | | trimyristate-dynasan | trioleate | |
| | | (DYNASAN 114 ®) | (SPAN ® 85) | |
| | | 4.08% squalene | 2.0% polysorbate 80 | |
| | | | (TWEEN ® 80) | |
| NP-15 | 0.4% DOTAP | 0.25% glyceryl | 0.25% sorbitan | |
| | | trimyristate-dynasan | trioleate | |
| | | (DYNASAN 114 ®) | (SPAN ® 85) | |
| | | 4.08% squalene | 2.0% polysorbate 80 | |
| | | | (TWEEN ® 80) | |
| NP-16 | 0.4% DOTAP | 5% squalene | 0.5% sorbitan | |
| | | | trioleate | |
| | | | (SPAN ® 85) | |
| | | | 2.0% polysorbate 80 | |
| | | | (TWEEN ® 80) | |
| NP-17 | 0.4% DOTAP | 5% squalene | 0.5% sorbitan | |
| | | | monostearate | |
| | | | (SPAN ® 60) | |
| | | | 2% polysorbate 80 | |
| | | | (TWEEN ® 80) | |
| NP-18 | 0.4% DOTAP | 0.25% glyceryl | 2% sorbitan trioleate | |
| | | trimyristate-dynasan | (SPAN ® 85) | |
| | | (DYNASAN 114 ®) | 2% polysorbate 80 | |
| | | 4.08% squalene | (TWEEN ® 80) | |
| NP-19 | 0.4% DOTAP | 0.25% glyceryl | 0.5% sorbitan | 1% aluminum hydroxide |
| | | trimyristate-dynasan | monostearate | |
| | | (DYNASAN 114 ®) | (SPAN ® 60) | |
| | | 4.75% Squalene | 0.5% polysorbate 80 | |
| | | | (TWEEN ® 80) | |
| NP-20 | 3.0% DOTAP | 0.25% glyceryl | 3.7% sorbitan | 1% aluminum hydroxide |
| | | trimyristate-dynasan | monostearate | |
| | | (DYNASAN 114 ®) | (SPAN ® 60) | |
| | | 3.75% Squalene | 3.7% polysorbate 80 | |
| | | | (TWEEN ® 80) | |
| NP-21 | 0.4% DOTAP | 4.3% Squalene | 0.5% sorbitan | 1% aluminum hydroxide |
| | | | trioleate | |
| | | | (SPAN ® 85) | |
| | | | 0.5% polysorbate 80 | |
| | | | (TWEEN ® 80 | |
| NP-22 | 0.4% DOTAP | 0.25% glyceryl | 2.0% polysorbate 80 | 1% aluminum hydroxide |
| | | trimyristate-dynasan | (TWEEN ® 80) | |
| | | (DYNASAN 114 ®) | | |
| | | 4.08% squalene | | |
| NP-23 | 0.4% DOTAP | 0.25% glyceryl | 0.5% sorbitan | 1% aluminum hydroxide |
| | | trimyristate-dynasan | trioleate | |
| | | (DYNASAN 114 ®) | (SPAN ® 85) | |
| | | 4.08% squalene | 2.0% polysorbate 80 | |
| | | | (TWEEN ® 80 | |
| NP-24 | 0.4% DOTAP | 0.25% glyceryl | 0.25% sorbitan | 1% aluminum hydroxide |
| | | trimyristate-dynasan | trioleate | |
| | | (DYNASAN 114 ®) | (SPAN ® 85) | |
| | | 4.08% squalene | 2.0% polysorbate 80 | |
| | | | (TWEEN ® 80) | |
| NP-25 | 0.4% DOTAP | 5% squalene | 0.5% sorbitan | 1% aluminum hydroxide |
| | | | trioleate | |
| | | | (SPAN ® 85) | |
| | | | 2.0% polysorbate 80 | |
| | | | (TWEEN ® 80) | |
| NP-26 | 0.4% DOTAP | 5% squalene | 0.5% sorbitan | 1% aluminum hydroxide |
| | | | monostearate | |
| | | | (SPAN ® 60) | |
| | | | 2% polysorbate 80 | |
| | | | (TWEEN ® 80) | |
| NP-27 | 0.4% DOTAP | 0.25% glyceryl | 2% sorbitan trioleate | 1% aluminum hydroxide |
| | | trimyristate-dynasan | (SPAN ® 85) | |
| | | (DYNASAN 114 ®) | 2% polysorbate 80 | |
| | | 4.08% squalene | (TWEEN ® 80) | |
| NP-28 | 0.5-5.0 mg/ml | 0.2-10% (v/v) | 0.01-2.5% (v/v) | |
| | DOTAP | squalene | polysorbate 80 | |
| | | | (TWEEN ® 80) | |
| NP-29 | 0.4% (w/w) DOTAP | 4.3% (w/w) squalene | 0.5% (w/w) sorbitan | |
| | | | trioleate | |
| | | | (SPAN ® 85) | |
| | | | 0.5% (w/w) | |
| | | | polysorbate 80 | |
| | | | (TWEEN ® 80) | |
| NP-30 | 30 mg/ml DOTAP | 37.5 mg/ml squalene | 37 mg/ml sorbitan | 10 mM sodium citrate |
| | chloride | | monostearate | |
| | | | (SPAN ® 60) | |
| | | | 37 mg/ml | |
| | | | polysorbate 80 | |
| | | | (TWEEN ® 80) | |
| NP-31 | 30 mg/ml DOTAP | 37.5 mg/ml squalene | 37 mg/ml sorbitan | 0.4 mg Fe/ml 5 nm |
| | chloride | | monostearate | oleic acid-coated |
| | | | (SPAN ® 60) | iron oxide nanopar- |
| | | | 37 mg/ml | ticles |
| | | | polysorbate 80 | 10 mM sodium citrate |
| | | | (TWEEN ® 80) | dihydrate |
| NP-32 | 0.8-1.6 mg/ml | 4.5% squalene | 0.5% (w/w) sorbitan | 10 mM sodium citrate |
| | DOTAP chloride | | trioleate | |
| | | | (SPAN 85 ®) | |
| | | | 0.5% (w/w) | |
| | | | polysorbate 80 | |
| | | | (TWEEN ® 80) | |
| NP-33 | 45-55 mol % ionizable | 35-42 mol % | 1.25-1.75 mol % | |
| | cationic lipid | cholesterol | PEG2000-DMG | |
| | 8-12 mol % | | | |
| | distearoylphos- | | | |
| | phatidylcholine | | | |
| | (DSPC) | | | |
| NP-34 | 50 mol % D-Lin- | 38.5% cholesterol | 1.5% PEG-lipid | |
| | MC3-DMA (MC3) | | | |
| | 10 mol % | | | |
| | distearoylphos- | | | |
| | phatidylcholine | | | |
| | (DSPC) | | | |
| NP-35 | 50 mol % Lipid H | 38.5% cholesterol | 1.5 mol % | |
| | (SM-102) | | PEG2000-DMG | |
| | 10 mol % | | | |
| | distearoylphos- | | | |
| | phatidylcholine | | | |
| | (DSPC) | | | |
| NP-36 | 30 mg/ml 1,2-dioleoyl- | 3.75% w/v glyceryl | 37 mg/ml sorbitan | 10 mM sodium citrate |
| | 3-trimethylammonium- | trimyristate-dynasan | monostearate, (2R)- | dihydrate. |
| | propane (DOTAP) | (DYNASAN 114 ®) | 2-[(2R,3R,4S)-3,4- | |
| | chloride | | Dihydroxyoxolan-2- | |
| | | | yl]-2-hydroxyethyl | |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-37 | 30 mg/ml 1,2-dioleoyl- | 3.75% w/v glyceryl | 37 mg/ml sorbitan | 0.2 mgFe/mL or 0.02% |
| | 3-trimethylammonium- | trimyristate-dynasan | monostearate, (2R)- | wFe/v of 5 to 15 nm |
| | propane (DOTAP) | (DYNASAN 114 ®) | 2-[(2R,3R,4S)-3,4- | diameter iron oxide |
| | chloride | | Dihydroxyoxolan-2- | nanoparticles |
| | | | yl]-2-hydroxyethyl | 10 mM sodium citrate |
| | | | octadecenoate, | dihydrate. |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |

In some embodiments, nanoparticles provided herein comprise: sorbitan monostearate (e.g., SPAN® 60), polysorbate 80 (e.g., TWEEN® 80), DOTAP, squalene, and no solid particles. In some embodiments, nanoparticles provided herein comprise: sorbitan monostearate (e.g., SPAN® 60), polysorbate 80 (e.g., TWEEN® 80), DOTAP, squalene, and iron oxide particles. In some embodiments, nanoparticles provided herein comprise an immune stimulant. In some embodiments, the immune stimulant is squalene. In some embodiments, the immune stimulant is Miglyol 810 or Miglyol 812. Miglyol 810 is a triglyceride ester of saturated caprylic and capric fatty acids and glycerol. Miglyol 812 is a triglyceride ester of saturated coconut/palmkemel oil derived caprylic and capric fatty acids and plant derived glycerol. In some embodiments, the immune stimulant can decrease the total amount of protein produced, but can increase the immune response to a composition provided herein. In some embodiments, the immune stimulant can increase the total amount of protein produced, but can decrease the immune response to a composition provided herein.

Nanoparticles provided herein can be of various average diameters in size. In some embodiments, nanoparticles provided herein are characterized as having an average diameter (z-average hydrodynamic diameter, measured by dynamic light scattering) ranging from about 20 nanometers (nm) to about 200 nm. In some embodiments, the z-average diameter of the nanoparticle ranges from about 20 nm to about 150 nm, from about 20 nm to about 100 nm, from about 20 nm to about 80 nm, from about 20 nm to about 60 nm. In some embodiments, the z-average diameter of the nanoparticle) ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In some embodiments, the z-average diameter of the nanoparticle is from about 40 nm to about 150 nm. In some embodiments, the z-average diameter of the nanoparticle is from about 40 nm to about 60 nm. In some embodiments, the nanoparticle is up to 100 nm in diameter. In some embodiments, the nanoparticle is 50 to 70 nm in diameter. In some embodiments, the nanoparticle is 40 to 80 nm in diameter. In some embodiments, the inorganic particle (e.g., iron oxide) within the hydrophobic core of the nanoparticle can be an average diameter (number weighted average diameter) ranging from about 3 nm to about 50 nm. For instance, the inorganic particle can have an average diameter of about 5 nm, about 10 nm, about 15 nm, about 20 nm, about 25 nm, about 30 nm, about 35 nm, about 40 nm, about 45 nm, or about 50 nm. In some embodiments, the ratio of esters and lipids yield a particle size between 30 nm and 200 nm. In some embodiments, the ratio of esters and lipids yield a particle size between 40 nm and 70 nm.

Nanoparticles provided herein may be characterized by the polydispersity index (PDI), which is an indication of their quality with respect to size distribution. In some embodiments, average polydispersity index (PDI) of the nanoparticles provided herein ranges from about 0.1 to about 0.5. In some embodiments, the average PDI of the nanoparticles can range from about 0.2 to about 0.5, from about 0.1 to about 0.4, from about 0.2 to about 0.4, from about 0.2 to about 0.3, or from about 0.1 to about 0.3.

In some embodiments, the nanoparticles provided herein comprise a net positive charge. In some embodiments, the nanoparticles provided herein comprise a net positive charge at a temperature of at least about 35 degrees Celsius up to 40 degrees Celsius. In some embodiments, the nanoparticles provided herein comprise a net positive charge when administered to a subject in vivo. Further provided herein are compositions, wherein the compositions comprise: nanoparticles, wherein the nanoparticles comprise: a hydrophobic core comprising lipids in liquid phase at 25 degrees Celsius; and nucleic acids encoding for a protein or an antibody, wherein the nucleic acids are complexed to the nanoparticles to form nucleic acid-nanoparticle complexes, and wherein the nucleic acid-nanoparticle complexes comprise a net positive charge at 37 degrees Celsius.

In some embodiments, nanoparticles provided herein comprise an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, nanoparticles provided herein comprise a hydrophilic surfactant-to-lipid ratio ranging from about 0.1:1 to about 2:1, from about 0.2:1 to about 1.5:1, from about 0.3:1 to about 1:1, from about 0.5:1 to about 1:1, or from about 0.6:1 to about 1:1. In some embodiments, the nanoparticles provided herein comprise a hydrophobic surfactant-to-lipid ratio ranging from about 0.1:1 to about 5:1, from about 0.2:1 to about 3:1, from about 0.3:1 to about 2:1, from about 0.5:1 to about 2:1, or from about 1:1 to about 2:1.

In some embodiments, nanoparticles provided herein comprise from about 0.2% to about 40% w/v liquid oil, from about 0.001% to about 10% w/v inorganic solid nanoparticle, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester.

In some embodiments, nanoparticles provided herein are made by homogenization and ultrasonication techniques. In some embodiments, a nanoparticle provided herein is admixed with a nucleic acid provided herein to form a nanoparticle-nucleic acid complex. In some embodiments, a plurality of nanoparticles provided herein are admixed with a plurality of nucleic acids provided herein to form a plurality of nanoparticle-nucleic acid complexes.

### (2) Nucleic Acids

Provided herein are compositions comprising a nucleic acid. In some embodiments, the nucleic acid is in complex with the nanoparticle. In some embodiments, the nucleic acid is in complex with the membrane of the nanoparticle. In some embodiments, the nucleic acid is in complex with a hydrophilic surface of the nanoparticle. In some embodiments, the hydrophilic surface of the nanoparticle comprises a cationic lipid for complexation with the nucleic acid. In some embodiments, the nucleic acid is within the nanoparticle. In some embodiments, the nucleic acid is within the hydrophobic core.

In some embodiments, the nucleic acid is deoxyribonucleic acid (DNA) or ribonucleic acid (RNA). The nucleic acid may be linear or include a secondary structure (e.g., a hair pin). In some embodiments, the nucleic acid is a polynucleotide comprising modified nucleotides or bases, and/or their analogs. A polynucleotide may comprise modified nucleotides, such as methylated nucleotides and their analogs. If present, modification to the nucleotide structure may be imparted before or after assembly of compositions provided herein. If desired, the nucleic acid can contain phosphoramidate, phosphorothioate, and/or methylphosphonate linkages. The RNA sequence can also be modified with respect to its codon usage, for example, to increase translation efficacy and half-life of the RNA. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription, for example, by using a Vaccinia Virus Capping Enzyme (VCE) comprising mRNA triphosphatase, guanylyl-transferase and guanine-7-methyltransferase, which catalyzes the construction of N7-monomethylated cap 0 structures. Cap structure can provide stability and translational efficacy to the RNA molecule. The 5′ cap of the RNA molecule can be further modified by a 2′-O-Methyltransferase which results in the generation of a cap 1 structure (m7Gppp [m2′-O]N), which may further increase translation efficacy. A cap 1 structure may also increase in vivo potency.

In some embodiments, compositions provided herein comprise one or more nucleic acids. In some embodiments, compositions provided herein comprise two or more nucleic acids. In some embodiments, compositions provided herein comprise at least one DNA. In some embodiments, compositions provided herein comprise at least one RNA. In some embodiments, compositions provided herein comprise at least one DNA and at least one RNA. In some embodiments, nucleic acids provided herein are present in an amount of above 5 ng to about 1 mg. In some embodiments, nucleic acids provided herein are present in an amount of up to about 25, 50, 75, 100, 150, 175 ng. In some embodiments, nucleic acids provided herein are present in an amount of up to about 1 mg. In some embodiments, nucleic acids provided herein are present in an amount of about 0.05 μg, 0.1 μg, 0.2 μg, 0.5, pg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, nucleic acids provided herein are present in an amount of 0.05 μg, 0.1 μg, 0.2 μg, 0.5, pg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, or 1 mg.

In some embodiments, the nucleic acid is at least about 200, 250, 500, 750, 1000, 2000, 3000, 4000, 5000, 6000, 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is up to about 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is about 7500, 10,000, 15,000, or 20,000 nucleotides in length.

### Nucleic Acids Encoding Proteins

Provided herein are compositions comprising a nucleic acid encoding for a protein, a functional variant, or a functional fragment thereof. In some embodiments, the protein is an antigen. In some embodiments, the protein is an antigen-binding protein, an antibody, an antibody fragment, or a nanobody. In some embodiments, the protein is a cytokine. In some embodiments, the protein is a recombinant protein. In some embodiments, the recombinant protein comprises two or more regions from two different proteins, for example, a fusion protein. In some embodiments, the recombinant protein comprises two microbial proteins or protein antigens. In some embodiments, the recombinant protein comprises two cancer-associated proteins. In some embodiments, the recombinant protein comprises an antigen from a cancer cell and an antigen from a lymphocyte, a macrophage, a natural killer cell, a neutrophil, a T cell, or a B cell.

Provided herein are compositions, wherein the compositions comprise: a self-replicating RNA and a nucleic acid encoding for a protein. Provided herein are compositions, wherein the compositions comprise: a carrier or nanoparticle provided herein; a self-replicating RNA; and a nucleic acid encoding for a protein. Provided further below are various nucleic acids that can be in complex with a nanoparticle or carrier provided herein.

### Nucleic Acids Encoding Infectious Disease Protein Antigens

Provided herein are infectious disease antigens and nucleic acids for recognition by a host subject. In some embodiments, compositions provided herein comprise a nucleic acid encoding for an antigen protein sequence. In some embodiments, compositions provided herein comprise at least one nucleic acid sequence comprising a sequence which encodes an antigen derived from a microorganism. In some embodiments, the microorganism is an infectious microorganism. Non-limiting examples of infectious microorganisms and infectious agents include but are not limited to: viruses such as adenoviruses, herpes simplex type 1 virus, herpes simplex type 2 virus, encephalitis virus, papillomavirus, varicella-zoster virus (VZV), Epstein-Barr virus (EBV), human cytomegalovirus (CMV), Chikungunya virus, human herpes virus type 8, human papillomavirus (HPV), BK virus, JC virus, smallpox, polio virus, hepatitis B virus, human bocavirus, parvovirus B19, human astrovirus, Norwalk virus, coxsackievirus, hepatitis A virus, poliovirus, rhinovirus, severe acute respiratory syndrome (SARS) virus, yellow fever virus, Dengue virus, West Nile virus, rubella virus, hepatitis E virus, human immunodeficiency virus (HIV), influenza virus (influenza A or influenza B), Guanarito virus, Junin virus, Lassa virus, Machupo virus, Sabii virus, Crimean-Congo hemorrhagic fever virus, Ebola virus, Marburg virus, measles virus, mumps virus, Parainfluenza virus, respiratory syncytial virus (RSV), human metapneumovirus, Hendra virus, Nipah virus, rabies virus, hepatitis D, rotavirus, orbivirus, coltivirus, banna virus, zika virus, hanta virus, West Nile virus, Middle East Respiratory Syndrome (MERS) coronavirus, Japanese encephalitis virus, and Eastern equine encephalitis; bacteria such as Acetobacter, Acinetobacter, Actinomyces, Agrobacterium, Anaplasma, Azorhizobia, Bacillus, Bacteroides, Bartonella, Bordetella, Borrelia, Brucella, Burkkolderia, Calymmatobacterium, Campylobacter, Chlamydia, Chlamydophila, Clostridium, Corynebacterium, Coxiella, Ehrlichia, Enterobacter, Enterococcus, Escherichia, Francisella, Fusobacterium, Gardnerella, Haemophilus, Helicobacter, Klebsiella, Lactobacillus, Legionella, Listeria, Methanobacterium, Microbacterium, Micrococcus, Moraxella, Mycobacterium, Mycoplasma, Neisseria, Pasteurella, Peptostreptococcus, Porphyromonas, Prevotella, Pseudomonas, Rhizobium, Rickettsia, Rochalimaea, Rothia, Salmonella, Shigella, Staphylococcus, Stenotrophomonas, Streptococcus, Streptococcus pneumoniae, Treponema, Vibrio, Walbachia, and Yersinia; fungi such as Aspergillus, Saccharomyces, Cryptococcus, Coccidioides, Neurospora, Histoplasma, Blastomyces; parasites such as Babesia sp., Cryptosporidium sp., Plasmodium sp., Toxoplasma sp. Plasmodium sp., Plasmodium falciparum, Plasmodium vivax, Cryptosporidium parvum, Cryptosporidium hominis, Eimeria sp., Eimeria tenella, Theileria sp., Theileria parva, Toxoplasma sp. Toxoplasma gondii, Trypanosoma brucei subspecies, Trypanosoma cruzi, Leishmania sp., and Leishmania major; and yeast such as Candida.

In some embodiments, the antigen is derived from a microorganism that causes a severe respiratory disease in mammalian populations. In some embodiments, the antigen is a surface protein or a transmembrane protein expressed on the surface of a microbial organism.

In some embodiments, the antigen is a viral antigen. In some embodiments, the viral antigen is a spike protein, a glycoprotein, or an envelope protein. In some embodiments, the viral antigen is a coronavirus antigen. In some embodiments, the coronavirus is a SARS-CoV-1 coronavirus, SARS-CoV-2 coronavirus, a Middle East Respiratory Syndrome (MERS) coronavirus, or a variant thereof. In some embodiments, the coronavirus antigen is a spike (S) protein antigen. In some embodiments, the coronavirus antigen is a pre-fusion spike protein antigen. Variants of the SARS-CoV-2 virus include the alpha, beta, delta, mu, and omicron variants. The Alpha (B.1.1.7), Beta (B.1.351, B.1.351.2, B.1.351.3), Delta (B.1.617.2, AY.1, AY.2, AY.3), Omicron (B.1.1.529), and Gamma (P.1, P.1.1, P.1.2) variants circulating in the United States are classified as variants of concern. SARS-CoV-2 structure, its components, along with variants and their various features are described further below.

Coronaviruses are single-stranded RNA-enveloped viruses that have four structural proteins, known as the S (spike), E (envelope), M (membrane), and N (nucleocapsid) proteins. The N protein holds the RNA genome, and the S, E, and M proteins together create the viral envelope. In SARS-CoV-2, the spike (S) protein facilitates viral attachment and fusion with the membrane of a host cell via the host cell receptor, angiotensin-converting enzyme 2 (ACE2). The S protein mediates viral cell entry into the host cell. The total length of SARS-CoV-2 spike is generally 1273 amino acids and includes a signal peptide (amino acids 1-13) located at the N-terminus, the S1 subunit (residues 14-685), and the S2 subunit (residues 686-1273); the last two regions are responsible for receptor binding and membrane fusion, respectively. In the S1 subunit, there is an N-terminal domain (14-305 residues) and a receptor-binding domain (RBD, 319-541 residues). The fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues) comprise the S2 subunit. Specifically, the HRT and HR2 are composed of a repetitive heptapeptide: HPPHCPC, where H is a hydrophobic or traditionally bulky residue, P is a polar or hydrophilic residue, and C is another charged residue. HRT and HR2 form a six-helical bundle (6-HB) referred to as the “stem helix” domain of the S2 protein. When the RBD binds to ACE2 on a host cell membrane, S2 changes conformation by inserting FP into the target cell membrane, exposing the pre-hairpin coiled-coil of the HRT domain and triggering interaction between the HR2 domain and HRT trimer to form the stem helix (6-HB), thus bringing the viral envelope and cell membrane into proximity for viral fusion and entry. The wild-type spike protein amino acid sequence is provided in SEQ ID NO: 1. The amino acid sequence corresponding to the stem helix is provided in SEQ ID NO: 2.

Coronaviruses regularly undergo antigenic drift, a type of genetic variation in viruses, arising from the accumulation of mutations in viral genes that encode for virus-surface proteins that host antibodies recognize. Several SARS-CoV-2 variants have been identified by public health agencies and are provided in Table 2 below.

TABLE 2
SARS-CoV-2 Variants.

| | | | | Additional spike | Earliest | |
|---|---|---|---|---|---|---|
| | Pango | | Nextstrain | amino acid | documented | Date of |
| WHO label | lineage* | GISAID clade | clade | changes monitored° | samples | designation |
| Alpha | B.1.1.7 ^{#} | GRY | 20I (V1) | N501Y | United | 18 Dec. 2020 |
| | | | | A570D | Kingdom, | |
| | | | | P681H | September 2020 | |
| | | | | T716I | | |
| | | | | S982A | | |
| | | | | D1118H | | |
| Beta | B.1.351 | GH/501Y.V2 | 20H (V2) | D80A | South Africa, | 18 Dec. 2020 |
| | | | | D215G | May 2020 | |
| | | | | K417N | | |
| | | | | A701V | | |
| | | | | N501Y | | |
| | | | | E484K | | |
| Gamma | P.1 | GR/501Y.V3 | 20J (V3) | L18F | Brazil, | 11 Jan. 2021 |
| | | | | T20N | November 2020 | |
| | | | | P26S | | |
| | | | | D138Y | | |
| | | | | R190S | | |
| | | | | K417T | | |
| | | | | E484K | | |
| | | | | N501Y | | |
| | | | | H655Y | | |
| | | | | T1027I | | |
| Delta | B.1.617.2^{§} | G/478K.V1 | 21A | T19R | India, | VOI: 4 Apr. 2021 |
| | | | | L452R | October 2020 | VOC: 11 May 2021 |
| | | | | T478K | | |
| | | | | P681R | | |
| | | | | D950N | | |
| Eta | B.1.525 | G/484K.V3 | 21D | | Multiple | 17 Mar. 2021 |
| | | | | | countries, | |
| | | | | | December 2020 | |
| Iota | B.1.526 | GH/253G.V1 | 21F | | United States of | 24 Mar. 2021 |
| | | | | | America, | |
| | | | | | November 2020 | |
| Kappa | B.1.617.1 | G/452R.V3 | 21B | | India, | 4 Apr. 2021 |
| | | | | | October 2020 | |
| Lambda | C.37 | GR/452Q.V1 | 21G | | Peru, | 14 Jun. 2021 |
| | | | | | December 2020 | |
| Mu | B.1.621 | GH | 21H | | Colombia, | 30 Aug. 2021 |
| | | | | | January 2021 | |
| Omicron | B.1.1.529 | GR/484A | 21K | A67V, Δ69-70, | November 2021 | VOC: 26 Nov. 2021 |
| | | | | T95I, G142D, | South Africa, Hong | |
| | | | | Δ143-145, Δ211, | Kong, Belgium, | |
| | | | | L212I, | Israel | |
| | | | | ins214EPE, | | |
| | | | | G339D, S371L, | | |
| | | | | S373P, S375F, | | |
| | | | | K417N, N440K, | | |
| | | | | G446S, S477N, | | |
| | | | | T478K, E484A, | | |
| | | | | Q493R, G496S, | | |
| | | | | Q498R, N501Y, | | |
| | | | | Y505H, T547K, | | |
| | | | | D614G, H655Y, | | |
| | | | | N679K, P681H, | | |
| | | | | N764K, D796Y, | | |
| | | | | N856K, Q954H, | | |
| | | | | N969K, L981F | | |

*includes all descendent lineages. The full list of Pango lineages can be found on the world wide web at: Error! Hyperlink reference not valid.cov-lineages.org/lineage_list.html and pango.network/faqs/

In some embodiments, the antigen is derived from a SARS-CoV-2 variant of concern (VOC). A variant of concern is a variant for which there is evidence of an increase in transmissibility, more severe disease, increased hospitalizations, or deaths, significant reduction in neutralization by antibodies generated during previous infection or vaccination, reduced effectiveness of treatments or vaccines, or diagnostic detection failures. Possible attributes of a variant of concern include, in addition to the possible attributes of a variant of interest, (i) evidence of impact on diagnostics, treatments, or vaccines, (ii) widespread interference with diagnostic test targets, (iii) evidence of substantially decreased susceptibility to one or more class of therapies, (iv) evidence of significant decreased neutralization by antibodies generated during previous infection or vaccination, (v) evidence of reduced vaccine-induced protection from severe disease, (vi) evidence of increased transmissibility, and (vii) evidence of increased disease severity.

In some embodiments, the antigen is derived from a SARS-CoV-2 variant of interest (VOI). A variant of interest is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity. Possible attributes of a variant of interest include (i) specific genetic markers that are predicted to affect transmission, diagnostics, therapeutics, or immune escape, (ii) evidence that it is the cause of an increased proportion of cases or unique outbreak clusters, and (iii) limited prevalence or expansion in the US or in other countries.

Further provided herein are compositions comprising a nucleic acid encoding a human immunodeficiency virus (HIV) protein. In some embodiments, the HIV protein is derived from a type 1 HIV or a type 2 HIV. In some embodiments, the HIV protein comprises an HIV envelope protein. In some embodiments, the HIV protein comprises an envelope glycoprotein (gp), a gag polyprotein or a fragment thereof, a reverse transcriptase (pol), a viral protease (PR), a capsid (CA), a nucleocapsid (NC), a tetherin, a matrix (MA) protein, an integrase, an RNAse protein, a transmembrane protein, a transactivator protein, a negative regulating factor, a viral infectivity protein, a virus protein r (vpr), a virus protein unique (vpu), a virus protein x (vpx), a tat/rev protein (tev), or any combination thereof. In some embodiments, the HIV protein is gp120, gp41. In some embodiments, compositions provided herein comprise a nucleic acid encoding the HIV env protein, wherein the nucleic acid sequence is at least 85% identical to SEQ ID NO: 180. In some embodiments, the nucleic acid encodes for an amino acid sequence that is at least 85% identical to SEQ ID NO: 182.

In some embodiments, the viral antigen is an influenza virus antigen. In some embodiments, the influenza virus antigen is a hemagglutinin antigen. Hemagglutinin (abbreviated HA) is a protein present on the surface of an influenza virus. On the viral surface, the hemagglutinin protein is present in homotrimers, each monomer of which is comprised of two subunits, HA1 and HA2, linked by a disulfide bond. Structurally, hemagglutinin proteins are comprised of several domains: a globular head domain, a stalk domain (also referred to as a stem or the stem protein), a transmembrane domain, and a cytoplasmic domain. Generally, during infection of a host cell (e.g., a eukaryotic cell such as a human cell) with an influenza virus, the hemagglutinin protein recognizes and binds to sialic acid of a receptor on the surface of a host cell facilitating attachment of the virus to the host cell. Following endocytosis of the virus and acidification of the endosome, the hemagglutinin protein undergoes a pH-dependent conformational change that allows for the hemagglutinin protein to facilitate fusion of the viral envelope with the endosome membrane of host cell and entry of the viral nucleic acid into the host cell. In general, influenza viruses are classified based on the amino acid sequence of the viral hemagglutinin protein and/or the amino acid sequence of the viral neuraminidase (NA). In some embodiments, nucleic acids provided herein comprise an antigen sequence encoding for an influenza hemagglutinin protein. In some embodiments, nucleic acids provided herein comprise an antigen sequence encoding for an influenza hemagglutinin protein stem region or a functional variant thereof. In some embodiments, the hemagglutinin antigen is of the subtype H1, H2, H3, H4, H5, H6, H7, H8, H9, H10, H11, H12, H13, H14, H15, H16, H17, or H18. The differences in amino acid sequences between hemagglutinin proteins of different subtypes are largely found within the sequence of the head domain of the protein. The amino acid sequence of the stem region is considered to be more conserved between hemagglutinin subtypes compared to sequence of the head domain. In some embodiments, the hemagglutinin antigen does not comprise a head domain (HA1). In some embodiments, the hemagglutinin antigen comprises a portion of the head domain (HA1). In some embodiments, the hemagglutinin antigen does not comprise a cytoplasmic domain. In some embodiments, the hemagglutinin antigen comprises a portion of the cytoplasmic domain. In some embodiments, the truncated hemagglutinin antigen. In some embodiments, the truncated hemagglutinin protein comprises a portion of the transmembrane domain. In some embodiments, the truncated hemagglutinin protein comprises a stem region or a functional fragment thereof.

In some embodiments, the viral antigen is a Varicella-Zoster Virus (VZV) antigen. In some embodiments, the VZV antigen is a glycoprotein E (gE) antigen, a glycoprotein B (gB) antigen, a glycoprotein H (gH) antigen, a glycoprotein L (gL) antigen, a glycoprotein N (gN) antigen, a glycoprotein I (gI) antigen. In some embodiments, the viral antigen is an Epstein-Barr virus (EBV) antigen. In some embodiments, the viral antigen is a herpes simplex virus (HSV) antigen. In some embodiments, the herpes simplex virus antigen is HSV1 or HSV2 antigen. In some embodiments, the HSV antigen is a glycoprotein B, glycoprotein E, glycoprotein L, glycoprotein M, or a glycoprotein I. In some embodiments, the viral antigen is a rabies virus antigen. In some embodiments, the rabies virus antigen is a nucleoprotein (N), a phosphoprotein (P), a matrix protein (M), a glycoprotein (G) or a polymerase (L) antigen. In some embodiments, the viral antigen is a cytomegalovirus (CMV, human betaherpesvirus 5) antigen. In some embodiments, the CMV antigen is a glycoprotein B. Non-limiting examples of viral antigens for inclusion include: Zika virus envelope protein (ZIKV E), Zika virus precursor membrane and envelope proteins (prM-ENV), SARS-CoV2 spike (S) protein and envelope (E) proteins, HIV p24 antigen and Nef protein, influenza virus hemagglutinin (HA) antigen (H2, H3, H5, H6, H7, H8 and H9), influenza virus neuraminidase, rubella E1 and E2 antigens, rotavirus VP7sc antigen, RSV M2 protein, cytomegalovirus envelope glycoprotein B, the S, M, and L proteins of hepatitis B virus, rabies glycoprotein, rabies nucleoprotein, Crimean-Congo hemorrhagic fever glycoprotein Gc and or Gn, Nipah henipavirus glycoprotein, Hendra virus glycoprotein, human papillomavirus E6 protein, human papillomavirus E7 protein, human papillomavirus L1 protein, or human papillomavirus L2 protein.

In some embodiments, the antigen is a viral antigen. In some embodiments, the antigen is a respiratory syncytial virus (RSV) antigen. In some embodiments, the antigen is an RSV glycoprotein (G), RSV-G. In some embodiments, the antigen is an RSV fusion (F) glycoprotein RSV-F. In some embodiments, the antigen is a zika virus antigen. In some embodiments, the zika virus antigen is an envelope (E) protein.

In some embodiments, the antigen is a bacterial antigen. In some embodiments, the bacterial antigen is a Mycobacterium tuberculosis antigen. In some embodiments, the Mycobacterium tuberculosis antigen is H37Rv, malate synthase, or MPT51. In some embodiments, the bacterial antigen is a Chlamydia trachomatis antigen. In some embodiments, the Chlamydia trachomatis antigen is a major outer membrane protein antigen. In some embodiments, the bacterial antigen is a Staphylococcus aureus antigen.

In some embodiments, the antigen is a parasite antigen. In some embodiments the parasite antigen is a Giardia lamblia antigen, a Leishmaniasis antigen, a Plasmodium falciparum antigen, a Toxoplasma gondii antigen, a Trichomonas vaginalis antigen, a Trypanosoma brucei antigen, a Trypanosoma cruzi antigen, a Schistosoma antigen, a Toxocara antigen, a Trichinella antigen, or a Babesia antigen.

In some embodiments, the antigen is a fungal antigen. In some embodiments, the fungal antigen is a Cryptococcus antigen, an Aspergillus antigen, a Coccidioides immitis antigen, a Coccidioides posadasii antigen, a Histoplasma capsulatum antigen, a Blastomyces dermatitidis antigen, a Pneumocystis jirovecii antigen, a Trichophyton antigen, a Microsporum antigen, or a Epidermophyton antigen. In some embodiments, the antigen is a yeast antigen. In some embodiments, the yeast antigen is a Candida antigen. In some embodiments, the microbial protein antigen is a major outer membrane protein, an envelope protein, an envelope glycoprotein, an E7 protein, an E6 oncoprotein, a haemagglutinin protein, a malate synthase protein, a nucleoprotein, an L protein, a transmembrane glycoprotein, a phosphoroprotein, an M2 protein, and RSV glycoprotein, an RSV fusion protein, a spike protein, a variant, or a derivative thereof. In some embodiments, the microbial protein antigen is a glycoprotein H, a glycoprotein L, a glycoprotein B, or a combination thereof. In some embodiments, microbial protein antigen is from a Chlamydia trachomatis bacteria, an enterovirus, a gamma herpesvirus, an alphaherpesvirus, a human papillomavirus virus (HPV), an influenza virus, a Mycobacterium tuberculosis bacteria, a Pseudomonas bacteria, aAcinetobacter bacteria, a Klebsiella bacteria, an Escherichia coli bacteria, a Serratia bacteria, a Streptococcus bacteria, a Shigella bacteria, a Campylobacter bacteria, a Staphylococcus bacteria, a Salmonellae bacteria, an Enterococcus bacteria, a Helicobacter pylori bacteria, a Neisseria gonorrhoeae bacteria, a Haemophilus influenzae bacteria, a Proteus bacteria, a rabies virus, a respiratory syncytial virus, a coronavirus, a severe acute respiratory syndrome (SARS) virus, a Varicella-Zoster Virus (VZV), or a Zika virus.

In some embodiments, nucleic acids provided herein encodes for an antigen listed in Table 3 or a fragment thereof. In some embodiments, the nucleic acid comprises at least 80%, 85%, 90%, 95%, 96%, 97%, 98%, or 99% sequence identity to a sequence which specifically binds an antigen listed in Table 3. In some embodiments, the nucleic acid provided herein comprises at least 80%, 85%, 90%, 95%, 96%, 97%, 98%, or 99% sequence similarity to an RNA sequence listed in Table 3. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated. Exemplary nucleic acid sequences encoding for exemplary viral and bacterial antigens are listed in Table 3.

TABLE 3
Infectious Microorganism Antigens and Sequences.

| Infectious | | Nucleic Acid | Amino Acid |
|---|---|---|---|
| Microorganism | Antigen Protein | SEQ ID NO: | SEQ ID NOS: |
| Chlamydia trachomatis | major outer | — | SEQ ID NO: 3 |
| | membrane protein | | |
| Epstein-Barr virus (EBV) | envelope | — | SEQ ID NO: 4 |
| (also referred to as human | glycoprotein B | | |
| gammaherpesvirus 4) | | | |
| Herpes simplex virus 1 | envelope | — | SEQ ID NO: 5 |
| (HSV1, also referred to | glycoprotein B | | |
| as Human | | | |
| alphaherpesvirus 1) | | | |
| Herpes simplex virus 2 | envelope | — | SEQ ID NO: 6 |
| (HSV2, also referred to | glycoprotein G | | |
| as Human | | | |
| alphaherpesvirus 2) | | | |
| HPV | E7 protein | — | SEQ ID NO: 7 |
| Human papilloma virus | E6 oncoprotein | — | SEQ ID NO: 8 |
| (HPV) | | | |
| Influenza A virus H3N2 | Influenza A | — | SEQ ID NO: 9 |
| (New York) variant | Haemagglutinin | | |
| | (HA) | | |
| Influenza B | Influenza B | — | SEQ ID NO: 10 |
| Haemagglutinin | virus HA | | |
| (HA) Lee 1940 Variant | | | |
| Influenza virus | Haemagglutinin | SEQ ID NO: 11 | SEQ ID NO: 12 |
| | (HA) | | |
| Mycobacterium | Malate synthase | — | SEQ ID NO: 13 |
| tuberculosis | | | |
| Mycobacterium | MPT51 (H37Rv) | — | SEQ ID NO: 14 |
| tuberculosis | | | |
| Rabies virus | nucleoprotein N | — | SEQ ID NO: 15 |
| Rabies virus | L protein | — | SEQ ID NO: 16 |
| Rabies virus | transmembrane | — | SEQ ID NO: 17 |
| | glycoprotein G | | |
| Rabies virus | phosphoprotein M1 | — | SEQ ID NO: 18 |
| Rabies virus | M2 protein | — | SEQ ID NO: 19 |
| Respiratory syncytial virus | RSV glycoprotein | SEQ ID NO: 20 | SEQ ID NO: 21 |
| (RSV) (also referred to as | (RSV-G) | | |
| Human orthopneumovirus) | | | |
| RSV | RSV fusion | SEQ ID NO: 22 | SEQ ID NO: 23 |
| | protein | | (partial) |
| | (RSV-F) | | SEQ ID NO: 24 |
| | | | (full length) |
| SARS-CoV-2 Delta | Delta.AY1-S2P- | SEQ ID NO: 25 | SEQ ID NO: 26 |
| | wtFur-newKozak | | |
| | Pre-fusion Spike | | |
| SARS-CoV-2 A.1 | Delta V5 Spike | SEQ ID NO: 27 | SEQ ID NO: 28 |
| SARS-CoV-2 A.1 | K995P-V996P Pre- | SEQ ID NO: 29 | SEQ ID NO: 30 |
| | fusion Spike | | |
| SARS-CoV-2 Alpha | B.1.1.7-PP-D614G | SEQ ID NO: 31 | SEQ ID NO: 32 |
| | Pre-fusion Spike | | |
| SARS-CoV-2 B.1 | D614G Spike | SEQ ID NO: 33 | SEQ ID NO: 34 |
| SARS-CoV-2 Beta | B.1.351-PP-D614G | SEQ ID NO: 35 | SEQ ID NO: 36 |
| | Pre-fusion Spike | | |
| SARS-CoV-2 Delta | Delta.AY1-S2P- | SEQ ID NO: 37 | SEQ ID NO: 38 |
| | wtFur Pre-fusion | | |
| | Spike | | |
| SARS-CoV-2 Omicron | Omicron- B.1.1.529 | SEQ ID NO: 39 | SEQ ID NO: 40 |
| | Spike | | |
| SARS-CoV-2 Omicron | Omicron- B.1.1.529 | — | SEQ ID NO: 41 |
| | Stem Helix (HR1 | | |
| | and HR2) | | |
| SARS-CoV-2 wild-type | Wuhan-Hu-1 -Full- | — | SEQ ID NO: 1 |
| | Length Wild-Type | | |
| | Spike Protein Amino | | |
| | Acid Sequence | | |
| SARS-CoV-2 wild-type | Wuhan-Hu-1 -Wild- | — | SEQ ID NO: 2 |
| | Type Spike Protein- | | |
| | Stem Helix [residues | | |
| | 913 to 1213] | | |
| severe acute respiratory | spike protein | — | SEQ ID NO: 42 |
| syndrome coronavirus 1 | | | |
| (SARS-CoV-1) | | | |
| Varicella-Zoster Virus | gE | SEQ ID NO: 43 | SEQ ID NO: 44 |
| (VZV) (also referred to | | | |
| as Human | | | |
| alphaherpesvirus 3) | | | |
| VZV | gB | — | SEQ ID NO: 45 |
| VZV | gH | — | SEQ ID NO: 46 |
| VZV | gL | — | SEQ ID NO: 47 |
| VZV | gN | — | SEQ ID NO: 48 |
| VZV | gI | SEQ ID NO: 49 | SEQ ID NO: 50 |
| Zika virus | Envelope (E) protein | SEQ ID NO: 51 | SEQ ID NO: 52 |

### Nucleic Acids Encoding Antibodies

Provided here is a composition comprising a nucleic acid coding for an antibody. In some embodiments, the antibody is a monoclonal antibody. Monoclonal antibodies or mAbs include intact molecules, as well as antibody fragments (such as, Fab and F(ab′)2 fragments) that are capable of specifically binding to an epitope of a protein or antigen. In some embodiments, the antibody is a murine antibody, a humanized antibody, or a fully human antibody.

In some embodiments, the antibody is an immunoglobulin (Ig) molecule. Immunoglobulin (Ig) molecules and immunologically active portions of immunoglobulin molecules (i.e., molecules that contain an antigen binding site that specifically bind an antigen) are comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of which are discussed below, and include but are not limited to a variety of forms, including full length antibodies and antigen-binding portions thereof; including, for example, an immunoglobulin molecule, a monoclonal antibody, a chimeric antibody, a CDR-grafted antibody, a human antibody, a humanized antibody, a single chain antibody, a Fab, a F(ab′), a F(ab′)2, a Fv antibody, fragments produced by a Fab expression library, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies. In some embodiments, the immunoglobulin molecule is an IgG, IgE, IgM, IgD, IgA, or an IgY isotype immunoglobulin molecule. In some embodiments, the antibody or immunoglobulin molecules provided herein are a specific subclass of immunoglobulin molecule. In some embodiments, the immunoglobulin molecule is an IgG1, an IgG2, an IgG3, an IgG4, an IgGA1, or an IgGA2 subclass immunoglobulin molecule. In a full-length antibody, each heavy chain is comprised of a heavy chain variable domain (abbreviated herein as HCVR or V_{H}) and a heavy chain constant region. The heavy chain constant region is comprised of three domains: C_{H}1, C_{H}2, and C_{H}3. Each light chain is comprised of a light chain variable domain (abbreviated herein LCVR as V_{L}) and a light chain constant region. The light chain constant region is comprised of one domain, CL. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. This structure is well-known to those skilled in the art. The chains are usually linked to one another via disulfide bonds. Furthermore, in humans, the light chain may comprise a kappa chain or a lambda chain. Complementarity Determining Regions (“CDRs”), i.e., CDR1, CDR2, and CDR3) are the amino acid residues of a heavy or light chain variable domain specific for antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (HI), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (HI), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. The exact boundaries of these CDRs have been defined differently according to different systems. The system described by Kabat (Kabat et al, Sequences of Proteins of Immunological Interest (National Institutes of Health, Bethesda, Md. (1987) and (1991)) not only provides an unambiguous residue numbering system applicable to any variable region of an antibody, but also provides the residue boundaries defining the three CDRs. These CDRs may be referred to as Kabat CDRs. Chothia and coworkers (Chothia & Lesk, J. Mol. Biol, 196:901-917 (1987) and Chothia et al., Nature 342:877-883 (1989)) found that certain sub-portions within Kabat CDRs adopt nearly identical peptide backbone conformations, in spite of great diversity at the level of amino acid sequence. These sub-portions were designated as L1, L2 and L3 or HI, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB). 9: 133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or assay result that particular residues or groups of residues or even entire CDRs do not significantly impact antigen binding. The alignment of the CDR sequences can be conducted using publicly available software such as BLAST, Align, and the international ImMunoGeneTics information system (IMGT). Those skilled in the art can determine the appropriate parameters for alignment, but the default parameters for BLAST are specifically contemplated. In some embodiments, an antibody described herein is originally generated by a non-human animal, for example, a sheep, a dog, a rabbit, a mouse, a rat, a primate, a goat, a llama, an alpaca, or a horse, against an antigen described herein and, optionally, humanized as described herein.

In some embodiments, nucleic acids provided herein encode for a recombinant antibody, a chimeric antibody, or a multivalent antibody. In some embodiments, the multivalent antibody is a bispecific antibody, a trispecific antibody, or a multispecific antibody. In some embodiments, the antibody or functional fragment is an antigen-binding fragment (Fab), and Fab2 a F(ab′), a F(ab′)2, an dAb, an Fc, a Fv, a disulfide linked Fv, a scFv, a tandem scFv, a free LC, a half antibody, a single domain antibody (dAb), a diabody, or a nanobody. In some embodiments, the nanobody comprises a heavy chain variable (V_{H}) region. In further embodiments, the heavy chain variable (V_{H}) region comprises three CDR regions.

In some embodiments, the antibody or functional fragment thereof binds to a microbial antigen. In some embodiments, the microbial antigen is a viral envelope protein. In some embodiments, the antibody or functional fragment thereof is a SARS-CoV-2 virus antibody. In some embodiments, the SARS-CoV-2 virus antibody is bamlanivimab, casirivimab, imdevimab, or sotrovimab. Exemplary amino acid sequences for SARS-CoV-2 antibodies are provided below in Table 4.

In some embodiments, a nucleic acid provided herein encode for a protein or antibody amino acid sequence or a functional fragment thereof listed in Table 4. In some embodiments, compositions provided herein comprise two or more nucleic acids coding different sequences listed in Table 4. In some embodiments, the nucleic acid provided herein codes for a protein or antibody amino acid sequence or a functional fragment thereof comprising at least 80%, 85%, 90%, 95%, 96%, 97%, 98%, or 99% sequence identity to a sequence listed in Table 4. In some embodiments, compositions provided herein comprise two or more nucleic acids coding different sequences listed in Table 4. In some embodiments, the nucleic acid provided herein codes for a protein, antibody, or fragment thereof comprising at least 80%, 85%, 90%, 95%, 96%, 97%, 98%, or 99% sequence similarity to a sequence listed Table 4.

TABLE 4
SARS-COV-2 Antibody Amino Acid Sequences.

| Antibody Name | | Heavy Chain Sequences |
|---|---|---|
| (Commercial Name) | Viral Antigen | Light Chain Sequences |
| bamlanivimab | Spike (S) | Heavy Chain: |
| (LY-CoV555) | glycoprotein | QVQLVQSGAEVKKPGSSVKVSCKASGGTFSNYAISWVRQAP |
| | receptor | GQGLEWMGRIIPILGIANYAQKFQGRVTITADKSTSTAYME |
| | binding | LSSLRSEDTAVYYCARGYYEARHYYYYYAMDVWGQGTAVTV |
| | domain (RBD) | SSASTKGPSVFPLAPSSKSTSGGTAALGCLVKDYFPEPVTV |
| | | SWNSGALTSGVHTFPAVLQSSGLYSLSSVVTVPSSSLGTQT |
| | | YICNVNHKPSNTKVDKRVEPKSCDKTHTCPPCPAPELLGGP |
| | | SVFLFPPKPKDTLMISRTPEVTCVVVDVSHEDPEVKFNWYV |
| | | DGVEVHNAKTKPREEQYNSTYRVVSVLTVLHQDWLNGKEYK |
| | | CKVSNKALPAPIEKTISKAKGQPREPQVYTLPPSREEMTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLSL |
| | | SPGK (SEQ ID NO: 53) |
| | | Light Chain: |
| | | DIQMTQSPSSLSASVGDRVTITCRASQSISSYLSWYQQKPG |
| | | KAPKLLIYAASSLQSGVPSRFSGSGSGTDFTLTITSLQPED |
| | | FATYYCQQSYSTPRTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC (SEQ ID NO: 54) |
| casirivimab | Spike (S) | Heavy Chain: |
| (REGEN-COVTM) | glycoprotein | QVQLVESGGGLVKPGGSLRLSCAASGFTFSDYYMSWIRQAP |
| | receptor | GKGLEWVSYITYSGSTIYYADSVKGRFTISRDNAKSSLYLQ |
| | binding | MNSLRAEDTAVYYCARDRGTTMVPFDYWGQGTLVTVSSAST |
| | domain (RBD) | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPG |
| | | (SEQ ID NO: 55) |
| | | Light Chain: |
| | | DIQMTQSPSSLSASVGDRVTITCQASQDITNYLNWYQQKPG |
| | | KAPKLLIYAASNLETGVPSRFSGSGSGTDFTFTISGLQPED |
| | | IATYYCQQYDNLPLTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC (SEQ ID NO: 56) |
| imdevimab | Spike (S) | Heavy Chain: |
| (REGEN-COV ™) | glycoprotein | QVQLVQSGAEVKKPGASVKVSCKASGYPFTSYGISWVRQAP |
| | receptor | GQGLEWMGWISTYQGNTNYAQKFQGRVTMTTDTSTTTGYME |
| | binding | LRRLRSDDTAVYYCARDYTRGAWFGESLIGGFDNWGQGTLV |
| | domain (RBD) | TVSSASTKGPSVFPLAPSSKSTSGGTAALGCLVKDYFPEPV |
| | | TVSWNSGALTSGVHTFPAVLQSSGLYSLSSVVTVPSSSLGT |
| | | QTYICNVNHKPSNTKVDKKVEPKSCDKTHTCPPCPAPELLG |
| | | GPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSRDELT |
| | | KNQVSLTCLVKGFYPSDIAVEWESNGQPENNYKTTPPVLDS |
| | | DGSFFLYSKLTVDKSRWQQGNVFSCSVLHEALHSHYTQKSL |
| | | SLSPGK (SEQ ID NO: 57) |
| | | Light Chain: |
| | | QSALTQPASVSGSPGQSITISCTGTSSDVGGYNYVSWYQQH |
| | | PGKAPKLMIYDVSKRPSGVSNRFSGSKSGNTASLTISGLQS |
| | | EDEADYYCNSLTSISTWVFGGGTKLTVLGQPKAAPSVTLFP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS (SEQ ID NO: 58) |
| sotrovimab | Spike (S) | Heavy Chain: |
| (Xevudy) | glycoprotein | QVQLVQSGAEVKKPGASVKVSCKASGYPFTSYGISWVRQAP |
| | receptor | GQGLEWMGWISTYQGNTNYAQKFQGRVTMTTDTSTTTGYME |
| | binding | LRRLRSDDTAVYYCARDYTRGAWFGESLIGGFDNWGQGTLV |
| | domain (RBD) | TVSSASTKGPSVFPLAPSSKSTSGGTAALGCLVKDYFPEPV |
| | | TVSWNSGALTSGVHTFPAVLQSSGLYSLSSVVTVPSSSLGT |
| | | QTYICNVNHKPSNTKVDKKVEPKSCDKTHTCPPCPAPELLG |
| | | GPSVFLFPPKPKDTLMISRTPEVTCVVVDVSHEDPEVKFNW |
| | | YVDGVEVHNAKTKPREEQYNSTYRVVSVLTVLHQDWLNGKE |
| | | YKCKVSNKALPAPIEKTISKAKGQPREPQVYTLPPSRDELT |
| | | KNQVSLTCLVKGFYPSDIAVEWESNGQPENNYKTTPPVLDS |
| | | DGSFFLYSKLTVDKSRWQQGNVFSCSVLHEALHSHYTQKSL |
| | | SLSPGK (SEQ ID NO: 59) |
| | | Light Chain: |
| | | EIVLTQSPGTLSLSPGERATLSCRASQTVSSTSLAWYQQKP |
| | | GQAPRLLIYGASSRATGIPDRFSGSGSGTDFTLTISRLEPE |
| | | DFAVYYCQQHDTSLTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC (SEQ ID NO: 60) |

*Sequences in Table 4 were determined by IMGT-monoclonal antibody database

In some embodiments, the antibody or functional fragment thereof is a Zika virus antibody. In some embodiments, the Zika virus antibody is ZIKV-117, Z3L1, Z20, Z23, ZV67, Z006, or 2A10G6. In some embodiments, the ZIKV-117 antibody or functional fragment comprises a heavy chain CDR1 amino acid sequence of GFTFKNYG (SEQ ID NO: 61), a heavy chain CDR2 amino acid sequence of VRYDGNNK (SEQ ID NO: 62), and a heavy chain CDR3 amino acid sequence of ARDPETFGGFDY (SEQ ID NO: 63), and a light chain CDR1 amino acid sequence of ESVSSN (SEQ ID NO: 64), light chain CDR2 amino acid sequence of GAS (SEQ ID NO: 65), and light chain CDR3 amino acid sequence of QQYYYSPRT (SEQ ID NO: 66).

In some embodiments, a nucleic acid provided herein codes for a protein or antibody sequence or a functional fragment thereof listed in Table 5. In some embodiments, compositions provided herein comprises two or more nucleic acids coding different sequences listed in Table 5. In some embodiments, the nucleic acid comprises at least 80%, 85%, 90%, 95%, 96%, 97%, 98%, or 99% sequence identity to a sequence listed in Table 5. In some embodiments, compositions provided herein comprise two or more nucleic acids coding different sequences listed in Table 5. In some embodiments, the nucleic acid provided herein comprises at least 80%, 85%, 90%, 95%, 96%, 97%, 98%, or 99% sequence similarity to a sequence listed Table 5. Exemplary nucleic acid sequences are listed in Table 5 below.

TABLE 5
Zika Virus Antibody Nucleic Acid Sequences.

| SEQ ID NO: | Nucleic Acid Sequence Name |
|---|---|
| SEQ ID NO: 67 | ZIKV-117 Full Length Antibody Sequence |
| SEQ ID NO: 68 | ZIKV-117 Heavy Chain Antibody Sequence |
| SEQ ID NO: 69 | ZIKV-117 Light Chain Antibody Sequence |

Provided herein are nucleic acids that encode a protein, an antibody, or an antibody fragment, wherein upon administration to a cell, population of cells, or a subject the protein, the antibody, or the antibody fragment effectively neutralizes a non-enveloped virus. In some embodiments, the non-enveloped virus is a Picornaviridae virus. In some embodiments, the Picornaviridae virus is an enterovirus. Further provided herein are nucleic acids that encode for a protein, an antibody, or an antibody fragment that specifically binds to an EV-D68 viral protein. In some embodiments, the EV-D68 viral protein is a VP1 capsid protein. In some embodiments, the nucleic acids encode for a viral protease.

### Nucleic Acids Encoding Cancer-Associated Proteins

Provided herein are compositions, wherein the compositions comprise: a lipid carrier provided herein; and one or more nucleic acids, wherein the one or more nucleic acids comprises a sequence encoding for an antigen. In some embodiments, the antigen is a cancer-associated protein (also referred to as a tumor protein antigen or tumor antigen). In some embodiments, nucleic acids provided herein encode for a cancer-associated protein. In some embodiments, the cancer-associated protein is a surface protein, a cytosolic protein, or a transmembrane protein. In some embodiments, the cancer-associated protein is a protein that is expressed by a cancer cell. In some embodiments, the cancer-associated protein is a protein that is expressed by a microbial organism that causes a cancer. For example, gamma herpesviruses such as Epstein-Barr virus can cause cancer in humans.

In some embodiments, nucleic acids provided herein encode for a protein expressed by a solid cancer cell or a blood cancer cell. In some embodiments, the solid cancer cell is a melanoma cell. In some embodiments, the protein expressed by the melanoma cell is not expressed by a non-cancer cell. In some embodiments, the protein expressed by a melanoma cell comprises a mutation in the amino acid sequence relative to a comparable amino acid sequence in a non-cancer cell. In some embodiments, nucleic acids provided herein encode for MAGE-A1 (SEQ ID NO: 70, SEQ ID NO: 73) or a functional fragment thereof. In some embodiments, nucleic acids provided herein encode for MAGE-A3 (SEQ ID NO: 71, SEQ ID NO: 74) or a functional fragment thereof. In some embodiments, nucleic acids provided herein encode for TRP-1 (SEQ ID NO: 72, SEQ ID NO: 75) or a functional fragment thereof. In some embodiments, nucleic acids provided herein encode for TRP-1 and MAGE-A1. In some embodiments, nucleic acids provided herein encode for TRP-1 and MAGE-A3. In some embodiments, nucleic acids provided herein encode for a tyrosinase. In some embodiments, nucleic acids provided herein comprise a sequence that is at least 85% identical to SEQ ID NOS: 70-72. In some embodiments, nucleic acids provided herein encode for an amino acid sequence listed in Table 6. In some embodiments, nucleic acids provided herein encode for an amino acid sequence that is at least 85% identical to any one of SEQ ID NOS: 73-124. In some embodiments, compositions provided herein comprise two or more, three or more, four or more, five or more, six or more, or up to seven or more nucleic acids coding different sequences listed in Table 6. In some embodiments, nucleic acids provided herein encoding for a protein sequence listed in Table 6 is used as part of a treatment or prevention of melanoma. In some embodiments, a nucleic acid provided herein encodes for a cancer-associated protein listed in Table 6. In some embodiments, compositions provided herein comprise two or more nucleic acids encoding for different sequences listed in Table 6. In some embodiments, nucleic acids provided herein encode for a cancer-associated protein sequence comprising at least 80%, 85%, 90%, 95%, 96%, 97%, 98%, or 99% sequence identity to a sequence listed in Table 6. In some embodiments, compositions provided herein comprise two or more nucleic acids encoding different sequences listed in Table 6. In some embodiments, the nucleic acid provided herein encodes for a cancer-associated protein or a functional fragment thereof comprising at least 80%, 85%, 90%, 95%, 96%, 97%, 98%, or 99% sequence similarity to a sequence listed Table 6. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated.

TABLE 6
Cancer-Associated Proteins.

| SEQ ID NO: | Reference Protein | Amino Acid Sequence |
|---|---|---|
| SEQ ID NO: 73 | MAGE-A1 | See Sequences Section |
| SEQ ID NO: 74 | MAGE-A3- melanoma- | See Sequences Section |
| | associated antigen 3 | |
| SEQ ID NO: 75 | TRP-1 (also known as | See Sequences Section |
| | 5,6-dihydroxyindole-2- | |
| | carboxylic acid oxidase, | |
| | DHICA oxidase, Melanoma | |
| | antigen glycoprotein 75, | |
| | Tyrosinase-related protein | |
| | 1, and TRYP-1) | |
| SEQ ID NO: 76 | Prostein | See Sequences Section |
| SEQ ID NO: 77 | Tyrosinase | KCDICTDEY |
| SEQ ID NO: 78 | Tyrosinase | YMDGTMSQV |
| SEQ ID NO: 79 | Tyrosinase | MLLAYLYQL |
| SEQ ID NO: 80 | Tyrosinase | AFLPWHRLF, |
| SEQ ID NO: 81 | Tyrosinase | SEIWRDIDF |
| SEQ ID NO: 82 | gp100/pMEL17 | YLEPGPVTA |
| SEQ ID NO: 83 | gp100/pMEL17 | KTWGQUWQV |
| SEQ ID NO: 84 | gp100/pMEL17 | ITDQVPFSV |
| SEQ ID NO: 85 | gp100/pMEL17 | VLYRYGSFSV |
| SEQ ID NO: 86 | gp100/pMEL17 | LLDGTATLRL |
| SEQ ID NO: 87 | gp100/pMEL17 | ALLAVGATK |
| SEQ ID NO: 88 | gp100/pMEL17 | MLGTHTMEV |
| SEQ ID NO: 89 | gp100/pMEL17 | LIYRRRLMK |
| SEQ ID NO: 90 | gp100/pMEL17 | ALNFPGSQK |
| SEQ ID NO: 91 | MART-1/MelanA | AAGIGILTV^{†} |
| SEQ ID NO: 92 | MART-1/MelanA | ILTVILGVL |
| SEQ ID NO: 93 | gp75/TRP-1 | MSLQRQFLR |
| SEQ ID NO: 94 | TRP-2 | SVYDFFVWL |
| SEQ ID NO: 95 | TRP-2 | LLGPGRPYR |
| SEQ ID NO: 96 | CEA | YLSGANLNL |
| SEQ ID NO: 97 | HER-2/neu | KIFGSLAFL |
| SEQ ID NO: 98 | HER-2/neu | VMAGVGSPYV |
| SEQ ID NO: 99 | HER-2/neu | IISAVVGIL |
| SEQ ID NO: 100 | PSMA | LLHETDSAV |
| SEQ ID NO: 101 | PSMA | ALFDIESK V |
| SEQ ID NO: 102 | MAGE-1 | EADPTGHSY |
| SEQ ID NO: 103 | MAGE-1 | SLFRAVITK |
| SEQ ID NO: 104 | MAGE-1 | SAYGEPRKL |
| SEQ ID NO: 105 | MAGE-2 | KMVELVHFL |
| SEQ ID NO: 106 | MAGE-2 | YLOLVFGIEV |
| SEQ ID NO: 107 | MAGE-3 | EVDPIGHLY |
| SEQ ID NO: 108 | MAGE-3 | FLWGPRALV |
| SEQ ID NO: 109 | MAGE-3 | MEVDPIGHLY |
| SEQ ID NO: 110 | BAGE | AARAVFLAL |
| SEQ ID NO: 111 | GAGE-1,2 | YRPRPRRY |
| SEQ ID NO: 112 | GnT-V | VLPDVFIRC |
| SEQ ID NO: 113 | NY-ESO-1 | QLSLLMWIT |
| SEQ ID NO: 114 | NY-ESO-1 | SLLMWITQC |
| SEQ ID NO: 115 | NY-ESO-1 | ASGPGGGAPR |
| SEQ ID NO: 116 | 43kD protein | QDLTMKYQIF |
| SEQ ID NO: 117 | p15 | (E)AYGLDFYIL |
| SEQ ID NO: 118 | Mutated beta-catenin | SYLDSGIHF^{‡} |
| SEQ ID NO: 119 | Mutated elongation | ETVSEQSNV^{§} |
| | factor 2 | |
| SEQ ID NO: 120 | Mutated CASP-8 | FPSDSWCYF |
| | (FLICE/MACH) | |
| SEQ ID NO: 121 | MUM-1 gene product | EEKLIVVLF^{¶} |
| | mutated across | |
| | intron/exon junction | |

^{†}AAGIGILTV is also recognized by HLA B45-1- restricted cytotoxic T lymphocyte.
^{‡}Phenylalanine (F) at position 9 is the result of mutation. The wild-type sequence is SYLDSGIHS (SEQ ID NO: 122).
^{§}Glutamine (Q) at position 6 is the result of somatic mutation. The wild-type sequence is ETVSEESNV (SEQ ID NO: 123).
^{¶}Isoleucine (I) at position 5 is the result of mutation. The wild-type sequence is EEKLSVVLF (SEQ ID NO: 124).

In some embodiments, a cancer-associated protein encoded by a nucleic acid provided herein comprises a cell membrane-contacting domain or functional fragment thereof. In some embodiments, the cell membrane-contacting domain comprises a transmembrane-binding domain, an outer cell membrane-contacting domain, or an inner cell membrane-contacting domain. In some embodiments, the cell membrane-contacting domain comprises a transmembrane-binding domain, an outer cell membrane-contacting domain, and an inner cell membrane-contacting domain. In some embodiments, the cancer-associated protein is a protein expressed by a melanoma cancer cell, a prostate cancer cell, a colon cancer cell, an ovarian cancer cell, a breast cancer cell, a pancreatic cancer cell, or a blood cell.

In some embodiments, a nucleic acid provided herein comprises a sequence encoding a dimer, trimer, or multimer of a cancer-associated protein provided herein. In some embodiments, nucleic acid provided herein comprises a sequence encoding an amino acid sequence that is at least about 500 amino acids in length or more. In some embodiments, nucleic acid provided herein comprises a sequence encoding an amino acid sequence that is at least about 200, 300, 400, 500, 750, 1000 or more amino acids in length or more. In some embodiments, nucleic acid provided herein comprises a sequence encoding one or more cancer-associated protein, wherein the one or more cancer-associated protein comprises a molecular weight of at least about 50 kilodaltons (kDa) or more, at least about 100 kilodaltons (kDa) or more, at least about 150 kilodaltons (kDa) or more, at least about 200 kilodaltons (kDa) or more, at least about 250 kilodaltons (kDa) or more, at least about 300 kilodaltons (kDa) or more, at least about 350 kilodaltons (kDa) or more, at least about 400 kilodaltons (kDa) or more, at least about 450 kilodaltons (kDa) or more, at least about 500 kilodaltons (kDa) or more, up to 1000 kDa or more. In some embodiments, nucleic acids provided herein comprise a sequence encoding one or more cancer-associated protein, wherein the one or more cancer-associated protein comprises a molecular weight of at least about 59 kDa or more.

In some embodiments, nucleic acids provided herein comprise a sequence encoding a cancer-associated protein associated with prostate cancer. In some embodiments, the cancer-associated protein with prostate cancer comprises prostein. In some embodiments, the cancer-associated protein is prostein. In some embodiments, the cancer-associated protein is at least about 50%, 60%, 70%, 80%, 90%, 95%, or 100% of full length prostein. In some embodiments, the prostein is human prostein. In some embodiments, the cancer-associated protein comprises an amino acid sequence that is at least about 80%, 85%, 90%, 95% or 100% identical to any one of SEQ ID NOS: 73-124.

### Nucleic Acids Encoding Cancer Antigen Binding Molecules

In some embodiments, a nucleic acid provided herein encodes for an antibody or functional fragment thereof that binds to a tumor antigen. In some embodiments, the antibody or functional fragment thereof is a cancer therapeutic antibody. In some embodiments, the cancer therapeutic antibody is atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab. Exemplary amino acid sequences for cancer therapeutic antibodies are provided below in Table 7.

In some embodiments, a nucleic acid provided herein codes for a protein or antibody amino acid sequence or a functional fragment thereof listed in Table 7. In some embodiments, compositions provided herein comprises two or more nucleic acids coding for different sequences listed in Table 7. In some embodiments, the nucleic acid provided herein codes for a protein or antibody amino acid sequence or a functional fragment thereof comprising at least 80%, 85%, 90%, 95%, 96%, 97%, 98%, or 99% sequence identity to a sequence listed in Table 7. In some embodiments, compositions provided herein comprise two or more nucleic acids coding different sequences listed in Table 7. In some embodiments, the nucleic acid provided herein codes for a protein, antibody, or a functional fragment thereof comprising at least 80%, 85%, 90%, 95%, 96%, 97%, 98%, or 99% sequence similarity to a sequence listed Table 7.

TABLE 7
Cancer Therapeutic Antibodies.

| SEQ | Antibody Name | | |
|---|---|---|---|
| ID | (Commercial | | |
| NO: | Name) | Tumor Antigen | Heavy Chain Amino Acid Sequences |
| 125 | atezolizumab | CD274 (programmed | EVQLVESGGGLVQPGGSLRLSCAASGFTFSDSWIHWVR |
| | (TECENTRIQ ®) | cell death 1 ligand 1, | QAPGKGLEWVAWISPYGGSTYYADSVKGRFTISADTSK |
| | | B7H1, B7-H1, PDL1, | NTAYLQMNSLRAEDTAVYYCARRHWPGGFDYWGQGTLV |
| | | PD-L1, PDCDIL1, B7 | TVSSASTKGPSVFPLAPSSKSTSGGTAALGCLVKDYFP |
| | | homolog 1, B7 | EPVTVSWNSGALTSGVHTFPAVLQSSGLYSLSSVVTVP |
| | | homologue 1) [Homo | SSSLGTQTYICNVNHKPSNTKVDKKVEPKSCDKTHTCP |
| | | sapiens] | PCPAPELLGGPSVFLFPPKPKDTLMISRTPEVTCVVVD |
| | | | VSHEDPEVKFNWYVDGVEVHNAKTKPREEQYASTYRVV |
| | | | SVLTVLHQDWLNGKEYKCKVSNKALPAPIEKTISKAKG |
| | | | QPREPQVYTLPPSREEMTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPGK |
| 126 | avelumab | CD274 | EVQLLESGGGLVQPGGSLRLSCAASGFTFSSYIMMWVR |
| | (BAVENCIO ®) | | QAPGKGLEWVSSIYPSGGITFYADTVKGRFTISRDNSK |
| | | | NTLYLQMNSLRAEDTAVYYCARIKLGTVTTVDYWGQGT |
| | | | LVTVSSASTKGPSVFPLAPSSKSTSGGTAALGCLVKDY |
| | | | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSRDELTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGK |
| 127 | bevacizumab | VEGFA | EVQLVESGGGLVQPGGSLRLSCAASGYTFTNYGMNWVR |
| | (AVASTIN ®) | (vascular | QAPGKGLEWVGWINTYTGEPTYAADFKRRFTFSLDTSK |
| | | endothelial growth | STAYLQMNSLRAEDTAVYYCAKYPHYYGSSHWYFDVWG |
| | | factor A, VEGF-A, | QGTLVTVSSASTKGPSVFPLAPSSKSTSGGTAALGCLV |
| | | VEGF) [Homo | KDYFPEPVTVSWNSGALTSGVHTFPAVLQSSGLYSLSS |
| | | sapiens] | VVTVPSSSLGTQTYICNVNHKPSNTKVDKKVEPKSCDK |
| | | | THTCPPCPAPELLGGPSVFLFPPKPKDTLMISRTPEVT |
| | | | CVVVDVSHEDPEVKFNWYVDGVEVHNAKTKPREEQYNS |
| | | | TYRVVSVLTVLHQDWLNGKEYKCKVSNKALPAPIEKTI |
| | | | SKAKGQPREPQVYTLPPSREEMTKNQVSLTCLVKGFYP |
| | | | SDIAVEWESNGQPENNYKTTPPVLDSDGSFFLYSKLTV |
| | | | DKSRWQQGNVFSCSVMHEALHNHYTQKSLSLSPGK |
| 128 | cemiplimab | PDCD1 (programmed | EVQLLESGGVLVQPGGSLRLSCAASGFTFSNFGMTWVR |
| | (LIBTAYO ®) | cell death 1, PD1, PD- | QAPGKGLEWVSGISGGGRDTYFADSVKGRFTISRDNSK |
| | | 1, CD279) [Homo | NTLYLQMNSLKGEDTAVYYCVKWGNIYFDYWGQGTLVT |
| | | sapiens] | VSSASTKGPSVFPLAPCSRSTSESTAALGCLVKDYFPE |
| | | | PVTVSWNSGALTSGVHTFPAVLQSSGLYSLSSVVTVPS |
| | | | SSLGTKTYTCNVDHKPSNTKVDKRVESKYGPPCPPCPA |
| | | | PEFLGGPSVFLFPPKPKDTLMISRTPEVTCVVVDVSQE |
| | | | DPEVQFNWYVDGVEVHNAKTKPREEQFNSTYRVVSVLT |
| | | | VLHQDWLNGKEYKCKVSNKGLPSSIEKTISKAKGQPRE |
| | | | PQVYTLPPSQEEMTKNQVSLTCLVKGFYPSDIAVEWES |
| | | | NGQPENNYKTTPPVLDSDGSFFLYSRLTVDKSRWQEGN |
| | | | VFSCSVMHEALHNHYTQKSLSLSLGK |
| 129 | cetuximab | EGFR (epidermal | QVQLKQSGPGLVQPSQSLSITCTVSGFSLTNYGVHWVR |
| | (ERBITUX ®) | growth factor receptor, | QSPGKGLEWLGVIWSGGNTDYNTPFTSRLSINKDNSKS |
| | | receptor tyrosine- | QVFFKMNSLQSNDTAIYYCARALTYYDYEFAYWGQGTL |
| | | protein kinase erbB-1, | VTVSAASTKGPSVFPLAPSSKSTSGGTAALGCLVKDYF |
| | | ERBB1, HER1, HER- | PEPVTVSWNSGALTSGVHTFPAVLQSSGLYSLSSVVTV |
| | | 1, ERBB) [Homo | PSSSLGTQTYICNVNHKPSNTKVDKRVEPKSCDKTHTC |
| | | sapiens] | PPCPAPELLGGPSVFLFPPKPKDTLMISRTPEVTCVVV |
| | | | DVSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSREEMTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPGK |
| 130 | daratumumab | CD38 (ADP-ribosy1 | EVQLLESGGGLVQPGGSLRLSCAVSGFTFNSFAMSWVR |
| | (DARZALEX ™; | cyclase 1, cyclic ADP- | QAPGKGLEWVSAISGSGGGTYYADSVKGRFTISRDNSK |
| | | ribose hydrolase 1, | NTLYLQMNSLRAEDTAVYFCAKDKILWFGEPVFDYWGQ |
| | DARZALEX; | cADPr hydrolase 1, | GTLVTVSSASTKGPSVFPLAPSSKSTSGGTAALGCLVK |
| | FASPRO ™) | T10) [Homo sapiens] | DYFPEPVTVSWNSGALTSGVHTFPAVLQSSGLYSLSSV |
| | | | VTVPSSSLGTQTYICNVNHKPSNTKVDKRVEPKSCDKT |
| | | | HTCPPCPAPELLGGPSVFLFPPKPKDTLMISRTPEVTC |
| | | | VVVDVSHEDPEVKFNWYVDGVEVHNAKTKPREEQYNST |
| | | | YRVVSVLTVLHQDWLNGKEYKCKVSNKALPAPIEKTIS |
| | | | KAKGQPREPQVYTLPPSREEMTKNQVSLTCLVKGFYPS |
| | | | DIAVEWESNGQPENNYKTTPPVLDSDGSFFLYSKLTVD |
| | | | KSRWQQGNVFSCSVMHEALHNHYTQKSLSLSPGK |
| 131 | dinutuximab | ganglioside GD2 | EVQLLQSGPELEKPGASVMISCKASGSSFTGYNMNWVR |
| | (UNITUXIN ™) | (disialoganglioside | QNIGKSLEWIGAIDPYYGGTSYNQKFKGRATLTVDKSS |
| | | GD2) [Homo sapiens] | STAYMHLKSLTSEDSAVYYCVSGMEYWGQGTSVTVSSA |
| | | | STKGPSVFPLAPSSKSTSGGTAALGCLVKDYFPEPVTV |
| | | | SWNSGALTSGVHTFPAVLQSSGLYSLSSVVTVPSSSLG |
| | | | TQTYICNVNHKPSNTKVDKRVEPKSCDKTHTCPPCPAP |
| | | | ELLGGPSVFLFPPKPKDTLMISRTPEVTCVVVDVSHED |
| | | | PEVKFNWYVDGVEVHNAKTKPREEQYNSTYRVVSVLTV |
| | | | LHQDWLNGKEYKCKVSNKALPAPIEKTISKAKGQPREP |
| | | | QVYTLPPSREEMTKNQVSLTCLVKGFYPSDIAVEWESN |
| | | | GQPENNYKTTPPVLDSDGSFFLYSKLTVDKSRWQQGNV |
| | | | FSCSVMHEALHNHYTQKSLSLSPGK |
| 132 | durvalumab | CD274 | EVQLVESGGGLVQPGGSLRLSCAASGFTFSRYWMSWVR |
| | (IMFINZI ™) | | QAPGKGLEWVANIKQDGSEKYYVDSVKGRFTISRDNAK |
| | | | NSLYLQMNSLRAEDTAVYYCAREGGWFGELAFDYWGQG |
| | | | TLVTVSSASTKGPSVFPLAPSSKSTSGGTAALGCLVKD |
| | | | YFPEPVTVSWNSGALTSGVHTFPAVLQSSGLYSLSSVV |
| | | | TVPSSSLGTQTYICNVNHKPSNTKVDKRVEPKSCDKTH |
| | | | TCPPCPAPEFEGGPSVFLFPPKPKDTLMISRTPEVTCV |
| | | | VVDVSHEDPEVKFNWYVDGVEVHNAKTKPREEQYNSTY |
| | | | RVVSVLTVLHQDWLNGKEYKCKVSNKALPASIEKTISK |
| | | | AKGQPREPQVYTLPPSREEMTKNQVSLTCLVKGFYPSD |
| | | | IAVEWESNGQPENNYKTTPPVLDSDGSFFLYSKLTVDK |
| | | | SRWQQGNVFSCSVMHEALHNHYTQKSLSLSPGK |
| 133 | elotuzumab | SLAMF7 (SLAM | EVQLVESGGGLVQPGGSLRLSCAASGFDFSRYWMSWVR |
| | (EMPLICITI ™) | family member 7, CD2 | QAPGKGLEWIGEINPDSSTINYAPSLKDKFIISRDNAK |
| | | subset 1, CS1, CD2- | NSLYLQMNSLRAEDTAVYYCARPDGNYWYFDVWGQGTL |
| | | like | VTVSSASTKGPSVFPLAPSSKSTSGGTAALGCLVKDYF |
| | | receptor- | PEPVTVSWNSGALTSGVHTFPAVLQSSGLYSLSSVVTV |
| | | activating cytotoxic | PSSSLGTQTYICNVNHKPSNTKVDKKVEPKSCDKTHTC |
| | | cells, CRACC, 19A24, | PPCPAPELLGGPSVFLFPPKPKDTLMISRTPEVTCVVV |
| | | CD319) [Homo | DVSHEDPEVKFNWYVDGVEVHNAKTKPREEQYNSTYRV |
| | | sapiens] | VSVLTVLHQDWLNGKEYKCKVSNKALPAPIEKTISKAK |
| | | | GQPREPQVYTLPPSRDELTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPGK |
| 134 | ipilimumab | CTLA4 (cytotoxic T- | QVQLVESGGGVVQPGRSLRLSCAASGFTFSSYTMHWVR |
| | (YERVOY ®) | lymphocyte-associated | QAPGKGLEWVTFISYDGNNKYYADSVKGRFTISRDNSK |
| | | protein 4, CD152) | NTLYLQMNSLRAEDTAIYYCARTGWLGPFDYWGQGTLV |
| | | [Homo sapiens] | TVSSASTKGPSVFPLAPSSKSTSGGTAALGCLVKDYFP |
| | | | EPVTVSWNSGALTSGVHTFPAVLQSSGLYSLSSVVTVP |
| | | | SSSLGTQTYICNVNHKPSNTKVDKRVEPKSCDKTHTCP |
| | | | PCPAPELLGGPSVFLFPPKPKDTLMISRTPEVTCVVVD |
| | | | VSHEDPEVKFNWYVDGVEVHNAKTKPREEQYNSTYRVV |
| | | | SVLTVLHQDWLNGKEYKCKVSNKALPAPIEKTISKAKG |
| | | | QPREPQVYTLPPSRDELTKNQVSLTCLVKGFYPSDIAV |
| | | | EWESNGQPENNYKTTPPVLDSDGSFFLYSKLTVDKSRW |
| | | | QQGNVFSCSVMHEALHNHYTQKSLSLSPGK |
| 135 | isatuximab | CD38 (ADP-ribosy1 | QVQLVQSGAEVAKPGTSVKLSCKASGYTFTDYWMQWVK |
| | (SARCLISA ®) | cyclase 1, cyclic ADP- | QRPGQGLEWIGTIYPGDGDTGYAQKFQGKATLTADKSS |
| | | ribose hydrolase 1, | KTVYMHLSSLASEDSAVYYCARGDYYGSNSLDYWGQGT |
| | | cADPr hydrolase 1, | SVTVSSASTKGPSVFPLAPSSKSTSGGTAALGCLVKDY |
| | | T10) [Homo sapiens] | FPEPVTVSWNSGALTSGVHTFPAVLQSSGLYSLSSVVT |
| | | | VPSSSLGTQTYICNVNHKPSNTKVDKKVEPKSCDKTHT |
| | | | CPPCPAPELLGGPSVFLFPPKPKDTLMISRTPEVTCVV |
| | | | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSRDELTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPGK |
| 136 | mogamulizumab | CCR4 (chemokine (C- | EVQLVESGGDLVQPGRSLRLSCAASGFIFSNYGMSWVR |
| | (POTELIGEO ®) | C motif) receptor 4, | QAPGKGLEWVATISSASTYSYYPDSVKGRFTISRDNAK |
| | | CC chemokine | NSLYLQMNSLRVEDTALYYCGRHSDGNFAFGYWGQGTL |
| | | receptor 4, CCR-4, | VTVSSASTKGPSVFPLAPSSKSTSGGTAALGCLVKDYF |
| | | CKR4, k5-5, CD194) | PEPVTVSWNSGALTSGVHTFPAVLQSSGLYSLSSVVTV |
| | | [Homo sapiens] | PSSSLGTQTYICNVNHKPSNTKVDKKVEPKSCDKTHTC |
| | | | PPCPAPELLGGPSVFLFPPKPKDTLMISRTPEVTCVVV |
| | | | DVSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSRDELTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPGK |
| 137 | necitumumab | EGFR (epidermal | QVQLQESGPGLVKPSQTLSLTCTVSGGSISSGDYYWSW |
| | (PORTRAZZA ™) | growth factor receptor, | IRQPPGKGLEWIGYIYYSGSTDYNPSLKSRVTMSVDTS |
| | | receptor tyrosine- | KNQFSLKVNSVTAADTAVYYCARVSIFGVGTFDYWGQG |
| | | protein kinase erbB-1, | TLVTVSSASTKGPSVLPLAPSSKSTSGGTAALGCLVKD |
| | | ERBB1, HER1, HER- | YFPEPVTVSWNSGALTSGVHTFPAVLQSSGLYSLSSVV |
| | | 1, ERBB) [Homo | TVPSSSLGTQTYICNVNHKPSNTKVDKRVEPKSCDKTH |
| | | sapiens] | TCPPCPAPELLGGPSVFLFPPKPKDTLMISRTPEVTCV |
| | | | VVDVSHEDPEVKFNWYVDGVEVHNAKTKPREEQYNSTY |
| | | | RVVSVLTVLHQDWLNGKEYKCKVSNKALPAPIEKTISK |
| | | | AKGQPREPQVYTLPPSREEMTKNQVSLTCLVKGFYPSD |
| | | | IAVEWESNGQPENNYKTTPPVLDSDGSFFLYSKLTVDK |
| | | | SRWQQGNVFSCSVMHEALHNHYTQKSLSLSPGK |
| 138 | nivolumab | PDCD1 (programmed | QVQLVESGGGVVQPGRSLRLDCKASGITFSNSGMHWVR |
| | (OPDIVO ®) | cell death 1, PD1, PD- | QAPGKGLEWVAVIWYDGSKRYYADSVKGRFTISRDNSK |
| | | 1, CD279) [Homo | NTLFLQMNSLRAEDTAVYYCATNDDYWGQGTLVTVSSA |
| | | sapiens] | STKGPSVFPLAPCSRSTSESTAALGCLVKDYFPEPVTV |
| | | | SWNSGALTSGVHTFPAVLQSSGLYSLSSVVTVPSSSLG |
| | | | TKTYTCNVDHKPSNTKVDKRVESKYGPPCPPCPAPEFL |
| | | | GGPSVFLFPPKPKDTLMISRTPEVTCVVVDVSQEDPEV |
| | | | QFNWYVDGVEVHNAKTKPREEQFNSTYRVVSVLTVLHQ |
| | | | DWLNGKEYKCKVSNKGLPSSIEKTISKAKGQPREPQVY |
| | | | TLPPSQEEMTKNQVSLTCLVKGFYPSDIAVEWESNGQP |
| | | | ENNYKTTPPVLDSDGSFFLYSRLTVDKSRWQEGNVFSC |
| | | | SVMHEALHNHYTQKSLSLSLGK |
| 139 | obinutuzumab | MS4A1 (membrane- | QVQLVQSGAEVKKPGSSVKVSCKASGYAFSYSWINWVR |
| | (GAZYVA ®) | spanning 4-domains | QAPGQGLEWMGRIFPGDGDTDYNGKFKGRVTITADKST |
| | | subfamily A member | STAYMELSSLRSEDTAVYYCARNVFDGYWLVYWGQGTL |
| | | 1, CD20) [Homo | VTVSSASTKGPSVFPLAPSSKSTSGGTAALGCLVKDYF |
| | | sapiens] | PEPVTVSWNSGALTSGVHTFPAVLQSSGLYSLSSVVTV |
| | | | PSSSLGTQTYICNVNHKPSNTKVDKKVEPKSCDKTHTC |
| | | | PPCPAPELLGGPSVFLFPPKPKDTLMISRTPEVTCVVV |
| | | | DVSHEDPEVKFNWYVDGVEVHNAKTKPREEQYNSTYRV |
| | | | VSVLTVLHQDWLNGKEYKCKVSNKALPAPIEKTISKAK |
| | | | GQPREPQVYTLPPSRDELTKNQVSLTCLVKGFYPSDIA |
| | | | VEWESNGQPENNYKTTPPVLDSDGSFFLYSKLTVDKSR |
| | | | WQQGNVFSCSVMHEALHNHYTQKSLSLSPGK |
| 140 | ofatumumab | MS4A1 (membrane- | EVQLVESGGGLVQPGRSLRLSCAASGFTFNDYAMHWVR |
| | (ARZERRA ® | spanning 4-domains | QAPGKGLEWVSTISWNSGSIGYADSVKGRFTISRDNAK |
| | KESIMPTA ®) | subfamily A member | KSLYLQMNSLRAEDTALYYCAKDIQYGNYYYGMDVWGQ |
| | | 1, CD20) [Homo | GTTVTVSSASTKGPSVFPLAPGSSKSTSGTAALGCLVK |
| | | sapiens] | DYFPEPVTVSWNSGALTSGVHTFPAVLQSSGLYSLSSV |
| | | | VTVPSSSLGTQTYICNVNHKPSNTKVDKKVEP |
| 141 | olaratumab | PDGFRA (platelet- | QLQLQESGPGLVKPSETLSLTCTVSGGSINSSSYYWGW |
| | (LARTRUVO ™) | derived growth factor | LRQSPGKGLEWIGSFFYTGSTYYNPSLRSRLTISVDTS |
| | | receptor alpha subunit, | KNQFSLMLSSVTAADTAVYYCARQSTYYYGSGNYYGWF |
| | | PDGFR2, CD140a) | DRWDQGTLVTVSSASTKGPSVFPLAPSSKSTSGGTAAL |
| | | [Homo sapiens] | GCLVKDYFPEPVTVSWNSGALTSGVHTFPAVLQSSGLY |
| | | | SLSSVVTVPSSSLGTQTYICNVNHKPSNTKVDKRVEPK |
| | | | SCDKTHTCPPCPAPELLGGPSVFLFPPKPKDTLMISRT |
| | | | PEVTCVVVDVSHEDPEVKFNWYVDGVEVHNAKTKPREE |
| | | | QYNSTYRVVSVLTVLHQDWLNGKEYKCKVSNKALPAPI |
| | | | EKTISKAKGQPREPQVYTLPPSREEMTKNQVSLTCLVK |
| | | | GFYPSDIAVEWESNGQPENNYKTTPPVLDSDGSFFLYS |
| | | | KLTVDKSRWQQGNVFSCSVMHEALHNHYTQKSLSLSPG |
| 142 | panitumumab | EGFR (epidermal | QVQLQESGPGLVKPSETLSLTCTVSGGSVSSGDYYWTW |
| | (VECTIBIX ®) | growth factor receptor, | IRQSPGKGLEWIGHIYYSGNTNYNPSLKSRLTISIDTS |
| | | receptor tyrosine- | KTQFSLKLSSVTAADTAIYYCVRDRVTGAFDIWGQGTM |
| | | protein kinase erbB-1, | VTVSSASTKGPSVFPLAPCSRSTSESTAALGCLVKDYF |
| | | ERBB1, HER1, HER- | PEPVTVSWNSGALTSGVHTFPAVLQSSGLYSLSSVVTV |
| | | 1, ERBB) [Homo | PSSNFGTQTYTCNVDHKPSNTKVDKTVERK |
| | | sapiens] | |
| 143 | pembrolizumab | PDCD1 (programmed | VQLVQSGVEVKKPGASVKVSCKASGYTFTNYYMYWVRQ |
| | (KEYTRUDA ®) | cell death 1, PD1, PD- | APGQGLEWMGGINPSNGGTNFNEKFKNRVTLTTDSSTT |
| | | 1, CD279) [Homo | TAYMELKSLQFDDTAVYYCARRDYRFDMGFDYWGQGTT |
| | | sapiens] | VTVSSASTKGPSVFPLAPCSRSTSESTAALGCLVKDYF |
| | | | PEPVTVSWNSGALTSGVHTFPAVLQSSGLYSLSSVVTV |
| | | | PSSSLGTKTYTCNVDHKPSNTKVDKRVESKYGPPCPPC |
| | | | PAPEFLGGPSVFLFPPKPKDTLMISRTPEVTCVVVDVS |
| | | | QEDPEVQFNWYVDGVEVHNAKTKPREEQFNSTYRVVSV |
| | | | LTVLHQDWLNGKEYKCKVSNKGLPSSIEKTISKAKGQP |
| | | | REPQVYTLPPSQEEMTKNQVSLTCLVKGFYPSDIAVEW |
| | | | ESNGQPENNYKTTPPVLDSDGSFFLYSRLTVDKSRWQE |
| | | | GNVFSCSVMHEALHNHYTQKSLSLSLGK |
| 144 | pertuzumab | ERBB2 (epidermal | EVQLVESGGGLVQPGGSLRLSCAASGFTFTDYTMDWVR |
| | (PERJETA ®) | growth factor receptor | QAPGKGLEWVADVNPNSGGSIYNQRFKGRFTLSVDRSK |
| | | 2, receptor tyrosine- | NTLYLQMNSLRAEDTAVYYCARNLGPSFYFDYWGQGTL |
| | | protein kinase erbB-2, | VTVSSASTKGPSVFPLAPSSKSTSGGTAALGCLVKDYF |
| | | EGFR2, HER2, HER- | PEPVTVSWNSGALTSGVHTFPAVLQSSGLYSLSSVVTV |
| | | 2, p185c-erbB2, NEU, | PSSSLGTQTYICNVNHKPSNTKVDKKVEPKSCDKTH |
| | | CD340) [Homo | |
| | | sapiens] | |
| 145 | ramucirumab | KDR (kinase insert | EVQLVQSGGGLVKPGGSLRLSCAASGFTFSSYSMNWVR |
| | (CYRAMZA ™) | domain receptor, | QAPGKGLEWVSSISSSSSYIYYADSVKGRFTISRDNAK |
| | | vascular endothelial | NSLYLQMNSLRAEDTAVYYCARVTDAFDIWGQGTMVTV |
| | | growth factor receptor | SSASTKGPSVLPLAPSSKSTSGGTAALGCLVKDYFPEP |
| | | 2, VEGFR2, VEGF- | VTVSWNSGALTSGVHTFPAVLQSSGLYSLSSVVTVPSS |
| | | R2, FLK1, CD309) | SLGTQTYICNVNHKPSNTKVDKRVEPKSCDKTHTCPPC |
| | | [Homo sapiens] | PAPELLGGPSVFLFPPKPKDTLMISRTPEVTCVVVDVS |
| | | | HEDPEVKFNWYVDGVEVHNAKTKPREEQYNSTYRVVSV |
| | | | LTVLHQDWLNGKEYKCKVSNKALPAPIEKTISKAKGQP |
| | | | REPQVYTLPPSREEMTKNQVSLTCLVKGFYPSDIAVEW |
| | | | ESNGQPENNYKTTPPVLDSDGSFFLYSKLTVDKSRWQQ |
| | | | GNVFSCSVMHEALHNHYTQKSLSLSPGK |
| 146 | rituximab | MS4A1 (membrane- | QVQLQQPGAELVKPGASVKMSCKASGYTFTSYNMHWVK |
| | (RITUXAN ®) | spanning 4-domains | QTPGRGLEWIGAIYPGNGDTSYNQKFKGKATLTADKSS |
| | | subfamily A member | STAYMQLSSLTSEDSAVYYCARSTYYGGDWYFNVWGAG |
| | | 1, CD20) [Homo | TTVTVSAASTKGPSVFPLAPSSKSTSGGTAALGCLVKD |
| | | sapiens] | YFPEPVTVSWNSGALTSGVHTFPAVLQSSGLYSLSSVV |
| | | | TVPSSSLGTQTYICNVNHKPSNTKVDKKVEPKSCDKTH |
| | | | TCPPCPAPELLGGPSVFLFPPKPKDTLMISRTPEVTCV |
| | | | VVDVSHEDPEVKFNWYVDGVEVHNAKTKPREEQYNSTY |
| | | | RVVSVLTVLHQDWLNGKEYKCKVSNKALPAPIEKTISK |
| | | | AKGQPREPQVYTLPPSRDELTKNQVSLTCLVKGFYPSD |
| | | | IAVEWESNGQPENNYKTTPPVLDSDGSFFLYSKLTVDK |
| | | | SRWQQGNVFSCSVMHEALHNHYTQKSLSLSPGK |
| 147 | trastuzumab | ERBB2 (epidermal | EVQLVESGGGLVQPGGSLRLSCAASGFNIKDTYIHWVR |
| | (HERCEPTIN ®) | growth factor receptor | QAPGKGLEWVARIYPTNGYTRYADSVKGRFTISADTSK |
| | | 2, receptor tyrosine- | NTAYLQMNSLRAEDTAVYYCSRWGGDGFYAMDYWGQGT |
| | | protein kinase erbB-2, | LVTVSSASTKGPSVFPLAPSSKSTSGGTAALGCLVKDY |
| | | EGFR2, HER2, HER- | FPEPVTVSWNSGALTSGVHTFPAVLQSSGLYSLSSVVT |
| | | 2, p185c-erbB2, NEU, | VPSSSLGTQTYICNVNHKPSNTKVDKKVEPKSCDKTHT |
| | | CD340) [Homo | CPPCPAPELLGGPSVFLFPPKPKDTLMISRTPEVTCVV |
| | | sapiens] | VDVSHEDPEVKFNWYVDGVEVHNAKTKPREEQYNSTYR |
| | | | VVSVLTVLHQDWLNGKEYKCKVSNKALPAPIEKTISKA |
| | | | KGQPREPQVYTLPPSREEMTKNQVSLTCLVKGFYPSDI |
| | | | AVEWESNGQPENNYKTTPPVLDSDGSFFLYSKLTVDKS |
| | | | RWQQGNVFSCSVMHEALHNHYTQKSLSLSPG |

*Sequences in Table 7 were determined by IMGT-monoclonal antibody database

As an alternative to, or in addition to the delivery of RNAs as antigens, combinations can be used, e.g., RNA antigens combined with RNAs that stimulate innate immune responses, or RNAs that launch oncolytic viruses, or live-attenuated viruses.

In certain embodiments, the bioactive agent in of a composition provided herein comprises a combination of RNA-encoded antigens with another RNA that can stimulate innate immune responses or can launch oncolytic viruses or live-attenuated viruses. Alternatively, compositions provided herein that contain RNA-encoded antigens can be combined with a formulation that contains another RNA that can stimulate innate immune responses or can launch oncolytic viruses or live-attenuated viruses.

### RNA Encoding Immunomodulatory Molecules

Provided herein are nucleic acid sensor engaging compositions referred to as pattern recognition receptor (PRR) agonists. In some embodiments, the PRR agonists are a nucleic acid. The nucleic acid may be single-stranded or double-stranded. The nucleic acid may be RNA or DNA. The nucleic acid may be linear or include a hairpin. In some embodiments, the PRR is an endosomal nucleic acid sensor. In some embodiments, the endosomal nucleic acid sensor is toll-like receptor (TLR). Exemplary TLR PRRs include TLR3, TLR7, TLR8, and TLR9. In some embodiments, the TLR PRR is TLR3. In some embodiments, the TLR3 agonist is RIBOXXOL, poly(I:C), or Hiltonol®. In some embodiments, the PRR is a DNA sensor. Exemplary DNA sensor PRRs include cyclic GMP-AMP synthase (cGAS). In some embodiments, the PRR is a retinoic acid-inducible gene I (RIG-I)-like receptor (RLR). In some embodiments, the RLR is RIG-I, melanoma differentiation-associated protein 5 (MDA5), or laboratory of genetics physiology 2 (LGP2). In some embodiments, the PRR agonist is a viral RNA sequence, or a functional variant thereof. In some embodiments, the PRR agonist comprises a triphosphate (PPP) group at the 5′ end. In some embodiments, the PRR agonist comprising a triphosphate (PPP) group at the 5′ end is an RNA molecule. In some embodiments, the PRR agonist comprises an uncapped diphosphate (PP) group at the 5′ end. In some embodiments, the PRR agonist comprises an uncapped diphosphate (PP) group at the 5′ end is an RNA molecule. In some embodiments, the PRR agonist comprises a 5′-terminal nucleotide having an unmethylated 2′-O position. In some embodiments, the PRR agonist binds to a carboxy-terminal domain (CTD) of an RLR. In some embodiments, the PRR agonist comprises nucleic acid base pairs which contact the helicase domain of an RLR. In some embodiments, the PRR agonist is an RLR agonist. In some embodiments, the RLR agonist is a RIG-I agonist. In some embodiments, the RIG-I agonist comprises a uridine rich stretch. In some embodiments, the RIG-I agonist comprises hepatitis C virus (HVC) RNA genome sequence, or a functional variant thereof. In some embodiments, the RIG-I agonist comprises Sendai virus RNA genome sequence, or a functional variant thereof. In some embodiments, the RIG-I agonist comprises West Nile virus (WNV) RNA genome sequence, or a functional variant thereof. In some embodiments, the RIG-I agonist comprises any RNA genome sequence, or a functional variant thereof.

In some embodiments, a composition herein includes a plurality of PRR agonists. In further embodiments, the plurality of PRR agonists have different sequences. In further embodiments, the plurality of PRR agonists comprise different RNA sequences. In further embodiments, the plurality of PRR agonists comprise different DNA sequences. In further embodiments, the plurality of PRR agonists comprise RNA and DNA sequences. In some embodiments, the PRR agonist comprises a nucleic acid coding a sequence listed in Table 8. In some embodiments, the PRR agonist comprises two or more nucleic acids coding different sequence listed in Table 8. In some embodiments, the PRR agonist is a nucleic acid comprising at least 80%, 85%, 90%, 95%, 96%, 97%, 98%, or 99% sequence identity to a sequence listed in Table 8. In some embodiments, the PRR agonist comprises two or more nucleic acids coding different sequence listed in Table 8. In some embodiments, the PRR agonist is a nucleic acid comprising at least 80%, 85%, 90%, 95%, 96%, 97%, 98%, or 99% sequence similarity to a sequence listed in Table 8. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated.

TABLE 8
PRR Agonists.

| SEQ | |
|---|---|
| ID | |
| NO: | SEQUENCES |
| 148 | RNA 5′ |
| | CCAUCCUGUUUUUUUCCCUUUUUUUUUUUCUUUUUUUUUUUUU |
| | UUUUUUUUUUUUUUUUUUUUUCUCCUUUUUUUUUCCUCUUUUU |
| | UUCCUUUUCUUUCCUUU 3′ |
| 184 | RNA 5′ |
| | GGCCAUCCUGUUUUUUUCCCUUUUUUUUUUUCUUUUUUUUUUU |
| | UUUUUUUUUUUUUUUUUUUUUUUCUCCUUUUUUUUUCCUCUUU |
| | UUUUCCUUUUCUUUCCUUUUCUA 3′ |
| 149 | RNA 5′ |
| | CCCCCCCCCCCCCCCCCCCCCCCCCCCCCCCCCCCCCCCC |
| | CCCCCCCCCCCCCCCCCCCC 3′ |
| 150 | RNA 5′ |
| | GGGGGGGGGGGGGGGGGGGGGGGGGGGGGGGGGGGGGGGGGGG |
| | GGGGGGGGGGGGGGGGGGGGGGGGGGGGGGGGGGGGGGGGGGG |
| | GGGGGGGGGGGGGG 3′ |
| 151 | DNA (bold) and RNA |
| | 5′ TGCTGCTGCT TGCAAGCAGC TTGATACCAG |
| | ACAAAGCUGG GAAUAGAAAC UUCGUAUUUU CAAAGUUUUC |
| | UUUAAUAUAU UGCAAAUAAU GCCUAACCAC CUAGGGCAGG |
| | AUUAGGGUUC CGGAGUUCAA CCAAUUAGUC CUUAAUCAGG |
| | GCACUGUAUC CGACU 3′ |
| 152 | RNA 5′ AGUCGGAUAC AGUGCCCUGA UUAAGGACUA |
| | AUUGGUUGAA CUCCGGAACC CUAAUCCUGC CCUAGGUGGU |
| | UAGGCAUUAU UUGCAAUAUA UUAAAGAAAA CUUUGAAAAU |
| | ACGAAGUUUC UAUUCCCAGC UUUGUCUGGU 3′ |
| 153 | RNA 5′ pppGGAUCGAUCGAUCGUUCGCGAUCGAUCGAUC |
| | C-3′ |
| 154 | RNA 5′ GACGAAGACC ACAAAACCAG AUAAAAAAAA |
| | AAAAAAAAAA AAAAAAAAUA AUUUUUUUUU UUUUUUUUUU |
| | UUUUUUUAUC UGGUUUUGUG GUCUUCGUC 3′ |
| 155 | RNA 5′ ppp-GGACGUACGUUUCGACGUACGUCC 3′ |
| 156 | RNA 5′- |
| | pppAGCAAAAGCAGGGUGACAAAGACAUAAUGGAUCCAAACAC |
| | UGUGUCAAGCUUUCAGGUAGAUUGCUUUCUUUGGCAUGUCCGC |
| | AAAC-3′ |
| 157 | DNA |
| | 5′pppTAATACGACTCACTATAGGCCATCCTGTTTTTTTCCCT |
| | TTTTTTTTTCTTTTTTTTTTTTTTTTTTTTTTTTTTTTTTTTT |
| | TTCTCCTTTTTTTTTCCTCTTTTTTTCCTTTTCTTTCCTTT-3 |

In some embodiments, nucleic acid PRR agonists provided herein comprise DNA. In some embodiments, nucleic acid PRR agonists provided herein comprise RNA. In some embodiments, nucleic acid PRR agonists provided herein comprise a DNA strand and an RNA strand, for example SEQ ID NO: 151. In some embodiments, nucleic acid PRR agonists disclosed herein may be present in a composition provided herein and are present in nanogram or microgram amounts. Exemplary amounts for PRR agonists disclosed herein include about 0.05, 0.1, 0.2, 0.3, 0.4, 0.5, 0.6, 0.7, 0.8, 0.9, 1, 1.2, 1.25, 1.5, 2, 3, 4, 5, 7.5, 10 or more μg. Exemplary amounts for PRR agonists disclosed herein include up to 0.1, 0.2, 0.3, 0.4, 0.5, 0.6, 0.7, 0.8, 0.9, 1, 1.2, 1.25, 1.5, 2, 3, 4, 5, 7.5, or 10 μg. Exemplary amounts for PRR agonists disclosed herein include at least 0.05, 0.1, 0.2, 0.3, 0.4, 0.5, 0.6, 0.7, 0.8, 0.9, 1, 1.2, 1.25, 1.5, 2,3, 4, 5, 7.5, or 10 g. Exemplary amounts for PRR agonists disclosed herein include 0.05 to 10, 0.1 to 5, 0.05 to 5, 0.1 to 5 μg. Additional exemplary amounts for PRR agonists disclosed herein include about 0.05, 0.1, 0.2, 0.5, 1, 5, 10, 12.5, 15, 25, 40, 50, 100, 125, 150, 175, 200, 250, 400, 500, 600, 700, 750, 1000, 1500, 2000, 3000, 4000, 5000 or more micrograms (μg). In some embodiments, the nucleic acid PRR agonist comprises one or more nucleic acids comprising a sequence that is at least 85% identical, 90% identical, 95% identical, 99% identical, or 100% identical to a sequence listed in Table 8 (SEQ ID NOS: 148-157, or 184). In some embodiments, the nucleic acid is at least about 15, 25, 50, 75, 100, 125, 150, 175, 200, 225, 250, 275, or 300 nucleotides in length. In some embodiments, the nucleic acid is up to about 15, 25, 50, 75, 100, 125, 150, 175, 200, 225, 250, 275, or 300 nucleotides in length. In some embodiments, the nucleic acid is 25-150, 25-300, or 50-150 nucleotides in length.

In some embodiments, the PRR agonist is a nucleic acid, such as an RNA or DNA. A variety of RNAs can be associated with the nanoparticles for delivery provided herein, including RNAs that modulate innate immune responses, RNAs that encode proteins or antigens, silencing RNAs, microRNAs, tRNAs, self-replicating RNAs, etc. In certain embodiments, the PRR agonist is a non-coding RNA, a TLR agonist, a RIG-I agonist, a saponin, a peptide, a protein, a carbohydrate, a carbohydrate polymer, a conjugated carbohydrate, a whole viral particle, a virus-like particle, viral fragments, cellular fragments, and combinations thereof. In certain embodiments, the nucleic acid is a TLR agonist or a RIG-I agonist. Exemplary TLR agonists include a TLR2, TLR3, TLR4, TLR7, TLR8, or TLR9 agonist. An exemplary TLR agonist for inclusion in a composition provided herein is, without limitation, is aTLR3 agonist, such as RIBOXXOL, poly(I:C) (Polyinosinic:polycytidylic acid, sodium salt, ((C₁₀H₁₀N₄NaO₇P)ₓ·(C₉H₁₁N₃NaO₇P)ₓ)), or Hiltonol®.

### Nucleic Acids Encoding Immune System Modulators

Provided herein are nucleic acids encoding an immune system modulator. An immune system modulator is an agent, cytokine, or protein that changes the level of an immune cell (e.g., B-cell, T-cell, antigen presenting cell, activated B-cell, activated T-cell, activated macrophage), changes the level of immunomodulatory molecules (e.g., inflammatory cytokines, chemokines), or a combination thereof. Modulation of immune response by such an immune system modulator can be a suppression of the immune response (immunosuppression or anti-inflammatory) in a subject or an increase the immune response (immunostimulatory or pro-inflammatory) in a subject relative to a subject that has not been administered a composition provided herein.

In some embodiments, a nucleic acid provided herein encodes for a cytokine. Cytokines are small proteins (generally about 5 to 20 kDa) that act through their corresponding target cytokine receptors to modulate immune responses, cell growth, and other cellular functions. Immune cells secrete cytokines and interferons to signal to other immune cells, e.g., to promote phagocytosis of a microorganism or infected cells, or induce inflammation at the site of an injury. In some embodiments, the cytokine is a pro-inflammatory cytokine. Non-limiting examples of pro-inflammatory cytokines include: interleukin-12 (IL-12), IL-18, IL-17, IL-10, TNF-alpha (TNF-α), interferon gamma (IFNγ), and granulocyte-macrophage colony stimulating factor (GM-CSF).

In some embodiments, the cytokine is an anti-inflammatory cytokine. Non-limiting examples of anti-inflammatory cytokines include: IL-4, IL-10, IL-11, IL-13, and IL-35.

In some embodiments, a nucleic acid provided herein encodes for an IL-12 family cytokine. IL-12 is generally secreted from B-cells and macrophages. IL-12 can induce proliferation of natural killer (NK) cells, increase interferon (IFN) production, and promote cell-mediated immune functions. IL-12 can induce naïve CD4+ T cells to differentiate into Th1 cells. The interleukin 12 (IL-12) family is comprised of 4 members, IL-12, IL-23, IL-27, and IL-35. IL-12, IL-23 and IL-27 are secreted by activated antigen presenting cells (APC) during antigen presentation to naïve T cells while IL-35 is a product of regulatory T and B cells. Each IL-12 family cytokine is composed of an α-subunit with a helical structure and a β-subunit structurally related to the extracellular regions of Type 1 cytokine receptors (e.g., soluble IL-6 receptor). The α-subunits are IL-12p35, IL-23p19 and IL-27p28 and the β subunits are IL-12p40 and Ebi3 and co-expression of both chains is necessary for secretion of the bioactive cytokine by an antigen presenting cell. Because the three alpha subunits (IL-12p35, IL-23p19 and IL-27p28) are structurally related, each can pair with either of the structurally homologous β subunits (IL-12p40 and Ebi3). In some embodiments, a nucleic acid provided herein encodes for an alpha and/or a beta chain of an IL-12 family cytokine. In some embodiments, nucleic acids provided herein encode for an IL-12p40. In some embodiments, nucleic acids provided herein encode for a Ebi3. In some embodiments, nucleic acids provided herein encode for an IL-12p35. In some embodiments, nucleic acids provided herein encode for an IL-23p19. In some embodiments, nucleic acids provided herein encode for an IL-27p28. In some embodiments, a nucleic acid provided herein encodes for (1) IL-12p35, IL-23p19, or IL-27p28; and (2) IL-12p40 or Ebi3.

In some embodiments, a nucleic acid provided herein encodes for IL-12, or a functional variant thereof. In some embodiments, a nucleic acid provided herein encodes a human IL-12A subunit, or a functional variant thereof. In some embodiments, a nucleic acid provided herein encodes a human IL-12B subunit, or a functional variant thereof. In some embodiments, a nucleic acid provided herein encodes a mouse IL-12a subunit, or a functional variant thereof. In some embodiments, a nucleic acid provided herein encodes a mouse IL-12b subunit or a functional variant thereof.

In some embodiments, a nucleic acid provided herein further comprises a sequence encoding a linker. In some embodiments, the linker comprises an amino acid sequence comprising VPGVGVPGVG (SEQ ID NO: 158), a fragment, or a variant thereof.

In some embodiments, a nucleic acid provided herein encodes a human IL-12B, a linker, and a human IL12A subunit. In some embodiments, a nucleic acid provided herein encodes a mouse IL-12b, a linker, and a mouse IL12a subunit. In some embodiments, a nucleic acid provided herein encodes for a mouse IL-12 comprising an amino acid sequence that is at least 85% identical to any one of SEQ ID NOS: 159-164, and any combination thereof. In some embodiments, the nucleic acid encodes for a fusion protein comprising a mouse IL-12b (p40) and a mouse IL-12a (p35) subunit, wherein the fusion protein further comprises an elastin linker between subunits. In some embodiments, a nucleic acid provided herein encodes for a mouse IL-12 fusion protein, wherein the nucleic acid comprises a sequence that is at least 85% identical to one of SEQ ID NOS: 165-166.

In some embodiments, a nucleic acid provided herein encodes for interferon gamma (IFNγ), or a functional variant thereof. In some embodiments, a nucleic acid provided herein encodes for a human IFNγ comprising an amino acid sequence that is at least 85% identical to SEQ ID NO: 167. In some embodiments, a nucleic acid provided herein encodes for a mouse IFNγ comprising an amino acid sequence that is at least 85% identical to SEQ ID NO: 168. Interferon is generally secreted from activated Th1 T cells and natural killer (NK) cells. IFNγ can induce expression of class I MHC molecule on the surface of somatic cells, induce class II MHC expression on antigen presenting cells (APCs) and somatic cells. IFNγ can induce activation of macrophages, neutrophils, and NK cells. In addition, IFNγ promotes cell-mediated immunity and antiviral responses.

In some embodiments, a nucleic acid provided herein encodes for IL-2, or a functional variant thereof. In some embodiments, a nucleic acid provided herein encodes for a human IL-2 comprising an amino acid sequence that is at least 85% identical to SEQ ID NO: 169. IL-2 is generally secreted or expressed by activated Th1 cells and NK cells. IL-2 can induce and enhance proliferation of B cells and activated T cells. IL-2 can also modulate NK cellular functions.

In some embodiments, a nucleic acid provided herein encodes for IL-15, or a functional variant thereof. In some embodiments, a nucleic acid provided herein encodes for a human IL-15 comprising an amino acid sequence that is at least 85% identical to SEQ ID NO: 170. IL-15 is generally expressed or secreted by mononuclear phagocytes (e.g., macrophages, monocytes, Kupffer cells, histiocytes, microglia, osteoclasts, dust cells, Langerhans cells, Hofbauer cells, intraglomerular mesangial cells sinusoidal lining cells, etc.). IL-15 induces proliferation of NK cells among other functions.

In some embodiments, a nucleic acid provided herein encodes for IL-18, or a functional variant thereof. In some embodiments, a nucleic acid provided herein encodes for a human IL-18 comprising an amino acid sequence that is at least 85% identical to SEQ ID NO: 171. IL-18 is generally expressed or secreted by macrophages and can indue interferon production and expression by T cells and NK cells.

In some embodiments, a nucleic acid provided herein encodes for IL-21, or a functional variant thereof. In some embodiments, a nucleic acid provided herein encodes for a human IL-21 comprising an amino acid sequence that is at least 85% identical to SEQ ID NO: 172. IL-21 is generally secreted by T cells such as Th2 cells, T follicular cells, and NK T cells. IL-12 induces cell proliferation and activates CD8+ T cell effector activity.

In some embodiments, a nucleic acid provided herein encodes for IL-23, or a functional variant thereof. In some embodiments, a nucleic acid provided herein encodes for a human IL-23 comprising an amino acid sequence that is at least 85% identical to SEQ ID NO: 173. IL-23 is generally secreted from or expressed by activated dendritic cells, macrophages, monocytes; innate lymphoid cells, y6 T cells; and B cells. IL-23 induces the development and differentiation of effector Th17 cells, and stimulates IL-17 production and expression, among other functions.

In some embodiments, a nucleic acid provided herein encodes for IL-27, or a functional variant thereof. In some embodiments, a nucleic acid provided herein encodes for a human IL-27 comprising an amino acid sequence that is at least 85% identical to SEQ ID NO: 174, SEQ ID NO: 175, or a combination thereof. IL-27 is composed of an α chain p28 and β chain Epstein-Barr induce gene-3 (EBI3). The p28 subunit is also called IL-30. IL-27 is generally expressed or secreted by antigen presenting cells. IL-27 can induce differentiation of T cells and upregulate IL-10 secretion.

In some embodiments, a nucleic acid provided herein encodes for IL-35, or a functional variant thereof. IL-35 is a dimeric protein composed of IL-12a and IL-270 chains, which are encoded by two separate genes—IL12A and EBI3 (Epstein-Barr virus-induced gene 3), respectively. In some embodiments, a nucleic acid provided herein encodes for a human IL-27 comprising an amino acid sequence that is at least 85% identical to SEQ ID NO: 159, SEQ ID NOS: 174, or a combination thereof. IL-35 is an immunosuppressive cytokine that blocks the development of Th1 and Th17 cells by limiting early T cell proliferation in a subject.

In some embodiments, a nucleic acid provided herein encodes for IL-39, or a functional variant thereof. IL-39 is a heterodimer of IL-23p19 and Epstein-Barr induced gene-3 (EBI3). IL-39 is a cytokine secreted by stimulated and activated B cells. IL-39 induces and/or expands neutrophils and can increase the secretion of B cell activation factor (BAFF), stimulating inflammation in a subject.

In some embodiments, nucleic acids provided herein encode for a cytokine listed in Table 1. In some embodiments, compositions provided herein comprise two or more nucleic acids encoding for different sequences listed in Table 9. In some embodiments, nucleic acids provided herein encode for a cancer-associated protein sequence comprising at least 80%, 85%, 90%, 95%, 96%, 97%, 98%, or 99% sequence identity to a sequence listed in Table 9. In some embodiments, compositions provided herein comprise two or more nucleic acids encoding different sequences listed in Table 9. In some embodiments, the nucleic acid provided herein encodes for a cancer-associated protein or a functional fragment thereof comprising at least 80%, 85%, 90%, 95%, 96%, 97%, 98%, or 99% sequence similarity to a sequence listed Table 9. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated. Table 9 lists cytokines and sequences that can be encoded by the nucleic acids provided herein.

TABLE 9
Cytokines.

| Name | SEQ ID NOS: |
|---|---|
| IL-12 | IL12A Amino Acid: SEQ ID NO: 159 |
| | IL12B Amino Acid: SEQ ID NO: 160 |
| | IL12a Mouse Amino Acid: SEQ ID NO: 161 |
| | IL12b Mouse Amino Acid: SEQ ID NO: 162 |
| | Human IL12B-A Fusion Protein Amino |
| | Acid Sequence: SEQ ID NO: 163 |
| | Mouse IL12b-a Fusion Protein Amino |
| | Acid Sequence: SEQ ID NO: 164 |
| | IL-12 fusion protein RNA sequences: |
| | SEQ ID NO: 165 (mouse IL-12), |
| | SEQ ID NO: 166 (human IL-12) |
| IFN-gamma | Human Amino Acid: SEQ ID NO: 167 |
| (IFNγ) | Mouse Amino Acid: SEQ ID NO: 168 |
| IL-2 | Human Amino Acid: SEQ ID NO: 169 |
| IL-15 | Human Amino Acid: SEQ ID NO: 170 |
| IL-18 | Human Amino Acid: SEQ ID NO: 171 |
| IL-21 | Human Amino Acid: SEQ ID NO: 172 |
| IL-23 | Human Amino Acid: SEQ ID NO: 173 |
| IL-27 | Human IL27A Amino Acid: SEQ ID NO: 174 |
| | Human IL-27B Amino Acid: SEQ ID NO: 175 |
| IL-35 | See, e.g., SEQ ID NO: 159 and SEQ ID NO: 174 |

In some embodiments, a nucleic acid provided herein comprises a nucleic acid sequence that is at least 85% identical to any one of SEQ ID NOS: 165-166. In some embodiments, a nucleic acid provided herein comprises a nucleic acid sequence that is at least 90%, 95%, 96%, 97%, 98%, or 99% identical to any one of SEQ ID NOS: 165-166. In some embodiments, a nucleic acid provided herein comprises any one of SEQ ID NOS: 165-166 or a functional fragment thereof. In some embodiments, a nucleic acid provided herein comprises a nucleic acid encoding for an amino acid sequence that is least 85% identical to any one of SEQ ID NOS: 159-164, 167-175. In some embodiments, a nucleic acid provided herein comprises a nucleic acid encoding for an amino acid sequence that is least 90%, 95%, 96%, 97%, 98%, or 99% identical to any one of SEQ ID NOS: 159-164, 167-175. In some embodiments, a nucleic acid provided herein comprises a nucleic acid encoding for any one of SEQ ID NOS: 159-164, 167-175.

In some embodiments, a nucleic acid provided herein encodes for interferon alpha (IFN-α) and/or interferon beta (IFN-β), or a functional variant thereof. IFN-α and IFN-β are generally secreted or expressed by macrophages, neutrophils, and somatic cells. Interferons can induce antiviral effects, induce expression of class I MHC molecules on the surface of somatic cells, activate NK cells and macrophages.

In some embodiments, a nucleic acid provided herein encodes for granulocyte-macrophage colony-stimulating factor (GM-CSF) or a functional variant thereof. GM-CSF is generally secreted or expressed by Th cells. GM-CSF induces the growth and differentiation of monocytes and dendritic cells.

In some embodiments, a nucleic acid provided herein encodes for IL-1α or a functional variant thereof. IL-1α is generally secrete or expressed by macrophages and other APCs. IL-1α co-stimulates APCs and T cells and induces inflammation, fever acute phase response, and hematopoiesis, among other functions.

In some embodiments, a nucleic acid provided herein encodes for IL-3 and/or IL-4, or a functional variant thereof. IL-3 and IL-4 are secreted by activated T cells. IL-3 induces the growth of hematopoietic progenitor cells. IL-4 can induce B-cell proliferation, eosinophil and mast cell growth, induces eosinophil and mast cell function, induces IgE and class II MHC molecule expression on B cells, and can inhibit cytokine production by monocytes and macrophages.

In some embodiments, a nucleic acid provided herein encodes for IL-5 or a functional variant thereof. IL-5 is generally secreted or expressed by Th2 cells and mast cells to induce eosinophil growth and function.

In some embodiments, a nucleic acid provided herein encodes for IL-6 or a functional variant thereof. IL-6 is generally secreted or expressed by activated Th2 cells, APCs, and somatic cells. IL-6 induces acute phase responses, B-cell proliferation, thrombopoiesis. IL-6 works synergistically with IL-1 and TNF on T cell activation.

In some embodiments, a nucleic acid provided herein encodes for IL-7 or a functional variant thereof. IL-7 is generally secreted or expressed by thymic stromal cells and marrow stromal cells to induce T cell and B cell lymphopoiesis.

In some embodiments, a nucleic acid provided herein encodes for IL-8 or a functional variant thereof. IL-8 is generally secreted or expressed by macrophages and somatic cells. IL-8 to act as a chemoattractant for neutrophils and T cells.

In some embodiments, a nucleic acid provided herein encodes for IL-9 or a functional variant thereof. IL-9 is generally secreted or expressed by T cells to induce hematopoiesis and can also have thymopoeitic effects.

In some embodiments, a nucleic acid provided herein encodes for IL-10 or a functional variant thereof. IL-10 is generally secreted or expressed by activated Th2 cells, CD8+ T cells, B cells, and macrophages. IL-10 inhibits cytokine production, promotes B cell proliferation and antibody production. IL-10 also suppresses cellular immunity and mast cell growth.

In some embodiments, a nucleic acid provided herein encodes for IL-11 or a functional variant thereof. IL-11 is generally secreted or expressed by stromal cells and mesenchymal cells. IL-11 can induce thrombopoiesis and stimulate megakaryocytopoiesis. IL-11 stimulates T-cell-dependent development of IgG-secreting B-cells in spleen.

In some embodiments, a nucleic acid provided herein encodes for IL-13 or a functional variant thereof. IL-13 is generally secreted or expressed by Th2 cells and can act synergistically with IL-4 to induce B-cell proliferation.

In some embodiments, a nucleic acid provided herein encodes for IL-17 (also called IL-17a) or a functional variant thereof. IL-17 is generally secreted or expressed by T-helper 17 (Th17) cells, a subset of CD4+ T-cell that secrete IL-17. IL-17 acts as a chemotaxis signal for monocytes and neutrophils to a site of inflammation. IL-17 mediates effects on stromal cells, resulting in production of inflammatory cytokines and recruitment of leukocytes (e.g., neutrophils), creating a link between innate and adaptive immunity.

In some embodiments, a nucleic acid provided herein encodes for IL-22 or a functional variant thereof. IL-22 is generally secreted by Th1, Th22, Th17, and γδ T cells; NK T cells; innate lymphoid cells (ILC3), neutrophils; and macrophages. IL-22 can improve cell survival and proliferation. IL-22 can also promote the synthesis of anti-microbial peptides such as S100, regenerating islet-derived protein 3-beta (Reg3β), regenerating islet-derived protein 3 gamma (Reg3γ), and defensins.

In some embodiments, a nucleic acid provided herein encodes for IL-25 (also called IL-17e) or a functional variant thereof. IL-25 is generally secreted by T cells, dendritic cells, macrophages, mast cells, basophils, eosinophils, epithelial cells and Paneth cells. IL-25 can induce NF-κB activation, the production of IL-8, and a neutrophil chemotaxis. IL-25 also activates eosinophil expansion.

In some embodiments, a nucleic acid provided herein encodes for macrophage inflammatory protein (MIP)-1α and/or MIP-1β, or a functional variant thereof. MIP-1α, also called chemokine (C-C motif) ligand 3 (CCL3), is secreted by macrophages. MIP-1β, also called chemokine (C-C motif) ligands 4 (CCL4) is secreted by lymphocytes and macrophages. MIP-1α and MIP-1β can activate granulocytes (e.g., neutrophils, eosinophils and basophils) to induce acute inflammation.

In some embodiments, a nucleic acid provided herein encodes for transforming growth factor beta (TGF-β) or a functional variant thereof. TGF-β is a multifunctional cytokine belonging to the transforming growth factor superfamily that includes three different mammalian isoforms (TGF-β1 to 3, HGNC symbols TGFB1, TGFB2, TGFB3) and many other signaling proteins. TGF-β proteins are generally produced and secreted by leukocytes, including T cells and monocytes. TGF-β can induce chemotaxis, IL-1 synthesis, IgA synthesis, and inhibit cell proliferation.

In some embodiments, a nucleic acid provided herein encodes for a tumor necrosis factor family protein. In some embodiments, a nucleic acid provided herein encodes for tumor necrosis factor-alpha (TNF-α) or a functional variant thereof. TNF-α is generally secreted by macrophages, mast cells, NK cells, and sensory neurons. TNF-α can induce cell death, induce inflammation, and activate pain signaling. In some embodiments, a nucleic acid provided herein encodes for tumor necrosis factor-beta (TNF-β) or a functional variant thereof. TNF-β, also called lymphotoxin-alpha (LT-α), is produced and secreted by lymphocytes. TNF-β has a number of different functions depending on the form that is secreted or expressed by a cell (e.g., a soluble homotrimer or as a cell surface protein heterotrimer-LTβ). TNF-β can induce cell death and induce inflammation.

In some embodiments, a nucleic acid provided herein encodes for a linker. In some embodiments, the linker is between an alpha and a beta chain of a cytokine. In some embodiments, the linker is between an alpha and a beta chain of an IL-12 family cytokine. In some embodiments, the linker is about 14 to 18 amino acids long.

### RNA Encoding for an RNA Polymerase

Provided herein are compositions comprising a self-replicating nucleic acid. In some embodiments, compositions provided herein comprise one or more nucleic acids. In some embodiments, compositions provided herein comprise two or more nucleic acids. In some embodiments, nucleic acids provided herein code for an RNA polymerase. In some embodiments, nucleic acids provided herein code for a viral RNA polymerase. In some embodiments, nucleic acids provided herein code for: (1) a viral RNA polymerase; and (2) a protein, antibody, or functional fragment thereof. In some embodiments, compositions provided herein comprise a first nucleic acid encoding for a viral RNA polymerase; and a second nucleic acid encoding for a protein, antibody, or functional fragment thereof. In some embodiments, the protein is a recombinant protein. In some embodiments, the functional fragment of the antibody binds to a protein, for example, a cancer-associated protein, a microbial antigen protein, or a portion thereof.

Provided herein are compositions comprising a self-replicating RNA. A self-replicating RNA (also called a replicon) includes any genetic element, for example, a plasmid, cosmid, bacmid, phage or virus that is capable of replication largely under its own control. Self-replication provides a system for self-amplification of the nucleic acids provided herein in mammalian cells. In some embodiments, the self-replicating RNA is single stranded. In some embodiments, the self-replicating RNA is double stranded.

An RNA polymerase provided herein can include but is not limited to: an alphavirus RNA polymerase, an Eastern equine encephalitis virus (EEEV) RNA polymerase, a Western equine encephalitis virus (WEEV), Venezuelan equine encephalitis virus (VEEV), Also, Chikungunya virus (CHIKV), Semliki Forest virus (SFV), or Sindbis virus (SINV). In some embodiments, the RNA polymerase is a VEEV RNA polymerase. In some embodiments, the nucleic acid encoding for the RNA polymerase comprises at least 85% identity to the nucleic acid sequence of SEQ ID NO: 176. In some embodiments, the nucleic acid encoding for the RNA polymerase comprises at least 90% identity to the nucleic acid sequence of SEQ ID NO: 176. In some embodiments, the nucleic acid encoding for the RNA polymerase comprises at least 95% identity to the nucleic acid sequence of SEQ ID NO: 176. In some embodiments, the nucleic acid encoding for the RNA polymerase comprises at least 99% identity to the nucleic acid sequence of SEQ ID NO: 176. In some embodiments, the nucleic acid encoding for the RNA polymerase is SEQ ID NO: 176.

In some embodiments, the amino acid sequence for VEEV RNA polymerase comprises at least 85% identity to RELPVLDSAA FNVECFKKYA CNNEYWETFK ENPIRLTEEN VVNYITKLKG P (SEQ ID NO: 177) or TQMRELPVLD SAAFNVECFK KYACNNEYWE TFKENPIRLT E (SEQ ID NO: 178). In some embodiments, the amino acid sequence for VEEV RNA polymerase comprises at least 90% identity to SEQ ID NO: 177, SEQ ID NO: 178, or SEQ ID NO: 179. In some embodiments, the amino acid sequence for VEEV RNA polymerase comprises at least 95% identity to SEQ ID NO: 177, SEQ ID NO: 178, or SEQ ID NO: 179. In some embodiments, the amino acid sequence for VEEV RNA polymerase comprises at least 99% identity to SEQ ID NO: 177, SEQ ID NO: 178, or SEQ ID NO: 179. In some embodiments, the amino acid sequence for VEEV RNA polymerase comprises any one of SEQ ID NOS: 177-179.

Provided herein are compositions and methods comprising replicon RNA (repRNA) encoding for one or more structural proteins from a non-enveloped virus. In some embodiments, the repRNA encodes a protease. In some embodiments, the repRNA encodes a 3CD protease. In some embodiments, the structural protein and the protease are co-expressed. In further embodiments, the repRNA comprises one or more open reading frames. In some embodiments, the open reading frames are separated by an internal ribosomal entry site (IRES). In some embodiments, the open reading frames are separated by a ribosomal skipping peptide sequence. In some embodiments the ribosomal skipping peptide sequence is from Thosea asigna virus (T2A).

### (3) Adjuvants

Provided herein compositions comprising an adjuvant and a nanoparticle provided herein. An adjuvant is any substance that increases the immunogenicity of a composition provided herein (e.g., NP-1 or NP-30 complexed with nucleic acids). In some embodiments, the adjuvant is a small molecule, a compound, a protein, a lipid, an oil, or a nucleic acid. In some embodiments, the small molecule is a protein expression enhancer. Non-limiting examples of protein expression enhancers include: a casein kinase inhibitor, a cyclin-dependent kinase (CDK) inhibitor, an extracellular signal-regulated kinase (ERK) inhibitor, a growth factor inhibitor, a glycogen synthase kinase inhibitor, an immune checkpoint inhibitor, a Janus kinase (JAK) inhibitor, a IκB kinase (IKK) inhibitor, a glycogen synthase kinase-3β (GSK-3β) inhibitor, a lipid kinase inhibitor, a mitogen-activated protein kinase (MAPK) family inhibitor, a phosphatidylinositol 4-kinase (PI4K) inhibitor, a polo-like kinase (PLK) inhibitor, a protein kinase D (PKD) inhibitor, a tyrosine kinase inhibitor, a T-lymphokine-activated killer cell-originated protein kinase (TOPK) inhibitor, a salt inducible kinase (SIK) inhibitor, or a Wnt signaling inhibitor.

In some embodiments, the adjuvant is a metal. In some embodiments, the adjuvant is aluminum or a derivative thereof. In some embodiments, the adjuvant comprises squalene. In some embodiments, the adjuvant is an oil-based adjuvant. In some embodiments, the adjuvant comprises a squalene-based oil-in-water nanoemulsion (e.g., AddaVax or MF59). In some embodiments, the adjuvant is a suspension of desiccated mycobacterium in paraffin oil and mannide monooleate (e.g., complete Freund's adjuvant, CFA).

In some embodiments, the adjuvant is a cytokine. In some embodiments, the cytokine is a cytokine listed in Table 9. In some embodiments, the cytokine comprises an amino acid sequence that is at least 85% identical, 90% identical, 95% identical, 99% identical, or 100% identical to any one of SEQ ID NOS: 159-164, 167-175. In some embodiments, the adjuvant is a nucleic acid encoding an IL-12, for example, a mouse IL-12 or a human IL-12. In some embodiments, the nucleic acid encoding IL-12 comprises a sequence that is at least 85% identical, 90% identical, 95% identical, 99% identical, or 100% identical to SEQ ID NO: 165 or SEQ ID NO: 166.

In some embodiments, the adjuvant is a pattern-recognition receptor (PRR) agonist. In some embodiments, the PRR agonist is a PRR agonist listed in Table 8. In some embodiments, the nucleic acid PRR agonist comprises one or more nucleic acids comprising a sequence that is at least 85% identical, 90% identical, 95% identical, 99% identical, or 100% identical to a sequence listed in Table 8 (SEQ ID NOS: 148-157, or 184). In some embodiments, the PRR agonist is a non-coding RNA, a TLR agonist, a RIG-I agonist, a saponin, a peptide, a protein, a carbohydrate, a carbohydrate polymer, a conjugated carbohydrate, a whole viral particle, a virus-like particle, viral fragments, cellular fragments, or a combination thereof.

### (4) Combination Compositions

Provided herein are compositions comprising nanoparticles (e.g., NP-30 or NP-1) and a nucleic acid encoding for a protein, an antibody, or a fragment thereof. In some embodiments, the protein is a recombinant protein. In some embodiments, the antibody binds to a protein, wherein the protein comprises a microbial antigen, a cancer-associated antigen, or a portion thereof. In some embodiments, nucleic acids provided herein are incorporated, associated with, or complexed a lipid carrier provided herein to form a lipid carrier-nucleic acid complex. The lipid carrier-nucleic acid complex is formed via non-covalent interactions or via reversible covalent interactions. The nucleic acids provided herein are in complex with the surface of the lipid nanoparticle provided herein.

Further provided herein is a nanoemulsion comprising a plurality of nanoparticles provided herein. In some embodiments, the nucleic acid further encodes for an RNA-dependent polymerase. In some embodiments, the RNA-dependent polymerase is a viral RNA polymerase. In some embodiments, the nucleic acid encoding for the RNA polymerase is on the same nucleic acid strand as the nucleic acid sequence encoding for the protein (e.g., cis). In some embodiments, the nucleic acid encoding for the RNA polymerase is on a different nucleic acid strand as the nucleic acid sequence encoding for the protein (e.g., trans). In some embodiments, the nucleic acid encoding for the RNA polymerase is a DNA molecule. In some embodiments, nucleic acid sequences encoding for a cancer-associated protein, a tumor antigen, a neoantigen, a cancer therapeutic antibody, or a functional fragment thereof are DNA or RNA molecules. In some embodiments, cancer-associated proteins and cancer therapeutic antibodies provided herein are encoded by DNA. Nanoparticles for inclusion include, without limitation, any one of NP-1 to NP-31, or any one of NP-1 to NP-37. Nucleic acids for inclusion include, without limitation, comprise a region comprising any one of, or a plurality of, SEQ ID NOS: 11, 20, 22, 25, 27, 29, 31, 33, 35, 37, 39, 43, 49, 51, 67-72, 148-157, 165-166, 176, 184; and/or encodes for an amino acid sequence set forth in any one of SEQ ID NOS: 1-10, 12-19, 21, 23-24, 26, 28, 30, 32, 34, 36, 38, 40-42, 44-48, 50, 52-66, 73-147, 158-164, 167-175, 177-179. In some instances, the nucleic acids further comprise a region encoding for an RNA polymerase, e.g., a region comprising a sequence of SEQ ID NO: 176. In some embodiments, compositions provided herein comprise a plurality of nucleic acids each encoding a protein antigen provided herein. The protein antigen encoded by each nucleic acid can be the same or different. In some embodiments, compositions provided herein are multivalent vaccine compositions. As one example, a composition provided herein can comprise a first nucleic acid encoding for an HIV Env protein antigen, a second nucleic acid encoding for a SARS spike protein antigen, and a third nucleic acid encoding for an enterovirus protein antigen.

Compositions provided herein can be characterized by an nitrogen:phosphate (N:P) molar ratio. The N:P ratio is determined by the amount of cationic lipid in the nanoparticle which contain nitrogen and the amount of nucleic acid used in the composition which contain negatively charged phosphates. A molar ratio of the lipid carrier to the nucleic acid can be chosen to increase the delivery efficiency of the nucleic acid, increase the ability of the nucleic acid-carrying nanoemulsion composition to elicit an immune response to the antigen, increase the ability of the nucleic acid-carrying nanoemulsion composition to elicit the production of antibody titers to the antigen in a subject. In some embodiments, compositions provided herein have a molar ratio of the lipid carrier to the nucleic acid can be characterized by the nitrogen-to-phosphate molar ratio, which can range from about 0.01:1 to about 1000:1, for instance, from about 0.2:1 to about 500:1, from about 0.5:1 to about 150:1, from about 1:1 to about 150:1, from about 1:1 to about 125:1, from about 1:1 to about 100:1, from about 1:1 to about 50:1, from about 1:1 to about 50:1, from about 5:1 to about 50:1, from about 5:1 to about 25:1, or from about 10:1 to about 20:1. In certain embodiments, the molar ratio of the lipid carrier to the nucleic acid, characterized by the nitrogen-to-phosphate (N:P) molar ratio, ranges from about 1:1 to about 150:1, from about 5:1 to about 25:1, or from about 10:1 to about 20:1. In some embodiments, the N:P molar ratio of the nanoemulsion composition is about 15:1. In some embodiments, the nanoparticle comprises a nucleic acid provided herein covalently attached to the membrane.

Compositions provided herein can be characterized by an oil-to-surfactant molar ratio. In some embodiments, the oil-to-surfactant ratio is the molar ratio of squalene: DOTAP, hydrophobic surfactant, and hydrophilic surfactant. In some embodiments, the oil-to-surfactant ratio is the molar ratio of squalene: DOTAP, sorbitan monostearate, and polysorbate 80. In some embodiments, the oil-to surfactant molar ratio ranges from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the oil-to-surfactant molar ratio is at least about 0.1:1, at least about 0.2:1, at least about 0.3:1, at least about 0.4:1, at least about 0.5:1, at least about 0.6:1, at least about 0.7:1. In some embodiments, the oil-to surfactant molar ratio is at least about 0.4:1 up to 1:1.

Compositions provided herein can be characterized by hydrophilic surfactant-to-lipid (e.g., cationic lipid) ratio. In some embodiments, the hydrophilic surfactant-to-lipid ratio ranges from about 0.1:1 to about 2:1, from about 0.2:1 to about 1.5:1, from about 0.3:1 to about 1:1, from about 0.5:1 to about 1:1, or from about 0.6:1 to about 1:1. Compositions provided herein can be characterized by hydrophobic surfactant-to-lipid (e.g., cationic lipid) ratio ranging. In some embodiments, the hydrophobic surfactant-to-lipid ratio ranges from about 0.1:1 to about 5:1, from about 0.2:1 to about 3:1, from about 0.3:1 to about 2:1, from about 0.5:1 to about 2:1, or from about 1:1 to about 2:1.

Provided herein is a dried composition comprising a sorbitan fatty acid ester, an ethoxylated sorbitan ester, a cationic lipid, an immune stimulant, and an RNA. Further provided herein are dried compositions, wherein the dried composition comprises sorbitan monostearate (e.g., SPAN® 60), polysorbate 80 (e.g., TWEEN® 80), DOTAP, and an RNA.

### (5) Thermally Stable, Dried, and Lyophilized Vaccines

Provided herein are dried or lyophilized compositions and vaccines. Further provided herein are pharmaceutical compositions comprising a dried or lyophilized composition provided herein that is reconstituted in a suitable diluent and a pharmaceutically acceptable carrier. In some embodiments, the diluent is aqueous. In some embodiments, the diluent is water.

A lyophilized composition is generated by a low temperature dehydration process involving the freezing of the composition, followed by a lowering of pressure, and removal of ice by sublimation. In certain cases, lyophilization also involves the removal of bound water molecules through a desorption process. In some embodiments, compositions and vaccine compositions provided herein are spray-dried. Spray drying is a process by which a solution is fed through an atomizer to create a spray, which is thereafter exposed to a heated gas stream to promote rapid evaporation. When sufficient liquid mass has evaporated, the remaining solid material in the droplet forms particles which are then separated from the gas stream (e.g., using a filter or a cyclone). Drying aids in the storage of the compositions and vaccine compositions provided herein at higher temperatures (e.g., greater than 4° C.) as compared to the sub-zero temperatures needed for the storage of existing mRNA vaccines. In some embodiments, dried compositions and lyophilized compositions provided herein comprise (a) a lipid carrier, wherein the lipid carrier is a nanoemulsion comprising: (i) a hydrophobic core; (ii) optionally, one or more inorganic nanoparticles; (iii) and one or more lipids; (b) one or more nucleic acids; and (c) at least one cryoprotectant. In some embodiments, the cryoprotectant is selected from the group consisting of: sucrose, maltose, trehalose, mannitol, glucose, and any combinations thereof. Additional examples of cryoprotectants include but are not limited to: dimethyl sulfoxide (DMSO), glycerol, propylene glycol, ethylene glycol, 3-O-methyl-D-glucopyranose (3-OMG), olyethylene glycol (PEG), 1,2-propanediol, acetamide, trehalose, formamide, sugars, proteins, and carbohydrates.

In some embodiments, compositions and methods provided herein comprise at least one cryoprotectant. Exemplary cryoprotectants for inclusion are, but not limited to, sucrose, maltose, trehalose, mannitol, or glucose, and any combinations thereof. In some embodiments, additional or alternative cryoprotectant for inclusion is sorbitol, ribitol, erthritol, threitol, ethylene glycol, or fructose. In some embodiments, additional or alternative cryoprotectant for inclusion is dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates. In some embodiments, the cryoprotectant is present at about 1% w/v to at about 20% w/v, preferably about 10% w/v to at about 20% w/v, and more preferably at about 10% w/v. In certain aspects of the disclosure, the cryoprotectant is sucrose. In some aspects of the disclosure, the cryoprotectant is maltose. In some aspects of the disclosure, the cryoprotectant is trehalose. In some aspects of the disclosure, the cryoprotectant is mannitol. In some aspects of the disclosure, the cryoprotectant is glucose. In some embodiments, the cryoprotectant is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the cryoprotectant is present in an amount of about 50 to about 500 mg. In some embodiments, the cryoprotectant is present in an amount of about 200 to about 300 mg. In some embodiments, the cryoprotectant is present in an amount of about 250 mg. In some embodiments, the cryoprotectant is present in amount of a lyophilized composition by weight of at least about 50, 55, 60, 65, 70, 75, 80, 85, 90, 95 or more percent. In some embodiments, the cryoprotectant is present in amount of a lyophilized composition by weight of about 95%. In some embodiments, the cryoprotectant is present in amount of a lyophilized composition by weight of 80 to 98%, 85 to 98%, 90 to 98%, or 94 to 96%. In some embodiments, the cryoprotectant is a sugar. In some embodiments, the sugar is sucrose, maltose, trehalose, mannitol, or glucose. In some embodiments, the sugar is sucrose. In some embodiments, the sucrose is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the sucrose is present in an amount of about 50 to about 500 mg. In some embodiments, the sucrose is present in an amount of about 200 to about 300 mg. In some embodiments, the sucrose is present in an amount of about 250 mg. In some embodiments, the sucrose is present in amount of a lyophilized composition by weight of at least about 50, 55, 60, 65, 70, 75, 80, 85, 90, 95 or more percent. In some embodiments, the sucrose is present in amount of a lyophilized composition by weight of about 95%. In some embodiments, the sucrose is present in amount of a lyophilized composition by weight of 80 to 98%, 85 to 98%, 90 to 98%, or 94 to 96%.

In some embodiments, the cryoprotectant is sucrose. In some embodiments, the cryoprotectant is at a concentration of at least about 0.1% w/v. In some embodiments, the cryoprotectant is at a concentration of about 1% w/v to at about 20% w/v. In some embodiments, the cryoprotectant is at a concentration of about 10% w/v to at about 20% w/v. In some embodiments, the cryoprotectant is at a concentration of about 10% w/v.

In some embodiments, compositions and vaccine compositions provided herein are thermally stable. A composition is considered thermally stable when the composition resists the action of heat or cold and maintains its properties, such as the ability to protect a nucleic acid molecule from degradation at given temperature. In some embodiments, compositions and vaccine compositions provided herein are thermally stable at about 25 degrees Celsius (° C.) or standard room temperature. In some embodiments, compositions and vaccine compositions provided herein are thermally stable at about 45° C. In some embodiments, compositions and vaccine compositions provided herein are thermally stable at about −20° C. In some embodiments, compositions and vaccine compositions provided herein are thermally stable at about 2° C. to about 8° C. In some embodiments, compositions and vaccine compositions provided herein are thermally stable at a temperature of at least about −80° C., at least about −20° C., at least about 0° C., at least about 2° C., at least about 4° C., at least about 6° C., at least about 8° C., at least about 10° C., at least about 20° C., at least about 25° C., at least about 30° C., at least about 37° C., up to 45° C. In some embodiments, compositions and vaccine compositions provided herein are thermally stable for at least about 5 day, at least about 1 week, at least about 2 weeks, at least about 1 month, up to 3 months. In some embodiments, compositions and vaccine compositions provided herein are stored at a temperature of at least about 4° C. up to 37° C. for at least about 5 day, at least about 1 week, at least about 2 weeks, at least about 1 month, up to 3 months. In some embodiments, compositions and vaccine compositions provided herein are stored at a temperature of at least about 20° C. up to 25° C. for at least about 5 day, at least about 1 week, at least about 2 weeks, at least about 1 month, up to 3 months.

Also provided herein are methods for preparing a lyophilized composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and lyophilizing the formulation to form a lyophilized composition.

Further provided herein are methods for preparing a spray-dried composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and spray drying the formulation to form a spray-dried composition.

Further provided herein are methods for reconstituting a lyophilized composition comprising: obtaining a lipid carrier, wherein the lipid carrier is a nanoemulsion comprising a hydrophobic core, one or more inorganic nanoparticles, and one or more lipids; incorporating one or more nucleic acid into the said lipid carrier to form a lipid carrier-nucleic acid complex; adding at least one cryoprotectant to the lipid carrier-nucleic acid complex to form a formulation; lyophilizing the formulation to form a lyophilized composition; and reconstituting the lyophilized composition in a suitable diluent.

Further provided herein are methods for reconstituting a spray-dried composition comprising: obtaining a lipid carrier, wherein the lipid carrier is a nanoemulsion comprising a hydrophobic core, one or more inorganic nanoparticles, and one or more lipids, incorporating one or more nucleic acid into the said lipid carrier to form a lipid carrier-nucleic acid complex; adding at least one cryoprotectant to the lipid carrier-nucleic acid complex to form a formulation; spray drying the formulation to form a spray-dried composition; and reconstituting the spray-dried composition in a suitable diluent.

### (6) Pharmaceutical Compositions

Provided herein is a lyophilized composition comprising a composition provided herein (e.g., NP-1 or NP-30 complexed with nucleic acids). Further provided herein is a suspension comprising a composition provided herein. In some embodiments, suspensions provided herein comprise a plurality of nanoparticles or compositions provided herein. In some embodiments, compositions provided herein are in a suspension, optionally a homogeneous suspension. In some embodiments, compositions provided herein are in an emulsion form.

Further provided herein is a pharmaceutical composition comprising a composition provided herein (e.g., NP-1 or NP-30 complexed with nucleic acids). In some embodiments, compositions provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

### (7) Dosing

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. Dosing may be for veterinary or human therapeutic uses. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g. ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human and non-human animal use.

### (8) Administration

Provided herein are compositions and pharmaceutical compositions for administering to a subject in need thereof. In some embodiments, the subject is a human. In some embodiments, the subject is a non-human animal. Further provided herein are compositions and pharmaceutical compositions for administration to an animal. In some embodiments, the animal is a domesticated animal or livestock. Further provided herein are compositions and pharmaceutical compositions for veterinary and therapeutic use in non-human animals. Subjects include, without limitation, domesticated animals, farmed animals and insects (including without limitation pigs, cows, horses, donkeys, mules, buffalo, bison, goats, sheep, pigs, ducks, geese, chicken, turkey, fish, camels, alpacas, llamas, rabbits, zebu, deer, guinea pigs, yaks, ferrets, birds, hedgehogs, rodents, turtles, amphibians, bees), wild animals, as well as humans. In some embodiments, pharmaceutical compositions provided here are in a form which allows for compositions provided herein to be administered to a subject.

In some embodiments, the administering is local administration or systemic administration. In some embodiments, a composition described herein is formulated for administration/for use in administration via an intratumoral, subcutaneous, intradermal, intramuscular, inhalation, intravenous, intraperitoneal, intracranial, intranasal, oral, intrathoracic, or intrathecal route. In some embodiments, the administering is every 1, 2, 4, 6, 8, 12, 24, 36, or 48 hours. In some embodiments, the administering is daily, weekly, or monthly. In some embodiments, the administering is repeated at least about every 28 days or at least about every 56 days. In some embodiments, a composition or pharmaceutical composition provided herein is administered to the subject by two doses. In some embodiments, a second dose of a composition or pharmaceutical composition provided herein is administered about 28 days after the first dose or about 56 after the first dose. In some embodiments, a third dose of a composition or pharmaceutical composition provided herein is administered to a subject. In some embodiments, the third dose of a composition or pharmaceutical composition provided herein is administered about 56 days after the second dose.

### (9) Safety and Efficacy

Reactogenicity refers to a subset of reactions that occur after vaccination, and are a physical manifestation of an over-reactive inflammatory response to a vaccine composition. Reactogenic responses to vaccination can include but is not limited to: pain at the site of injection, fever, myalgia, headache, arthralgia, myocarditis, pericarditis, shortness of breath, lymphadenopathy, thromboembolism, anaphylactic reaction, hepatotoxicity, autoimmune hepatitis, acute liver disease or liver injury. Vaccine antigens and immune enhancers (as adjuvants) injected into the muscle are recognized by immune cells as potential pathogens resulting in the production of cytokines and chemokines. The passage of some of those cytokines in the bloodstream, as well as the production of other systemic factors by immune blood cells or distant organs (e.g., liver), contribute to the development of general symptoms (fever, myalgia, headache etc.) in the vaccinee. The magnitude of inflammatory responses have been correlated with the magnitude of adaptive immune responses and the overall incidence of reactogenicity symptoms. The compositions and methods provided here reduce reactogenic symptoms to a vaccine when administered to a subject relative to a comparable subject that has been administered a vaccine.

Provided herein is a method of inducing an immune response in a subject to an antigen, a microbial organism, or a cancer, wherein the method comprises administering to the subject a composition provided herein (e.g., NP-1 or NP-30 complexed with nucleic acids), wherein the composition comprises one or more markers of a favorable safety profile. In some embodiments a favorable safety profile is a set of parameters that reduce the incidence of adverse events in a population of subjects that are administered the composition relative to a comparable composition that has at least one difference in the composition. Adverse events are any untoward medical occurrence caused by a medical treatment, which injures a patient. In some cases, adverse events are severe adverse events that result in a hospital stay longer than 24 hours, a life-threatening medical event, or death. Non-limiting examples of adverse events include myocarditis, pericarditis, convulsions, blood dyscrasias, or allergic bronchospasm. The safety profile of a composition provided herein (e.g., NP-1 or NP-30 complexed with nucleic acids) can be determined in a human population or a test animal population. A favorable safety profile can include one or more parameters including but not limited to: a low or no reported toxicity; high tolerability at nucleic acid doses above 10 μg; a low or no reported incidence of adverse events in the subject following administration of the composition; a low severity of any reported adverse events in the subject following administration of the composition; a low or no reported incidence of pain at the injection site, redness, and swelling; a low or no reported incidence of fever, fatigue, headache, chills, vomiting, diarrhea, new or worsened muscle pain, and new or worsened joint pain; a low or no reported incidence of abnormal hematology; a low or no reported incidence of cardiovascular events; a low or no reported incidence of chest pain; low or no reported incidence of myocarditis; low or no reported incidence of cardiac inflammation; low or no reported incidence of myocardial injury; a low or no reported incidence of seizure; a low or no reported incidence of liver injury; or a low or no reported incidence of kidney failure, as reported by the subject, a practitioner, or a physician.

A favorable safety profile of a composition provided herein (e.g., NP-1 or NP-30 complexed with nucleic acids) can include a low expression of reactogenic biomarkers relative to comparable compositions or a reference level. In some embodiments, a reference level is the level of the reactogenic biomarker in a subject with systemic inflammation. Reactogenicity is the physical manifestation of the systemic inflammatory response induced by a pharmaceutical composition (e.g., a vaccine composition). Reactogenicity results in both local and systemic inflammation. The systemic inflammation in response to vaccination is the cause of adverse events associated with vaccines, including anaphylactic reactions, diseases diagnosed after vaccination (e.g., myocarditis or pericarditis), and autoimmune events. In some embodiments, a composition provided herein (e.g. NP-1 or NP-30 complexed with nucleic acids) when administered to a subject reduces the level or activity of at least one reactogenic biomarker relative to the level or activity of the reactogenic biomarker in a subject with systemic inflammation. Non-limiting examples of reactogenic biomarkers include: type-I interferons, interferon-gamma (IFN-γ), interferon-α2, interleukin-6 (IL-6), interleukin-1β(IL-1β), IL-27p28, MyD88, toll-like receptor 7 (TLR7), TLR8, caspase-1, NOD-LRR-and pyrin domain-containing protein 3 (NLRP3), IL-18, IL-33, monocyte chemoattractant protein-1 (MCP1) or CCL2, CXCL10, C-reactive protein (CRP), sera soluble ST2, creatine kinase-MB (CK-MB), troponin I (cTNI), interleukin-1 receptor agonist (IL-iRA) antibodies, C-reactive protein (CRP), matrix metalloproteases (MMPs), circulating free spike protein, and troponin T (cTNT). In some embodiments, a composition provided herein (e.g., NP-1 or NP-30 complexed with nucleic acids) when administered to a subject modulates the level or activity of at least one biomarker listed in Table 21 or in Table 22 relative to the level or activity of the biomarker in a subject that has not been administered the composition. Methods of detecting and quantifying the level of a reactogenic biomarker include, e.g., immunoassays, PCR, absorbance assays, sequencing, digital ELISA (single molecular array, Simoa), or mass spectrometry. As one example, cardiac troponins are detected in the serum of a subject by the use of monoclonal antibodies to epitopes of cTnI and cTnT. Cardiac troponin is a serological marker of cardiac damage. In some embodiments, a composition provided herein (e.g., NP-1 or NP-30 complexed with nucleic acids) generates a local immune response in a subject and maintains cardiac troponin I levels below a reference level for at least about 24 hours following administration. In some embodiments, the reference level for cTNI is 0.04 ng/mL. In some embodiments, a composition provided herein maintains cardiac troponin I levels below the reference level for at least about 48 hours following administration or more, 72 hours following administration or more, 96 hours following administration or more, 120 hours following administration or more, 144 hours following administration or more, 168 hours following administration or more, 192 hours following administration or more, 216 hours following administration or more, 240 hours following administration or more, 264 hours following administration or more, 288 hours following administration or more, 312 hours following administration or more, 336 hours following administration or more, up to 576 hours.

As yet another example, interferon alpha2 (IFN-α2) can be measured in a blood sample or a cerebral spinal fluid sample from a subject. In some embodiments, a composition provided herein (e.g., NP-1 or NP-30 complexed with nucleic acids) generates a local immune response in a subject and maintains IFN-α2 levels in the serum below 10,000 femtograms/mL (fg/mL) for a period of at least about 24 hours following administration. In some embodiments, a composition provided herein maintains interferon alpha2 (IFN-α2) levels below the reference level for at least about 24 hours following administration or more, at least about 48 hours following administration or more, 72 hours following administration or more, 96 hours following administration or more, 120 hours following administration or more, 144 hours following administration or more, 168 hours following administration or more, 192 hours following administration or more, 216 hours following administration or more, 240 hours following administration or more, 264 hours following administration or more, 288 hours following administration or more, 312 hours following administration or more, 336 hours following administration or more, up to 576 hours.

Further provided herein are compositions (e.g., NP-1 or NP-30 complexed with nucleic acids on the surface of the nanoparticles) that have reduced reactogenicity as compared with reactogenic nanoparticles. Reactogenic nanoparticles are any nanoparticle composition that comprises one or more of the following characteristics or functional properties: (1) a neutral net charge (e.g., 0 net charge) at 37 degrees Celsius; (2) comprise nucleic acids encapsulated within the core of the reactogenic nanoparticle; (3) comprise an average Z-diameter greater than 60 nm; (4) when administered to a subject, the reactogenic nanoparticle compositions has a higher level of a nucleic acid biodistribution ratio relative a composition provided herein; (5) when administered to a subject, the reactogenic nanoparticle increases the level of cardiac troponin in the subject relative to a composition provided herein or relative to a control subject that has not been administered the reactogenic nanoparticle; (6) when administered to a subject, the reactogenic nanoparticle increases the level of interferon alpha in the subject relative to a composition provided herein or relative to a control subject that has not been administered the reactogenic nanoparticle. In some embodiments, a reactogenic nanoparticle composition comprises a solid core at 25 degrees Celsius.

Further provided herein are compositions that have a reduced reactogenicity relative to reactogenic nanoparticles (e.g., nanoparticles that comprise nucleic acids within the core). In some embodiments, the reduced reactogenicity of a composition provided herein is characterized by a lower level or activity of systemic interferons (e.g., in the serum of a subject) relative to the level of systemic interferons in a subject that has been administered a reactogenic nanoparticle composition. In some embodiments, the reduced reactogenicity comprises a lower level or activity of cardiac troponin in the subject relative to a level or activity of cardiac troponin in a subject that has been administered a reactogenic nanoparticle provided herein. In some embodiments, the cardiac troponin level is determined by obtaining a blood sample from a subject and performing an immunoassay.

Provided herein are methods for generating a local immune response within a tissue in a subject. Further provided herein is a method for generating a local innate immune response and an adaptive immune response to an antigen, a microbial organism, or a cancer cell. In some embodiments, the method comprises administering to a tissue in a subject a composition provided herein (e.g., NP-1 or NP-30 complexed with nucleic acids). In some embodiments, the composition comprises: nanoparticles, wherein the nanoparticles comprise: a hydrophobic core comprising lipids in liquid phase at 25 degrees Celsius; and nucleic acids, wherein the nucleic acids are complexed to the nanoparticles to form nucleic acid-nanoparticle complexes. In some embodiments, the administering of the composition generates a local immune response within a tissue. In some embodiments, the tissue is at the site of administration (e.g., an intramuscular injection site or skeletal muscle tissue). In some embodiments, the tissue is not within or not adjacent to the tissue administered the composition (e.g., a liver tissue, a spleen tissue, an ovarian tissue, a testis tissue, a lung tissue, a brain tissue, or a heart tissue).

Compositions provided herein (e.g., NP-1 or NP-30 complexed with nucleic acids) can be administered to any appropriate tissue in the subject. In some embodiments, the tissue is a muscle tissue, an epithelial tissue, an endothelial tissue, a mucosal tissue, a thecal tissue, or a tumor. In some embodiments, the administering is intramuscular administration, intradermal administration, transdermal administration, sublingual administration, buccal administration, intranasal administration, inhalation administration, intrathecal administration, or intratumoral administration. The local immune response can be characterized by various parameters in the tissue of administration provided herein, including, but not limited to interferon levels, immunostimulatory biomarkers, cytokines, and cells. In some embodiments, the local immune response generated by a composition provided herein is characterized by an increase in the level of an immunostimulatory marker in a tissue relative to a reference level. In some embodiments, the local immune response is characterized by an increase in the level or activity of interferon in the tissue relative to the level or activity of interferon in a spleen or a liver of the subject. In some embodiments, the tissue is not within or adjacent to the tissue administered the composition (e.g., a liver tissue, a spleen tissue, an ovarian tissue, a testis tissue, a lung tissue, a brain tissue, or a heart tissue). In some embodiments, the local immune response generated by a composition provided herein is characterized by immune cell recruitment to the tissue. In some embodiments, the local immune response is characterized by localization of the nucleic acids to a tissue that is within or adjacent to a site of administration. In some embodiments, the local immune response is characterized by the nucleic acid biodistribution ratio. The nucleic acid biodistribution ratio is determined by the formula: x1/x2=nucleic acid biodistribution ratio, wherein x1 is the level of the nucleic acids in a tissue that is not within or not adjacent to the tissue that was administered the composition, and wherein x2 is the level of the nucleic acids in the tissue administered the composition. In some embodiments, the level of nucleic acids is determined by polymerase chain reaction (PCR), real time PCR (RT-PCR), spectrophotometry (e.g., NanoDrop®, ThermoFisher), microarray (e.g., Counter® Analysis System, NanoString), or an in vivo imaging assay. As one example, a fluorescent dye can be conjugated to a composition provided herein, followed by administration of the composition to a subject (e.g., a test animal). An in vivo imaging system can be used to determine the biodistribution of the composition by tracking the fluorescent dye in various organs in the subject.

In some embodiments, a composition provided herein comprises a nucleic acid biodistribution ratio between 0 and 0.01 (e.g., 0<nucleic acid biodistribution ratio ≤0.01) when the composition is administered to a tissue in a subject. In some embodiments, a composition provided herein comprises a nucleic acid biodistribution ratio between 0 and 0.1 (e.g., 0≤nucleic acid biodistribution ratio ≤0.1) when the composition is administered to a tissue in a subject. In some embodiments, a composition provided herein comprises a nucleic acid biodistribution ratio between 0 and 1 (e.g., 0<nucleic acid biodistribution ratio ≤1) when the composition is administered to a tissue in a subject. Therefore, a biodistribution ratio between 0 and 1 indicates that a composition provided herein has a favorable safety profile, reduces reactogenicity, and produces a local immune response in a subject relative to a comparable nucleic acid composition (e.g., a reactogenic nanoparticle composition, lipid nanoparticles (LNPs)).

In some embodiments, a composition provided herein (e.g., NP-30/repRNA) comprises a nucleic acid biodistribution ratio that the lower than a reactogenic nanoparticle composition. In some embodiments, a composition provided herein comprises a nucleic acid biodistribution ratio of 0.01 or more, 0.1 or more, 0.2 or more, 0.3 or more, 0.4 or more, 0.5 or more, 0.6 or more, 0.7 or more, 0.8 or more, 0.9 or more, up to 1.0.

Compositions provided herein can increase the level or activity of an immunostimulatory biomarker in the tissue of a subject relative to the level or activity of the immunostimulatory biomarker in the tissue prior to administration or the level or activity of the immunostimulatory biomarker in a healthy subject. In some embodiments, the immunostimulatory biomarker has an increased expression in the muscle tissue when the composition provided herein is administered intramuscularly. Non-limiting examples of immunostimulatory biomarkers include: CD86, H2-ab1, H2-eb1, ITGAL/Cd11a, ITGAM/Cd11b, Fcγ receptor, CD28, and Class I MHC-mediated antigen processing and presentation pathway proteins. The local immune response can also be determined by the immune cell composition within the tissue. For example, pDCs, monocyte dendritic cells, conventional dendritic cells, neutrophils, macrophages, and NK cells can be recruited to the tissue following administration of a composition provided herein.

Methods of detecting or quantifying an immune response can be used to determine the safety and efficacy of a composition administered to a subject provided herein. Immunoassays, e.g., ELISA, ELISPOT assays, Western blots, immunohistology, flow cytometry, or microscopy can be used to detect and/or quantify immune cells, antibodies, cytokines, and any other factor associated with an immune response. For example, flow cytometry can be used to detect and/or quantify CD4⁺ T cells and CD8⁺ T cells in a sample or specific subsets of immune cells provided herein. Flow cytometry can also be used to determine the cellular composition in a tissue from a subject provided herein. The cellular composition can be used to determine whether a composition generates a local or a systemic immune response in a subject in a particular tissue (e.g., muscle, liver, or spleen). In some embodiments, a composition provided herein increases CD8⁺ T cell proliferation in a subject in response to an antigen provided herein. Methods of detecting and quantifying gene expression can also be used to determine the efficacy and safety of a compositions provided herein.

Further provided herein are methods of generating an immune response in a subject, wherein the immune response comprises antibody neutralization of a microbial antigen or a tumor. Methods for assessing the presence of antibody neutralization of a microbial antigen or a tumor can be accomplished, e.g., by cellular impedance, live cell imaging assays, or immunoassays. Cellular impedance assays include wells or plates with gold impedance biosensor arrays that measure the flow of electric current within a well that has been seeded with cells. Impedance is measured before, during, and after infection (e.g., a viral infection). During active microbial infection, the interaction between the cells and the biosensors become weak and a small impedance of electric current (or increased flow of electric current) is detected as compared to cells that are not infected by a microorganism. Real-time impedance measurements can be used to track changes in cell number, cell size, cell-substrate attachment strength, and cell-cell interactions (i.e. barrier function). Because each of these parameters changes during a typical viral cytopathic effect (CPE), impedance provides a very sensitive readout of host cell health throughout the full continuum of a viral infection. Real-time impedance measurements in the presence and absence of a composition provided herein is useful to determine the effect of antibody function and suppression of CPE. Antibody-mediated suppression of the CPE is readily detected as changes in both the kinetics and magnitude of the impedance signal. Plotting the value of the impedance signal at various time points as a function of antibody concentration can produce a dose response curve to yield IC₅₀ measurements and determine the percentage of neutralization relative to control readings.

Provided herein are methods of modulating infectivity of a virus (e.g., SARS-CoV-2). In some embodiments, the methods comprise: contacting a cell or a population of cells with a virus or a viral antigen; contacting the cell or the population of cells with a composition provided herein; and identifying the presence or absence of one or more of: (1) viral neutralization; (2) antibody production; (3) viral plaques; and/or (4) cellular impedance relative to a comparable cell or a population of cells that have not been contacted with the composition provided herein. In some embodiments, the methods comprise: contacting a cell or a population of cells with a virus or a viral antigen; contacting the cell or the population of cells with a composition provided herein; and measuring one or more of (1) viral neutralization; (2) antibody production; (3) viral plaques; and/or (4) cellular impedance relative to a comparable cell or a population of cells that have not been contacted with the composition provided herein. In some embodiments, the compositions provided herein increase viral neutralization; increase antibody production, reduce viral plaques, and/or increase cellular impedance relative to a comparable cell or a population of cells that have not been contacted with the composition provided herein. In some embodiments, the identifying or the measuring of (1) viral neutralization; (2) antibody production; (3) viral plaques; and/or (4) cellular impedance comprises a real time cellular impedance assay and/or live cell imaging assays.

### (10) Therapeutic Applications

Provided herein are methods of treating a disease in a subject. In some embodiments, compositions described herein are used for the treatment of an infection. In some embodiments, the infection is a viral infection.

In some embodiments, compositions described herein are used for the reduction of severity of an infection in a subject. In some embodiments, compositions described herein provide for reduction of severity or duration of symptoms associated with an infection in a subject. In some embodiments, the subject is at risk of developing a viral infection. In some embodiments, the viral infection is an RSV infection, an HIV infection, a CMV infection, a SARS infection, a SARS-CoV-2 infection, a rabies infection, a HPV infection, a Varicella Zoster virus infection, shingles, a Herpes simplex 1 infection, a Herpes simplex 2 infection, or influenza. In some embodiments, the subject has, is suspected of having, or is at risk of developing a bacterial infection. In some embodiments, the bacterial infection is tuberculosis, chlamydia, gonorrhea, strep throat, or a Staphylococcus aureus infection. In some embodiments, the subject has or is suspected of having a fungal infection. In some embodiments, the subject has or is suspected of having a parasitic infection. In some embodiments, the parasitic infection is malaria. In some embodiments, the subject is at risk of developing an infectious disease or disorder. In some embodiments, the subject has contracted an infectious disease by way of contact with another infected subject. In some embodiments, the subject has contracted an infectious disease from contaminated drinking water. In some embodiments, the subject has contracted the infectious disease from a different species carrying the microorganism. Further provided herein are methods, wherein the composition is administered to a mammal. Further provided herein are methods, wherein the composition is administered to a human subject. Further provided herein are methods, wherein the composition is administered for veterinary uses. Further provided herein are methods, wherein the composition is administered to a livestock or a domesticated animal.

Provided herein are methods for modulating an immune response, wherein the methods comprise: administering to a subject having a cancer a composition provided herein. In some embodiments, the cancer is a solid cancer or a blood cancer. In some embodiments, the blood cancer is lymphoma or leukemia. In some embodiments, the subject has, is at risk for developing, or is suspected of having a skin cancer. In some embodiments, the skin cancer is a basal cell cancer, a melanoma, a Merkel cell cancer, a squamous cell carcinoma, a cutaneous lymphoma, a Kaposi sarcoma, or a skin adnexal cancer. In some embodiments, the subject has, is at risk for developing, or is suspected of having a pancreatic cancer. In some embodiments, the pancreatic cancer is a pancreatic adenocarcinoma, a pancreatic exocrine cancer, a pancreatic neuroendocrine cancer, an islet cell cancer, or a pancreatic endocrine cancer. In some embodiments, the subject has, is at risk for developing, or is suspected of having a colon cancer, a prostate cancer, an ovarian cancer, or a breast cancer. Further provided herein are methods of treating a subject with a cancer, wherein the cancer expresses a TRP-1 protein, a prostein protein, a MAGE-A1 protein, a MAGE-A3 protein, or a combination thereof, the method comprising administering to the subject having a cancer a composition provided herein (e.g., NP-1 or NP-30 complexed to a nucleic acid encoding TRP-1, prostein, MAGE-A1, or MAGE-A3).

### (11) Kits

In some embodiments, a formulation of a composition described herein is prepared in a single container for administration. In some embodiments, a formulation of a composition described herein is prepared in two containers for administration, separating the nucleic acid and/or the compound provided herein from the nanoparticle carrier.

As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some implementations, the containers are RNase free.

Provided herein is kit, wherein the kit comprises: a first container comprising: a composition comprising nanoparticles, wherein the nanoparticles comprises a hydrophobic core, wherein the hydrophobic core comprises an oil in liquid phase at 25 degrees Celsius; and a second container comprising: a nucleic acid encoding for a protein or a functional fragment thereof.

In some embodiments, the nanoparticles comprise a hydrophobic core comprising lipids in liquid phase at 25 degrees Celsius. In some embodiments, the nanoparticles comprise a cationic lipid, an oil, surfactants, and optionally, an inorganic particle. In some embodiments, the nucleic acid further codes for a RNA polymerase. In some embodiments, the RNA polymerase is a Venezuelan equine encephalitis virus (VEEV) RNA polymerase. In some embodiments, the nucleic acid sequence encoding for the RNA polymerase comprises the sequence of SEQ ID NO: 176. In some embodiments, the first container is lyophilized. In some embodiments, the kit further comprises an adjuvant provided herein.

### Exemplary Embodiments

Provided herein are methods for generating a local immune response, wherein the methods comprise: administering to a tissue in a subject a composition, wherein the composition comprises: nanoparticles, wherein the nanoparticles comprise: a hydrophobic core comprising lipids in liquid phase at 25 degrees Celsius; and nucleic acids, wherein the nucleic acids are complexed to the nanoparticles to form nucleic acid-nanoparticle complexes, and wherein the administering of the composition provides for a local immune response within a tissue. Further provided herein are methods, wherein the administering is intramuscular administration, intradermal administration, transdermal administration, sublingual administration, buccal administration, intranasal administration, inhalation administration, intrathecal administration, or intratumoral administration. Further provided herein are methods, wherein the local immune response is characterized by an increase in the level of at least one immunostimulatory marker in the tissue relative to a reference level. Further provided herein are methods, wherein the reference level is the level of the immunostimulatory marker in a subject that has not been administered the composition or the level of the immunostimulatory marker in the subject prior to administration of the composition to the tissue. Further provided herein are methods, wherein the at least one immunostimulatory marker comprises: cluster of differentiation 86 (CD86), H-2 class II histocompatibility antigen, A beta chain (H2-ab1), H-2 class II histocompatibility antigen, I-E beta chain (H2-eb1), integrin alpha L chain (ITGAL or CDT 1a), Integrin alpha M (ITGAM, CR3A, or CD11b), Fcg receptor (FcγR), cluster of differentiation 28 (CD28) or a combination thereof. Further provided herein are methods, wherein the local immune response is characterized by recruitment of immune cells to the tissue. Further provided herein are methods, wherein the immune cells comprise a population of MHC-II^{lo} macrophages, a population of plasmacytoid dendritic cells (pDCs), a population of monocyte dendritic cells (MoDCs), a population of neutrophils, a population of NK cells, or a combination thereof. Further provided herein are methods, wherein the tissue is a muscle tissue, an epithelial tissue, an endothelial tissue, a mucosal tissue, a nasal cavity tissue, a thecal tissue, or a tumor. Further provided herein are methods, wherein the local immune response is characterized by a lower level or activity of systemic interferons relative to a reference level. Further provided herein are methods, wherein the reference level comprises the level or activity of systemic interferons in a subject with systemic inflammation or the level or activity of systemic interferons in a subject that has been administered a reactogenic nanoparticle composition. Further provided herein are methods, wherein the systemic interferon comprises interferon-alpha 2 (IFN-α2) or interferon gamma (IFN-γ). Further provided herein are methods, wherein the local immune response is characterized by a lower level of cardiac troponin in the blood of the subject relative to a reference level. Further provided herein are methods, wherein the reference level is the level of cardiac troponin in the blood of subject that has systemic inflammation, myocarditis, or the level of cardiac troponin in the blood of a subject that has been administered a reactogenic nanoparticle composition. Further provided herein are methods, wherein the cardiac troponin comprises troponin I (cTNI) or troponin T (cTNT). Further provided herein are methods, wherein the local immune response is characterized by a higher level of interferon in the tissue administered the composition relative to the level of interferon in a heart, a spleen, or a liver of the subject. Further provided herein are methods, wherein the local immune response is characterized by localization of the nucleic acids in the tissue, wherein the tissue is within or adjacent to a site of administration. Further provided herein are methods, wherein the nanoparticles are characterized as having a z-average diameter particle size measurement of about 40 nm to about 300 nm when measured by dynamic light scattering. Further provided herein are methods, wherein the nanoparticles are characterized as having a z-average diameter particle size measurement of about 40 nm up to 150 nm when measured by dynamic light scattering. Further provided herein are methods, wherein prior to forming a complex with the nucleic acids, the nanoparticles are characterized as having a z-average diameter particle size measurement of about 40 nm up to 60 nm when measured by dynamic light scattering. Further provided herein are methods, wherein the nucleic acid-nanoparticle complexes are characterized as having a z-average diameter particle size measurement of up to about 600 nm when measured by dynamic light scattering. Further provided herein are methods, wherein the nucleic acid-lipid nanoparticle complexes comprise a z-average diameter particle size measurement of at least two-fold greater than that of the nanoparticles when measured by dynamic light scattering. Further provided herein are methods, wherein the surface comprises one or more cationic lipid selected from the group consisting of: 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate, A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate, ALC-0315, ((4-hydroxybutyl)azanediyl)bis(hexane-6,1-diyl)bis(2-hexyldecanoate), ALC-0159, 2-[(polyethylene glycol)-2000]-N,N-ditetradecylacetamide, β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol, BAME-016B, bis(2-(dodecyldisulfanyl)ethyl) 3,3′-((3-methyl-9-oxo-10-oxa-13,14-dithia-3,6-diazahexacosyl)azanediyl)dipropionate, BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide, cKK-E12, 3,6-bis(4-(bis(2-hydroxydodecyl)amino)butyl)piperazine-2,5-dione, DC-Cholesterol, 30-[N-(N′,N′-dimethylaminoethane)-carbamoyl]cholesterol, DLin-MC3-DMA, (6Z,9Z,28Z,31Z)-heptatriaconta-6,9,28,31-tetraen-19-yl 4-(dimethylamino) butanoate, DOPE, 1,2-dioleoyl-sn-glycero-3-phosphoethanolamine, DOSPA, 2,3-dioleyloxy-N-[2-(sperminecarboxamido)ethyl]-N,N-dimethyl-1-propanaminium trifluoroacetate, DSPC, 1,2-distearoyl-sn-glycero-3-phosphocholine, ePC, ethylphosphatidylcholine, FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate, Lipid H (SM-102), heptadecan-9-yl 8-((2-hydroxyethyl)(6-oxo-6-(undecyloxy)hexyl)amino) octanoate, OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate), PEG2000-DMG, (R)-2,3-bis(myristoyloxy)propyl-1-(methoxy poly(ethylene glycol)2000) carbamate, and or N1,N3,N5-tris(3-(didodecylamino)propyl)benzene-1,3,5-tricarboxamide (TT3). Further provided herein are methods, wherein the liquid oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, solanesol, soy lecithin, soybean oil, sunflower oil, a triglyceride, or vitamin E. Further provided herein are methods, wherein the triglyceride is capric triglyceride, caprylic triglyceride, a caprylic and capric triglyceride, a triglyceride ester, or myristic acid triglycerin. Further provided herein are methods, wherein the nanoparticles further comprise a surfactant. Further provided herein are methods, wherein the surfactant is a hydrophobic surfactant. Further provided herein are methods, wherein the surfactant is a hydrophilic surfactant. Further provided herein are methods, wherein the surfactant is an amphiphilic surfactant. Further provided herein are methods, wherein the hydrophobic surfactant comprises sorbitan monolaurate, sorbitan monopalmitate, sorbitan monostearate, sorbitan tristearate, sorbitan monooleate, or sorbitan trioleate. Further provided herein are methods, wherein the hydrophilic surfactant comprises polyoxyethylene sorbitan ester (polysorbate), polyoxyethylene sorbitan monooleate, polyoxyethylene sorbitan monostearate, polyoxyethylene sorbitan monopalmitate, polyoxyethylene sorbitan monolaurate. Further provided herein are methods, wherein the amphiphilic surfactant comprises sodium dodecyl sulfate (SDS). Further provided herein are methods, wherein the hydrophobic core further comprises a phosphate-terminated lipid. Further provided herein are methods, wherein the phosphate-terminated lipid comprises trioctylphosphine oxide (TOPO) or distearyl phosphatidic acid (DSPA). Further provided herein are methods, wherein the hydrophobic core further comprises a phosphorous-terminated surfactant, a carboxylate-terminated surfactant, a sulfate-terminated surfactant, or an amine-terminated surfactant. Further provided herein are methods, wherein the carboxylate-terminated surfactant comprises oleic acid. Further provided herein are methods, wherein the amine-terminated surfactant comprises oleylamine. Further provided herein are methods, wherein the hydrophobic core further comprises an inorganic particle. Further provided herein are methods, wherein the inorganic particle comprises a metal. Further provided herein are methods, wherein the metal comprises iron oxide, magnetite (Fe₃O₄), maghemite (y-Fe₂O₃), wüstite (FeO), hematite (alpha (α)-Fe₂O₃), aluminum hydroxide, or aluminum oxyhydroxide. Further provided herein are methods, wherein the inorganic particle is coated with a surfactant or a capping ligand. Further provided herein are methods, wherein the inorganic particle is coated with trioctylphosphine oxide (TOPO) or distearyl phosphatidic acid (DSPA). Further provided herein are methods, wherein the nanoparticles comprise a hydrophilic-lipophilic balance (HLB) value of at least about 8 up to 11. Further provided herein are methods, wherein the nanoparticles comprise an oil-to-surfactant molar ratio ranging from at least about 0.4:1 up to 1:1. Further provided herein are methods, wherein the nanoparticles comprise a nitrogen:phosphate (N:P) molar ratio from at least about 5:1 up to 30:1. Further provided herein are methods, wherein the nanoparticles comprise an average polydispersity index (PDI) from at least about 0.1 to about 0.3. Further provided herein are methods, wherein the nucleic acids are in complex with the nanoparticles. Further provided herein are methods, wherein the nucleic acids each encode for one or more bioactive agent. Further provided herein are methods, wherein the one or more bioactive agent comprises a protein, an antibody, an antibody fragment, a cytokine, or an immune system modulator. Further provided herein are methods, wherein the composition comprises at least one nucleic acid encoding for an RNA polymerase. Further provided herein are methods, wherein the composition comprises at least one nucleic acid comprising a region encoding for Venezuelan equine encephalitis virus (VEEV). Further provided herein are methods, wherein the composition further comprises an adjuvant. Further provided herein are methods, wherein the composition is lyophilized prior to administration to the subject. Further provided herein are methods, wherein the composition is formulated as a suspension. Further provided herein are methods, wherein the composition is in a liquid, semi-liquid, solution, propellant, or powder dosage form. Further provided herein are methods, wherein the nanoparticles are in aqueous solution. Further provided herein are methods, wherein the composition is administered to a mammal. Further provided herein are methods, wherein the composition is administered to a human subject. Further provided herein are methods, wherein the composition is administered for veterinary uses. Further provided herein are methods, wherein the composition is administered to a livestock or a domesticated animal. Provided herein are methods wherein the methods comprise: administering to a tissue in a subject a composition, wherein the composition comprises: administering to a tissue in a subject a composition, wherein the composition comprises: nanoparticles, wherein the nanoparticles comprise: a hydrophobic core comprising lipids in liquid phase at 25 degrees Celsius; and nucleic acids encoding for a protein, wherein the nucleic acids encoding for a protein are complexed to the nanoparticles to form nucleic acid-nanoparticle complexes, wherein the composition comprises a nucleic acid biodistribution ratio between 0 and 0.1 (0≤nucleic acid biodistribution ratio ≤0.1) when administered to a subject, and wherein the nucleic acid biodistribution ratio is determined by the formula:

$$\frac{x 1}{x 2} = nucleic acid biodistribution ratio$$

wherein x1 is the level of the nucleic acids in a tissue that is not within or not adjacent to the tissue that was administered the composition, wherein x2 is the level of the nucleic acids in the tissue administered the composition. Further provided herein are methods, wherein the level of nucleic acids is determined by polymerase chain reaction (PCR), spectrophotometry, microarray, or an in vivo imaging assay. Further provided herein are methods, wherein the composition comprises a lower nucleic acid biodistribution ratio relative to a nucleic acid biodistribution ratio of a reactogenic nanoparticle composition. Further provided herein are methods, wherein the reactogenic nanoparticle composition comprises one or more characteristics from (i)-(iii): (i) nucleic acids encapsulated in a core of the reactogenic nanoparticle composition; (ii) a net neutral charge; (iii) an average Z-diameter greater than 60 nm. Further provided herein are methods, the administering is via intramuscular injection. Further provided herein are methods, wherein the tissue is a muscle tissue. Further provided herein are methods, wherein the tissue that is not within or not adjacent to the tissue administered the composition is a liver tissue, a spleen tissue, an ovarian tissue, a testis tissue, a lung tissue, a brain tissue, or a heart tissue. Further provided herein are methods, wherein the nucleic acids encode for a protein. Further provided herein are methods, wherein the protein is a microbial protein antigen or a cancer-associated protein. Further provided herein are methods, wherein the microbial protein antigen is a microbial protein antigen listed in Table 3. Further provided herein are methods, wherein the microbial protein antigen is from a Chlamydia trachomatis bacteria, an enterovirus, a gamma herpesvirus, an alphaherpesvirus, a human papillomavirus virus (HPV), an influenza virus, a Mycobacterium tuberculosis bacteria, a Pseudomonas bacteria, a Acinetobacter bacteria, a Klebsiella bacteria, an Escherichia coli bacteria, a Serratia bacteria, a Streptococcus bacteria, a Shigella bacteria, a Campylobacter bacteria, a Staphylococcus bacteria, a Salmonellae bacteria, an Enterococcus bacteria, a Helicobacter pylori bacteria, a Neisseria gonorrhoeae bacteria, a Haemophilus influenzae bacteria, a Proteus bacteria, a rabies virus, a respiratory syncytial virus, a coronavirus, an enterovirus, a severe acute respiratory syndrome (SARS) virus, a Varicella-Zoster Virus (VZV), or a Zika virus. Further provided herein are methods, wherein the nucleic acid comprises a region comprising a sequence that is at least 85% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 90% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 95% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 99% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising any one of SEQ ID NOS: 11, 20, 22, 25, 27, 29, 31, 33, 35, 37, 39, 43, 49, 51, 67, 180, 181, 183, 185-192. Further provided herein are methods, wherein the cancer-associated protein is a cancer-associated protein listed in Table 6 or Table 7. Further provided herein are methods, wherein the cancer-associated protein is a Melanoma Antigen Gene (MAGE) protein, a Tyrosinase-related protein 1 (TRP-1) protein, prostein, tyrosinase, a glycoprotein 100 (gp100) protein, a melanoma antigen recognized by T cells 1 (MART) protein, a glycoprotein 75 (gp75) protein, a Tyrosinase-related protein 2 (TRP-2) protein, a Carcinoembryonic antigen (CEA) protein, a human epidermal growth factor receptor (HER-2), a prostate-specific membrane antigen (PSMA) protein, a B melanoma antigen (BAGE) protein, a G antigen (GAGE) protein, a cancer/testis antigen protein, a 43kD protein (p43), a p15, a beta catenin protein, a CAP-8 protein, a multiple myeloma 1 (MUM-1) protein, a mucin (MUC) protein, a variant, or a derivative thereof. Further provided herein are methods, wherein the nucleic acid comprises a region comprising a sequence that is at least 85% identical to any one of SEQ ID NOS: 70-72. Further provided herein are methods, wherein the nucleic acid comprises a region comprising a sequence that is at least 90% identical to any one of SEQ ID NOS: 70-72. Further provided herein are methods, wherein the nucleic acid comprises a region comprising a sequence that is at least 95% identical to any one of SEQ ID NOS: 70-72. Further provided herein are methods, wherein the nucleic acid comprises a region comprising a sequence that is at least 99% identical to any one of SEQ ID NOS: 70-72. Further provided herein are methods, wherein the nucleic acid comprises a region comprising any one of SEQ ID NOS: 70-72. Further provided herein are methods, wherein the nucleic acid comprises a region encoding for an amino acid sequence that is least 85% identical to any one of SEQ ID NOS: 73-147. Further provided herein are methods, wherein the nucleic acid comprises a region encoding for an amino acid sequence that is least 90% identical to any one of SEQ ID NOS: 73-147. Further provided herein are methods, wherein the nucleic acid comprises a region encoding for an amino acid sequence that is least 95% identical to any one of SEQ ID NOS: 73-147. Further provided herein are methods, wherein the nucleic acid comprises a region encoding for an amino acid sequence that is least 99% identical to any one of SEQ ID NOS: 73-147. Further provided herein are methods, wherein the nucleic acid comprises a region encoding for an amino acid sequence comprising any one of SEQ ID NOS: 73-147. Further provided herein are methods, wherein the protein is an antibody or a functional variant thereof. Further provided herein are methods, wherein the antibody or functional variant thereof comprises an antibody listed in Table 4 or Table 5. Further provided herein are methods, wherein the antibody or functional variant thereof comprises wherein the antibody or functional variant thereof comprises bamlanivimab, casirivimab, imdevimab, sotrovimab, atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, trastuzumab, fragments, derivatives, or variants thereof. Further provided herein are methods, wherein the antibody or functional variant thereof comprises an amino acid sequence that is at least 85% identical to any one of SEQ ID NOS: 52-66, 68-69. Further provided herein are methods, wherein the antibody or functional variant thereof comprises an amino acid sequence that is at least 90% identical to any one of SEQ ID NOS: 52-66, 68-69. Further provided herein are methods, wherein the antibody or functional variant thereof comprises an amino acid sequence that is at least 95% identical to any one of SEQ ID NOS: 52-66, 68-69. Further provided herein are methods, wherein the antibody or functional variant thereof comprises an amino acid sequence that is at least 99% identical to any one of SEQ ID NOS: 52-66, 68-69. Further provided herein are methods, wherein the antibody or functional variant thereof comprises any one of SEQ ID NOS: 52-66, 68-69. Further provided herein are methods, wherein the nanoparticles are characterized as having a z-average diameter particle size measurement of about 40 nm to about 300 nm when measured by dynamic light scattering. Further provided herein are methods, wherein the nanoparticles are characterized as having a z-average diameter particle size measurement of about 40 nm up to 150 nm when measured by dynamic light scattering. Further provided herein are methods, wherein prior to forming a complex with the nucleic acids, the nanoparticles are characterized as having a z-average diameter particle size measurement of about 40 nm up to 60 nm when measured by dynamic light scattering. Further provided herein are methods, wherein the nucleic acid-nanoparticle complexes are characterized as having a z-average diameter particle size measurement of up to about 600 nm when measured by dynamic light scattering. Further provided herein are methods, wherein the nucleic acid-lipid nanoparticle complexes comprise a z-average diameter particle size measurement of at least two-fold greater than that of the nanoparticles when measured by dynamic light scattering. Further provided herein are methods, wherein the surface comprises one or more cationic lipid selected from the group consisting of: 1,2-dioleoyloxy-3 (trimethylammonium)propane (DOTAP), 30-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; (3-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12′Z,12′″″Z)-tetrakis (octadeca-9,12-dienoate); PEG2000-DMG, (R)-2,3-bis(myristoyloxy)propyl-1-(methoxy poly(ethylene glycol)2000) carbamate; and or N1,N3,N5-tris(3-(didodecylamino)propyl)benzene-1,3,5-tricarboxamide (TT3). Further provided herein are methods, wherein the liquid oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, solanesol, soy lecithin, soybean oil, sunflower oil, a triglyceride, or vitamin E. Further provided herein are methods, wherein the triglyceride is capric triglyceride, caprylic triglyceride, a caprylic and capric triglyceride, a triglyceride ester, or myristic acid triglycerin. Further provided herein are methods, wherein the nanoparticles further comprise a surfactant. Further provided herein are methods, wherein the surfactant is a hydrophobic surfactant. Further provided herein are methods, wherein the surfactant is a hydrophilic surfactant. Further provided herein are methods, wherein the surfactant is an amphiphilic surfactant. Further provided herein are methods, wherein the hydrophobic surfactant comprises sorbitan monolaurate, sorbitan monopalmitate, sorbitan monostearate, sorbitan tristearate, sorbitan monooleate, or sorbitan trioleate. Further provided herein are methods, wherein the hydrophilic surfactant comprises polyoxyethylene sorbitan ester (polysorbate), polyoxyethylene sorbitan monooleate, polyoxyethylene sorbitan monostearate, polyoxyethylene sorbitan monopalmitate, polyoxyethylene sorbitan monolaurate. Further provided herein are methods, wherein the amphiphilic surfactant comprises sodium dodecyl sulfate (SDS). Further provided herein are methods, wherein the hydrophobic core further comprises a phosphate-terminated lipid. Further provided herein are methods, wherein the phosphate-terminated lipid comprises trioctylphosphine oxide (TOPO) or distearyl phosphatidic acid (DSPA). Further provided herein are methods, wherein the hydrophobic core further comprises a phosphorous-terminated surfactant, a carboxylate-terminated surfactant, a sulfate-terminated surfactant, or an amine-terminated surfactant. Further provided herein are methods, wherein the carboxylate-terminated surfactant comprises oleic acid. Further provided herein are methods, wherein the amine-terminated surfactant comprises oleylamine. Further provided herein are methods, wherein the hydrophobic core further comprises an inorganic particle. Further provided herein are methods, wherein the inorganic particle comprises a metal. Further provided herein are methods, wherein the metal comprises iron oxide, magnetite (Fe₃O₄), maghemite (y-Fe₂O₃), wüstite (FeO), hematite (alpha (α)-Fe₂O₃), aluminum hydroxide, or aluminum oxyhydroxide. Further provided herein are methods, wherein the inorganic particle is coated with a surfactant or a capping ligand. Further provided herein are methods, wherein the inorganic particle is coated with trioctylphosphine oxide (TOPO) or distearyl phosphatidic acid (DSPA). Further provided herein are methods, wherein the nanoparticles comprise a hydrophilic-lipophilic balance (HLB) value of at least about 8 up to 11. Further provided herein are methods, wherein the nanoparticles comprise an oil-to-surfactant molar ratio ranging from at least about 0.4:1 up to 1:1. Further provided herein are methods, wherein the nanoparticles comprise a nitrogen:phosphate (N:P) molar ratio from at least about 5:1 up to 30:1. Further provided herein are methods, wherein the nanoparticles comprise an average polydispersity index (PDI) from at least about 0.1 to about 0.3. Further provided herein are methods, wherein the nucleic acids are in complex with the nanoparticles. Further provided herein are methods, wherein the nucleic acids each encode for one or more bioactive agent. Further provided herein are methods, wherein the one or more bioactive agent comprises a protein, an antibody, an antibody fragment, a cytokine, or an immune system modulator. Further provided herein are methods, wherein the composition comprises at least one nucleic acid encoding for an RNA polymerase. Further provided herein are methods, wherein the composition comprises at least one nucleic acid comprising a region encoding for Venezuelan equine encephalitis virus (VEEV). Further provided herein are methods, wherein the composition further comprises an adjuvant. Further provided herein are methods, wherein the composition is lyophilized prior to administration to the subject. Further provided herein are methods, wherein the composition is formulated as a suspension. Further provided herein are methods, wherein the composition is in a liquid, semi-liquid, solution, propellant, or powder dosage form. Further provided herein are methods, wherein the nanoparticles are in aqueous solution. Further provided herein are methods, wherein the composition is administered to a mammal. Further provided herein are methods, wherein the composition is administered to a human subject. Further provided herein are methods, wherein the composition is administered for veterinary uses. Further provided herein are methods, wherein the composition is administered to a livestock or a domesticated animal. Provided herein are methods wherein the methods comprise: administering to a tissue in a subject a composition, wherein the composition comprises: administering to a tissue in a subject a composition, wherein the composition comprises: nanoparticles, wherein the nanoparticles comprise: a hydrophobic core comprising lipids in liquid phase at 25 degrees Celsius; and nucleic acids encoding for a protein, wherein the nucleic acids encoding for a protein are complexed to the nanoparticles to formnucleic acid-nanoparticle complexes, wherein the composition comprises a nucleic acid biodistribution ratio between 0 and 1 (0<nucleic acid biodistribution ratio ≤1) when administered to a subject, and wherein the nucleic acid biodistribution ratio is determined by the formula:

$$\frac{x 1}{x 2} = nucleic acid biodistribution ratio$$

wherein x1 is the level of the nucleic acids in a tissue that is not within or not adjacent to the tissue that was administered the composition,
wherein x2 is the level of the nucleic acids in the tissue administered the composition. Further provided herein are methods, wherein the level of nucleic acids is determined by polymerase chain reaction (PCR), spectrophotometry, microarray, or an in vivo imaging assay. Further provided herein are methods, wherein the composition comprises a lower nucleic acid biodistribution ratio relative to a nucleic acid biodistribution ratio of a reactogenic nanoparticle composition. Further provided herein are methods, wherein the reactogenic nanoparticle composition comprises one or more characteristics from (i)-(iii): (i) nucleic acids encapsulated in a core of the reactogenic nanoparticle composition; (ii) a net neutral charge; (iii) an average Z-diameter greater than 60 nm. Further provided herein are methods, the administering is via intramuscular injection. Further provided herein are methods, wherein the tissue is a muscle tissue. Further provided herein are methods, wherein the tissue that is not within or not adjacent to the tissue administered the composition is a liver tissue, a spleen tissue, an ovarian tissue, a testis tissue, a lung tissue, a brain tissue, or a heart tissue. Further provided herein are methods, wherein the nucleic acids encode for a protein. Further provided herein are methods, wherein the protein is a microbial protein antigen or a cancer-associated protein. Further provided herein are methods, wherein the microbial protein antigen is a microbial protein antigen listed in Table 3. Further provided herein are methods, wherein the microbial protein antigen is from a Chlamydia trachomatis bacteria, an enterovirus, a gamma herpesvirus, an alphaherpesvirus, a human papillomavirus virus (HPV), an influenza virus, a Mycobacterium tuberculosis bacteria, a Pseudomonas bacteria, a Acinetobacter bacteria, a Klebsiella bacteria, an Escherichia coli bacteria, a Serratia bacteria, a Streptococcus bacteria, a Shigella bacteria, a Campylobacter bacteria, a Staphylococcus bacteria, a Salmonellae bacteria, an Enterococcus bacteria, a Helicobacter pylori bacteria, a Neisseria gonorrhoeae bacteria, a Haemophilus influenzae bacteria, a Proteus bacteria, a rabies virus, a respiratory syncytial virus, a coronavirus, a severe acute respiratory syndrome (SARS) virus, a Varicella-Zoster Virus (VZV), or a Zika virus. Further provided herein are methods, wherein the nucleic acid comprises a region comprising a sequence that is at least 85% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 90% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 95% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 99% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising any one of SEQ ID NOS: 11, 20, 22, 25, 27, 29, 31, 33, 35, 37, 39, 43, 49, 51, 67, 180, 181, 183, 185-192. Further provided herein are methods, wherein the microbial protein antigen is a major outer membrane protein, an envelope protein, an envelope glycoprotein, an E7 protein, an E6 oncoprotein, a haemagglutinin protein, a malate synthase protein, a nucleoprotein, an L protein, a transmembrane glycoprotein, a phosphoroprotein, an M2 protein, and RSV glycoprotein, an RSV fusion protein, a spike protein, a variant, or a derivative thereof. Further provided herein are methods, wherein the cancer-associated protein is a cancer-associated protein listed in Table 6 or Table 7. Further provided herein are methods, wherein the cancer-associated protein is a Melanoma Antigen Gene (MAGE) protein, a Tyrosinase-related protein 1 (TRP-1) protein, prostein, tyrosinase, a glycoprotein 100 (gp100) protein, a melanoma antigen recognized by T cells 1 (MART) protein, a glycoprotein 75 (gp75) protein, a Tyrosinase-related protein 2 (TRP-2) protein, a Carcinoembryonic antigen (CEA) protein, a human epidermal growth factor receptor (HER-2), a prostate-specific membrane antigen (PSMA) protein, a B melanoma antigen (BAGE) protein, a G antigen (GAGE) protein, a cancer/testis antigen protein, a 43kD protein (p43), a p15, a beta catenin protein, a CAP-8 protein, a multiple myeloma 1 (MUM-1) protein, a mucin (MUC) protein, a variant, or a derivative thereof. Further provided herein are methods, wherein the nucleic acid comprises a region comprising a sequence that is at least 85% identical to any one of SEQ ID NOS: 70-72. Further provided herein are methods, wherein the nucleic acid comprises a region comprising a sequence that is at least 90% identical to any one of SEQ ID NOS: 70-72. Further provided herein are methods, wherein the nucleic acid comprises a region comprising a sequence that is at least 95% identical to any one of SEQ ID NOS: 70-72. Further provided herein are methods, wherein the nucleic acid comprises a region comprising a sequence that is at least 99% identical to any one of SEQ ID NOS: 70-72. Further provided herein are methods, wherein the nucleic acid comprises a region comprising any one of SEQ ID NOS: 70-72. Further provided herein are methods, wherein the nucleic acid comprises a region encoding for an amino acid sequence that is least 85% identical to any one of SEQ ID NOS: 73-147. Further provided herein are methods, wherein the nucleic acid comprises a region encoding for an amino acid sequence that is least 90% identical to any one of SEQ ID NOS: 73-147. Further provided herein are methods, wherein the nucleic acid comprises a region encoding for an amino acid sequence that is least 95% identical to any one of SEQ ID NOS: 73-147. Further provided herein are methods, wherein the nucleic acid comprises a region encoding for an amino acid sequence that is least 99% identical to any one of SEQ ID NOS: 73-147. Further provided herein are methods, wherein the nucleic acid comprises a region encoding for an amino acid sequence comprising any one of SEQ ID NOS: 73-147. Further provided herein are methods, wherein the protein is an antibody or a functional variant thereof. Further provided herein are methods, wherein the antibody or functional variant thereof comprises an antibody listed in Table 4 or Table 5. Further provided herein are methods, wherein the antibody or functional variant thereof comprises wherein the antibody or functional variant thereof comprises bamlanivimab, casirivimab, imdevimab, sotrovimab, atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, trastuzumab, fragments, derivatives, or variants thereof. Further provided herein are methods, wherein the antibody or functional variant thereof comprises an amino acid sequence that is at least 85% identical to any one of SEQ ID NOS: 52-66, 68-69. Further provided herein are methods, wherein the antibody or functional variant thereof comprises an amino acid sequence that is at least 90% identical to any one of SEQ ID NOS: 52-66, 68-69. Further provided herein are methods, wherein the antibody or functional variant thereof comprises an amino acid sequence that is at least 95% identical to any one of SEQ ID NOS: 52-66, 68-69. Further provided herein are methods, wherein the antibody or functional variant thereof comprises an amino acid sequence that is at least 99% identical to any one of SEQ ID NOS: 52-66, 68-69. Further provided herein are methods, wherein the antibody or functional variant thereof comprises any one of SEQ ID NOS: 52-66, 68-69. Further provided herein are methods, wherein the nanoparticles are characterized as having a z-average diameter particle size measurement of about 40 nm to about 300 nm when measured by dynamic light scattering. Further provided herein are methods, wherein the nanoparticles are characterized as having a z-average diameter particle size measurement of about 40 nm up to 150 nm when measured by dynamic light scattering. Further provided herein are methods, wherein prior to forming a complex with the nucleic acids, the nanoparticles are characterized as having a z-average diameter particle size measurement of about 40 nm up to 60 nm when measured by dynamic light scattering. Further provided herein are methods, wherein the nucleic acid-nanoparticle complexes are characterized as having a z-average diameter particle size measurement of up to about 600 nm when measured by dynamic light scattering. Further provided herein are methods, wherein the nucleic acid-lipid nanoparticle complexes comprise a z-average diameter particle size measurement of at least two-fold greater than that of the nanoparticles when measured by dynamic light scattering. Further provided herein are methods, wherein the surface comprises one or more cationic lipid selected from the group consisting of: 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; 0-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″″Z)-tetrakis (octadeca-9,12-dienoate); PEG2000-DMG, (R)-2,3-bis(myristoyloxy)propyl-1-(methoxy poly(ethylene glycol)2000) carbamate; and or N1,N3,N5-tris(3-(didodecylamino)propyl)benzene-1,3,5-tricarboxamide (TT3). Further provided herein are methods, wherein the liquid oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, solanesol, soy lecithin, soybean oil, sunflower oil, a triglyceride, or vitamin E. Further provided herein are methods, wherein the triglyceride is capric triglyceride, caprylic triglyceride, a caprylic and capric triglyceride, a triglyceride ester, or myristic acid triglycerin. Further provided herein are methods, wherein the nanoparticles further comprise a surfactant. Further provided herein are methods, wherein the surfactant is a hydrophobic surfactant. Further provided herein are methods, wherein the surfactant is a hydrophilic surfactant. Further provided herein are methods, wherein the surfactant is an amphiphilic surfactant. Further provided herein are methods, wherein the hydrophobic surfactant comprises sorbitan monolaurate, sorbitan monopalmitate, sorbitan monostearate, sorbitan tristearate, sorbitan monooleate, or sorbitan trioleate. Further provided herein are methods, wherein the hydrophilic surfactant comprises polyoxyethylene sorbitan ester (polysorbate), polyoxyethylene sorbitan monooleate, polyoxyethylene sorbitan monostearate, polyoxyethylene sorbitan monopalmitate, polyoxyethylene sorbitan monolaurate. Further provided herein are methods, wherein the amphiphilic surfactant comprises sodium dodecyl sulfate (SDS). Further provided herein are methods, wherein the hydrophobic core further comprises a phosphate-terminated lipid. Further provided herein are methods, wherein the phosphate-terminated lipid comprises trioctylphosphine oxide (TOPO) or distearyl phosphatidic acid (DSPA). Further provided herein are methods, wherein the hydrophobic core further comprises a phosphorous-terminated surfactant, a carboxylate-terminated surfactant, a sulfate-terminated surfactant, or an amine-terminated surfactant. Further provided herein are methods, wherein the carboxylate-terminated surfactant comprises oleic acid. Further provided herein are methods, wherein the amine-terminated surfactant comprises oleylamine. Further provided herein are methods, wherein the hydrophobic core further comprises an inorganic particle. Further provided herein are methods, wherein the inorganic particle comprises a metal. Further provided herein are methods, wherein the metal comprises iron oxide, magnetite (Fe₃O₄), maghemite (y-Fe₂O₃), wüstite (FeO), hematite (alpha (α)-Fe₂O₃), aluminum hydroxide, or aluminum oxyhydroxide. Further provided herein are methods, wherein the inorganic particle is coated with a surfactant or a capping ligand. Further provided herein are methods, wherein the inorganic particle is coated with trioctylphosphine oxide (TOPO) or distearyl phosphatidic acid (DSPA). Further provided herein are methods, wherein the nanoparticles comprise a hydrophilic-lipophilic balance (HLB) value of at least about 8 up to 11. Further provided herein are methods, wherein the nanoparticles comprise an oil-to-surfactant molar ratio ranging from at least about 0.4:1 up to 1:1. Further provided herein are methods, wherein the nanoparticles comprise a nitrogen:phosphate (N:P) molar ratio from at least about 5:1 up to 30:1. Further provided herein are methods, wherein the nanoparticles comprise an average polydispersity index (PDI) from at least about 0.1 to about 0.3. Further provided herein are methods, wherein the nucleic acids are in complex with the nanoparticles. Further provided herein are methods, wherein the nucleic acids each encode for one or more bioactive agent. Further provided herein are methods, wherein the one or more bioactive agent comprises a protein, an antibody, an antibody fragment, a cytokine, or an immune system modulator. Further provided herein are methods, wherein the composition comprises at least one nucleic acid encoding for an RNA polymerase. Further provided herein are methods, wherein the composition comprises at least one nucleic acid comprising a region encoding for Venezuelan equine encephalitis virus (VEEV). Further provided herein are methods, wherein the composition further comprises an adjuvant. Further provided herein are methods, wherein the composition is lyophilized prior to administration to the subject. Further provided herein are methods, wherein the composition is formulated as a suspension. Further provided herein are methods, wherein the composition is in a liquid, semi-liquid, solution, propellant, or powder dosage form. Further provided herein are methods, wherein the nanoparticles are in aqueous solution. Further provided herein are methods, wherein the composition is administered to a mammal. Further provided herein are methods, wherein the composition is administered to a human subject. Further provided herein are methods, wherein the composition is administered for veterinary uses. Further provided herein are methods, wherein the composition is administered to a livestock or a domesticated animal. Provided herein are methods wherein the methods comprise: administering to a tissue in a subject a composition, wherein the composition comprises: administering to a tissue in a subject a composition, wherein the composition comprises: nanoparticles, wherein the nanoparticles comprise: a hydrophobic core comprising lipids in liquid phase at 25 degrees Celsius; and nucleic acids encoding for a protein, wherein the nucleic acids encoding for a protein are complexed to the nanoparticles to form nucleic acid-nanoparticle complexes, wherein the composition comprises a nucleic acid biodistribution ratio that is less than the biodistribution ratio of a reactogenic nanoparticle composition (nucleic acid biodistribution ratio ≤nucleic acid biodistribution ratio of the reactogenic nanoparticle composition) when administered to a subject, and wherein the nucleic acid biodistribution ratio is determined by the formula:

$$\frac{x 1}{x 2} = nucleic acid biodistribution ratio$$

wherein x1 is the level of the nucleic acids in a tissue that is not within or not adjacent to the tissue that was administered the composition, wherein x2 is the level of the nucleic acids in the tissue administered with the composition. Further provided herein are methods, wherein the level of nucleic acids is determined by polymerase chain reaction (PCR), spectrophotometry, microarray, or an in vivo imaging assay. Further provided herein are methods, wherein the composition comprises a lower nucleic acid biodistribution ratio relative to a nucleic acid biodistribution ratio of a reactogenic nanoparticle composition. Further provided herein are methods, wherein the reactogenic nanoparticle composition comprises one or more characteristics from (i)-(iii): (i) nucleic acids encapsulated in a core of the reactogenic nanoparticle composition; (ii) a net neutral charge; (iii) an average Z-diameter greater than 60 nm. Further provided herein are methods, the administering is via intramuscular injection. Further provided herein are methods, wherein the tissue is a muscle tissue. Further provided herein are methods, wherein the tissue that is not within or not adjacent to the tissue administered the composition is a liver tissue, a spleen tissue, an ovarian tissue, a testis tissue, a lung tissue, a brain tissue, or a heart tissue. Further provided herein are methods, wherein the nucleic acids encode for a protein. Further provided herein are methods, wherein the protein is a microbial protein antigen or a cancer-associated protein. Further provided herein are methods, wherein the microbial protein antigen is a microbial protein antigen listed in Table 3. Further provided herein are methods, wherein the nucleic acid comprises a region comprising a sequence that is at least 85% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 90% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 95% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 99% identical to any one SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising any one of SEQ ID NOS: 11, 20, 22, 25, 27, 29, 31, 33, 35, 37, 39, 43, 49, 51, 67, 180, 181, 183, 185-192. Further provided herein are methods, wherein the microbial protein antigen is a major outer membrane protein, an envelope protein, an envelope glycoprotein, an E7 protein, an E6 oncoprotein, a haemagglutinin protein, a malate synthase protein, a nucleoprotein, an L protein, a transmembrane glycoprotein, a phosphoroprotein, an M2 protein, and RSV glycoprotein, an RSV fusion protein, a spike protein, a variant, or a derivative thereof. Further provided herein are methods, wherein the microbial protein antigen is from a Chlamydia trachomatis bacteria, an enterovirus, a gamma herpesvirus, an alphaherpesvirus, a human papillomavirus virus (HPV), an influenza virus, a Mycobacterium tuberculosis bacteria, a Pseudomonas bacteria, a Acinetobacter bacteria, a Klebsiella bacteria, an Escherichia coli bacteria, a Serratia bacteria, a Streptococcus bacteria, a Shigella bacteria, a Campylobacter bacteria, a Staphylococcus bacteria, a Salmonellae bacteria, an Enterococcus bacteria, a Helicobacter pylori bacteria, a Neisseria gonorrhoeae bacteria, a Haemophilus influenzae bacteria, a Proteus bacteria, a rabies virus, a respiratory syncytial virus, a coronavirus, a severe acute respiratory syndrome (SARS) virus, a Varicella-Zoster Virus (VZV), or a Zika virus. Further provided herein are methods, wherein the cancer-associated protein is a cancer-associated protein listed in Table 6 or Table 7. Further provided herein are methods, wherein the cancer-associated protein is a Melanoma Antigen Gene (MAGE) protein, a Tyrosinase-related protein 1 (TRP-1) protein, prostein, tyrosinase, a glycoprotein 100 (gp100) protein, a melanoma antigen recognized by T cells 1 (MART) protein, a glycoprotein 75 (gp75) protein, a Tyrosinase-related protein 2 (TRP-2) protein, a Carcinoembryonic antigen (CEA) protein, a human epidermal growth factor receptor (HER-2), a prostate-specific membrane antigen (PSMA) protein, a B melanoma antigen (BAGE) protein, a G antigen (GAGE) protein, a cancer/testis antigen protein, a 43kD protein (p43), a p15, a beta catenin protein, a CAP-8 protein, a multiple myeloma 1 (MUM-1) protein, a mucin (MUC) protein, a variant, or a derivative thereof. Further provided herein are methods, wherein the nucleic acid comprises a region comprising a sequence that is at least 85% identical to any one of SEQ ID NOS: 70-72. Further provided herein are methods, wherein the nucleic acid comprises a region comprising a sequence that is at least 90% identical to any one of SEQ ID NOS: 70-72. Further provided herein are methods, wherein the nucleic acid comprises a region comprising a sequence that is at least 95% identical to any one of SEQ ID NOS: 70-72. Further provided herein are methods, wherein the nucleic acid comprises a region comprising a sequence that is at least 99% identical to any one of SEQ ID NOS: 70-72. Further provided herein are methods, wherein the nucleic acid comprises a region comprising any one of SEQ ID NOS: 70-72. Further provided herein are methods, wherein the nucleic acid comprises a region encoding for an amino acid sequence that is least 85% identical to any one of SEQ ID NOS: 73-147. Further provided herein are methods, wherein the nucleic acid comprises a region encoding for an amino acid sequence that is least 90% identical to any one of SEQ ID NOS: 73-147. Further provided herein are methods, wherein the nucleic acid comprises a region encoding for an amino acid sequence that is least 95% identical to any one of SEQ ID NOS: 73-147. Further provided herein are methods, wherein the nucleic acid comprises a region encoding for an amino acid sequence that is least 99% identical to any one of SEQ ID NOS: 73-147. Further provided herein are methods, wherein the nucleic acid comprises a region encoding for an amino acid sequence comprising any one of SEQ ID NOS: 73-147. Further provided herein are methods, wherein the protein is an antibody or a functional variant thereof. Further provided herein are methods, wherein the antibody or functional variant thereof comprises an antibody listed in Table 4 or Table 5. Further provided herein are methods, wherein the antibody or functional variant thereof comprises wherein the antibody or functional variant thereof comprises bamlanivimab, casirivimab, imdevimab, sotrovimab, atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, trastuzumab, fragments, derivatives, or variants thereof. Further provided herein are methods, wherein the antibody or functional variant thereof comprises an amino acid sequence that is at least 85% identical to any one of SEQ ID NOS: 52-66, 68-69. Further provided herein are methods, wherein the antibody or functional variant thereof comprises an amino acid sequence that is at least 90% identical to any one of SEQ ID NOS: 52-66, 68-69. Further provided herein are methods, wherein the antibody or functional variant thereof comprises an amino acid sequence that is at least 95% identical to any one of SEQ ID NOS: 52-66, 68-69. Further provided herein are methods, wherein the antibody or functional variant thereof comprises an amino acid sequence that is at least 99% identical to any one of SEQ ID NOS: 52-66, 68-69. Further provided herein are methods, wherein the antibody or functional variant thereof comprises any one of SEQ ID NOS: 52-66, 68-69. Further provided herein are methods, wherein the nanoparticles are characterized as having a z-average diameter particle size measurement of about 40 nm to about 300 nm when measured by dynamic light scattering. Further provided herein are methods, wherein the nanoparticles are characterized as having a z-average diameter particle size measurement of about 40 nm up to 150 nm when measured by dynamic light scattering. Further provided herein are methods, wherein prior to forming a complex with the nucleic acids, the nanoparticles are characterized as having a z-average diameter particle size measurement of about 40 nm up to 60 nm when measured by dynamic light scattering. Further provided herein are methods, wherein the nucleic acid-nanoparticle complexes are characterized as having a z-average diameter particle size measurement of up to about 600 nm when measured by dynamic light scattering. Further provided herein are methods, wherein the nucleic acid-lipid nanoparticle complexes comprise a z-average diameter particle size measurement of at least two-fold greater than that of the nanoparticles when measured by dynamic light scattering. Further provided herein are methods, wherein the surface comprises one or more cationic lipid selected from the group consisting of: 1,2-dioleoyloxy-3 (trimethylammonium)propane (DOTAP), 30-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-TH-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″″Z)-tetrakis (octadeca-9,12-dienoate); PEG2000-DMG, (R)-2,3-bis(myristoyloxy)propyl-1-(methoxy poly(ethylene glycol)2000) carbamate; and or N1,N3,N5-tris(3-(didodecylamino)propyl)benzene-1,3,5-tricarboxamide (TT3). Further provided herein are methods, wherein the liquid oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, solanesol, soy lecithin, soybean oil, sunflower oil, a triglyceride, or vitamin E. Further provided herein are methods, wherein the triglyceride is capric triglyceride, caprylic triglyceride, a caprylic and capric triglyceride, a triglyceride ester, or myristic acid triglycerin. Further provided herein are methods, wherein the nanoparticles further comprise a surfactant. Further provided herein are methods, wherein the surfactant is a hydrophobic surfactant. Further provided herein are methods, wherein the surfactant is a hydrophilic surfactant. Further provided herein are methods, wherein the surfactant is an amphiphilic surfactant. Further provided herein are methods, wherein the hydrophobic surfactant comprises sorbitan monolaurate, sorbitan monopalmitate, sorbitan monostearate, sorbitan tristearate, sorbitan monooleate, or sorbitan trioleate. Further provided herein are methods, wherein the hydrophilic surfactant comprises polyoxyethylene sorbitan ester (polysorbate), polyoxyethylene sorbitan monooleate, polyoxyethylene sorbitan monostearate, polyoxyethylene sorbitan monopalmitate, polyoxyethylene sorbitan monolaurate. Further provided herein are methods, wherein the amphiphilic surfactant comprises sodium dodecyl sulfate (SDS). Further provided herein are methods, wherein the hydrophobic core further comprises a phosphate-terminated lipid. Further provided herein are methods, wherein the phosphate-terminated lipid comprises trioctylphosphine oxide (TOPO) or distearyl phosphatidic acid (DSPA). Further provided herein are methods, wherein the hydrophobic core further comprises a phosphorous-terminated surfactant, a carboxylate-terminated surfactant, a sulfate-terminated surfactant, or an amine-terminated surfactant. Further provided herein are methods, wherein the carboxylate-terminated surfactant comprises oleic acid. Further provided herein are methods, wherein the amine-terminated surfactant comprises oleylamine. Further provided herein are methods, wherein the hydrophobic core further comprises an inorganic particle. Further provided herein are methods, wherein the inorganic particle comprises a metal. Further provided herein are methods, wherein the metal comprises iron oxide, magnetite (Fe₃O₄), maghemite (y-Fe₂O₃), wüstite (FeO), hematite (alpha (α)-Fe₂O₃), aluminum hydroxide, or aluminum oxyhydroxide. Further provided herein are methods, wherein the inorganic particle is coated with a surfactant or a capping ligand. Further provided herein are methods, wherein the inorganic particle is coated with trioctylphosphine oxide (TOPO) or distearyl phosphatidic acid (DSPA). Further provided herein are methods, wherein the nanoparticles comprise a hydrophilic-lipophilic balance (HLB) value of at least about 8 up to 11. Further provided herein are methods, wherein the nanoparticles comprise an oil-to-surfactant molar ratio ranging from at least about 0.4:1 up to 1:1. Further provided herein are methods, wherein the nanoparticles comprise a nitrogen:phosphate (N:P) molar ratio from at least about 5:1 up to 30:1. Further provided herein are methods, wherein the nanoparticles comprise an average polydispersity index (PDI) from at least about 0.1 to about 0.3. Further provided herein are methods, wherein the nucleic acids are in complex with the nanoparticles. Further provided herein are methods, wherein the nucleic acids each encode for one or more bioactive agent. Further provided herein are methods, wherein the one or more bioactive agent comprises a protein, an antibody, an antibody fragment, a cytokine, or an immune system modulator. Further provided herein are methods, wherein the composition comprises at least one nucleic acid encoding for an RNA polymerase. Further provided herein are methods, wherein the composition comprises at least one nucleic acid comprising a region encoding for Venezuelan equine encephalitis virus (VEEV). Further provided herein are methods, wherein the composition further comprises an adjuvant. Further provided herein are methods, wherein the composition is lyophilized prior to administration to the subject. Further provided herein are methods, wherein the composition is formulated as a suspension. Further provided herein are methods, wherein the composition is in a liquid, semi-liquid, solution, propellant, or powder dosage form. Further provided herein are methods, wherein the nanoparticles are in aqueous solution. Further provided herein are methods, wherein the composition is administered to a mammal. Further provided herein are methods, wherein the composition is administered to a human subject. Further provided herein are methods, wherein the composition is administered for veterinary uses. Further provided herein are methods, wherein the composition is administered to a livestock or a domesticated animal. Provided herein are methods for reducing the reactogenicity of a vaccine composition in a subject, the method comprising: administering to a subject a vaccine composition, wherein the vaccine composition comprises: nanoparticles, wherein the nanoparticles comprise: a hydrophobic core comprising lipids in liquid phase at 25 degrees Celsius; and nucleic acids encoding for a microbial protein antigen or nucleic acids encoding for a cancer-associated protein, wherein the nucleic acids encoding for the microbial protein antigen are complexed to the nanoparticles to form nucleic acid-nanoparticle complexes, and wherein the administering to the subject reduces systemic inflammation compared to administration of a reactogenic nanoparticle composition, thereby reducing the reactogenicity of the vaccine composition. Further provided herein are methods, wherein the reactogenic nanoparticle compositions comprises one or more characteristics from (i)-(iii): (i) nucleic acids encapsulated in a core of the reactogenic nanoparticle composition; (ii) a net neutral charge; and (iii) an average Z-diameter greater than 60 nm. Further provided herein are methods, wherein the nucleic acids comprise a nucleic acid comprising a self-replicating RNA. Further provided herein are methods, wherein the self-replicating RNA is present in an amount of at least about 10 μg up to 200 μg. Further provided herein are methods, wherein the self-amplifying RNA comprises SEQ ID NO: 176. Further provided herein are methods, wherein the nucleic acids are in complex with a surface of the nanoparticles. Further provided herein are methods, wherein the nucleic acids encode for a protein. Further provided herein are methods, wherein the protein comprises a microbial protein antigen or a cancer-associated protein. Further provided herein are methods, wherein the microbial protein antigen is a microbial protein antigen listed in Table 3. Further provided herein are methods, wherein the microbial protein antigen is a major outer membrane protein, an envelope protein, an envelope glycoprotein, an E7 protein, an E6 oncoprotein, a haemagglutinin protein, a malate synthase protein, a nucleoprotein, an L protein, a transmembrane glycoprotein, a phosphoroprotein, an M2 protein, and RSV glycoprotein, an RSV fusion protein, a spike protein, a variant, or a derivative thereof. Further provided herein are methods, wherein the microbial protein antigen is from a Chlamydia trachomatis bacteria, an enterovirus, a gamma herpesvirus, an alphaherpesvirus, a human papillomavirus virus (HPV), an influenza virus, a Mycobacterium tuberculosis bacteria, a Pseudomonas bacteria, a Acinetobacter bacteria, a Klebsiella bacteria, an Escherichia coli bacteria, a Serratia bacteria, a Streptococcus bacteria, a Shigella bacteria, a Campylobacter bacteria, a Staphylococcus bacteria, a Salmonellae bacteria, an Enterococcus bacteria, a Helicobacter pylori bacteria, a Neisseria gonorrhoeae bacteria, a Haemophilus influenzae bacteria, a Proteus bacteria, a rabies virus, a respiratory syncytial virus, a coronavirus, a severe acute respiratory syndrome (SARS) virus, a Varicella-Zoster Virus (VZV), or a Zika virus. Further provided herein are methods, wherein the nucleic acid comprises a region comprising a sequence that is at least 85% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 90% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 95% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 99% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising any one of SEQ ID NOS: 11, 20, 22, 25, 27, 29, 31, 33, 35, 37, 39, 43, 49, 51, 67, 180, 181, 183, 185-192. Further provided herein are methods, wherein the cancer-associated protein is a cancer-associated protein listed in Table 6 or Table 7. Further provided herein are methods, wherein the cancer-associated protein is a Melanoma Antigen Gene (MAGE) protein, a Tyrosinase-related protein 1 (TRP-1) protein, prostein, tyrosinase, a glycoprotein 100 (gp100) protein, a melanoma antigen recognized by T cells 1 (MART) protein, a glycoprotein 75 (gp75) protein, a Tyrosinase-related protein 2 (TRP-2) protein, a Carcinoembryonic antigen (CEA) protein, a human epidermal growth factor receptor (HER-2), a prostate-specific membrane antigen (PSMA) protein, a B melanoma antigen (BAGE) protein, a G antigen (GAGE) protein, a cancer/testis antigen protein, a 43kD protein (p43), a p15, a beta catenin protein, a CAP-8 protein, a multiple myeloma 1 (MUM-1) protein, a mucin (MUC) protein, a variant, or a derivative thereof. Further provided herein are methods, wherein the nucleic acid comprises a region comprising a sequence that is at least 85% identical to any one of SEQ ID NOS: 70-72. Further provided herein are methods, wherein the nucleic acid comprises a region comprising a sequence that is at least 90% identical to any one of SEQ ID NOS: 70-72. Further provided herein are methods, wherein the nucleic acid comprises a region comprising a sequence that is at least 95% identical to any one of SEQ ID NOS: 70-72. Further provided herein are methods, wherein the nucleic acid comprises a region comprising a sequence that is at least 99% identical to any one of SEQ ID NOS: 70-72. Further provided herein are methods, wherein the nucleic acid comprises a region comprising any one of SEQ ID NOS: 70-72. Further provided herein are methods, wherein the nucleic acid comprises a region encoding for an amino acid sequence that is least 85% identical to any one of SEQ ID NOS: 73-147. Further provided herein are methods, wherein the nucleic acid comprises a region encoding for an amino acid sequence that is least 90% identical to any one of SEQ ID NOS: 73-147. Further provided herein are methods, wherein the nucleic acid comprises a region encoding for an amino acid sequence that is least 95% identical to any one of SEQ ID NOS: 73-147. Further provided herein are methods, wherein the nucleic acid comprises a region encoding for an amino acid sequence that is least 99% identical to any one of SEQ ID NOS: 73-147. Further provided herein are methods, wherein the nucleic acid comprises a region encoding for an amino acid sequence comprising any one of SEQ ID NOS: 73-147. Further provided herein are methods, wherein the protein is an antibody or a functional variant thereof. Further provided herein are methods, wherein the antibody or functional variant thereof comprises an antibody listed in Table 4 or Table 5. Further provided herein are methods, wherein the antibody or functional variant thereof comprises wherein the antibody or functional variant thereof comprises bamlanivimab, casirivimab, imdevimab, sotrovimab, atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, trastuzumab, fragments, derivatives, or variants thereof. Further provided herein are methods, wherein the antibody or functional variant thereof comprises an amino acid sequence that is at least 85% identical to any one of SEQ ID NOS: 52-66, 68-69. Further provided herein are methods, wherein the antibody or functional variant thereof comprises an amino acid sequence that is at least 90% identical to any one of SEQ ID NOS: 52-66, 68-69. Further provided herein are methods, wherein the antibody or functional variant thereof comprises an amino acid sequence that is at least 95% identical to any one of SEQ ID NOS: 52-66, 68-69. Further provided herein are methods, wherein the antibody or functional variant thereof comprises an amino acid sequence that is at least 99% identical to any one of SEQ ID NOS: 52-66, 68-69. Further provided herein are methods, wherein the antibody or functional variant thereof comprises any one of SEQ ID NOS: 52-66, 68-69. Further provided herein are methods, wherein the administering results in a lower level of cardiac troponin-I (cTNI) compared to administration of a reactogenic nanoparticle composition. Further provided herein are methods, wherein the administering results in a lower level of systemic interferons relative to the level of systemic interferons in a subject that was administered the reactogenic nanoparticle composition. Further provided herein are methods, wherein the administering is intramuscular administration, intradermal administration, transdermal administration, sublingual administration, buccal administration, intranasal administration, inhalation administration, intrathecal administration, or intratumoral administration. Further provided herein are methods, wherein the nanoparticles comprise a cationic lipid on a surface of the nanoparticle. Further provided herein are methods, wherein the nanoparticles comprise a positive net charge at about 35 degrees Celsius. Further provided herein are methods, wherein the nanoparticles are characterized as having a z-average diameter particle size measurement of about 40 nm to about 300 nm when measured by dynamic light scattering. Further provided herein are methods, wherein the nanoparticles are characterized as having a z-average diameter particle size measurement of about 40 nm up to 150 nm when measured by dynamic light scattering. Further provided herein are methods, wherein prior to forming a complex with the nucleic acids, the nanoparticles are characterized as having a z-average diameter particle size measurement of about 40 nm up to 60 nm when measured by dynamic light scattering. Further provided herein are methods, wherein the nucleic acid-nanoparticle complexes are characterized as having a z-average diameter particle size measurement of up to about 600 nm when measured by dynamic light scattering. Further provided herein are methods, wherein the nucleic acid-lipid nanoparticle complexes comprise a z-average diameter particle size measurement of at least two-fold greater than that of the nanoparticles when measured by dynamic light scattering. Further provided herein are methods, wherein the surface comprises one or more cationic lipid selected from the group consisting of: 1,2-dioleoyloxy-3 (trimethylammonium)propane (DOTAP), 30-[N-(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-TH-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 30-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′41 ,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″″Z)-tetrakis (octadeca-9,12-dienoate); PEG2000-DMG, (R)-2,3-bis(myristoyloxy)propyl-1-(methoxy poly(ethylene glycol)2000) carbamate; and or N1,N3,N5-tris(3-(didodecylamino)propyl)benzene-1,3,5-tricarboxamide (TT3). Further provided herein are methods, wherein the liquid oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, solanesol, soy lecithin, soybean oil, sunflower oil, a triglyceride, or vitamin E. Further provided herein are methods, wherein the triglyceride is capric triglyceride, caprylic triglyceride, a caprylic and capric triglyceride, a triglyceride ester, or myristic acid triglycerin. Further provided herein are methods, wherein the nanoparticles further comprise a surfactant. Further provided herein are methods, wherein the surfactant is a hydrophobic surfactant. Further provided herein are methods, wherein the surfactant is a hydrophilic surfactant. Further provided herein are methods, wherein the surfactant is an amphiphilic surfactant. Further provided herein are methods, wherein the hydrophobic surfactant comprises sorbitan monolaurate, sorbitan monopalmitate, sorbitan monostearate, sorbitan tristearate, sorbitan monooleate, or sorbitan trioleate. Further provided herein are methods, wherein the hydrophilic surfactant comprises polyoxyethylene sorbitan ester (polysorbate), polyoxyethylene sorbitan monooleate, polyoxyethylene sorbitan monostearate, polyoxyethylene sorbitan monopalmitate, polyoxyethylene sorbitan monolaurate. Further provided herein are methods, wherein the amphiphilic surfactant comprises sodium dodecyl sulfate (SDS). Further provided herein are methods, wherein the hydrophobic core further comprises a phosphate-terminated lipid. Further provided herein are methods, wherein the phosphate-terminated lipid comprises trioctylphosphine oxide (TOPO) or distearyl phosphatidic acid (DSPA). Further provided herein are methods, wherein the hydrophobic core further comprises a phosphorous-terminated surfactant, a carboxylate-terminated surfactant, a sulfate-terminated surfactant, or an amine-terminated surfactant. Further provided herein are methods, wherein the carboxylate-terminated surfactant comprises oleic acid. Further provided herein are methods, wherein the amine-terminated surfactant comprises oleylamine. Further provided herein are methods, wherein the hydrophobic core further comprises an inorganic particle. Further provided herein are methods, wherein the inorganic particle comprises a metal. Further provided herein are methods, wherein the metal comprises iron oxide, magnetite (Fe₃O₄), maghemite (y-Fe₂O₃), wüstite (FeO), hematite (alpha (α)-Fe₂O₃), aluminum hydroxide, or aluminum oxyhydroxide. Further provided herein are methods, wherein the inorganic particle is coated with a surfactant or a capping ligand. Further provided herein are methods, wherein the inorganic particle is coated with trioctylphosphine oxide (TOPO) or distearyl phosphatidic acid (DSPA). Further provided herein are methods, wherein the nanoparticles comprise a hydrophilic-lipophilic balance (HLB) value of at least about 8 up to 11. Further provided herein are methods, wherein the nanoparticles comprise an oil-to-surfactant molar ratio ranging from at least about 0.4:1 up to 1:1. Further provided herein are methods, wherein the nanoparticles comprise a nitrogen:phosphate (N:P) molar ratio from at least about 5:1 up to 30:1. Further provided herein are methods, wherein the nanoparticles comprise an average polydispersity index (PDI) from at least about 0.1 to about 0.3. Further provided herein are methods, wherein the nucleic acids are in complex with the nanoparticles. Further provided herein are methods, wherein the nucleic acids each encode for one or more bioactive agent. Further provided herein are methods, wherein the one or more bioactive agent comprises a protein, an antibody, an antibody fragment, a cytokine, or an immune system modulator. Further provided herein are methods, wherein the composition comprises at least one nucleic acid encoding for an RNA polymerase. Further provided herein are methods, wherein the composition comprises at least one nucleic acid comprising a region encoding for Venezuelan equine encephalitis virus (VEEV). Further provided herein are methods, wherein the composition further comprises an adjuvant. Further provided herein are methods, wherein the composition is lyophilized prior to administration to the subject. Further provided herein are methods, wherein the composition is formulated as a suspension. Further provided herein are methods, wherein the composition is in a liquid, semi-liquid, solution, propellant, or powder dosage form. Further provided herein are methods, wherein the nanoparticles are in aqueous solution. Further provided herein are methods, wherein the composition is administered to a mammal. Further provided herein are methods, wherein the composition is administered to a human subject. Further provided herein are methods, wherein the composition is administered for veterinary uses. Further provided herein are methods, wherein the composition is administered to a livestock or a domesticated animal.

Provided herein are methods for treating a disease or a condition in a subject, the method comprising: administering to a tissue in a subject a composition, wherein the composition comprises: nanoparticles, wherein the nanoparticles comprise: a hydrophobic core comprising lipids in liquid phase at 25 degrees Celsius; and nucleic acids, wherein the nucleic acids are complexed to the nanoparticles to form nucleic acid-lipid nanoparticle complexes, and wherein the administering of the composition provides for a local immune response within a tissue, thereby treating the disease of the condition in the subject. Further provided herein are methods, wherein the administering is intramuscular administration, intradermal administration, transdermal administration, sublingual administration, buccal administration, intranasal administration, inhalation administration, intrathecal administration, or intratumoral administration. Further provided herein are methods, wherein the local immune response is characterized by an increase in the level of at least one immunostimulatory marker in the tissue relative to a reference level. Further provided herein are methods, wherein the reference level is the level of the immunostimulatory marker in a subject that has not been administered the composition or the level of the immunostimulatory marker in the subject prior to administration of the composition to the tissue. Further provided herein are methods, wherein the at least one immunostimulatory marker comprises: cluster of differentiation 86 (CD86), H-2 class II histocompatibility antigen, A beta chain (H2-ab1), H-2 class II histocompatibility antigen, I-E beta chain (H2-eb1), integrin alpha L chain (ITGAL or CD11a), Integrin alpha M (ITGAM, CR3A, or CD11b), Fcγ receptor (FcγR), cluster of differentiation 28 (CD28) or a combination thereof. Further provided herein are methods, wherein the local immune response is characterized by recruitment of immune cells to the tissue. Further provided herein are methods, wherein the immune cells comprise a population of MHC-II^{lo} macrophages, a population of plasmacytoid dendritic cells (pDCs), a population of monocyte dendritic cells (MoDCs), a population of neutrophils, a population of NK cells, or a combination thereof. Further provided herein are methods, the tissue is a muscle tissue, an epithelial tissue, an endothelial tissue, a mucosal tissue, a nasal cavity tissue, a thecal tissue, or a tumor. Further provided herein are methods, wherein the disease or condition is a cancer or a microbial infection. Further provided herein are methods, wherein the cancer is a skin cancer, a pancreatic cancer, a colon cancer, a prostate cancer, an ovarian cancer, or a breast cancer. Further provided herein are methods, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are methods, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are methods, wherein the composition is administered to a mammal. Further provided herein are methods, wherein the composition is administered to a human subject. Further provided herein are methods, wherein the composition is administered for veterinary uses. Further provided herein are methods, wherein the composition is administered to a livestock or a domesticated animal.

Provided herein are methods for generating an immune response in a subject to a protein antigen, the method comprising: administering to a tissue in a subject a composition, wherein the composition comprises: nanoparticles, wherein the nanoparticles comprise: a hydrophobic core comprising lipids in liquid phase at 25 degrees Celsius; and nucleic acids, wherein the nucleic acids encode for at least one protein antigen, and wherein the nucleic acids are complexed to the nanoparticles to form nucleic acid-lipid nanoparticle complexes, and wherein the administering of the composition provides for a local immune response within a tissue, thereby generating an immune response in the subject to the at least one protein antigen. Further provided herein are methods, wherein the protein antigen comprises a microbial protein antigen or a cancer-associated protein. Further provided herein are methods, wherein the microbial protein antigen is a microbial protein antigen listed in Table 3. Further provided herein are methods, wherein the microbial protein antigen is a major outer membrane protein, an envelope protein, an envelope glycoprotein, an E7 protein, an E6 oncoprotein, a haemagglutinin protein, a malate synthase protein, a nucleoprotein, an L protein, a transmembrane glycoprotein, a phosphoroprotein, an M2 protein, and RSV glycoprotein, an RSV fusion protein, a spike protein, a variant, or a derivative thereof. Further provided herein are methods, wherein the microbial protein antigen is from a Chlamydia trachomatis bacteria, an enterovirus, a gamma herpesvirus, an alphaherpesvirus, a human papillomavirus virus (HPV), an influenza virus, a Mycobacterium tuberculosis bacteria, a Pseudomonas bacteria, a Acinetobacter bacteria, a Klebsiella bacteria, an Escherichia coli bacteria, a Serratia bacteria, a Streptococcus bacteria, a Shigella bacteria, a Campylobacter bacteria, a Staphylococcus bacteria, a Salmonellae bacteria, an Enterococcus bacteria, a Helicobacter pylori bacteria, a Neisseria gonorrhoeae bacteria, a Haemophilus influenzae bacteria, a Proteus bacteria, a rabies virus, a respiratory syncytial virus, a coronavirus, a severe acute respiratory syndrome (SARS) virus, a Varicella-Zoster Virus (VZV), or a Zika virus. Further provided herein are methods, wherein the nucleic acid comprises a region comprising a sequence that is at least 85% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 90% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 95% identical to any one of SEQ ID NOS: 11, 20, 22, 25, 27, 29, 31, 33, 35, 37, 39, 43, 49, 51, 67, 180, 181, 183, 185-192. Further provided herein are methods, wherein the nucleic acid comprises a region comprising a sequence that is at least 99% identical to any one of SEQ ID NOS: 11, 20, 22, 25, 27, 29, 31, 33, 35, 37, 39, 43, 49, 51, 67, 180, 181, or 183. Further provided herein are methods, wherein the nucleic acid comprises a region comprising any one of SEQ ID NOS: 11, 20, 22, 25, 27, 29, 31, 33, 35, 37, 39, 43, 49, 51, 67, 180, 181, or 183. Further provided herein are methods, wherein the cancer-associated protein is a cancer-associated protein listed in Table 6 or Table 7. Further provided herein are methods, wherein the cancer-associated protein is a Melanoma Antigen Gene (MAGE) protein, a Tyrosinase-related protein 1 (TRP-1) protein, prostein, tyrosinase, a glycoprotein 100 (gp100) protein, a melanoma antigen recognized by T cells 1 (MART) protein, a glycoprotein 75 (gp75) protein, a Tyrosinase-related protein 2 (TRP-2) protein, a Carcinoembryonic antigen (CEA) protein, a human epidermal growth factor receptor (HER-2), a prostate-specific membrane antigen (PSMA) protein, a B melanoma antigen (BAGE) protein, a G antigen (GAGE) protein, a cancer/testis antigen protein, a 43kD protein (p43), a p15, a beta catenin protein, a CAP-8 protein, a multiple myeloma 1 (MUM-1) protein, a mucin (MUC) protein, a variant, or a derivative thereof. Further provided herein are methods, wherein the nucleic acid comprises a region comprising a sequence that is at least 85% identical to any one of SEQ ID NOS: 70-72. Further provided herein are methods, wherein the nucleic acid comprises a region comprising a sequence that is at least 90% identical to any one of SEQ ID NOS: 70-72. Further provided herein are methods, wherein the nucleic acid comprises a region comprising a sequence that is at least 95% identical to any one of SEQ ID NOS: 70-72. Further provided herein are methods, wherein the nucleic acid comprises a region comprising a sequence that is at least 99% identical to any one of SEQ ID NOS: 70-72. Further provided herein are methods, wherein the nucleic acid comprises a region comprising any one of SEQ ID NOS: 70-72. Further provided herein are methods, wherein the nucleic acid comprises a region encoding for an amino acid sequence that is least 85% identical to any one of SEQ ID NOS: 73-147. Further provided herein are methods, wherein the nucleic acid comprises a region encoding for an amino acid sequence that is least 90% identical to any one of SEQ ID NOS: 73-147. Further provided herein are methods, wherein the nucleic acid comprises a region encoding for an amino acid sequence that is least 95% identical to any one of SEQ ID NOS: 73-147. Further provided herein are methods, wherein the nucleic acid comprises a region encoding for an amino acid sequence that is least 99% identical to any one of SEQ ID NOS: 73-147. Further provided herein are methods, wherein the nucleic acid comprises a region encoding for an amino acid sequence comprising any one of SEQ ID NOS: 73-147. Further provided herein are methods, wherein the administering is intramuscular administration, intradermal administration, transdermal administration, sublingual administration, buccal administration, intranasal administration, inhalation administration, intrathecal administration, or intratumoral administration. Further provided herein are methods, wherein the local immune response is characterized by an increase in the level of at least one immunostimulatory marker in the tissue relative to a reference level. Further provided herein are methods, wherein the reference level is the level of the immunostimulatory marker in a subject that has not been administered the composition or the level of the immunostimulatory marker in the subject prior to administration of the composition to the tissue. Further provided herein are methods, wherein the at least one immunostimulatory marker comprises: cluster of differentiation 86 (CD86), H-2 class II histocompatibility antigen, A beta chain (H2-ab1), H-2 class II histocompatibility antigen, I-E beta chain (H2-eb1), integrin alpha L chain (ITGAL or CD11a), Integrin alpha M (ITGAM, CR3A, or CD11b), Fcγ receptor (FcγR), cluster of differentiation 28 (CD28) or a combination thereof. Further provided herein are methods, wherein the local immune response is characterized by recruitment of immune cells to the tissue. Further provided herein are methods, wherein the immune cells comprise a population of MHC-II^{lo} macrophages, a population of plasmacytoid dendritic cells (pDCs), a population of monocyte dendritic cells (MoDCs), a population of neutrophils, a population of NK cells, or a combination thereof. Further provided herein are methods, wherein the tissue is a muscle tissue, an epithelial tissue, an endothelial tissue, a mucosal tissue, a nasal cavity tissue, a thecal tissue, or a tumor. Further provided herein are methods, wherein the subject has, is suspected of having, at risk for developing, or diagnosed with a cancer or a microbial infection. Further provided herein are methods, wherein the cancer is a skin cancer, a pancreatic cancer, a colon cancer, a prostate cancer, an ovarian cancer, or a breast cancer. Further provided herein are methods, wherein the pancreatic cancer is a pancreatic adenocarcinoma, a pancreatic exocrine cancer, a pancreatic neuroendocrine cancer, an islet cell cancer, or a pancreatic endocrine cancer. Further provided herein are methods, wherein the skin cancer is a basal cell cancer, a melanoma, a Merkel cell cancer, a squamous cell carcinoma, a cutaneous lymphoma, a Kaposi sarcoma, or a skin adnexal cancer. Further provided herein are methods, wherein the composition is administered to a mammal. Further provided herein are methods, wherein the composition is administered to a human subject. Further provided herein are methods, wherein the composition is administered for veterinary uses. Further provided herein are methods, wherein the composition is administered to a livestock or a domesticated animal.

Provided herein are compositions, wherein the compositions comprise: nanoparticles, wherein the nanoparticles comprise: a hydrophobic core comprising lipids in liquid phase at 25 degrees Celsius; and nucleic acids encoding for a human immunodeficiency virus-1 (HIV-1) envelope (env) protein or a functional variant thereof, wherein the nucleic acids encoding for an HIV-1 env protein or a functional variant thereof are complexed to the nanoparticles to form nucleic acid-nanoparticle complexes. Further provided herein are compositions, wherein the nucleic acids encoding for a human immunodeficiency virus-1 (HIV-1) envelope (env) protein or a functional variant thereof comprise SEQ ID NO: 180 or SEQ ID NO: 181 Further provided herein are compositions, wherein the nucleic acids encoding for a human immunodeficiency virus-1 (HIV-1) envelope (env) protein or a functional variant thereof further comprise a sequence encoding a viral RNA polymerase. Further provided herein are compositions, wherein the nucleic acids comprise a nucleic acid that comprises a sequence that is at least 85% identical to SEQ ID NO: 176.

Provided herein are methods for generating an immune response in a subject to an HIV protein antigen, the methods comprising: administering to the subject a composition comprising: nanoparticles, wherein the nanoparticles comprise: a hydrophobic core comprising lipids in liquid phase at 25 degrees Celsius; and nucleic acids encoding for a human immunodeficiency virus-1 (HIV-1) envelope (env) protein or a functional variant thereof, wherein the nucleic acids encoding for an HIV-1 env protein or a functional variant thereof are complexed to the nanoparticles to form nucleic acid-nanoparticle complexes, thereby generating an immune response to an HIV protein antigen. Further provided herein are methods, wherein the composition is administered to a mammal. Further provided herein are methods, wherein the composition is administered to a human subject. Further provided herein are methods, wherein the composition is administered for veterinary uses. Further provided herein are methods, wherein the composition is administered to a livestock or a domesticated animal.

### EXAMPLES

### Example 1: Nanoparticle Formulations

NP-30 was prepared. Briefly, the oil phase (squalene, Span 60, and DOTAP) was sonicated for 30 min in a 65° C. water bath. Separately, the aqueous phase, containing Tween 80 and sodium citrate dihydrate solution in water, was prepared with continuous stirring until all components were dissolved. The oil and aqueous phases were then mixed and emulsified using a VWR 200 homogenizer (VWR International), and the crude colloid was subsequently processed by passaging through a microfluidizer at 137895 kPa with an LM10 microfluidizer equipped with an H10Z 100-μm ceramic interaction chamber (Microfluidics) until the Z-average hydrodynamic diameter, measured by dynamic light scattering (Malvern Zetasizer Nano S), reached 50±5 nm with a 0.2 polydispersity index. The microfluidized NP-30 was terminally filtered with a 200-nm pore-size polyethersulfone filter and stored at 2° to 8° C.

An LNP was prepared. Briefly, lipid components were dissolved in ethanol at a ratio of 50:10:38:2 (Ionizable lipid (SM-102): Helper Lipid (DSPC): Cholesterol: DMG-PEG 2000) and mixed with RNA buffer at pH 4.5 at an N:P 5.5 using a glass micromixer chip. After mixing the formulations were dialyzed against PBS (pH 7.4) for 16-24 hours. Formulated LNPs were concentrated using Amicon Ultra™ centrifugal filter devices (EMD Millipore, Billerica, MA) and stored at 5° C. RNA encapsulation was quantified using a Ribogreen™ assay using Triton to disrupt formulated LNPs, all LNPs had 92±9% (N=20) encapsulation. Particle size (87±18 nm Average, N=20), PDI (0.19±0.08 Average, N=20), and Zeta potential (8±5 mV average, N=20) were measured using a Malvern Zetasizer Ultra.

### Example 2: Self-Replicating mRNA Construct

A plasmid encoding a T7 promoter followed by the 5′ and 3′ UTRs and nonstructural genes of Venezuelan equine encephalitis virus (VEEV) strain TC-83 was generated using standard DNA synthesis and cloning methods. The VEEV replicon mRNA backbone is set forth in SEQ ID NO: 176.

### Example 3: An Alphavirus-Based RNA Vaccine by a Muscle-Localizing Cationic Nanocarrier Enhances the Local Immune Environment and Elicits an Antibody Response without Triggering Systemic Cytokine Production

NP-30 and LNP nanoparticle formulations were generated as provided in Example 1. NP-30 or LNP formulations were complexed with repRNA encoding SARS-CoV-2 spike (S) protein (SEQ ID NO: 1).

In vivo animal treatments were performed. 6-8 weeks old female C57BL/6, Mx1-GFP Tg (The Jackson Laboratory, #033219), Ai9 Tg (The Jackson Laboratory, #007909), and LSL-Luc Tg (The Jackson Laboratory) reporter mice were used for in vivo studies. The reporter mice were developed using a Cre/loxP-mediated genomic recombination mechanism that drives stable expression of reporter genes (luciferase (Luc) and tdTomato, respectively). As such, these strains allow for the detection of cells and tissues that received repRNA-expressing Cre recombinase before organ and tissue harvest. For in vivo neutralization of interferon alpha and beta receptor subunit 1 (IFNAR1) signaling, mice were injected with 2 mg of anti-IFNAR1 polyclonal antibody (clone MAR1-5A3, BioXcell, #BE0241) intraperitoneally. For repRNA vaccination, the vaccine was prepared as describe above using NP-30 complexed with repRNA encoding the full-length spike of SARS-CoV2 (repRNA-CoV2S). The vaccines were delivered by intramuscular (IM) injection into the quadriceps. Animals received 2 doses of a 1 microgram (pg) or 10 μg dose of repRNA-CoV2S or remained unvaccinated (naive). The vaccine was delivered in a volume of 50 μL.

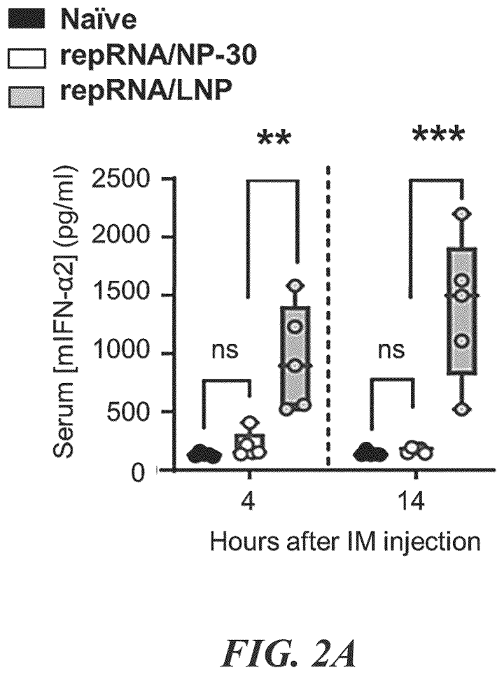

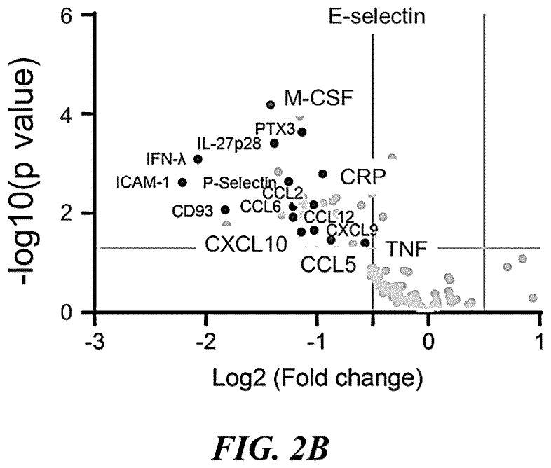

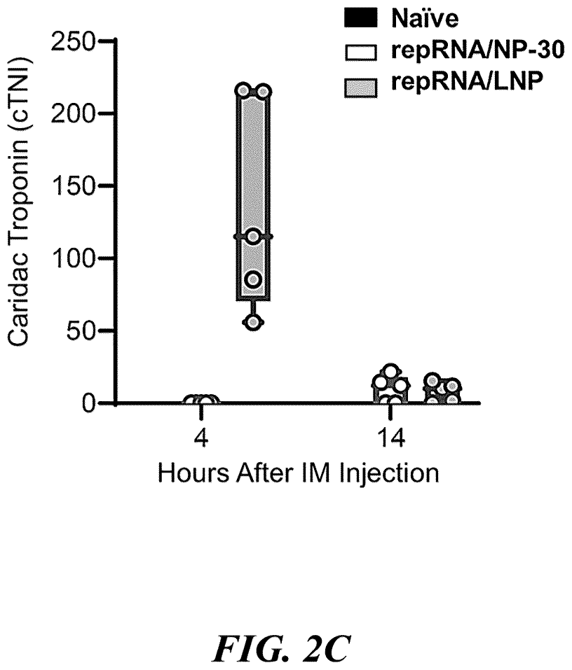

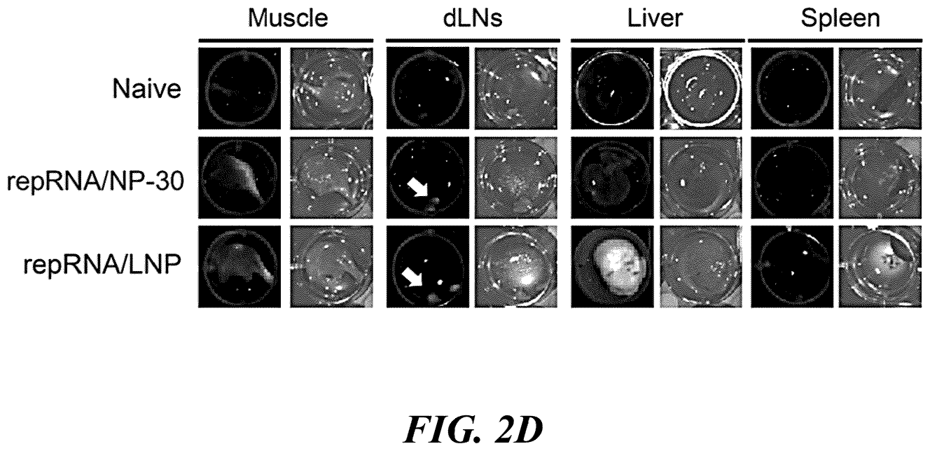

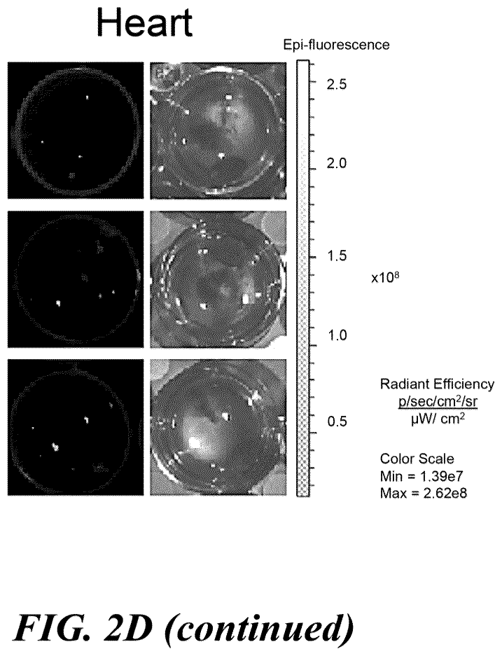

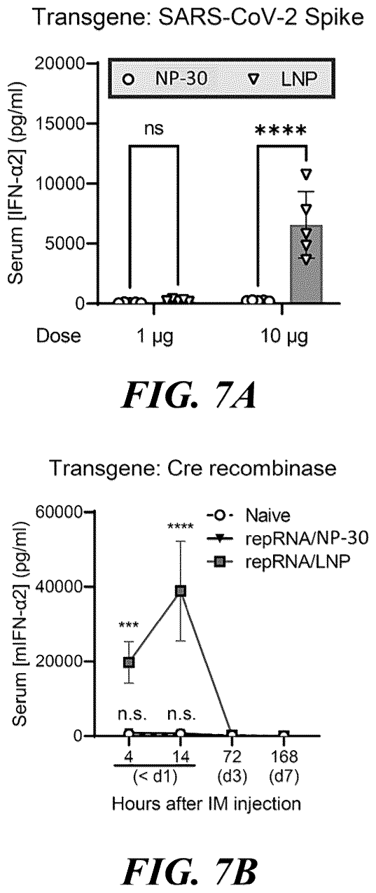

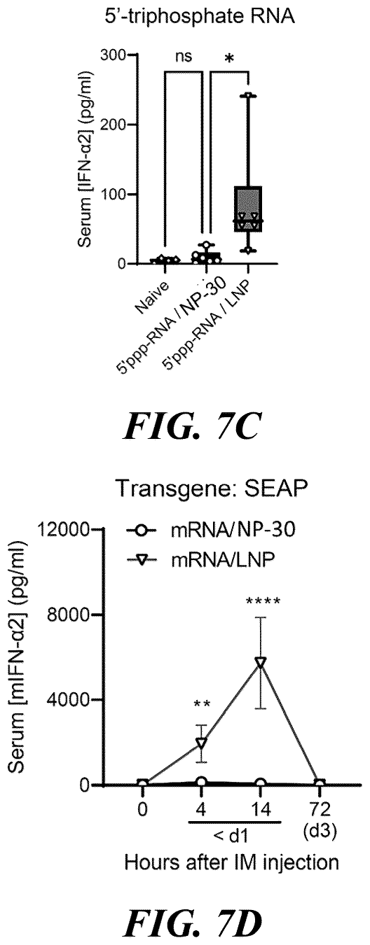

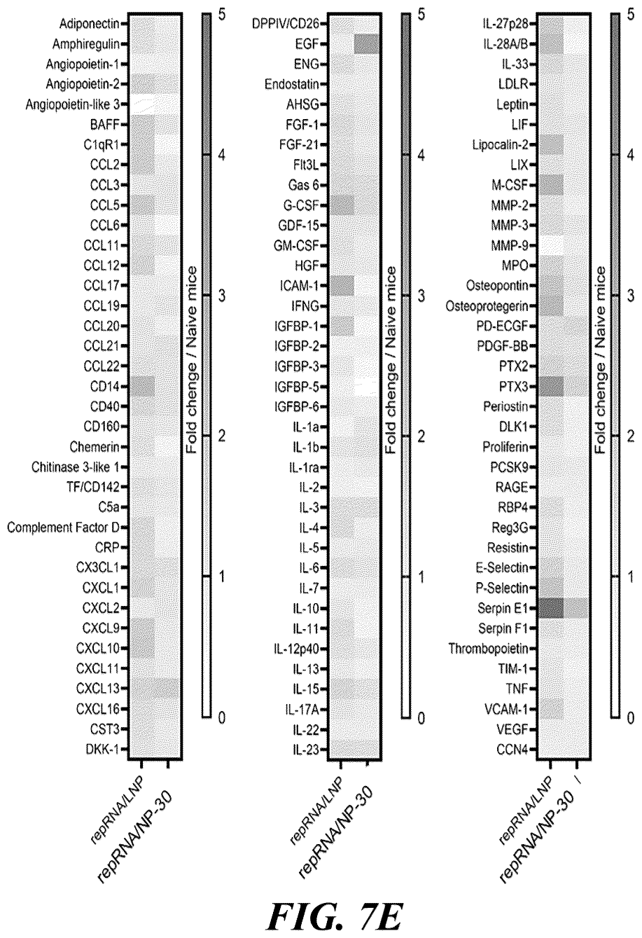

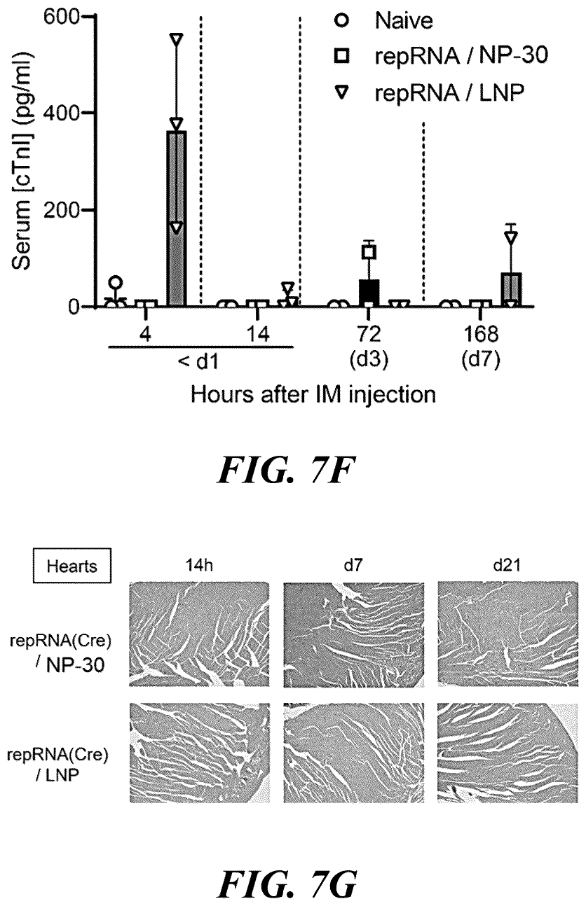

Systemic (blood-borne) type I IFN levels in the animals transfected with repRNA delivered by NP-30 and LNP were measured. The repRNA/LNP-injected mice showed abundant serum type I IFN levels within 1 day after intramuscular (IM) injection (FIG. 2A). However, only minor levels of serum type I IFNs were detected by 14 hours in repRNA/NP-30-injected mice. These findings were reproducible in similar experiments using repRNA expressing different transgenes (SARS-CoV-2 Spike and Cre recombinase) (FIG. 7A-7B), non-replicating 5′-triphosphate RNA (FIG. 7C), and non-replicating 5′-capped mRNA (FIG. 7D) with extended time points. A low 1 μg dose of repRNA/LNP induced only minor systemic type I IFN (FIG. 7A), indicating that innate response to repRNA is dose-dependent. Similar to type I IFN, many chemokines (e.g., CCL2 and CXCL10), selective cytokines (IFN-1 and IL-27p28), and C-reactive protein (CRP), a protein that is previously implicated in reactogenic response to vaccination in humans, were detected at lower levels in sera of repRNA/NP-30-injected mice compared to those of repRNA/LNP-injected mice (FIG. 2B and FIG. 7E). IM injection of repRNA/NP-30 did not induce detectable systemic inflammatory responses.

An increased risk of myocarditis and/or pericarditis have been associated with mRNA/LNP vaccination. Assays were performed to assess cardiovascular inflammation and markers of cardiac damage. First, proteins associated with cardiovascular disease (PTX5, selectin families, and ICAM-1) were measured and detected in sera of the repRNA/LNP-injected mice relative to repRNA/NP-30-injected mice (FIG. 2B and FIG. 7E). A serological marker for cardiac damage, cardiac troponin-I (cTNI) was measured. The repRNA/LNP-injected mice showed transient elevation of cTNI levels at 4 hours, whereas repRNA/NP-30-injected mice showed only minor elevation (FIG. 2C and FIG. 7F-7G).

To corroborate the absence of the systemic innate response in repRNA/NP-30-injected mice, IFN stimulation was detected in response element (ISRE) reporter mice (Mx1-GFP Tg). Tissues were extracted after 24 hours and GFP signaling (type I/III IFN response) in the extracted tissues was analyzed by In vivo imaging system (IVIS). IVIS analysis of the extracted tissues from ISRE reporter mice showed that the GFP signal was detectable in muscle and draining lymph nodes (dLNs) in both repRNA/NP-30 and repRNA/LNP-injected mice at 24 hours after IM injection (FIG. 2D). In contrast, the signal was not detectable in the heart and spleen of animals from both groups 24 hours after IM injection (FIG. 2D). The highest GFP signal was detected in the liver of repRNA/LNP-injected mice but not repRNA/NP-30-injected mice 24 hours after IM injection (FIG. 2D). The liver of repRNA/NP-30-injected mice showed little GFP signal showing the absence of systemic type I/III IFNs 24 hours after IM injection of repRNA. NP-30 delivered repRNA without triggering a systemic IFN response.

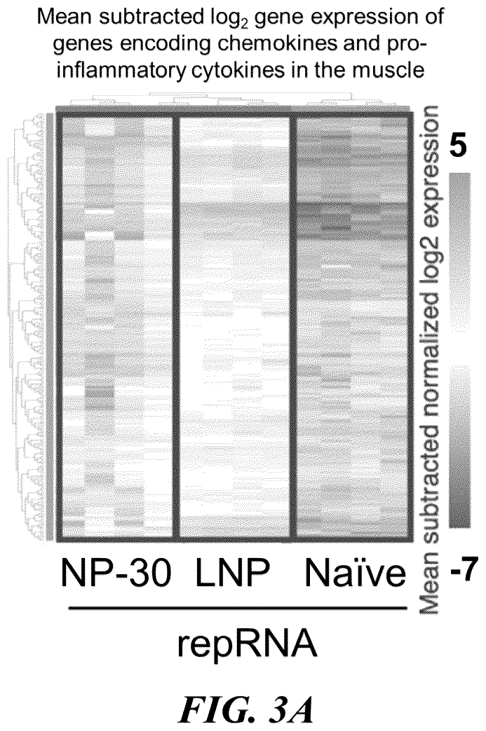

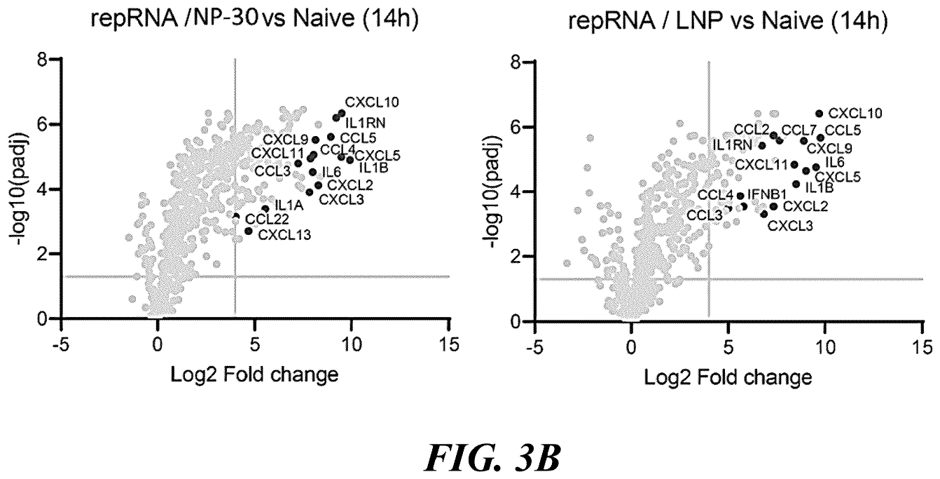

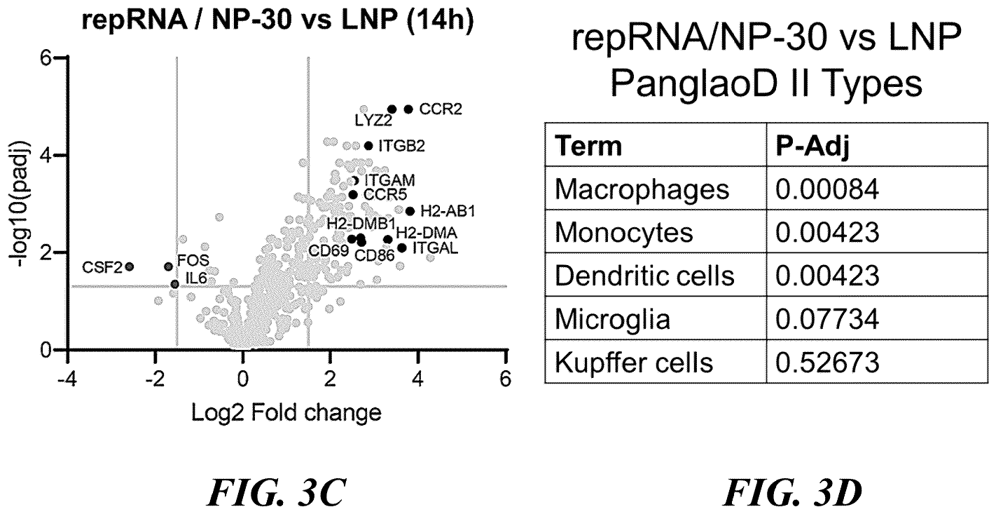

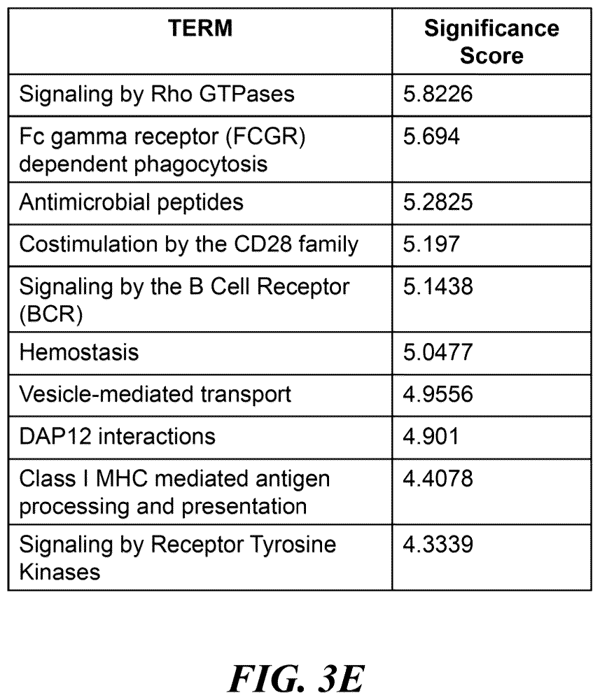

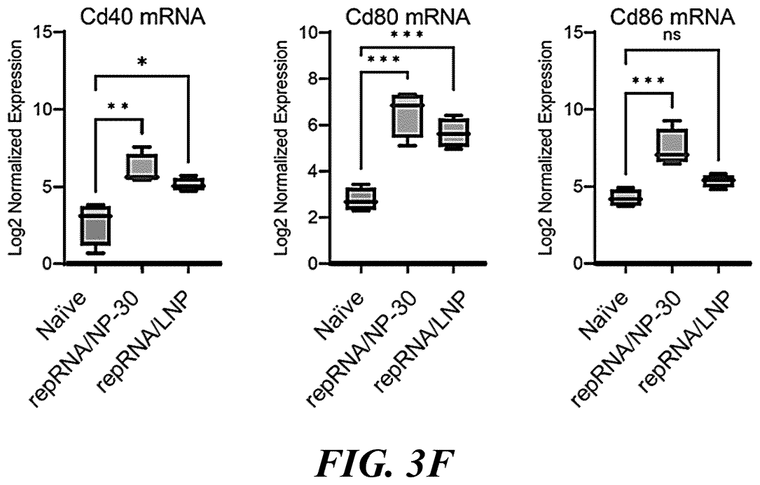

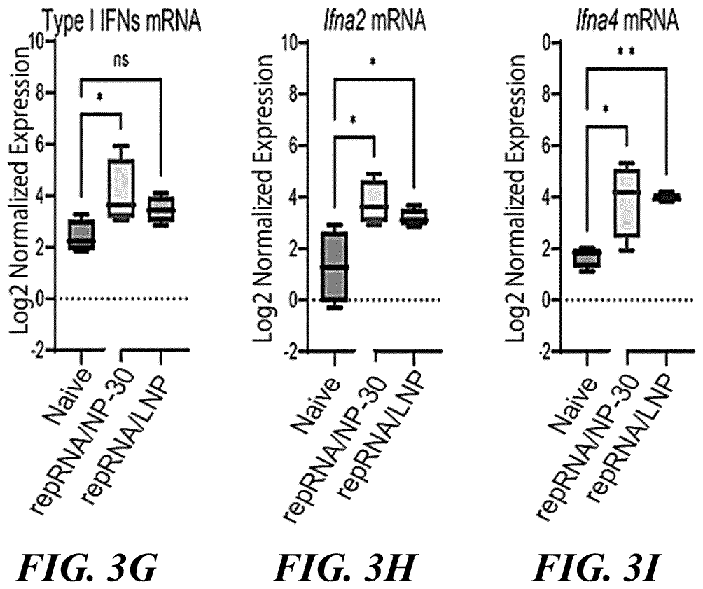

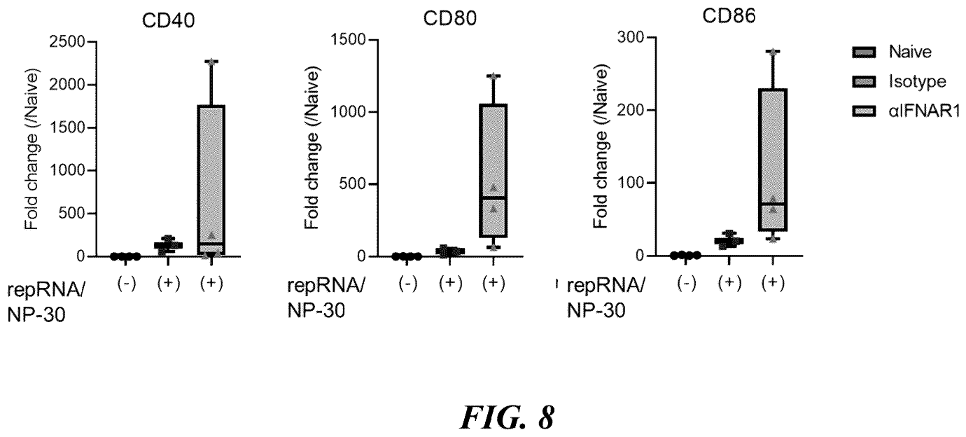

The differential early host response to LNP- or NP-30-delivered repRNA was assayed in the muscle. NanoString analysis was performed using a Mouse Host Response 773-gene panel of the RNA extracted from muscle tissue of naïve mice or mice receiving LNP or NP-30-formulated repRNA 14 hours after IM injection (FIG. 8). Several of the measured gene transcripts were up-regulated by repRNA delivered by NP-30 and LNP compared to naïve samples (FIGS. 3A-3B), which included genes for chemokines and pro-inflammatory cytokines. The expression of inflammatory genes in the muscle of repRNA/NP-30 and repRNA/LNP-injected mice was compared. Of the 192 gene transcripts that were differentially expressed in these groups, many of them (188 genes) were preferentially expressed by repRNA/NP-30 (FIG. 3C). PanglaoDB Cell Types analysis of the differentially expressed genes between the LNP and NP-30 groups was performed. Antigen-presenting cells (APCs) such as macrophages, monocytes, and dendritic cells (DCs) (e.g., H2-ab1, H2-eb1, Itga1/Cd11a, Itgam Cd11b) were enriched in repRNA/NP-30-injected muscle (FIG. 3D). NanoString annotation analysis of the differentially expressed genes showed that gene transcripts for Fc gamma receptor (FCGR)-dependent phagocytosis, co-stimulation by the CD28 family, and Class I MHC-mediated antigen processing & presentation pathway were enriched in the muscle of repRNA/NP-30-injected mice over those of repRNA/LNP-injected mice (FIG. 3E). Co-stimulatory molecules (FIG. 3F) and multiple types of interferons, except for IFN-β, (FIG. 3G) were also induced by repRNA/NP-30 over repRNA/LNP (FIGS. 3H-3I). Blockade of type I IFN signaling did not impair, and if anything, up-regulated the local induction of genes encoding co-stimulatory molecules in repRNA/NP-30-injected mice (FIG. 8), suggesting the dispensable role of type I IFN signaling in the activation of innate immune cells in the muscles of repRNA/NP-30-delivered mice in vivo. Overall, IM injection of repRNA/NP-30 activated the local innate immune environment in the injected muscle to a higher degree than repRNA/LNP.

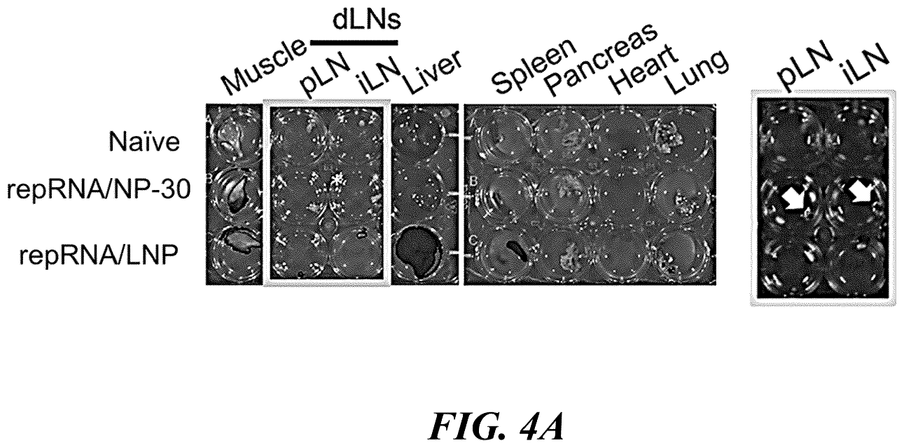

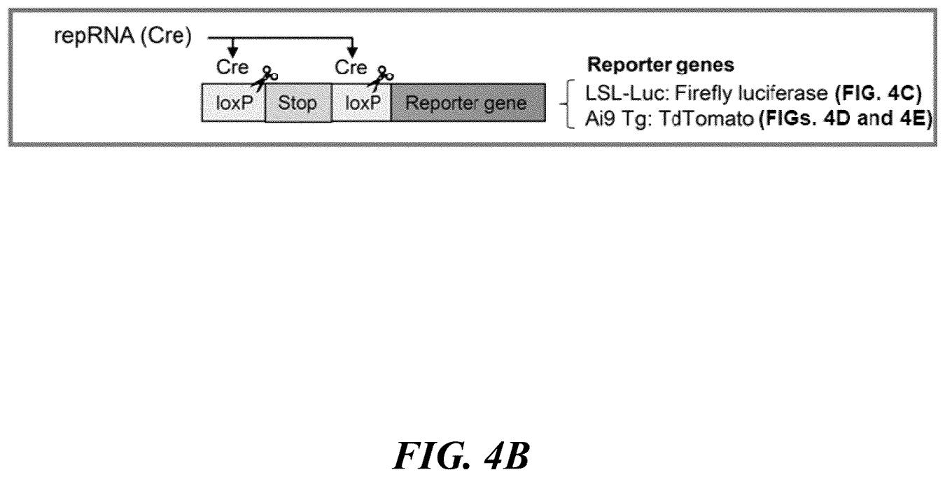

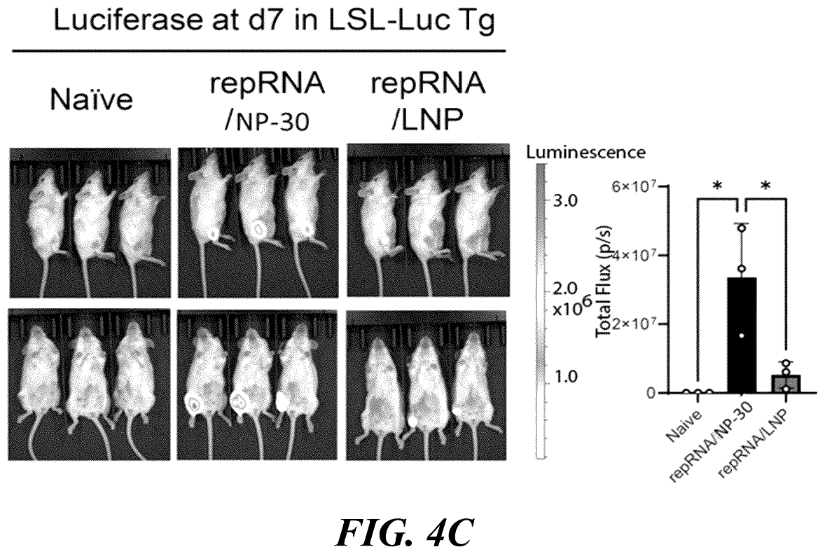

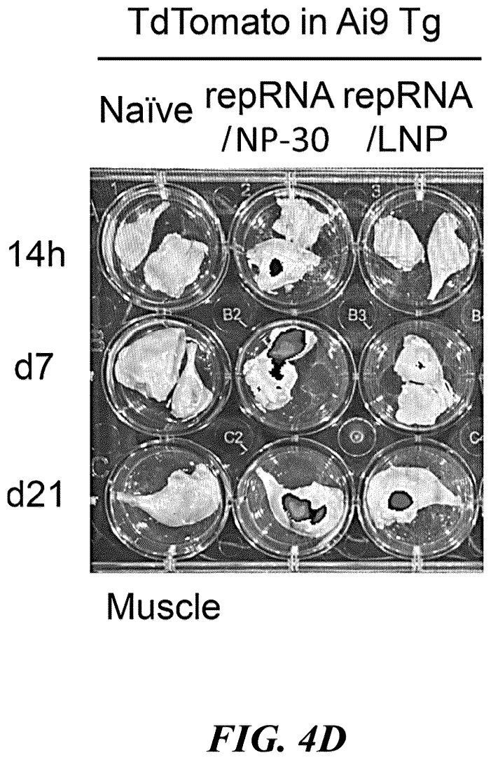

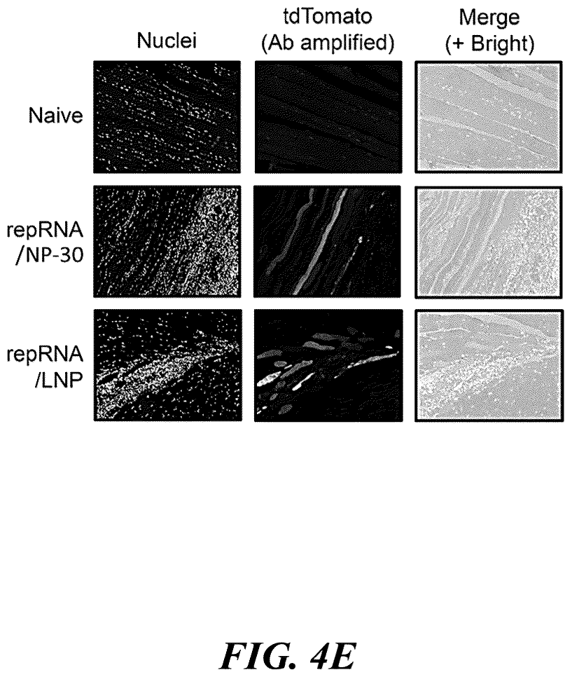

Biodistribution was determined for NP-30 and LNPs. NP-30 and LNP were each conjugated with a fluorescent dye, XenoLight DiR (herein, NP-30-DiR and LNP-DiR, respectively), formulated them with repRNA, and IM injected them into C57BL/6 mice 24 hours before harvesting muscle, draining popliteal and inguinal lymph nodes (pLN and iLN, respectively), liver, spleen, pancreas, heart, and lung for IVIS analyses. A higher level of NP-30-DiR signal was detected in the muscle and a relatively weaker level in the dLNs by 24 hours after the injection, but not detectable in any of the other sampled organs (FIG. 4A). LNP-DiR signal was detected in multiple tissues, including the dLNs, liver, spleen, and to a lesser degree in the lung (FIG. 4A). A broad biodistribution of LNP was observed and detected in multiple tissues throughout the body. NP-30 remained in the local injection site.

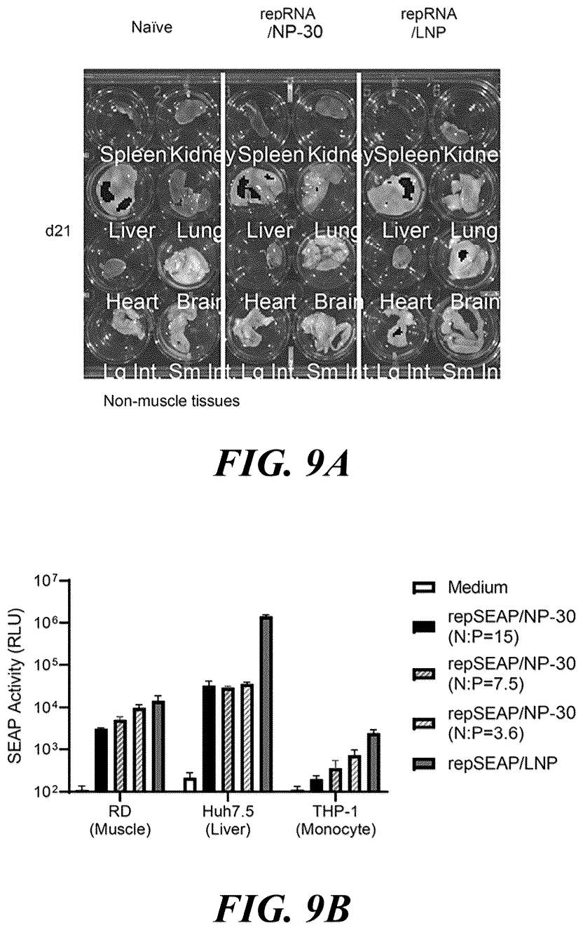

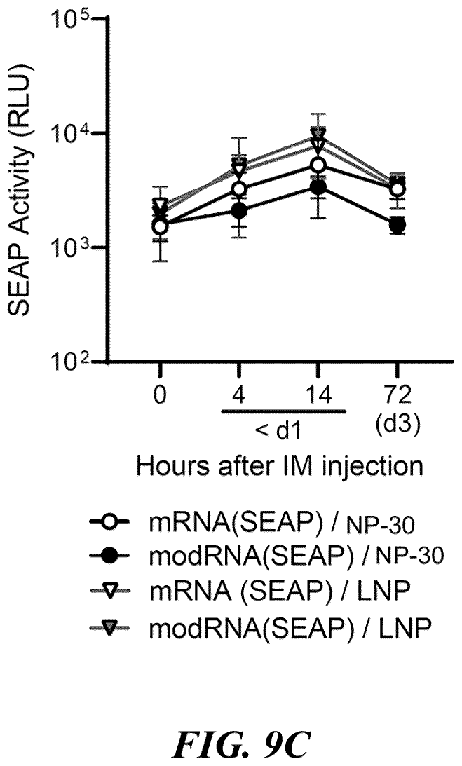

Next, reporter mice (LSL-Luc Tg and Ai9 Tg) were employed to determine transgene expression patterns following nanoparticle biodistribution A Cre/loxP-mediated genomic recombination approach was used to drive the expression of reporter genes (luciferase and tdTomato, respectively), for the detection of cells and tissues that received repRNA expressing Cre recombinase prior to harvest (FIG. 4B). Seven days after IM injection of repRNA expressing Cre recombinase, the luciferase-expressing area was restricted to the injected leg of both repRNA/NP-30 and repRNA/LNP-injected LSL-Luc Tg mice (FIG. 4C). The total transfected area was more extensive in repRNA/NP-30-injected mice compared to repRNA/LNP-injected mice. A similar observation was made in the extracted tissue of Ai9 Tg mice after the repRNA delivery. In the muscles of repRNA/NP-30-injected Ai9 Tg mice, the transfected region was detected as early as 14 hours after IM injection and maximized at day 7 after IM injection (FIG. 4D). In the muscle of repRNA/LNP-injected mice, the total transfected area was minimally detectable by day 7 and became evident at day 21. In contrast to the lipid biodistribution observations above, no other tissues showed robust reporter gene expression (FIG. 9A). The LNP formulation mediated efficient transfection of repRNA into multiple cell types in vitro, including muscle, liver, and monocyte cell lines (FIG. 9B), confirming that the lack of reporter gene detection in extra muscular tissues was not due to impaired transfection of those cells in vivo. Transgene expression of mRNA by using NP-30 compared to LNP in mice at multiple time points following IM injection was also assayed. (FIG. 9C).

To further analyze what cells are transfected by repRNA/NP-30 and repRNA/LNP in the muscle, muscle sections of Ai9 mice that received repRNA were analyzed. Immunofluorescence assays revealed that both NP-30 and LNP deliver repRNA mainly to myocytes, and mononuclear cellular infiltration with some transgene expression was observed in the proximity of the transfected myocytes (FIG. 4E). NP-30 stayed within the muscle while LNP disseminated throughout the body, but in both cases, the repRNA expression was restricted to the muscle.

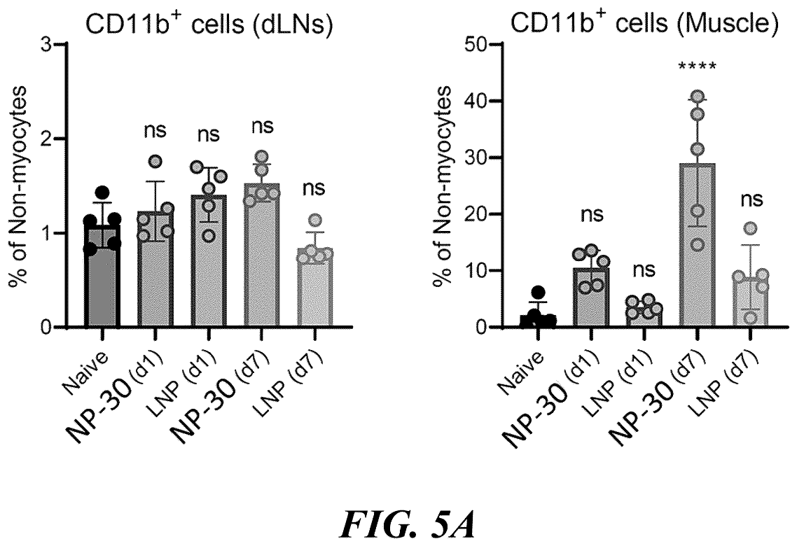

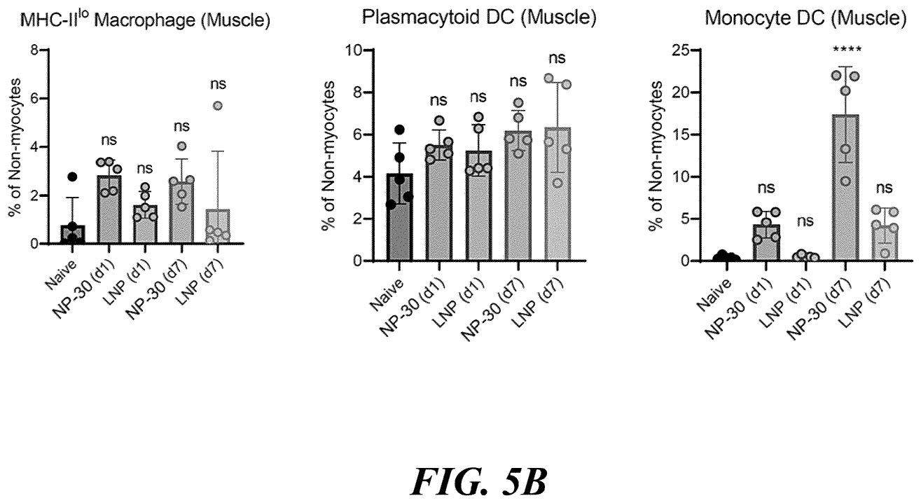

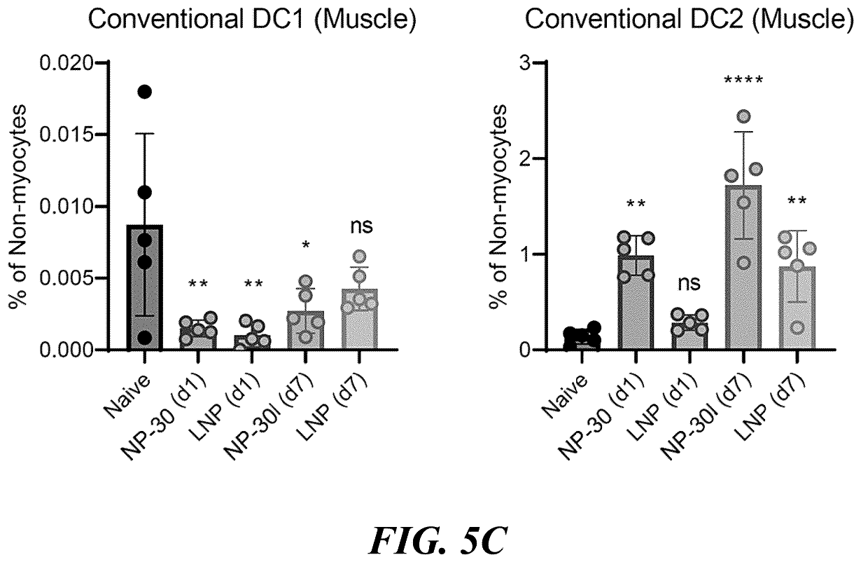

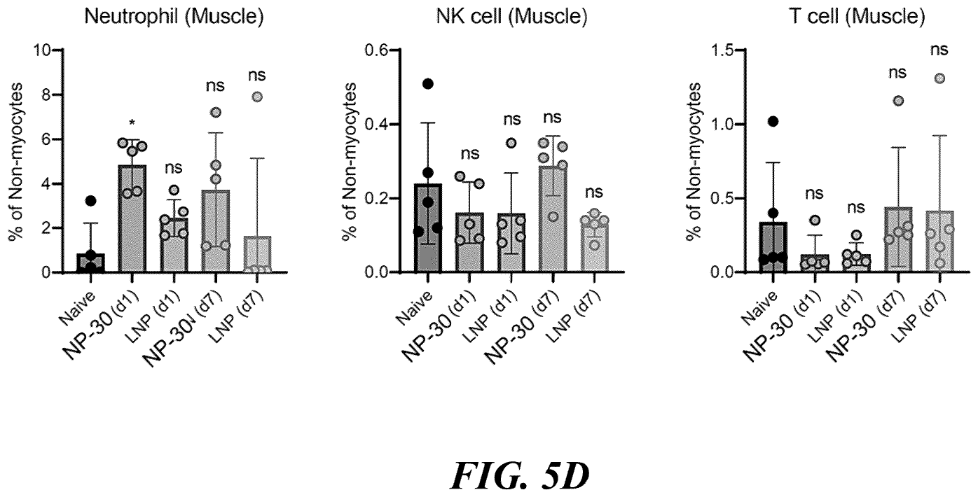

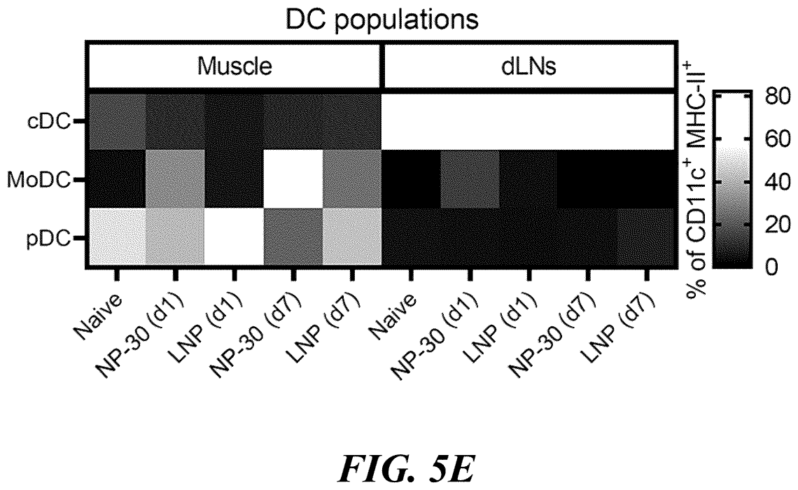

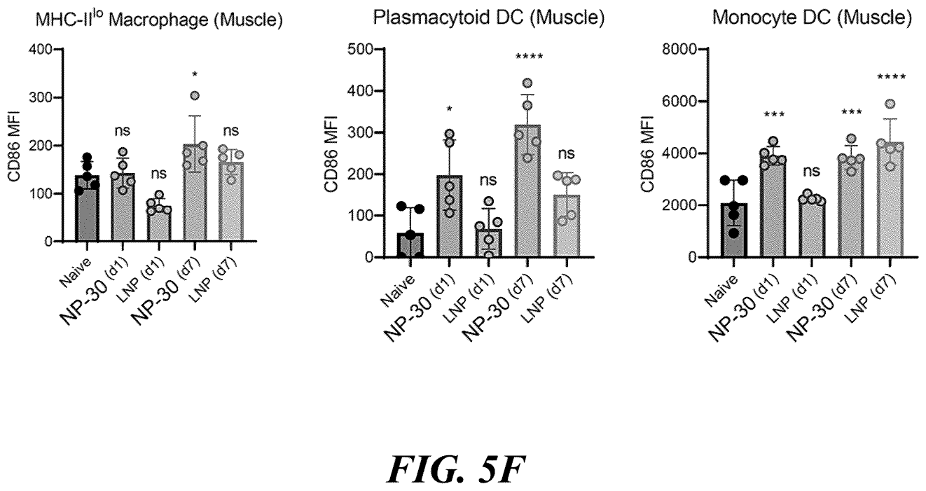

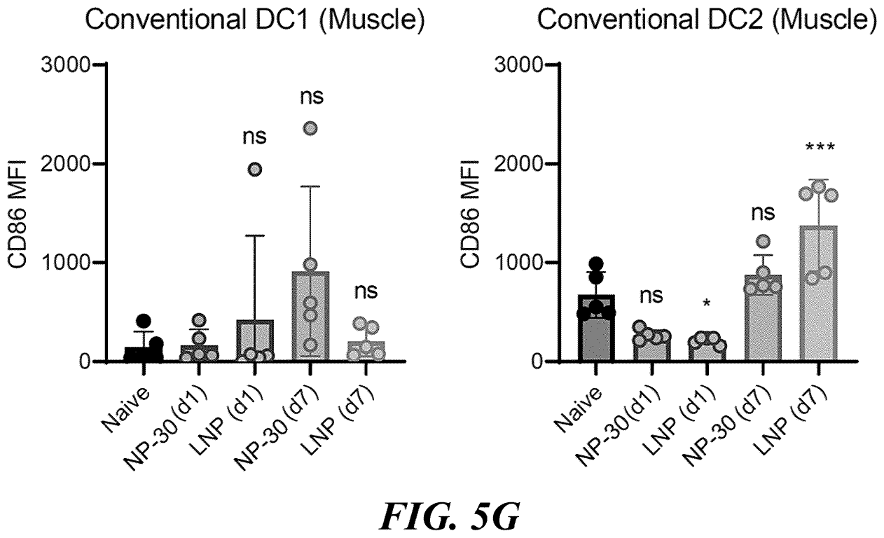

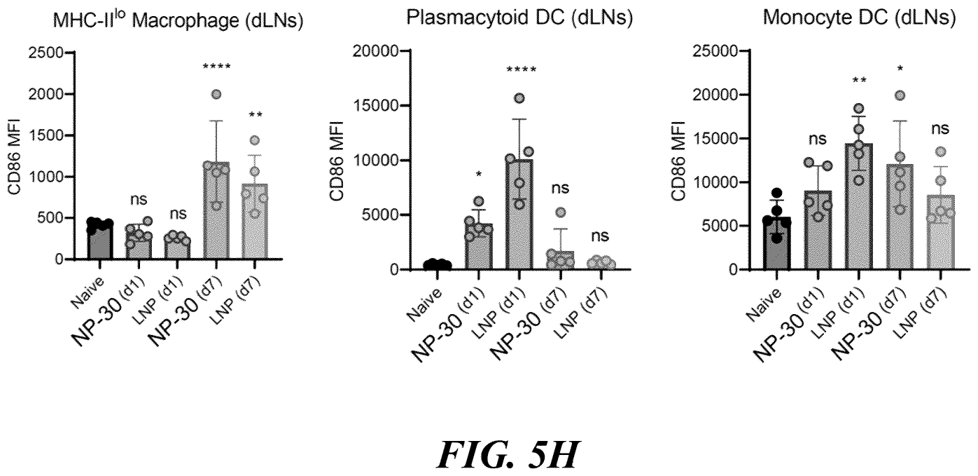

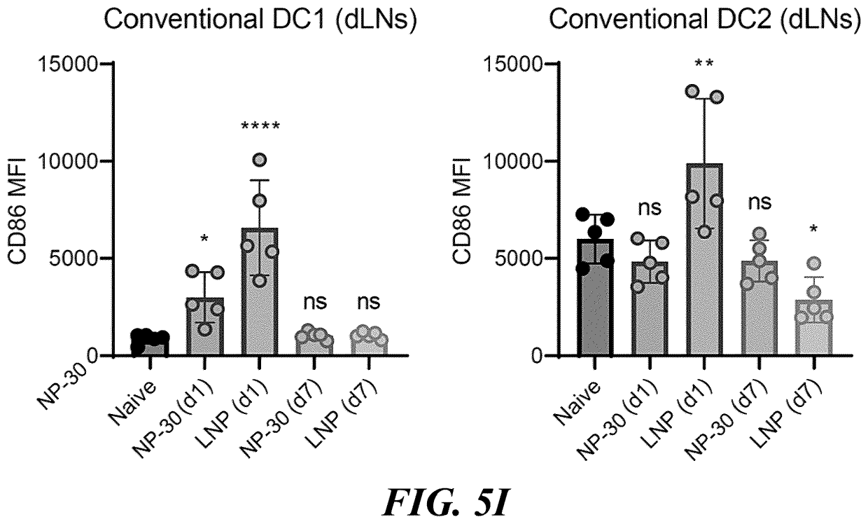

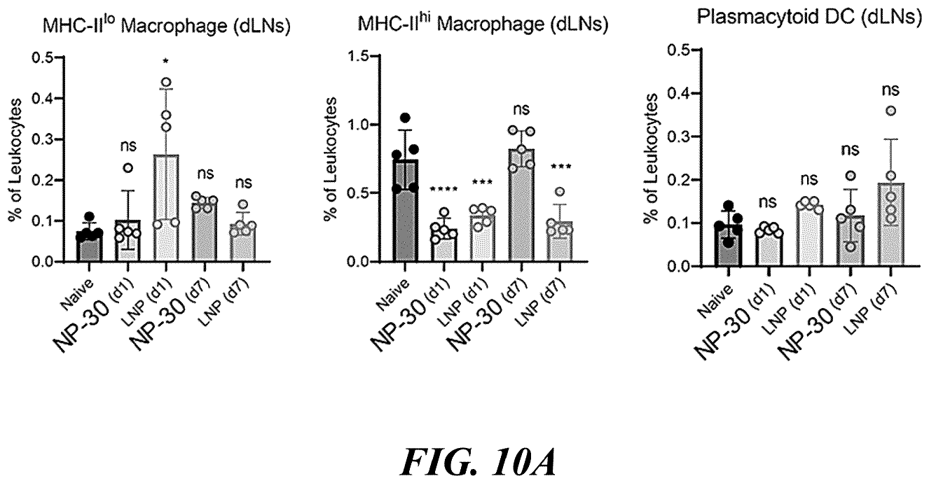

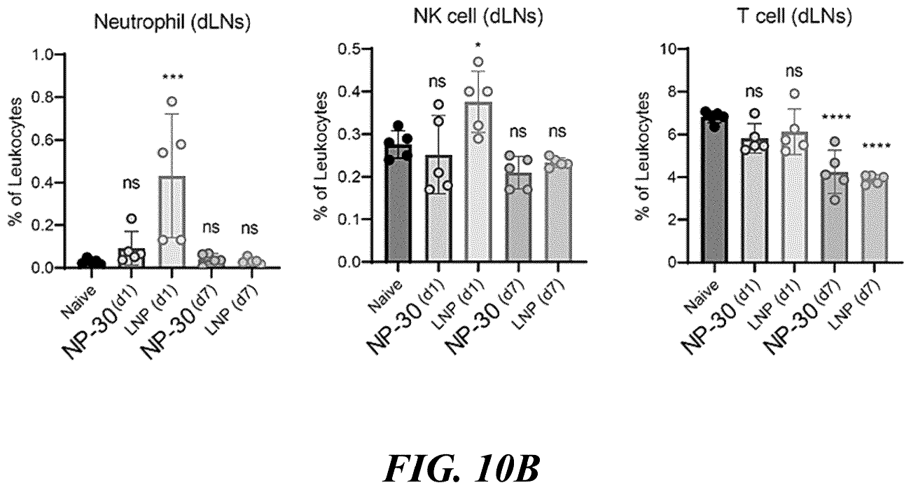

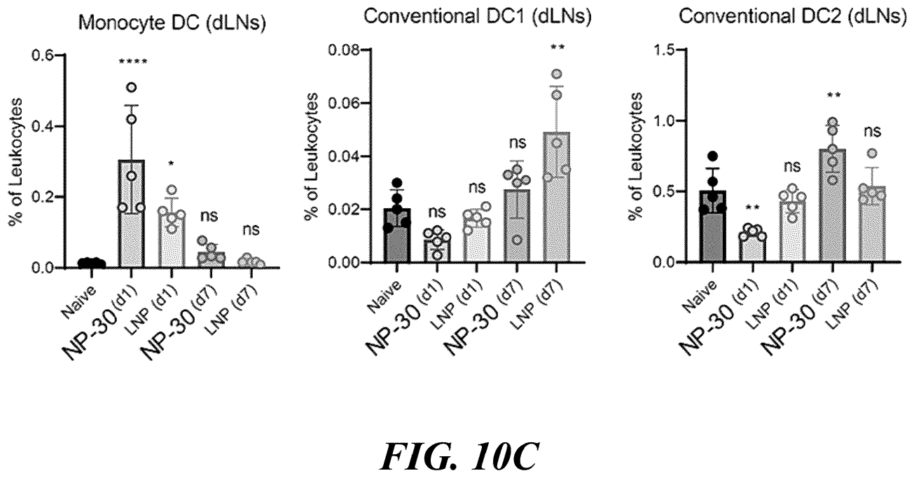

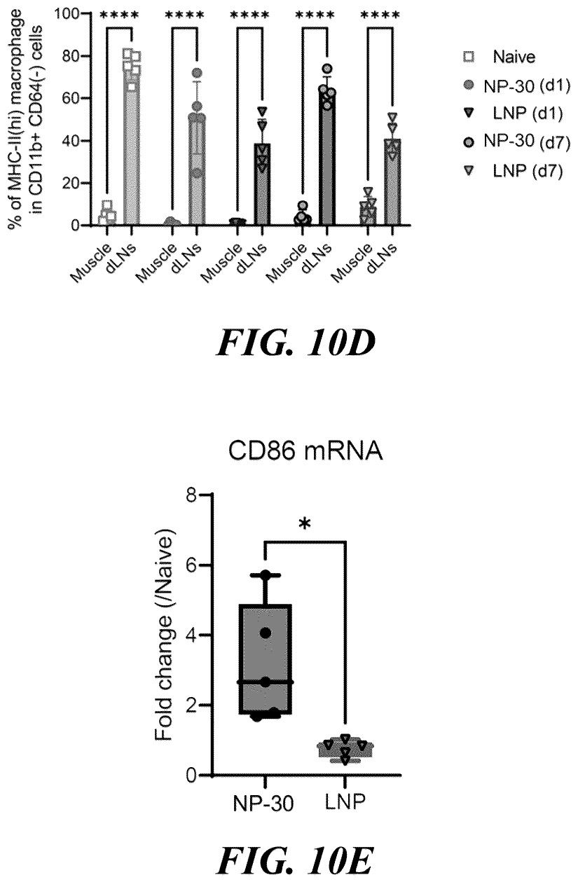

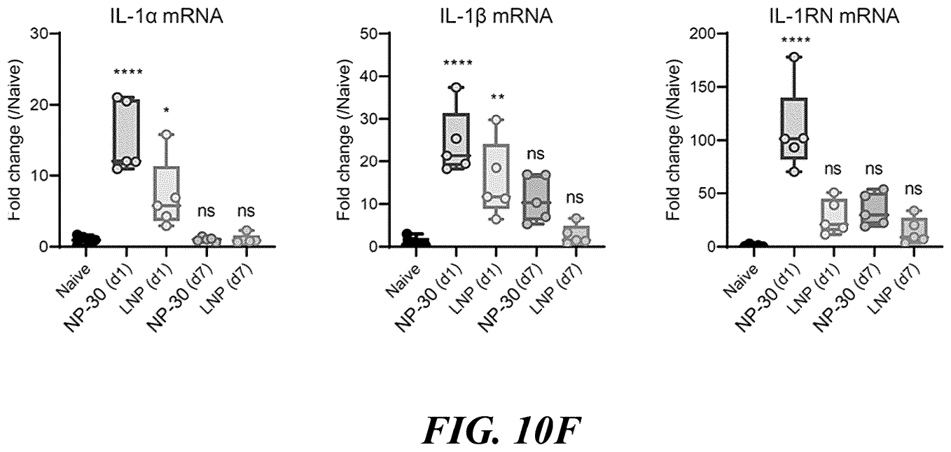

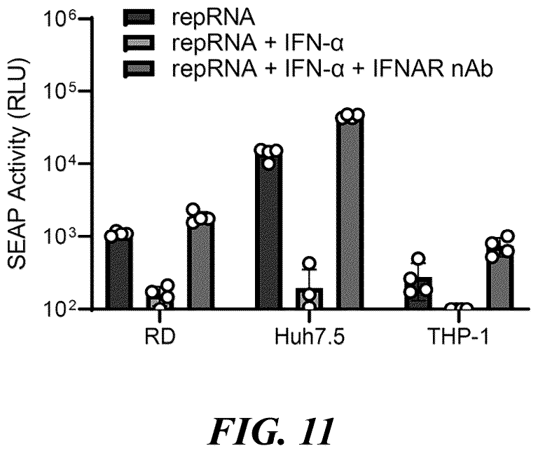

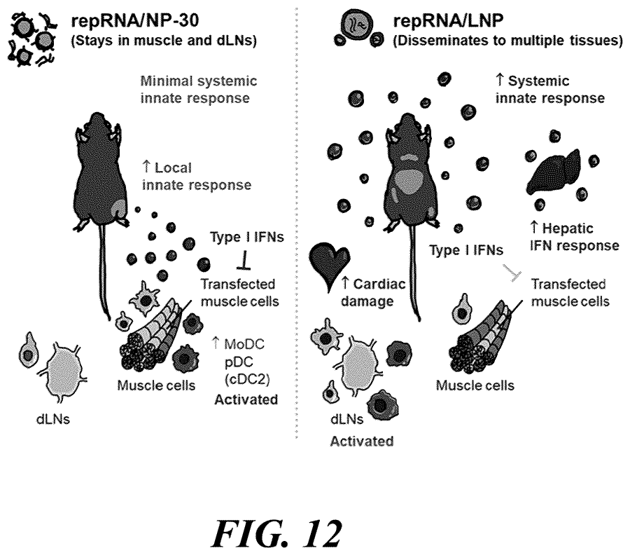

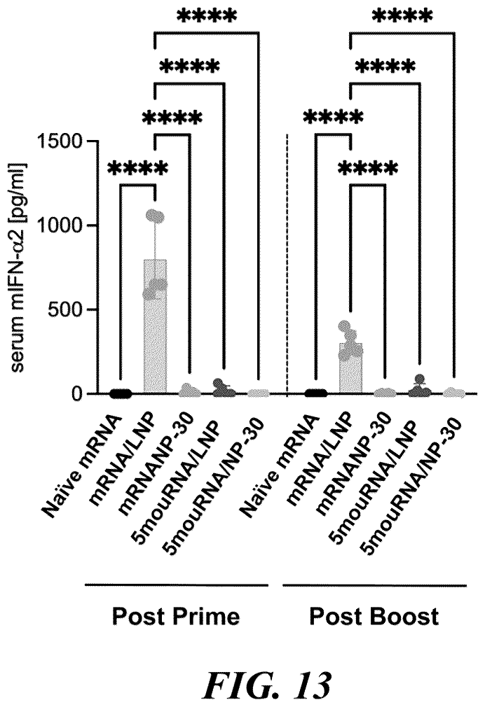

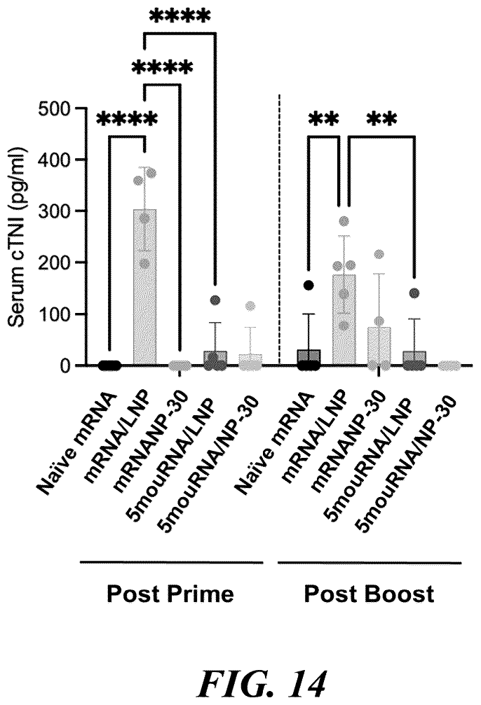

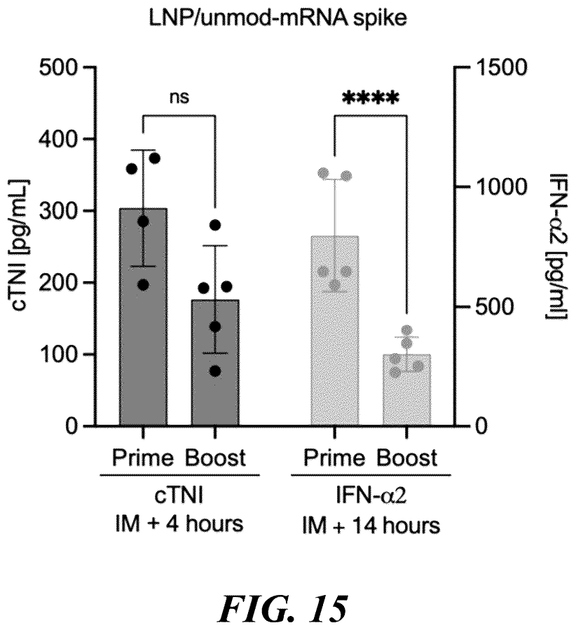

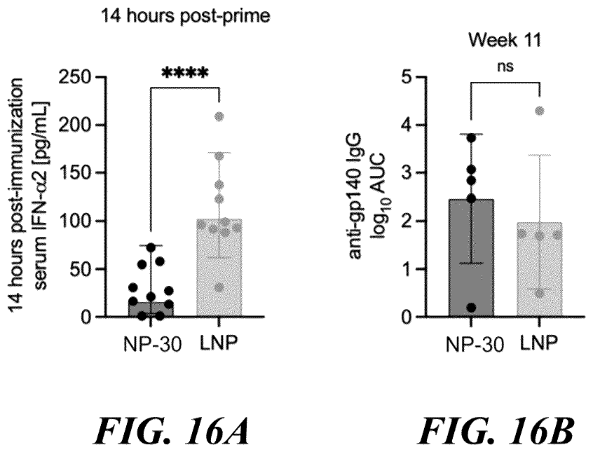

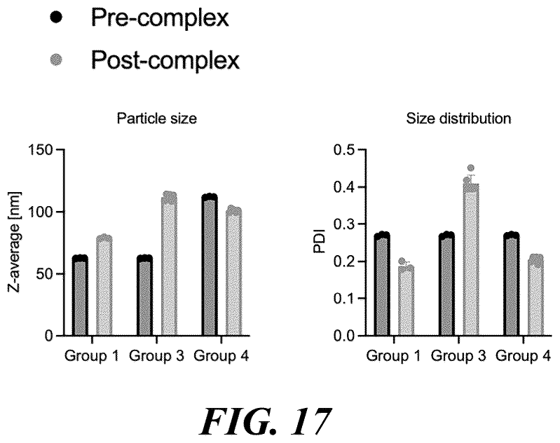

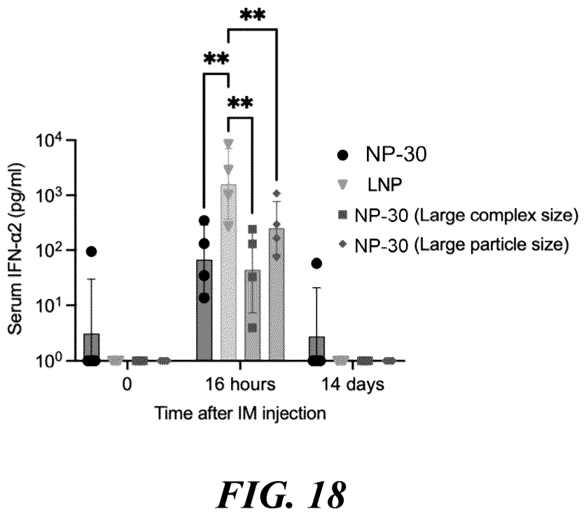

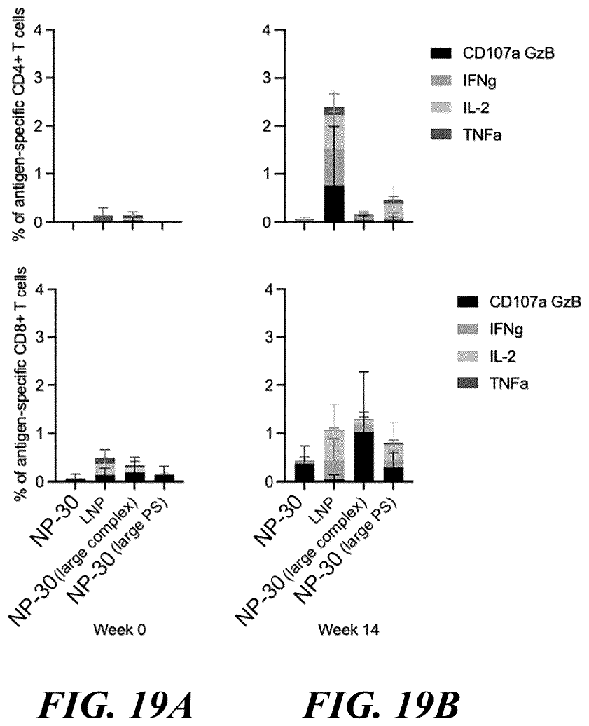

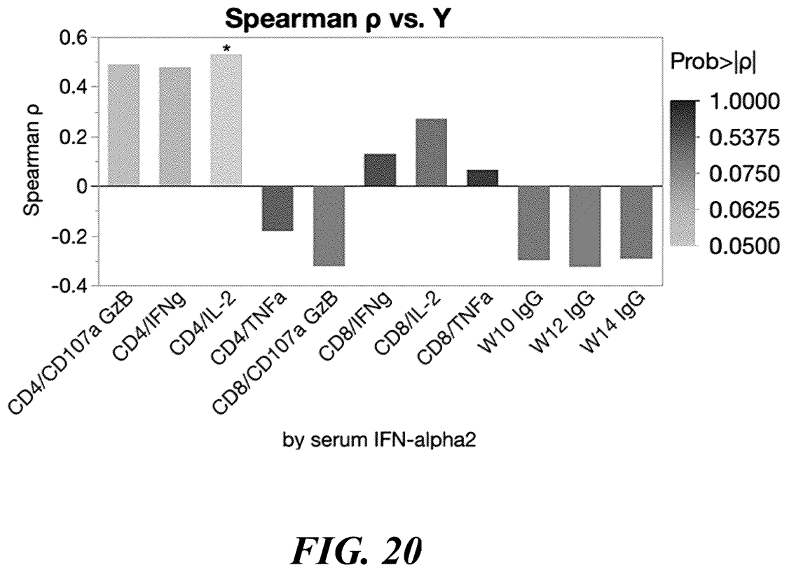

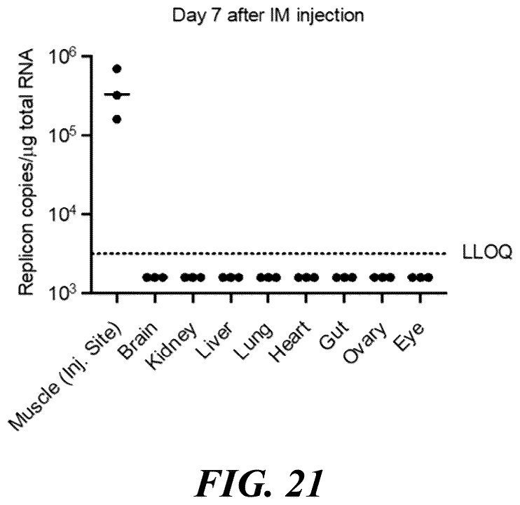

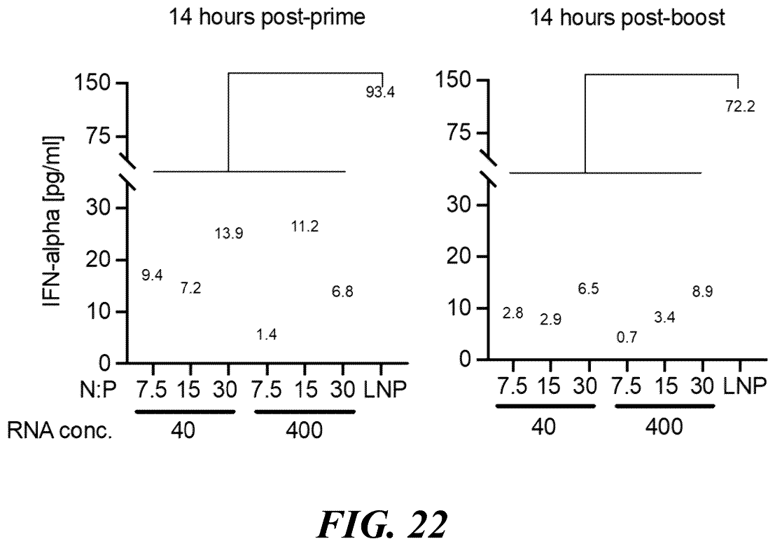

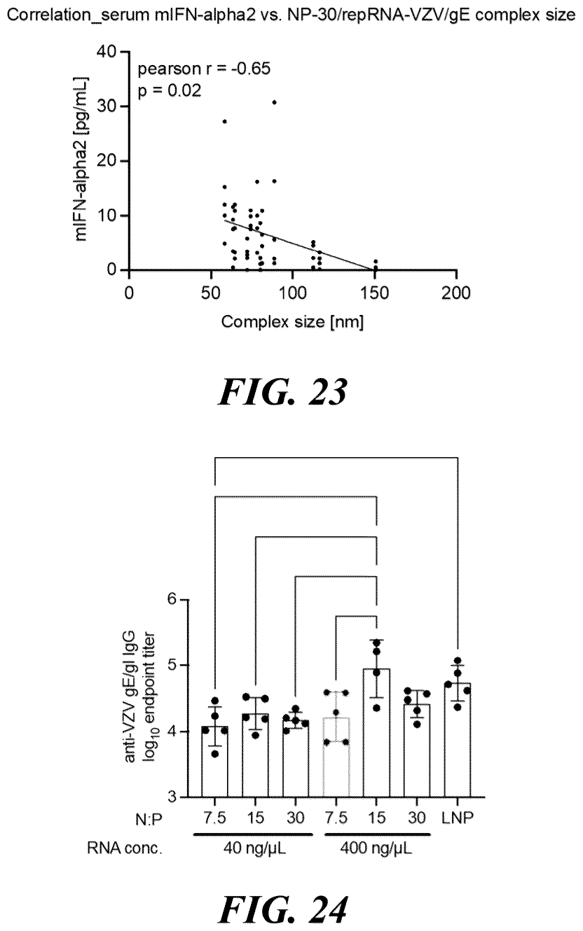

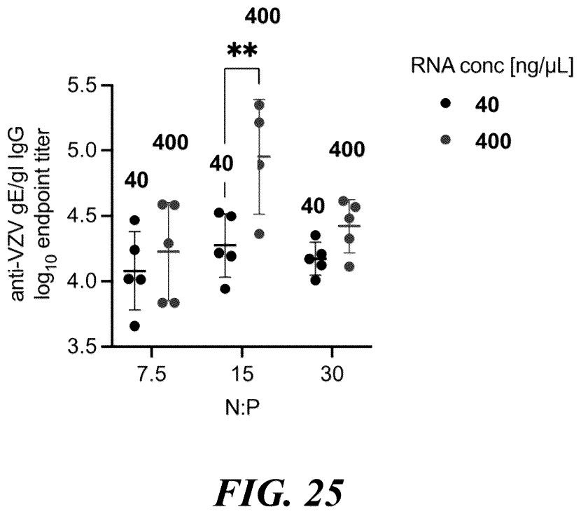

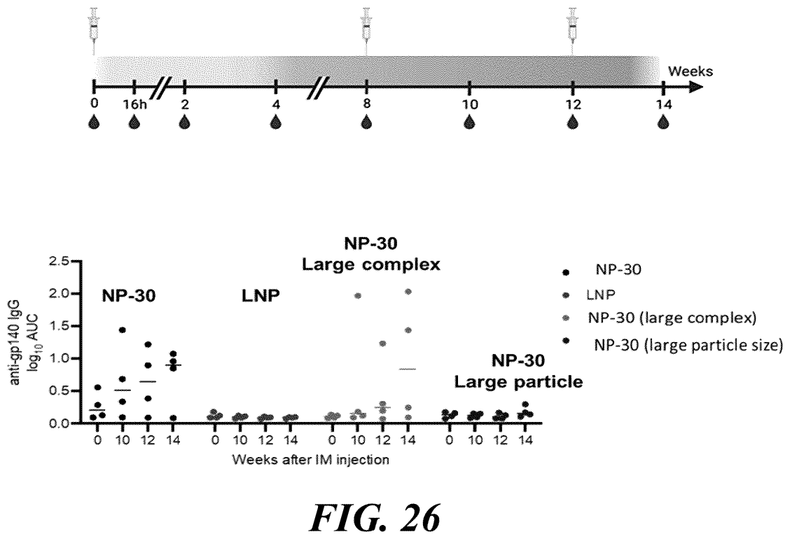

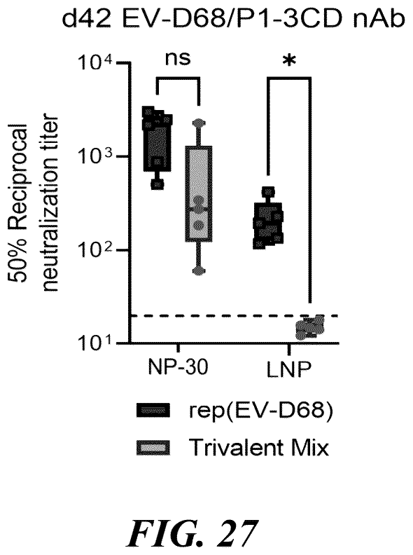

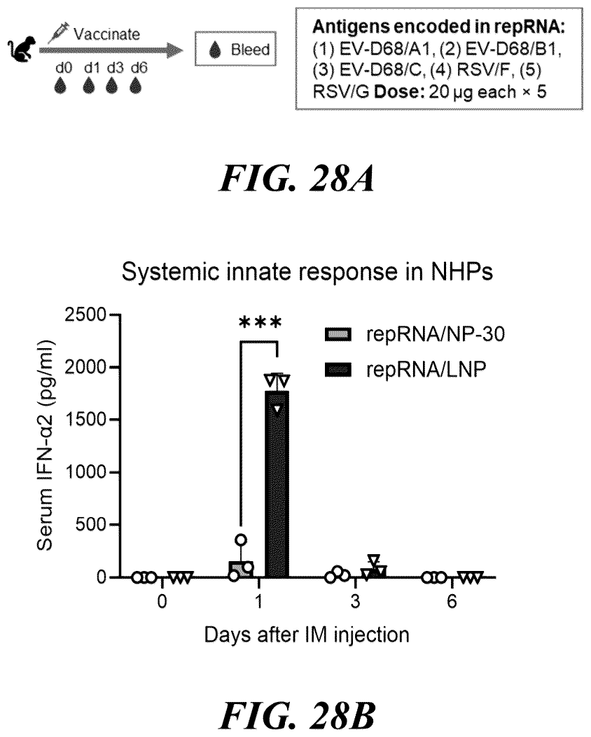

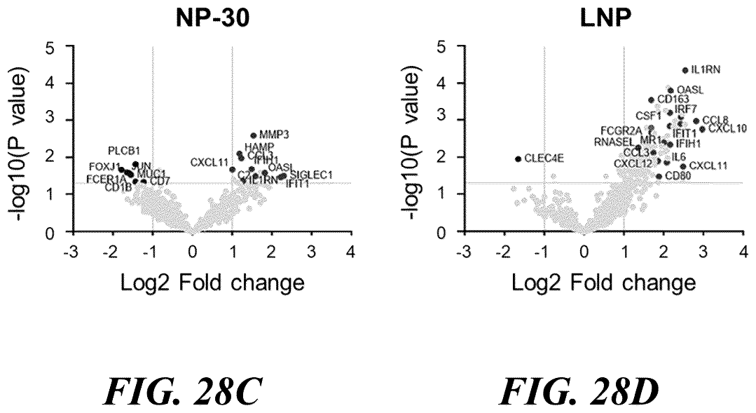

Immune cell activation in response to repRNA/NP-30 or repRNA/LNP was assayed. C57BL/6 mice received IM injection of repRNA formulated with NP-30 or LNP, then non-myocytes were analyzed by enzymatically removing myocytes. Flow cytometric analysis of the isolated non-myocytes revealed that CD11b⁺ cells were significantly expanded in the muscles of repRNA/NP-30-delivered mice compared to those of repRNA/LNP-delivered mice (FIG. 5A). At the cell type level, repRNA/NP-30 induced the expansion of monocyte dendritic cells (MoDCs) (CD64⁺ CD11c⁺ MHC-II), conventional DC2s (cDC2; CD11c⁺ MHC-II XCR1⁻ CD11b⁺) and neutrophils (CD11b⁺ Ly6G^{hi}Ly6C^{lo}) in the muscle, whereas repRNA/LNP induced only a modest expansion of cDC2 in the muscles by day 7 after IM injection (FIGS. 5B-5G). The repRNA/NP-30 minimally affected the immune cell composition in dLNs except for MoDCs, while repRNA/LNP induced the expansion of cDC, neutrophils and NK cells (Ly6G⁻ CD64⁻ CD3⁻ NK1.1⁺) in dLNs at day 1 after IM injection (FIG. 10A). Class II MHC was not expressed by the majority of macrophages (CD11b+CD64-CD11c⁻) in the muscle, while it was highly expressed by this cell type in dLNs (FIG. 10B), suggesting the unique innate immune cell environment in the muscle. Likewise, the composition of DC (CD11c⁺ MHC-II⁺) populations was significantly different between muscles and dLNs (FIG. 5B, FIG. 5E, and FIGS. 5H-5I). In the steady state, DC in the muscle was mainly comprised of plasmacytoid DC (pDC; CD11c⁺ MHC-II CD64⁻ PDCA-1⁺) (FIG. 5B), whereas DCs in the dLNs were predominantly comprised of conventional type DCs (FIG. 10A-10C). The repRNA derived from NP-30 formulations significantly altered the DC composition in the muscle from pDC dominant to MoDC dominant by day 7 (FIG. 5B), while it did not affect DC composition in dLNs (FIG. 5E and FIG. 10D). RepRNA/LNP also affected the DC composition but to a lesser extent relative to repRNA/NP-30 in the muscle (FIGS. 5B-5C, 5E).

Next markers of T cell activation were assayed, including CD86. CD86 mRNA was induced by repRNA/NP-30 in overall non-myocytes 1 day after IM injection (FIGS. 5F-5G, FIGS. 10A-10D). FIG. 10E shows the fold change in CD86 expression levels in non-myocytes isolated from the muscles of mice administered repRNAs complexed with either LNP or NP-30 were determined by qRT-PCR and shown as a box plot. X-axis: Conditions; Y-axis: fold change (relative to control animals). MHC-II^{lo} macrophage, pDC, and MoDC were activated over time in the muscles of repRNA/NP-30 relative to repRNA/LNP-injected mice. RepRNA delivered by LNP induced activation of MoDC and cDC2 only at day 7, suggesting a difference in the kinetics of immune cell activation in the muscle between the two formulations (FIGS. 5F-5G). Of note, repRNA/LNP substantially induced the activation of all the analyzed cell types except for MHC-II^{lo} macrophage in dLNs at day 1, and the activation levels were reverted to background levels by day 7 (FIGS. 5F-5G). The repRNA/NP-30 activated innate immune cells in the muscle, while repRNA/LNP preferentially activated immune cells in the dLNs. Furthermore, interleukin-1 family cytokines transcripts were measured via PCR from mice treated with NP-30 and LNP on days 1 and day 7 (FIG. 10F). Initially, on day 1, both NP-30 and LNP treated animal had increased IL-1α and IL-1βmRNA levels. NP-30 also increased IL-1RN mRNA production on day 1. However, by day 7, IL-1 family cytokine mRNA was not significant compared to control naïve mice (FIG. 10F)

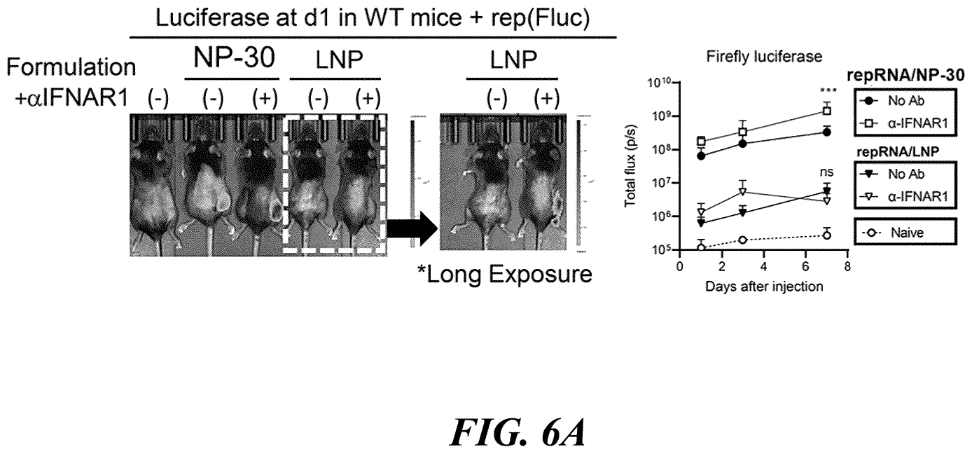

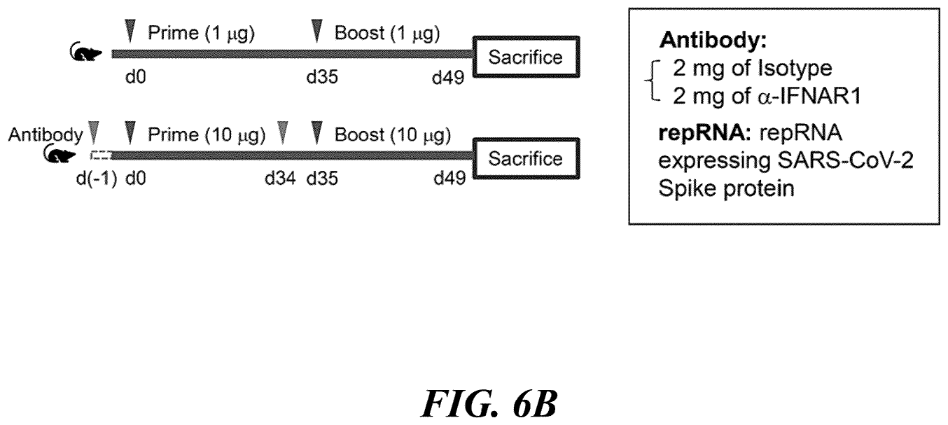

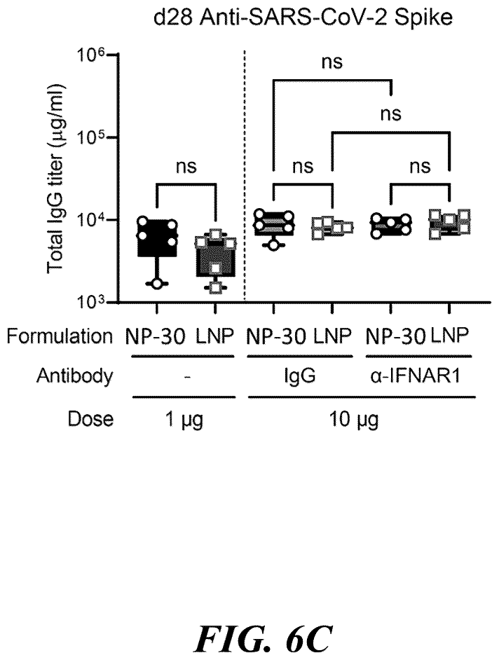

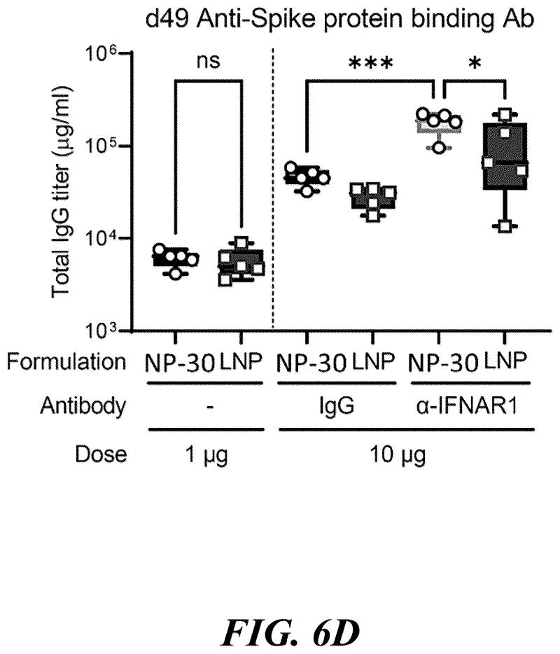

The immunogenicity in response to NP-30 and LNP was assayed. Blockade of type I IFN signaling in repRNA/NP-30 and repRNA/LNP-injected mice showed greater reporter gene expression in the muscle-injected site at day 1, but the extent of expression was much greater with the NP-30 formulation (FIG. 6A). For NP-30 injected animals, protein expression was still restricted to the local injection site even in the absence of type I IFN signaling, despite the preconditioning of multiple cell types with type I IFN inhibited protein expression in vitro (FIG. 1I). Therefore, type I IFN signaling did not alter biodistribution. Antibody response was assayed for four weeks after the prime and two weeks after the second dose of repRNA encoding SARS-CoV2 Spike protein delivered via NP-30 or LNP (FIG. 6B). At day 28 before the boost dose, repRNA/NP-30 and repRNA/LNP induced comparable antibody response at both doses (FIG. 6C). α-IFNAR1 antibody treatment did not affect the antibody response after the prime (FIG. 6C). At day 49 (two weeks after the boost), repRNA delivered by both formulations induced comparable levels of spike-binding antibody response at the 1 μg dose (FIG. 6D). At the 10 μg dose, where repRNA/NP-30 induced only minor systemic innate response, repRNA/NP-30-injected mice still induced better immunogenic responses than repRNA/LNP-injected mice (FIG. 6D).

Large dose RNA vaccination was safely achieved in mice by delivering self-amplifying replicon RNA (repRNA) with muscle-localizing cationic nanocarrier, NP-30. In vivo delivery of repRNA by NP-30 highly up-regulated muscle local innate immune response without inducing systemic cytokine production, activated innate immune cells were expanded in the muscle, and surprisingly still produced an adaptive immune response. In contrast, repRNA delivered by lipid nanoparticles (LNP) showed broader biodistribution, leading to a systemic inflammatory state. The results provided above show that in vivo delivery of repRNA by a muscle-localizing formulation, NP-30, is an effective approach for safe and immunogenic vaccination through a unique mechanism distinct from others delivered by LNP. Surprisingly and unexpectedly, the NP-30 muscle-localizing platform promoted immunogenic vaccination without triggering systemic cytokine production. FIG. 12 shows a schematic summarizing the mechanism of action for repRNA/NP-30 in comparison with repRNA/LNP.

### Example 4: Additional Methods for Detecting an Immune Response

The assays provided in Example 3 and methods of detecting and quantifying a local and/or systemic immune response are described herein. Assays include immunosorbent assays, cytokine array analysis, in vivo imaging for biodistribution, PCR, flow cytometry, histology, and secreted embryonic alkaline phosphatase (SEAP) reporter assays.

### Enzyme-Linked Immunosorbent Assay (ELISA)

(a) IFN-α2 ELISA: Serum IFN-α2 levels were measured by using Lumikine Xpress IFN-α 2.0 ELISA kit (Invivogen) according to the manufacturer's instruction.
(b) Serum cTNI ELISA: Serum cTNI levels were measured by using cTNI ELISA kit (MyBiosource, #MBS766175).
(c) SARS-CoV-2 binding antibody ELISA: Antigen-specific IgG responses were detected in sera by ELISA and performed as previously described. Briefly, ELISA plates were coated with 1 μg/ml recombinant SARS-CoV-2 S protein, and serially diluted serum samples were added and detected via anti-monkey IgG-HRP (Southern Biotech, Birmingham, AL). Plates were developed using a TMB substrate (source) and were analyzed at 450 nm (ELX808, Bio-Tek Instruments Inc). IgG serum concentrations were determined from a standard curve, as previously described.

### Cytokine Array Analysis

Sera from mice that received IM injection of 10 μg of repRNA/NP-30 or repRNA/LNP were used for cytokine array. The array was performed using Proteome Profiler Mouse XL Cytokine Array (R&D systems #ARY028) according to the manufacturer's instructions. Briefly, membranes were blocked for one hour at room temperature. Meanwhile, samples were incubated with detection antibodies for one hour at room temperature. Then, the blocked membranes were incubated in the sample/detection antibody mixtures overnight at 4° C. Next day, the membranes were washed three times and incubated with diluted Streptavidin-HRP for 30 min. at room temperature. After washing three times, the membranes were incubated with Chemi Reagent Mix, and each spot was visualized on ChemiDoc™ touch imaging system (Bio-Rad). The signal intensities of each spot were calculated using ImageJ software, and each value was normalized against the mean values of reference spots (pre-defined by the manufacturer).

### In Vivo Imaging System (IVIS)

For ex vivo IVIS analysis, mice were IP injected with D-luciferin. 5 min later, mice were sacrificed, and the tissues were isolated. Extracted tissues were soaked in PBS and analyzed by IVIS. Fluorescent images were obtained by using specific filters, and bioluminescent images were obtained by the open filter.

For in vivo IVIS analysis, mice were anesthetized, their hair was clipped, and IP injected with D-luciferin. After 5 min of the injection, ventral and dorsal photon emissions were analyzed during 5 s exposure, and the average total flux values of the region of interest were used for analyses.

RNA Analysis Using RNA Isolated from Muscle Tissues

RNA isolation: Muscle tissues were isolated from mice, and RNA was isolated using Fibrous RNeasy Mini Kit (QIAGEN, #74704). Briefly, the pieces of the muscle injected site were homogenized in RLT buffer (a component of RNAeasy™ Mini Kit) using TissueLyzer™ LT (QIAGEN). Cell debris was removed by centrifuge, and supernatant was transferred to the RNAeasy mini column. After a centrifuge, followed by one-time wash of the column with RW buffer, DNA was digested on the column using DNAse II by incubation for 30 min at 55 deg. Columns were washed with RW buffer once, then with RPE buffer twice, and the final elusion was done with nuclease-free water.

Realtime RT-PCR: Up to 1 μg of RNA was reverse transcribed using Invitrogen™ SuperScript™ First-Strand Synthesis System for RT-PCR (Invitrogen, #11904018) according to the manufacturer's instruction. Transcribed cDNA libraries were diluted at 1:5 in nuclease-free water and used for quantitative real-time RT-PCR using SYBR Green™ Master Mix.

### Immunofluorescence and Histological Analysis

Mice were sacrificed at the endpoint, and tissues were harvested and fixed using buffered zinc formalin at room temperature overnight. For histological analyses, formalin-fixed tissues were paraffin-embedded, and 3 μm sections were cut. For standard histological analyses, sections were stained with Hematoxylin-Eosin. For immunofluorescence, sections were deparaffinized with Histo-Clear II (Electron Microscopy Sciences #6411101) for 10 min, 100% Ethanol for 10 min, 90% ethanol for 3 min, 70% ethanol for 3 min, then washed with PBS for 5 min. The sections were then washed with PBS for 5 min three times and permeabilized with 0.5% Triton/PBS for 30 min at room temperature, followed by blocking with 1% Normal Goat Serum for 30 min at room temperature. The blocked sections were incubated with RFP Polyclonal Antibody (Thermo Fisher #600-401-379-RTU) to amplify tdTomato signal overnight at 4° C. The next day, the sections were washed with 0.3% Triton/PBS and treated with Goat anti-Rabbit (Alexa Fluor 488-conjugated) for at least 2 h at room temperature. After the staining, sections were washed with 0.3% Triton/PBS and treated with TrueView Auto fluorescence kit (Vector Laboratories #SP-8400) according to the manufacturer's instruction. Before mounting with the reagent provided by the kit, sections were counterstained with Hoechst 33342 (1:10,000 dilution in PBS) (Thermo Fisher Scientific #H3570).

### Flow Cytometry

Single cell suspensions were stained with antibodies anti-CD11c (1:200 dilution, BV510), anti-Ly6C (1:200 dilution, BV605), anti-Ly6G (1:200 dilution, BV785), anti-CD64 (1:200 dilution, BV421), anti-MHC-II (1:400 dilution, AF700), anti-CD11b (1:200 dilution, APC-F750), anti-XCR1 (1:200 dilution, BV650), anti-CD3 (1:200 dilution, BUV737), anti-CD86 (1:200 dilution, PE), anti-CD4 (1:200 dilution, BUV395), anti-PDCA-1 (1:200 dilution, BV711), anti-CD8 (1:200 dilution, BUV805), anti-NK1.1 (1:200 dilution, PerCP-Cy5.5) and data were acquired on FACSymphony (BD). The acquired data were analyzed by FlowJo (BD).

### Quantitative Real-Time RT-PCR

Real-time PCR was performed using Power SYBR Green PCR master mix reagent (Applied biosystems, #4367659) with specific primer sets.

### SEAP Assays

SEAP activity was measured by NovaBright™ Phospha-Light™ EXP Assay Kit for SEAP reporter gene detection kit (Invitrogen #N10578) according to the manufacturer's instructions.

### Example 5: Immunogenicity of Sars-Cov-2 Expressing Unmodified mRNA Vs. Modified mRNA, Formulated with Np-30 Vs. Lnp

Stock RNA and NP-30 were diluted to working concentration for each immunization according to tables below. NP-30/repRNA complexes for groups 3 and 5 were prepared by pipetting 150 μL diluted repRNA solution to 150 μL of corresponding diluted NP-30 solution. The mixture was pipetted up and down to ensure complete mixing. NP-30/repRNA complexes were incubated at room temperature for 30 minutes before injection. Groups 2 and 4 were immunized with LNPs prepared as described in Example 1.

Table 10 provides a description of reagents used in the assays provided herein.

TABLE 10
Reagents and Concentrations.

| Description | Concentration | |
|---|---|---|
| NP-1 w/o Fe (NP-30) | 30000 | ng/μL (DOTAP) |
| LNP with SM-102 i-lipid | 200 | ng/μL (RNA) |
| (Moderna-like) | | |
| 5moU-mRNA encoding SARS-CoV-2S | 1000 | ng/μL (RNA) |
| (E484K, N501Y) | | |
| unmod-mRNA encoding SARS-CoV-2S | 1000 | ng/μL (RNA) |
| (E484K, N501Y) | | |

Treatment groups are provided below in Table 11 and the schedule is provided in Table 12.

TABLE 11
Treatment Groups.

| | | | Dose | Volume | |
|---|---|---|---|---|---|
| Group | N | Treatment | [μg] | [μL] | Route |
| 1 | 5 | naive | 10 | 50 | IM |
| 2 | 5 | LNP-unmod-COV-mRNA | 10 | 50 | IM |
| 3 | 5 | NP-30/unmod-mRNA | 10 | 50 | IM |
| 4 | 5 | LNP-mod-COV-mRNA | 10 | 50 | IM |
| 5 | 5 | NP-30/mod-mRNA | 10 | 50 | IM |

TABLE 12
Vaccination Schedule.

| Group | Day | Week | Procedure |
|---|---|---|---|
| 1, 2, 3, 4 | −14 | −2 | Acclimation started |
| 1 | −7 | −1 | lower GI, rectal |
| 2 | −6 | −1 | weck-cel, BAL |
| 3 | −6 | −1 | |
| 4 | −5 | −1 | |
| 1, 2 | 0 | 0 | Immunization |
| 3, 4 | 0 | 0 | Immunization |
| 1, 2 | 1 (16 h) | 0 | bleed |
| 3, 4 | 1 (16 h) | 0 | bleed |
| 1, 2, 3, 4 | 14 | 2 | bleed |
| 1, 2 | 28 | 4 | bleed, BAL, |
| 3, 4 | 28 | 4 | Weck-Cels |
| 1, 2 | 56 | 8 | Immunization, bleed, |
| | | | Weck-cels |
| 3, 4 | 56 | 8 | Immunization, bleed, |
| | | | Weck-cels |
| 1, 2 | 70 | 10 | Blood, BAL, Weck- |
| 3, 4 | 70 | 10 | Cels |
| 1, 2 | 84 | 12 | Immunization, bleed, |
| 3, 4 | 84 | 12 | Weck-cels |
| 1 | 98 | 14 | Blood, Lower GI, |
| 2 | 98 | 14 | Weck-Cels, BAL |
| 3 | 98 | 14 | |
| 4 | 98 | 14 | |

Prime immunizations were prepared according to Table 13.

TABLE 13
Prime Immunizations.

| | | RNA | | | | |
|---|---|---|---|---|---|---|
| | | dose | | Inj vol | | |
| Group | N | [μg] | Route | [μL] | N:P | Description |
| 1 | 5 | 0 | IM | 50 | 0 | Naive |
| 2 | 5 | 10 | IM | 50 | 15 | LNP + unmodified |
| | | | | | | COV-mRNA |
| 3 | 5 | 10 | IM | 50 | 15 | NP-30 + unmodified |
| | | | | | | mRNA |
| 4 | 5 | 10 | IM | 50 | 15 | LNP + modified mRNA |
| 5 | 5 | 10 | IM | 50 | 15 | NP-30 + modified |
| | | | | | | mRNA |

Fifteen microliters of serum was diluted in 15 microliters of DMEM/10% FBS buffer (1:2 dilution) from each animal in Groups 1-5. Serum interferon-alpha 2 (IFN-α2) levels were measured and shown in (FIG. 13). Mice immunized with 10 μg unmodified mRNA formulated in LNPs had significantly higher levels of serum IFN-α2 than mice immunized with the same dose of unmodified mRNA formulated in NP-30. There were no significant differences in IFN-α2 levels between naive mice and NP-30/unmod-mRNA immunized mice.

Next, systemic cardiac troponin levels (cTNI) were measured to assess the systemic effects of NP-30 and LNP with unmodified or modified mRNA on the heart (FIG. 14). 10 μL of sera were diluted in 90 μL of Sample Dilution Buffer (1:10) for each condition. mRNA/LNP had a significant increase in serum cTNI as compared with naïve mRNA and modified mRNA delivered by LNP (FIG. 15). Interestingly, unmodified and modified RNA could be administered to animals with NP-30 and did not have significant effects on cTNI levels post prime or post boost.

Overall, LNP systemically induced biomarkers associated with higher vaccine reactogenicity and cardiac injury as compared to NP-30 formulated mRNA. LNP formulated unmod-mRNA also induced higher levels of adaptive immune responses including both humoral and T cell responses.

### Example 6: Comparing Formulation-Dependent Immunogenicity of Reprna-Encoding Hiv-1 Env in Balb/C Mice

Various HIV Env expressing repRNA constructs were compared for induction of binding and neutralizing antibodies. However, TZM-bl neutralization assay showed no measurable neutralizing antibody responses.

Using NP-1 and/or NP-30 to formulate repRNA, the effect of immunization route on immune responses was evaluated. Moreover, a recombinant BG505 SOSIP.664 protein vaccine can be adjuvanted with a NP-formulated repRNA expressing IL-12. The objectives of this study are summarized below.

Formulation-dependent-NP-30 vs. LNP immune responses by repRNA expressing full-length Env of BG505 isolate were measured. In addition, intramuscular (IM) and intradermal (ID) administration was compared for NP formulations. The repRNA-encoding mouse IL-12 to adjuvant a BG505 SOSIP.664 recombinant protein vaccine was also evaluated.

BABL/c mice (females; 6-8 weeks old; n=10/group) were immunized with repRNA-HIV-1 Env by route and formulation indicated in table labeled as randomized group order (Table 14). Immunization and bleed schedule is provided in Table 15.

TABLE 14
Immunization Conditions

| | | Dose | |
|---|---|---|---|
| Group # | Description | [μg] | Route |
| 1 | NP-30/repRNA-Env FL (IM) | 2.5 | IM |
| 2 | BG505 SOSIP.664 protein/AddaVax | 2.5 | IM |
| 3 | BG505 SOSIP.664 protein + | 2.5 protein/ | IM |
| | NP-30/repRNA-mIL12 | 1 repRNA | |
| 4 | LNP/repRNA-Env FL | 2.5 | IM |
| 5 | NP-30/repRNA-Env FL (ID) | 2.5 | ID |

TABLE 15
Immunization Schedule.

| Day | | Week | Procedure | Assays performed for: |
|---|---|---|---|---|
| 0 | | 0 | prime and | |
| | | | pre-bleeds | |
| 14 | h | | bleed | IFN-alpha2 |
| 28 | | 4 | boost | |
| 4 | h-postboost | | Bleed | IgG, IFN-alpha2, c-TNI |

| | | | |
|---|---|---|---|
| 29 | 4 | vaginal washes | IgG, IgA |
| 42 | 6 | bleed/vaginal | IgG, IgA, B/T cells |
| | | washes/spleen* | |
| 61 | 9 | bleed/boost | IgG |
| 75 | 11 | terminal | IgG, B/T cells |
| | | bleed/spleens | |

*5 mice in each group were sacrificed to measure T cell responses by ELISpot

Stock RNA and NP-30 were diluted to working concentration according to Table 16 below.

TABLE 16
Vaccine Conditions and Concentrations.

| | | Protein | RNA | | | | |
|---|---|---|---|---|---|---|---|
| | | dose | dose | | Inj vol | | |
| Group | N | [μg] | [μg] | Route | [μL] | N:P | Description |
| 1 | 10 | 0.0 | 2.5 | IM | 50 | 15 | NP-30/repRNA-Env FL |
| 2 | 10 | 2.5 | 0.0 | IM | 50 | n/a | BG505 SOSIP. 664 |
| | | | | | | | protein/AddaVax |
| 3 | 10 | 2.5 | 1.0 | IM | 50 | 15 | BG505 SOSIP. 664 |
| | | | | | | | protein + NP- |
| | | | | | | | 30/repRNA-mIL-12 |
| 4 | 10 | 0.0 | 2.5 | IM | 50 | 6 | LNP/repRNA-Env FL |
| 5 | 10 | 0.0 | 2.5 | ID | 50 | 15 | NP-30/repRNA-Env FL |

NP/repRNA complexes were prepared by pipetting 300 μL diluted repRNA solution to corresponding diluted NP solution. The mixture was pipetted up and down to ensure complete mixing. NP/repRNA complexes were incubated at room temperature for 30 minutes before injection. Mice were immunized and serum was collected according to the schedule in Table 15.

SOSIP protein coating antigen at 1 μg/mL in carbonate buffer was prepared. ELISA plates were coated, sealed and stored at 4 degrees C. overnight. the coating solution was decanted and the plates were tapped dry. Plates were washed, blocked, and serum was added to the ELISA assay buffer. Plates were washed and prepared for absorbance readings according to manufacturer's instructions. Plates were read at 450 nm on a SPECTRAMAX i3 plate reader.

Serum mouse IFN-α2 levels in BALB/c mice were measured 14 hours post IM immunization (FIG. 16A). LNP/repRNA induced significantly higher serum IFN-α2 compared to NP-30/repRNA. The NP-30/repRNA group was not significantly different compared to group immunized with AddaVax-adjuvanted protein. Mice immunized with LNPs had significantly higher levels of serum IFN-α2 than both NP-30/repRNA and AddaVax adjuvanted BG505 SOSIP.664 protein vaccinated mice. Antibody responses in NP-30/repRNA, LNP/repRNA and AddaVax/BG505 SOSIP.664 groups increased in magnitude after each booster immunization.

NP-30 and LNP induced similar levels of anti-gp140 IgG antibodies 11 weeks post-immunization (FIG. 16B), indicating that NP-30 has a reduced systemic IFN-α2 but can still produce an antibody response to HIV antigen challenge.

### Example 7: Formulation-Dependent Toleragenicity and Immunogenicity in Pigtailed Macaques Immunized with Rep RNA-BG505 SOSIP.664-FL

The safety and immunogenicity of NP-30 repRNA (SEQ ID NO: 181) vaccines expressing HIV-1 Envelope immunogen (SEQ ID NO: 182) was evaluated in pigtailed macaques.

First, repRNA and NP-30 were diluted to working concentration for each immunization according to Table 17 below.

TABLE 17
Vaccine Concentrations and Conditions.

| | | | | | | RNA | |
|---|---|---|---|---|---|---|---|
| | | | | | | concentration | |
| | | RNA | | | | BEFORE | |
| | | dose | | Inj vol | | complexing | |
| Group | N | [μg] | Route | [μL] | N:P | [ng/μL] | Description |
| 1 | 4 | 25 | IM | 500 | 15 | 112 | NP-30/RepRNA (SEQ ID NO: 181) |
| | | | | | | | Standard Size Emulsion |
| 2 | 4 | 25 | IM | 500 | 5.7 | N/A | LNP/RepRNA (SEQ ID NO: 181) |
| 3 | 4 | 25 | IM | 500 | 15 | 952.2 | Large complex NP-30/RepRNA |
| | | | | | | | (SEQ ID NO: 181) |
| 4 | 4 | 25 | IM | 500 | 15 | 117.0 | NP-30/RepRNA (SEQ ID NO: 181) |
| | | | | | | | Large Size Emulsion |

The NP-30/repRNA complexes for groups 1 and 4 were prepared by pipetting 1200 μL diluted repRNA solution to 1200 μL of corresponding diluted NP-30 solution. The mixture was pipetted up and down to ensure complete mixing. The NP-30/repRNA complex for group 3 was prepared by pipetting 135 μL diluted repRNA solution to 135 μL of corresponding diluted NP-30 solution. The NP-30/repRNA complexes were incubated at room temperature for 30 minutes before injection. Then, 240 μL of group 3 complex was diluted 10-fold in 11% sucrose/5 mM citrate buffer to bring the final volume up to 2400 μL.

The mean particle size and distribution (PDI) of the NP-30 formulation was determined before and after complexing with repRNA was quantified by dynamic light scattering. The Z average diameter and PDI increased post-complex for Groups 1, 3, and 4 (FIG. 17).

Pigtail macaques were administered repRNA vaccines in Table 17. Animals were bled and IFN-α2 levels were measured in the serum of each animal 16 hours after IM injection. LNP induced significantly higher levels of serum IFN-α2 as compared with NP-30 groups. All groups returned to baseline serum IFN-α2 levels 14 days post IM injection (FIG. 18). Increasing the NP-30/repRNA complex size, governed by mixing conditions, resulted in a noticeable decrease in serum IFN-α2 levels, indicating lower systemic reactogenicity of the vaccine in the pigtail macaques.

NP-30/repRNA (group 1) induced a robust antibody responses in 3 of 4 animals tested. NP-30/repRNA-large complex (group 3) induced robust antibody responses in 2 of 4 animals. LNP (group 2) and NP-30-large particle size (group 4) did not induce significant binding antibody responses above baseline.

T cell responses were examined in all treatment groups. LNP and large particle size NP-30 induced antigen specific CD4⁺ T cell responses in the lower gastrointestinal (GI) tract. All formulations induced CD8⁺ T cell responses in the lower gastrointestinal tract. Furthermore, NP-30 groups were characterized by a higher frequency of CD107a granzyme B (GzB) phenotype than LNP (FIG. 19A-19B).

Spearman's rank correlations between serum IFN-α2 levels 16 hours post immunization and adaptive immune responses were quantified for NP-30 (FIG. 20). There was a significant positive correlation between CD4/IL-2 positive T cells in the lower GI tract and serum IFN-α2 levels one day following immunization. There was also a positive correlation for CD4/CD107a GzB and CD4/IFN-gamma positive T cell response.

### Example 8: Np-1 Biodistribution

Eight week-old C57BL/6 mice were injected in the leg muscle with repRNA/NP-1 and assessed for biodistribution of the repRNA. At 7 days post-injection, the leg muscle (injection site), gut, liver, lung, brain, kidney, ovaries (fallopian tubes), heart and eye were collected and RNA was purified from organ homogenates. Replicon copy numbers per pg RNA in each organ were quantified by qRT-PCR using primers for the sub-genomic RNA (FIG. 21). Present to high levels of repRNA at the muscle injection site was observed. Surprisingly, repRNA was absent from all other organs, as RNA levels were below the lower limit of quantification. These data show the specific localization of repRNA to the injection site when delivered by NP-1.

### Example 9: Comparison of Np-30 and LNP Delivery of repRNA Encoding the Varicella-Zoster Virus Ge Protein (repRNA-VZV/gE)

The effect of NP-30 and LNP formulations repRNA formulations on early innate (serum IFN-alpha2) and adaptive immune responses were examined using a repRNA encoding the gE protein of VZV (repRNA-VZV/gE).

The repRNA-VZV/gE was complexed to NP-30 at different conditions as indicated in Table 18 below or encapsulated in lipid nanoparticles (LNPs).

TABLE 18
Nanoparticle Compositions Assayed.

| | | | | | | RNA | | |
|---|---|---|---|---|---|---|---|---|
| | | | | | | concentration | | |
| | | | | | | before | | |
| | | Dose | | Inj vol | | complexing | | test |
| # | N | [μg] | Route | [μL] | N:P | with NP-30 | Description | article |
| 1 | 5 | 1 | IM | 50 | 30 | 400 | NP-30 + RNA | repRNA- |
| | | | | | | | high conc | VZV/gE |
| | | | | | | | complex/hi | |
| | | | | | | | N:P | |
| 2 | 5 | 1 | IM | 50 | 15 | 400 | NP-30 + RNA | |
| | | | | | | | high conc | |
| | | | | | | | complex/mid | |
| | | | | | | | N:P | |
| 3 | 5 | 1 | IM | 50 | 30 | 40 | NP-30 + RNA | |
| | | | | | | | low conc | |
| | | | | | | | complex/hi | |
| | | | | | | | N:P | |
| 4 | 5 | 1 | IM | 50 | 5.5 | Not | HFS-230-714- | |
| | | | | | | applicable | LNP-031 | |
| 5 | 5 | 1 | IM | 50 | 7.5 | 40 | NP-30 + RNA | |
| | | | | | | | low conc | |
| | | | | | | | complex/low | |
| | | | | | | | N:P | |
| 6 | 5 | 1 | IM | 50 | 7.5 | 400 | NP-30 + RNA | |
| | | | | | | | high conc | |
| | | | | | | | complex/low | |
| | | | | | | | N:P | |
| 7 | 5 | 1 | IM | 50 | 15 | 40 | NP-30 + RNA | |
| | | | | | | | low conc | |
| | | | | | | | complex/mid | |
| | | | | | | | N:P | |

The effect of mixing conditions above on the physicochemical properties of NP-30/repRNA-VZV/gE complex were measured. In addition, the effect of NP-30/repRNA-VZV/gE complex structure or LNP/repRNA-VZV/gE on the following immune responses in female C571B1/6 mice (6-8 weeks old) were measured: (a) serum type I IFN (IFN-alpha2) 14 hours after IM injection; and (b) anti-VZV binding IgG responses.

Particle size characteristics of NP-30/repRNA-VZV/gE complexes and LNP/repRNA-VZV/gE were measured and shown in FIG. 22. LNP/repRNA-VZV/gE had a z-average particle diameter of 104.6 nm and PDI of 0.15 as measured by dynamic light scattering (DLS). All NP-30/repRNA-VZV/gE complexes were diluted to 7.5 ng DOTAP/μl before analysis by DLS. Complex size decreased as a function of N:P. Complexes formed at 400 ng/μl RNA resulted in larger particle size than 40 ng/μL. Size distribution (PDI) of complexes formed at 400 ng/μl was higher than 40 ng/μL. These results are summarized in Table 19.

TABLE 19
Z-average Before Complexing RNA with Nanoparticles.

RNA concentration before complexing

| | |
|---|---|
| 40 ng/μL | 400 ng/μL |

| | |
|---|---|
| N:P | Z-average [nm]/PDI |

| | | |
|---|---|---|
| 7.5 | 78.16/0.232 | 116.46/0.353 |
| 15 | 64.46/0.229 | 88.58/0.347 |
| 30 | 58.25/0.208 | 81.22/0.408 |

Next, formulation-dependent induction of serum IFN-alpha2 was determined. Approximately 14 hours after prime and booster IM injections, blood was collected to analyze IFN-alpha2 levels in serum. Serum was diluted 1:5, 1:3 or 1:2 fold and assayed using a mouse IFN-alpha2 ELISA kit (Invivogen). A standard curve was generated using recombinant IFN-alpha2 was used to interpolate concentration in samples. Statistical comparisons between groups performed by ordinary one-way ANOVA with Tukey's multiple comparisons test. Interestingly, only the LNP formulation induced elevated levels of serum IFN-alpha that were statistically significant compared to all other groups (FIG. 22), while NP-30 formulations exhibited much lower levels of IFN-α2. Although serum IFN-alpha2 levels between groups receiving different NP-30/repRNA-VZV/gE complexes were not significantly different, a significant inverse correlation between serum IFN-alpha2 and complex size was observed (FIG. 23).

Vaccine-antibody responses were measured using an ELISA. All groups mounted antigen (gE/gI)-specific antibody responses as determined by ELISA analyses of sera collected 14 days post-booster immunization (day 42 post-prime) (FIG. 24). Mice immunized with the NP-30/repRNA-VZV/gE vaccine complexed at N:P of 15 and RNA concentration of 400 ng/μL showed the highest mean endpoint titer.

Comparison of antibody responses between complexing concentration at each N:P showed that the mean titer was slightly higher in mice immunized with complexes formed at the higher 400 ng/μL concentration (FIG. 25). Complexes formed at 400 ng/μL, then diluted 10-fold before injection, were characterized by a larger overall complex size and PDI than complexes formed at 40 ng/μL. Mean antibody titer at N:P of 15 was significantly higher in group immunized with the higher concentration (400 ng/μL) complex than lower complex (40 ng/μL).

### Example 10: Binding IGG Responses Against BG505 SOSIP.664 Trimer in Non-human Primates Immunized With a REPRNA Vaccine Expressing Membrane-Bound and Disulfide Stabilized HIV-1 BG505 Trimer

Binding IgG responses against the HIV antigens were determined for various nanoparticle formulations.

FIG. 26 shows a graphical comparison of nanoparticle complex size and differences between NP-30 and LNP. When repRNA was encapsulated in LNP formulation, or complexed with NP-30 at an N:P ratio of 15 and 100 ng/μL concentration labeled above as “NP-30”, or 1000 ng/μL to produce a large complex vaccine labeled as “NP-30 (large complex)”, or with a large particle size NP-30 labeled as “NP-30 (large particle size)”. Particle size before and after complexing is plotted in FIG. 17. Immunization and blood collection schedules are shown in FIG. 26 (top). Non-human primates were immunized by IM injection on weeks 0 (prime), 8 (first boost) and 12 (second boost). NP-30 improved IgG binding to anti-gp140.

### Example 11: Immunogenicity of Monovalent and Trivalent EV-D68 Vaccines Formulated With NP-30 or Lipid Nanoparticles (LNPS)

A plasmid encoding a VEEV replicon mRNA backbone and single and combination viral antigens were prepared. Four formulations were prepared: (1) an NP-30-formulated monovalent EV-D68 vaccine; (2) a lipid nanoparticle (LNP) formulated monovalent EV-D68 vaccine; (3) an NP-30-formulated trivalent vaccine; and (4) an LNP-formulated trivalent vaccine. Briefly, the LNP was prepared using lipid components dissolved in ethanol at a ratio of 50:10:38:2 (Ionizable lipid (SM-102): Helper Lipid (DSPC): Cholesterol: DMG-PEG 2000) and mixed with RNA buffer at pH 4.5 at an N:P 5.5. The NP-30 and LNP trivalent vaccines were formulated using the antigen RNA sequence for EV-D68 virus-like particle (VLP) (SEQ ID NO: 183), RSV-F (SEQ ID NO: 22), and Influenza virus H3 antigen (SEQ ID NO: 11).

C57BL/6 mice were primed and boosted 28 days apart via intramuscular injection with the vaccine. The EV-D68 vaccine was administered at a dose of 3.3 μg. The trivalent vaccine was administered at a dose of 10 μg. On day 42, animals were bled and EV-D68 neutralizing antibody responses were assayed. The response was calculated as 50% reciprocal neutralization titer which refers to reciprocal dilution of serum required to inhibit viral infection by 50%. FIG. 27 shows that the NP-30-formulated trivalent vaccine produced superior response in neutralization titer relative to the LNP formulations. Furthermore, the NP-30-formulated trivalent vaccine produced a similar response in neutralization as NP-30-formulated EV-D68 vaccine.

### Example 12: Vaccination of Non-Human Primates with NP-30 and LNP

A plasmid encoding a VEEV replicon mRNA backbone and the EVD68 viral antigens and/or RSV antigens were prepared. The carrier formulations listed in Table 20 were prepared and complexed with the nucleic acids.

TABLE 20
Carrier-repRNA Complex Formulations.

| Formulation | Carrier | Antigens encoded | |
|---|---|---|---|
| # | Type | by repRNA | Dose |
| 1 | NP-30 | EV-D68/A1; EV-D68/B1; | 100 μg in total |
| | | EV-D68 C; RSV/F; | (20 μg for each |
| | | and RSV/G | antigen) |
| 2 | LNP | EV-D68/A1; EV-D68/B1; | 100 μg in total |
| | | EV-D68 C; RSV/F; | (20 μg for each |
| | | and RSV/G | antigen) |

Non-human primates (NHPs) were vaccinated by intramuscular injection on day 0 (FIG. 28A) with either the NP-30 or the LNP vaccine at a total dose of 100 μg multivalent repRNA vaccines. Sequences are provided in Table 23.

To determine the impact of each vaccine on the systemic innate immune response in NHPs, serum IFN-alpha 2 levels were measured from the immunized NHPs on days 0, 1, 3, and 6 (FIG. 28B). Serum IFN-alpha 2 levels were elevated in animals treated with the LNP vaccine. Serum IFN-alpha 2 levels remained stable for animals treated with the NP-30 vaccine.

Peripheral blood mononuclear cells (PBMCs) were isolated and the number of gene transcripts were measured by NanoString® (NanoString Technologies, Seattle, WA, USA) and the NHP Immunology panel (NanoString) in all animals. Gene transcripts were significantly changed in PBMCs isolated from the immunized NHPs on 1 day after the immunization (FIGS. 28C-28D). A list of the upregulated and downregulated genes in non-human primate PBMCs from animals treated with repRNA/NP-30 are provided in Table 21 below and shown in FIG. 28C.

TABLE 21
Gene expression changes in non-human primates
treated with repRNA/NP-30 multivalent vaccines.

| | | Upregulated 1 | Downregulated 1 |
|---|---|---|---|
| Gene | | day following | day following |
| Name | Protein Name | treatment | treatment |
| PLCB1 | phospholipase C beta 1 | − | + |
| FOXJ1 | forkhead box J1 | − | + |
| FCER1A | Fc epsilon receptor Ia | − | + |
| CD1B | Cluster of differentiation | − | + |
| | 1b molecule | | |
| MUC1 | Mucin 1 | − | + |
| CD7 | Cluster of differentiation | − | + |
| | 7 molecule | | |
| JUN | Jun proto-oncogene, | − | + |
| | AP-1 transcription | | |
| | factor subunit | | |
| CXCL11 | C-X-C motif chemokine | + | − |
| | ligand 11 | | |
| MMP3 | matrix | + | − |
| | metallopeptidase 3 | | |
| HAMP | hepcidin antimicrobial | + | − |
| | peptide | | |
| CCL3 | C-C motif chemokine | + | − |
| | ligand 3 | | |
| IFN1 | interferon, type 1, | + | − |
| | cluster | | |
| OASL | 2′-5′- | + | − |
| | oligoadenylate | | |
| | synthetase like | | |
| SIGLEC1 | sialic acid binding | + | − |
| | Ig like lectin 1 | | |
| IL1RN | Interleukin 1 receptor | + | − |
| | antagonist | | |
| IFIT1 | interferon induced | + | − |
| | protein with | | |
| | tetratricopeptide | | |
| | repeats 1 | | |
| C2 | Complement C2 | + | − |

A list of the upregulated an downregulated genes in non-human primate PBMCs from animals treated with repRNA/LNP are provided in Table 22 below.

TABLE 22
Gene expression changes in non-human primates
treated with repRNA/LNP multivalent vaccines.

| | | Upregulated 1 | Downregulated 1 |
|---|---|---|---|
| Gene | | day following | day following |
| Name | Protein Name | treatment | treatment |
| CLEC4E | C-type lectin domain | − | + |
| | family 4 member E | | |
| CXCL12 | C-X-C motif chemokine | + | − |
| | ligand 12 | | |
| CCL3 | C-C motif chemokine | + | − |
| | ligand 3 | | |
| MR1 | major histocompatibility | + | − |
| | complex, class I-related | | |
| RNASEL | ribonuclease L | + | − |
| FCGR2A | Fc gamma receptor IIa | + | − |
| CSF1 | colony stimulating | + | − |
| | factor 1 | | |
| CD80 | Cluster of differentiation | + | − |
| | 80 | | |
| CXCL11, | C-X-C motif chemokine | + | − |
| | ligand 11 | | |
| IL6 | Interleukin 6 | + | − |
| IFIT1 | interferon induced | + | − |
| | protein with | | |
| | tetratricopeptide | | |
| | repeats 1 | | |
| IFIH1 | interferon induced with | + | − |
| | helicase C domain 1 | | |
| CXCL10 | C-X-C motif chemokine | + | − |
| | ligand 10 | | |
| CCL8 | C-C motif chemokine | + | − |
| | ligand 8 | | |
| IRF7, | interferon regulatory | + | − |
| | factor 7 | | |
| OASL | 2′-5′- | + | − |
| | oligoadenylate | | |
| | synthetase like | | |
| IL1RN | Interleukin 1 receptor | + | − |
| | antagonist | | |
| CD163 | Cluster of | + | − |
| | differentiation 163 | | |

TABLE 23
DNA and RNA Sequences Encoding the
Antigens of the RepRNA vaccine

| | RNA encoding | DNA encoding |
|---|---|---|
| | antigen | antigen |
| Antigen | SEQ ID NO: | SEQ ID NO: |
| EV-D68/B1 | 187 | 188 |
| P1-IRES-Virus | | |
| Like Particle (VLP) | | |
| EV-D68 C | 189 | 190 |
| P1-IRES-3CD | | |
| EV-D68/A1 | 191 | 192 |
| P1-IRES-3CD | | |
| RSV-F | 22 | 186 |
| RSV- G | 20 | 185 |

As shown in the tables above and in FIG. 28D, total transcripts for many chemokines and IFN-stimulated genes were elevated for NHPs treated with repRNA-LNP as compared to repRNA-NP-30.

What is claimed is:

1. A method for generating a local immune response, the method comprising:
administering to a tissue in a subject a composition, wherein the composition comprises:
nanoparticles, wherein the nanoparticles comprise: a hydrophobic core comprising lipids in liquid phase at 25 degrees Celsius; and
nucleic acids, wherein the nucleic acids are complexed to the nanoparticles to form nucleic acid-nanoparticle complexes,
wherein the administering of the composition provides for a local immune response within a tissue,
wherein the local immune response is characterized by an increase in a level of at least one immunostimulatory marker in the tissue relative to a reference level,
wherein the reference level is the level of the immunostimulatory marker in a subject that has not been administered the composition or the level of the immunostimulatory marker in the subject prior to administration of the composition to the tissue,
and wherein the at least one immunostimulatory marker comprises: cluster of differentiation 86 (CD86), H-2 class II histocompatibility antigen, A beta chain (H2-ab1), H-2 class II histocompatibility antigen, I-E beta chain (H2-eb1), integrin alpha L chain (ITGAL or CD11a), Integrin alpha M (ITGAM, CR3A, or CD11b), Fcγ receptor (FcγR), cluster of differentiation 28 (CD28) or a combination thereof.

2. The method of claim 1, wherein the administering is intramuscular administration, intradermal administration, transdermal administration, sublingual administration, buccal administration, intranasal administration, inhalation administration, intrathecal administration, or intratumoral administration.

3. The method of claim 1, wherein the local immune response is characterized by recruitment of immune cells to the tissue.

4. The method of claim 3, wherein the immune cells comprise a population of MHC-II^{lo} macrophages, a population of plasmacytoid dendritic cells (pDCs), a population of monocyte dendritic cells (MoDCs), a population of neutrophils, a population of NK cells, or a combination thereof.

5. The method of claim 1, wherein the tissue is a muscle tissue, an epithelial tissue, an endothelial tissue, a mucosal tissue, a nasal cavity tissue, a thecal tissue, or a tumor.

6. The method of claim 1, wherein the local immune response is characterized by a lower level or activity of systemic interferons relative to a reference level, wherein the reference level comprises the level or activity of systemic interferons in a subject with systemic inflammation or the level or activity of systemic interferons in a subject that has been administered a reactogenic nanoparticle composition.

7. The method of claim 1, wherein the local immune response is characterized by a lower level of cardiac troponin in the blood of the subject relative to a reference level, wherein the reference level is the level of cardiac troponin in the blood of a subject that has systemic inflammation, myocarditis, or the level of cardiac troponin in the blood of a subject that has been administered a reactogenic nanoparticle composition.

8. The method of claim 7, wherein the cardiac troponin comprises troponin I (cTNI) or troponin T (cTNT).

9. The method of claim 1, wherein the local immune response is characterized by a higher level of interferon in the tissue administered the composition relative to the level of interferon in a heart, a spleen, or a liver of the subject.

10. The method of claim 1, wherein the local immune response is characterized by localization of the nucleic acids in the tissue, wherein the tissue is within or adjacent to a site of administration.

11. The method of claim 1, wherein prior to forming a complex with the nucleic acids, the nanoparticles are characterized as having a z-average diameter particle size measurement of about 40 nm up to 60 nm when measured by dynamic light scattering.

12. The method of claim 1, wherein the nucleic acids are complexed to a surface of the nanoparticles, wherein the surface comprises one or more cationic lipid selected from the group consisting of 1,2-dioleoyloxy-3 (trimethylammonium)propane, 30-[N-(N′,N′-dimethylaminoethane) carbamoyl]cholesterol, dimethyldioctadecylammonium, 1,2-dimyristoyl 3-trimethylammoniumpropane, dipalmitoyl(C16:0)trimethyl ammonium propane, distearoyltrimethylammonium propane, N-[1-(2,3-dioleyloxy)propyl]N,N,Ntrimethylammonium, chloride, N,N-dioleoyl-N,N-dimethylammonium chloride, 1,2-dioleoyl-sn-glycero-3-ethylphosphocholine, 1,2-dioleoyl-3-dimethylammonium-propane, and 1,2-dilinoleyloxy-3-dimethylaminopropane,1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol), tetrakis(8-methylnonyl) 3,3′,3″,3′″-(((methylazanediyl) bis(propane-3,1 diyl))bis (azanetriyl))tetrapropionate, decyl (2-(dioctylammonio)ethyl) phosphate, ethyl 5,5-di((Z)-heptadec-8-en-1-yl)-1-(3-(pyrrolidin-1-yl)propyl)-2,5-dihydro-1H-imidazole-2-carboxylate, ((4-hydroxybutyl)azanediyl)bis(hexane-6,1-diyl)bis(2-hexyldecanoate), 2-[(polyethylene glycol)-2000]-N,N-ditetradecylacetamide, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol, , bis(2-(dodecyldisulfanyl)ethyl) 3,3′-((3-methyl-9-oxo-10-oxa-13,14-dithia-3,6-diazahexacosyl)azanediyl)dipropionate, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide, 3,6-bis(4-(bis(2-hydroxydodecyl)amino)butyl)piperazine-2,5-dione, 30-[N-(N′,N′-dimethylaminoethane)-carbamoyl]cholesterol, (6Z,9Z,28Z,31Z)-heptatriaconta-6,9,28,31-tetraen-19-yl 4-(dimethylamino) butanoate, 1,2-dioleoyl-sn-glycero-3-phosphoethanolamine, 2,3-dioleyloxy-N-[2-(sperminecarboxamido)ethyl]-N,N-dimethyl-1-propanaminium trifluoroacetate, 1,2-distearoyl-sn-glycero-3-phosphocholine, ethylphosphatidylcholine, hexa(octan-3-yl) 9,9′,9″,9′41 ,9″″,9′″″-((((benzene-1,3,5-tricarbonyl)yris(azanediyl)) tris (propane-3,1-diyl)) tris(azanetriyl))hexanonanoate, heptadecan-9-yl 8-((2-hydroxyethyl)(6-oxo-6-(undecyloxy)hexyl)amino) octanoate, (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″″Z)-tetrakis (octadeca-9,12-dienoate), and N1,N3,N5-tris(3-(didodecylamino)propyl)benzene-1,3,5-tricarboxamide.

13. The method of claim 1, wherein the hydrophobic core comprises a liquid oil, wherein the liquid oil comprises α-tocopherol, coconut oil, grapeseed oil, lauroyl polyoxylglyceride, mineral oil, monoacylglycerol, palm kernel oil, olive oil, paraffin oil, peanut oil, propolis, squalene, solanesol, soy lecithin, soybean oil, sunflower oil, a triglyceride, or vitamin E.

14. The method of claim 1, wherein the nanoparticles further comprise a hydrophobic surfactant and a hydrophilic surfactant.

15. The method of claim 1, wherein the hydrophobic core further comprises an inorganic particle, wherein the inorganic particle comprises a metal, optionally wherein the metal comprises iron oxide, magnetite (Fe₃O₄), maghemite (y-Fe₂O₃), wüstite (FeO), hematite (alpha (α)-Fe₂O₃), aluminum hydroxide, or aluminum oxyhydroxide.

16. The method of claim 1, wherein the nucleic acids are in complex with the nanoparticles.

17. The method of claim 1, wherein the nucleic acids each encode for one or more bioactive agent, optionally wherein the one or more bioactive agent comprises a protein, an antibody, an antibody fragment, a cytokine, or an immune system modulator.

18. The method of claim 1, wherein the composition comprises at least one nucleic acid encoding for an RNA polymerase, optionally wherein the RNA polymerase is a Venezuelan equine encephalitis virus (VEEV) RNA polymerase.

19. The method of claim 1, wherein the composition is lyophilized prior to administration to the subject.

20. The method of claim 1, wherein the composition is formulated as a suspension, a liquid, a semi-liquid, a solution, a propellant, or a powder dosage form.
